SEQUENCE LISTING (293 sequences)

SEQ ID NO: 1 - protein, 25 aa, Artificial Sequence
  Synthetic Construct
  VARIANT 1, 6: Xaa = Ala, Asp, Phe, His, Ile, Leu, Asn, Pro, Ser, Thr, Val, or Tyr
  VARIANT 2, 3, 5, 7, 9, 10, 11, 12, 13, 15, 16, 17, 18, 19,20, 21, 23, 24: Xaa = Ala, Asp, Gly, His, Ile, Leu, Asn, Pro, Arg, Ser, Thr, or Val
XXXLXXXGXXXXXPXXXXXXXCXXS

SEQ ID NO: 2 - protein, 25 aa, Artificial Sequence [sequence omitted]

SEQ ID NO: 3 - protein, 25 aa, Artificial Sequence
  Synthetic Construct
QVQLVQSGAEVKKLGASVKVSCKAS

SEQ ID NO: 4 - protein, 25 aa, Artificial Sequence
  Synthetic Construct
QVQLVQSGAEVKKPGASVKVSCKVS

SEQ ID NO: 5 - protein, 25 aa, Artificial Sequence
  Synthetic Construct
QMQLVQSGAEVKKTGSSVKVSCKAS

SEQ ID NO: 6 - protein, 25 aa, Artificial Sequence [sequence omitted]

SEQ ID NO: 7 - protein, 25 aa, Artificial Sequence
  Synthetic Construct
QMQLVQSGPEVKKPGTSVKVSCKAS

SEQ ID NO: 8 - protein, 25 aa, Artificial Sequence
  Synthetic Construct
QVQLVQSGSELKKPGASVKVSCKAS

SEQ ID NO: 9 - protein, 25 aa, Artificial Sequence
  Synthetic Construct
EVQLVQPGAELRKPGASVKVSCKAS

SEQ ID NO: 10 - protein, 25 aa, Artificial Sequence
  Synthetic Construct
EVQLVQPGAELRNPGASVKVSCKAS

SEQ ID NO: 11 - protein, 25 aa, Artificial Sequence
  Synthetic Construct
EVQLVQPGAELRKPGASLKVSCKAS

SEQ ID NO: 12 - protein, 25 aa, Artificial Sequence
  Synthetic Construct
QVQLVQPGAELRKPGALLKVSCKAS

SEQ ID NO: 13 - protein, 25 aa, Artificial Sequence
  Synthetic Construct
EVQLVQSGAEVKKPGESLRISCKGS

SEQ ID NO: 14 - protein, 25 aa, Artificial Sequence
  Synthetic Construct
EVQLVQSGAEVKKPGESLKISCKGS

SEQ ID NO: 15 - protein, 25 aa, Artificial Sequence
  Synthetic Construct
EVQLVQSGAEVKKPGATVKISCKVS

SEQ ID NO: 16 - protein, 25 aa, Artificial Sequence [sequence omitted]

SEQ ID NO: 17 - protein, 25 aa, Artificial Sequence
  Synthetic Construct
EVQLVESGGGLVQPGGSLRLSCAAC

SEQ ID NO: 18 - protein, 25 aa, Artificial Sequence
  Synthetic Construct
EVQVVESGGGLVQPGGSLRLSCAAS

SEQ ID NO: 19 - protein, 25 aa, Artificial Sequence
  Synthetic Construct
EVQVVESGGGLVQAGGSLRLSCAAS

SEQ ID NO: 20 - protein, 25 aa, Artificial Sequence
  Synthetic Construct
EVQLVESGGGLVQAGGSLRLSCAAS

SEQ ID NO: 21 - protein, 25 aa, Artificial Sequence
  Synthetic Construct
EVQLVKSGGGLVQPGGSLRLSCAAS

SEQ ID NO: 22 - protein, 25 aa, Artificial Sequence
  Synthetic Construct
EVHLVESGGGLVQPGGALRLSCAAS

SEQ ID NO: 23 - protein, 25 aa, Artificial Sequence
  Synthetic Construct
EVQLVESGEGLVQPGGSLRLSCAAS

SEQ ID NO: 24 - protein, 25 aa, Artificial Sequence
  Synthetic Construct
EVQLVESGGGLIQPGGSLRLSCAAS

SEQ ID NO: 25 - protein, 25 aa, Artificial Sequence
  Synthetic Construct
EVQLVETGGGLIQPGGSLRLSCAAS

SEQ ID NO: 26 - protein, 25 aa, Artificial Sequence
  Synthetic Construct
EVQLVESGGGLVQPGGSLKLSCAAS

SEQ ID NO: 27 - protein, 25 aa, Artificial Sequence
  Synthetic Construct
EVQLLESGGGLVQPGGSLRLSCAAS

SEQ ID NO: 28 - protein, 25 aa, Artificial Sequence
  Synthetic Construct
EVQLVESAGALVQPGGSLRLSCAAS

SEQ ID NO: 29 - protein, 25 aa, Artificial Sequence
  Synthetic Construct
EVQLVESAGGLVQPGGSLRLSCAAS

SEQ ID NO: 30 - protein, 25 aa, Artificial Sequence
  Synthetic Construct
EVQLVESGGGLVQPGRSLRLSCTAS

SEQ ID NO: 31 - protein, 25 aa, Artificial Sequence
  Synthetic Construct
EVQLVESGGGLVKPGRSLRLSCTAS

SEQ ID NO: 32 - protein, 25 aa, Artificial Sequence
  Synthetic Construct
EVQLVESGGGLVQPGPSLRLSCTAS

SEQ ID NO: 33 - protein, 25 aa, Artificial Sequence [sequence omitted]

SEQ ID NO: 34 - protein, 25 aa, Artificial Sequence
  Synthetic Construct
EVQLVESGGGLVQPGGSLRLSCSAS

SEQ ID NO: 35 - protein, 25 aa, Artificial Sequence
  Synthetic Construct
QVQLVESGGGLVQPGGSLRLSCSAS

SEQ ID NO: 36 - protein, 25 aa, Artificial Sequence
  Synthetic Construct
EVQLVESGGGVVRPGGSLRLSCAAS

SEQ ID NO: 37 - protein, 25 aa, Artificial Sequence
  Synthetic Construct
EVQLVESGGGVVRPGGSLRLSFAAS

SEQ ID NO: 38 - protein, 25 aa, Artificial Sequence
  Synthetic Construct
EVQLVESGGVVVQPGGSLRLSCAAS

SEQ ID NO: 39 - protein, 25 aa, Artificial Sequence
  Synthetic Construct
EVQLVESGGGVVQPGGSLRLSCAAS

SEQ ID NO: 40 - protein, 25 aa, Artificial Sequence
  Synthetic Construct
QVQLVESGGGVVQPGRSLRLSCAAS

SEQ ID NO: 41 - protein, 25 aa, Artificial Sequence
  Synthetic Construct
QVQLVDSGGGVVQPGRSLRLSCAAS

SEQ ID NO: 42 - protein, 25 aa, Artificial Sequence
  Synthetic Construct
QVQLVESGGGVVQPGGSLRLSCAAS

SEQ ID NO: 43 - protein, 25 aa, Artificial Sequence
  Synthetic Construct
QVQLVESGGGLVQPGGSLRLSCAAS

SEQ ID NO: 44 - protein, 25 aa, Artificial Sequence
  Synthetic Construct
QVQLVESGGGLVQAGGSLRLSCAAS

SEQ ID NO: 45 - protein, 25 aa, Artificial Sequence
  Synthetic Construct
QVQLVESGGGLVQAGGSLRHSCAAS

SEQ ID NO: 46 - protein, 25 aa, Artificial Sequence
  Synthetic Construct
QVQLVESVGGLVQDGGSLRLSCAAS

SEQ ID NO: 47 - protein, 25 aa, Artificial Sequence
  Synthetic Construct
QLQLVESGGGLVQPGGSLRLSCAAS

SEQ ID NO: 48 - protein, 25 aa, Artificial Sequence
  Synthetic Construct
QLQLVESGGGLVQPGGSLRVSCAAS

SEQ ID NO: 49 - protein, 25 aa, Artificial Sequence
  Synthetic Construct
QVQLVETGGGLVQPGGSLRLSCAAS

SEQ ID NO: 50 - protein, 25 aa, Artificial Sequence
  Synthetic Construct
QVQLVESGGGLVQPGVSLRLSCAAS

SEQ ID NO: 51 - protein, 25 aa, Artificial Sequence
  Synthetic Construct
EVQLVESGGGLVKPGGSLRLSCAAS

SEQ ID NO: 52 - protein, 25 aa, Artificial Sequence
  Synthetic Construct
EVQLVESGGALVKPGGSLRLSCAAS

SEQ ID NO: 53 - protein, 25 aa, Artificial Sequence
  Synthetic Construct
QVQLVESGGGLVKPGGSLRLSCAAS

SEQ ID NO: 54 - protein, 25 aa, Artificial Sequence
  Synthetic Construct
QVQLLESGGGLVKPGGSLRLSCAAS

SEQ ID NO: 55 - protein, 25 aa, Artificial Sequence
  Synthetic Construct
QVTLKESGPVLVKPTETLTLTCTVS

SEQ ID NO: 56 - protein, 25 aa, Artificial Sequence
  Synthetic Construct
QVTLKESGPVLVKPTETLTLTCTIS

SEQ ID NO: 57 - protein, 25 aa, Artificial Sequence
  Synthetic Construct
QITLKESGPTLVKPTQTLTLTCTFS

SEQ ID NO: 58 - protein, 25 aa, Artificial Sequence
  Synthetic Construct
QITLKESGPTLVKPTQTLTLTRTFS

SEQ ID NO: 59 - protein, 25 aa, Artificial Sequence
  Synthetic Construct
QVTLKESGPTLVKPTQTLTLTCTFS

SEQ ID NO: 60 - protein, 25 aa, Artificial Sequence
  Synthetic Construct
QVTLKESGPALVKPTQTLTLTCTFS

SEQ ID NO: 61 - protein, 25 aa, Artificial Sequence
  Synthetic Construct
QVTLKESGPVLVKPTQTLTLTCTFS

SEQ ID NO: 62 - protein, 25 aa, Artificial Sequence
  Synthetic Construct
QVTLRESGPALVKPTQTLTLTCTFS

SEQ ID NO: 63 - protein, 25 aa, Artificial Sequence
  Synthetic Construct
RVTLRESGPALVKPTQTLTLTCTFS

SEQ ID NO: 64 - protein, 25 aa, Artificial Sequence
  Synthetic Construct
QVTLRESGPALVKPTQTLTLTCAFS

SEQ ID NO: 65 - protein, 25 aa, Artificial Sequence
  Synthetic Construct
EVQVQESGPGLVKPSQALSLTCTAS

SEQ ID NO: 66 - protein, 25 aa, Artificial Sequence
  Synthetic Construct
QVQRQESGPGLVKPSQMLSLTCTAS

SEQ ID NO: 67 - protein, 25 aa, Artificial Sequence
  Synthetic Construct
EVQLQESGPGLVKPSQMLSLTCTLS

SEQ ID NO: 68 - protein, 25 aa, Artificial Sequence
  Synthetic Construct
QVQLQESGPGLVKPSQTLSLTCTAS

SEQ ID NO: 69 - protein, 25 aa, Artificial Sequence
  Synthetic Construct
QVQLQESGPGLVKPSDTLSLTCAVS

SEQ ID NO: 70 - protein, 25 aa, Artificial Sequence
  Synthetic Construct
QVQLQESGPGLVKPSETLSLTCAVS

SEQ ID NO: 71 - protein, 25 aa, Artificial Sequence
  Synthetic Construct
QVQLQESGPGLVKPSQTLSLTCAVS

SEQ ID NO: 72 - protein, 25 aa, Artificial Sequence
  Synthetic Construct
QVQLQESGPGLVKPSQTLSLTCTVS

SEQ ID NO: 73 - protein, 25 aa, Artificial Sequence
  Synthetic Construct
QVRLQESGPGLVKPSQTLSLTCTVS

SEQ ID NO: 74 - protein, 25 aa, Artificial Sequence
  Synthetic Construct
QVQLQDSGPGLVKPSQTLSLTCTVS

SEQ ID NO: 75 - protein, 25 aa, Artificial Sequence
  Synthetic Construct
QVQLQESGPGLLKPSQTLSLTCTVS

SEQ ID NO: 76 - protein, 25 aa, Artificial Sequence
  Synthetic Construct
EVQLQESGPGLVKPSQTLSLTCTVS

SEQ ID NO: 77 - protein, 25 aa, Artificial Sequence
  Synthetic Construct
QVQLQESGPGLVKPSDTLSLTCTVS

SEQ ID NO: 78 - protein, 25 aa, Artificial Sequence
  Synthetic Construct
QVQLQESGPGLVKPSETLSLTCTVS

SEQ ID NO: 79 - protein, 25 aa, Artificial Sequence
  Synthetic Construct
QVQLQESGPGLVKPSETLSLTCTVT

SEQ ID NO: 80 - protein, 25 aa, Artificial Sequence
  Synthetic Construct
QLQLQESGPGLVKPSETLSLTCTVS

SEQ ID NO: 81 - protein, 25 aa, Artificial Sequence
  Synthetic Construct
QLQLQESGPGLVKPSETPSLTCTVS

SEQ ID NO: 82 - protein, 25 aa, Artificial Sequence
  Synthetic Construct
RLQLQESGPGLVKPSETLSLTCTVS

SEQ ID NO: 83 - protein, 25 aa, Artificial Sequence [sequence omitted]

SEQ ID NO: 84 - protein, 25 aa, Artificial Sequence
  Synthetic Construct
EVQLQESGPGLLKPSQTLSLTCAVY

SEQ ID NO: 85 - protein, 25 aa, Artificial Sequence
  Synthetic Construct
QVQLQESGPGLVKPSQTLSLTCAVY

SEQ ID NO: 86 - protein, 25 aa, Artificial Sequence
  Synthetic Construct
QVQLQESGPGLVKPSETLSLTCAVY

SEQ ID NO: 87 - protein, 25 aa, Artificial Sequence
  Synthetic Construct
QVQLQQSGPGLVKPSQTLSLTCAIS

SEQ ID NO: 88 - protein, 25 aa, Artificial Sequence
  Synthetic Construct
QLQLQESGSGLVKPSQTLSLTCAVS

SEQ ID NO: 89 - protein, 25 aa, Artificial Sequence
  Synthetic Construct
QVQLQESGPGLVKPSGTLSLTCAVS

SEQ ID NO: 90 - protein, 25 aa, Artificial Sequence
  Synthetic Construct
QVQLQESGPGLVKPPGTLSLTCAVS

SEQ ID NO: 91 - protein, 25 aa, Artificial Sequence
  Synthetic Construct
QVQLQELGPGLVKPPGTLSLTCAVS

SEQ ID NO: 92 - protein, 25 aa, Artificial Sequence
  Synthetic Construct
QVQLQESGPGLVKPPGTLSLTCAIS

SEQ ID NO: 93 - protein, 25 aa, Artificial Sequence
  Synthetic Construct
DIQMTQSPSSLSASVGDRVTITCQA

SEQ ID NO: 94 - protein, 25 aa, Artificial Sequence
  Synthetic Construct
DIQMTQSPSSVSASVGDRVTITCRA

SEQ ID NO: 95 - protein, 25 aa, Artificial Sequence
  Synthetic Construct
DIQMTQSPSSLSASVGDRVTITCRA

SEQ ID NO: 96 - protein, 25 aa, Artificial Sequence
  Synthetic Construct
AIQMTQSPSSLSASVGDRVTITCRA

SEQ ID NO: 97 - protein, 25 aa, Artificial Sequence
  Synthetic Construct
DIQMTQSPSAMSASVGDRVTITCRA

SEQ ID NO: 98 - protein, 25 aa, Artificial Sequence
  Synthetic Construct
NIQMTQSPSAMSASVGDRVTITCRA

SEQ ID NO: 99 - protein, 25 aa, Artificial Sequence
  Synthetic Construct
AIQLTQSPSSLSASVGDRVTITCRA

SEQ ID NO: 100 - protein, 25 aa, Artificial Sequence
  Synthetic Construct
DIQMTQSPSTLSASVGDRVTITCRA

SEQ ID NO: 101 - protein, 25 aa, Artificial Sequence
  Synthetic Construct
DIQMTQSPSTLSASVGDRVTIICRA

SEQ ID NO: 102 - protein, 25 aa, Artificial Sequence
  Synthetic Construct
DIQLTQSPSFLSASVGDRVTITCRA

SEQ ID NO: 103 - protein, 25 aa, Artificial Sequence
  Synthetic Construct
VIWMTQSPSLLSASTGDRVTISCRM

SEQ ID NO: 104 - protein, 25 aa, Artificial Sequence
  Synthetic Construct
AIWMTQSPSLLSASTGDRVTISCRM

SEQ ID NO: 105 - protein, 25 aa, Artificial Sequence
  Synthetic Construct
AIRMTQSPSSFSASTGDRVTITCRA

SEQ ID NO: 106 - protein, 25 aa, Artificial Sequence
  Synthetic Construct
AIRMTQSPFSLSASVGDRVTITCWA

SEQ ID NO: 107 - protein, 25 aa, Artificial Sequence
  Synthetic Construct
EIVLTQSPDFQSVTPKEKVTITCRA

SEQ ID NO: 108 - protein, 25 aa, Artificial Sequence
  Synthetic Construct
ETTLTQSPAFMSATPGDKVNISCKA

SEQ ID NO: 109 - protein, 25 aa, Artificial Sequence
  Synthetic Construct
EIVLTQSPATLSLSPGERATLSCRA

SEQ ID NO: 110 - protein, 25 aa, Artificial Sequence
  Synthetic Construct
EIVLTQSPGTLSLSPGERATLSCRA

SEQ ID NO: 111 - protein, 25 aa, Artificial Sequence
  Synthetic Construct
EIVLTQSPATLSLSPGERATLSCGA

SEQ ID NO: 112 - protein, 25 aa, Artificial Sequence
  Synthetic Construct
EIVMTQSPATLSVSPGERATLSCRA

SEQ ID NO: 113 - protein, 25 aa, Artificial Sequence
  Synthetic Construct
EIVMTQSPATLSLSPGERATLSCRA

SEQ ID NO: 114 - protein, 25 aa, Artificial Sequence
  Synthetic Construct
DIVMTQSPDSLAVSLGERATINCKS

SEQ ID NO: 115 - protein, 25 aa, Artificial Sequence
  Synthetic Construct
DVVMTQSPLSLPVTLGQPASISCRS

SEQ ID NO: 116 - protein, 25 aa, Artificial Sequence
  Synthetic Construct
DIVMTQTPLSSPVTLGQPASISCRS

SEQ ID NO: 117 - protein, 25 aa, Artificial Sequence
  Synthetic Construct
DIVMTQSPLSLPVTPGEPASISCRS

SEQ ID NO: 118 - protein, 25 aa, Artificial Sequence
  Synthetic Construct
DIVMTQTPLSLSVTPGQPASISCKS

SEQ ID NO: 119 - protein, 25 aa, Artificial Sequence
  Synthetic Construct
DIVMTQTPLSLPVTPGEPASISCRS

SEQ ID NO: 120 - protein, 25 aa, Artificial Sequence
  Synthetic Construct
EIVMTQTPLSLSITPGEQASMSCRS

SEQ ID NO: 121 - protein, 25 aa, Artificial Sequence
  Synthetic Construct
EIVMTQTPLSLSITPGEQASISCRS

SEQ ID NO: 122 - protein, 24 aa, Artificial Sequence
  Synthetic Construct
RPVLTQPPSLSASPGATARLPCTL

SEQ ID NO: 123 - protein, 24 aa, Artificial Sequence
  Synthetic Construct
QPVLTQPTSLSASPGASARFTCTL

SEQ ID NO: 124 - protein, 24 aa, Artificial Sequence
  Synthetic Construct
QAVLTQPSSLSASPGASASLTCTL

SEQ ID NO: 125 - protein, 24 aa, Artificial Sequence
  Synthetic Construct
QAVLTQPASLSASPGASASLTCTL

SEQ ID NO: 126 - protein, 24 aa, Artificial Sequence
  Synthetic Construct
QPVLTQPTSLSASPGASARLTCTL

SEQ ID NO: 127 - protein, 24 aa, Artificial Sequence
  Synthetic Construct
QPVLTQPPSSSASPGESARLTCTL

SEQ ID NO: 128 - protein, 24 aa, Artificial Sequence
  Synthetic Construct
QPVLTQPSSHSASSGASVRLTCML

SEQ ID NO: 129 - protein, 24 aa, Artificial Sequence
  Synthetic Construct
QAGLTQPPSVSKGLRQTATLTCTG

SEQ ID NO: 130 - protein, 24 aa, Artificial Sequence
  Synthetic Construct
QSVLTQPPSASGTPGQRVTISCSG

SEQ ID NO: 131 - protein, 24 aa, Artificial Sequence
  Synthetic Construct
QSVLTQPPSVSEAPRQRVTISCSG

SEQ ID NO: 132 - protein, 24 aa, Artificial Sequence
  Synthetic Construct
QSVVTQPPSVSGAPGQRVTISCTG

SEQ ID NO: 133 - protein, 24 aa, Artificial Sequence
  Synthetic Construct
QSVLTQPPSVSGAPGQRVTISCTG

SEQ ID NO: 134 - protein, 24 aa, Artificial Sequence
  Synthetic Construct
QSVLTQPPSVSAAPGQKVTISCSG

SEQ ID NO: 135 - protein, 24 aa, Artificial Sequence
  Synthetic Construct
QSALTQPPSVSGSPGQSVTISCTG

SEQ ID NO: 136 - protein, 24 aa, Artificial Sequence
  Synthetic Construct
QSALTQPRSVSGSPGQSVTISCTG

SEQ ID NO: 137 - protein, 24 aa, Artificial Sequence
  Synthetic Construct
QSALTQPPSASGSPGQSVTISCTG

SEQ ID NO: 138 - protein, 24 aa, Artificial Sequence
  Synthetic Construct
QSALTQPPSASRSPGQSVTISCTG

SEQ ID NO: 139 - protein, 24 aa, Artificial Sequence
  Synthetic Construct
QSALTQPASVSGSPGQSITISCTG

SEQ ID NO: 140 - protein, 24 aa, Artificial Sequence
  Synthetic Construct
QSALTQPPFVSGAPGQSVTISCTG

SEQ ID NO: 141 - protein, 24 aa, Artificial Sequence
  Synthetic Construct
NFMLTQPHSVSESPGKTVTISCTR

SEQ ID NO: 142 - protein, 24 aa, Artificial Sequence
  Synthetic Construct
NFMLTQPHSVSESPGKTVTISCTG

SEQ ID NO: 143 - protein, 24 aa, Artificial Sequence
  Synthetic Construct
SYELTQPHSVSVATAQMARITCGG

SEQ ID NO: 144 - protein, 24 aa, Artificial Sequence
  Synthetic Construct
SYVLTQPPSVSVAPGQTARITCGG

SEQ ID NO: 145 - protein, 24 aa, Artificial Sequence
  Synthetic Construct
SYVLTQPPSVSVAPGKTARITCGG

SEQ ID NO: 146 - protein, 24 aa, Artificial Sequence
  Synthetic Construct
SYELTQPLSVSVALGQTARITCGG

SEQ ID NO: 147 - protein, 24 aa, Artificial Sequence
  Synthetic Construct
SYELTQPLSVSVALGQAARITCGG

SEQ ID NO: 148 - protein, 24 aa, Artificial Sequence
  Synthetic Construct
SYELTQPPSVSVSPGQTASITCSG

SEQ ID NO: 149 - protein, 24 aa, Artificial Sequence
  Synthetic Construct
SYELTQPPSVSVSPGQTARITCSG

SEQ ID NO: 150 - protein, 24 aa, Artificial Sequence
  Synthetic Construct
SYELMQPPSVSVSPGQTARITCSG

SEQ ID NO: 151 - protein, 24 aa, Artificial Sequence
  Synthetic Construct
SYELTQPPSVSVSLGQMARITCSG

SEQ ID NO: 152 - protein, 24 aa, Artificial Sequence
  Synthetic Construct
SYELTQPSSVSVSPGQTARITCSG

SEQ ID NO: 153 - protein, 24 aa, Artificial Sequence
  Synthetic Construct
SYELTQLPSVSVSPGQTARITCSG

SEQ ID NO: 154 - protein, 24 aa, Artificial Sequence
  Synthetic Construct
SSELTQDPAVSVALGQTVRITCQG

SEQ ID NO: 155 - protein, 24 aa, Artificial Sequence
  Synthetic Construct
SSGPTQVPAVSVALGQMARITCQG

SEQ ID NO: 156 - protein, 24 aa, Artificial Sequence
  Synthetic Construct
QAVVTQEPSLTVSPGGTVTLTCGS

SEQ ID NO: 157 - protein, 24 aa, Artificial Sequence
  Synthetic Construct
QTVVTQEPSLTVSPGGTVTLTCAS

SEQ ID NO: 158 - protein, 24 aa, Artificial Sequence
  Synthetic Construct
QTVVTQEPSFSVSPGGTVTLTCGL

SEQ ID NO: 159 - protein, 24 aa, Artificial Sequence
  Synthetic Construct
QSVVTQEPSLSGSPGGTVTLTCAL

SEQ ID NO: 160 - protein, 24 aa, Artificial Sequence
  Synthetic Construct
QPVLTQSSSASASLGSSVKLTCTL

SEQ ID NO: 161 - protein, 24 aa, Artificial Sequence
  Synthetic Construct
QLVLTQSPSASASLGASVKLTCTL

SEQ ID NO: 162 - protein, 24 aa, Artificial Sequence
  Synthetic Construct
LPVLTQPPSASALLGASIKLTCTL

SEQ ID NO: 163 - protein, 24 aa, Artificial Sequence
  Synthetic Construct
QPVLTQPPSASASLGASVTLTCTL

SEQ ID NO: 164 - protein, 11 aa, Artificial Sequence
  Synthetic Construct
RNGGGGLFLDY

SEQ ID NO: 165 - protein, 11 aa, Artificial Sequence
  Synthetic Construct
VPPLYKSDGVY

SEQ ID NO: 166 - protein, 16 aa, Artificial Sequence
  Synthetic Construct
DLFGVPTEYPGQEFHP

SEQ ID NO: 167 - protein, 13 aa, Artificial Sequence
  Synthetic Construct
QGVIATSLKYLEV

SEQ ID NO: 168 - protein, 16 aa, Artificial Sequence
  Synthetic Construct
IFVRGGSFYPADEYDY

SEQ ID NO: 169 - protein, 14 aa, Artificial Sequence
  Synthetic Construct
TGVRLNYGSRAEDY

SEQ ID NO: 170 - protein, 12 aa, Artificial Sequence
  Synthetic Construct
GYPWDESCDYDN

SEQ ID NO: 171 - protein, 11 aa, Artificial Sequence
  Synthetic Construct
RNGGGGLLLDY

SEQ ID NO: 172 - protein, 8 aa, Artificial Sequence
  Synthetic Construct
HHPYETPL

SEQ ID NO: 173 - protein, 18 aa, Artificial Sequence
  Synthetic Construct
DSPVRGYVCSDSLSTMDY

SEQ ID NO: 174 - protein, 9 aa, Artificial Sequence
  Synthetic Construct
PLSRGSMDY

SEQ ID NO: 175 - protein, 7 aa, Artificial Sequence
  Synthetic Construct
RRDGTDY

SEQ ID NO: 176 - protein, 6 aa, Artificial Sequence
  Synthetic Construct
VPPLPR

SEQ ID NO: 177 - protein, 7 aa, Artificial Sequence
  Synthetic Construct
LIAGVRY

SEQ ID NO: 178 - protein, 7 aa, Artificial Sequence
  Synthetic Construct
VMRGVNY

SEQ ID NO: 179 - protein, 7 aa, Artificial Sequence
  Synthetic Construct
RWGGRLY

SEQ ID NO: 180 - protein, 16 aa, Artificial Sequence
  Synthetic Construct
IFQRGTSYYPADEFDY

SEQ ID NO: 181 - protein, 5 aa, Artificial Sequence
  Synthetic Construct
SRGPS

SEQ ID NO: 182 - protein, 11 aa, Artificial Sequence
  Synthetic Construct
MVITTTPEYDY

SEQ ID NO: 183 - skipped

SEQ ID NO: 184 - protein, 16 aa, Artificial Sequence
  Synthetic Construct
SYSGGYYNFRESQYYS

SEQ ID NO: 185 - protein, 16 aa, Artificial Sequence
  Synthetic Construct
NLFGVPTEYPGQEFHP

SEQ ID NO: 186 - protein, 9 aa, Artificial Sequence
  Synthetic Construct
KHFSYVEDF

SEQ ID NO: 187 - protein, 6 aa, Artificial Sequence
  Synthetic Construct
VPPFPS

SEQ ID NO: 188 - protein, 15 aa, Artificial Sequence
  Synthetic Construct
RSGGGWSEKLVAYDY

SEQ ID NO: 189 - protein, 16 aa, Artificial Sequence
  Synthetic Construct
IFVRGGSYYPADEYDY

SEQ ID NO: 190 - protein, 6 aa, Artificial Sequence
  Synthetic Construct
LFHNDY

SEQ ID NO: 191 - protein, 10 aa, Artificial Sequence
  Synthetic Construct
RIGFSSTNNY

SEQ ID NO: 192 - protein, 16 aa, Artificial Sequence
  Synthetic Construct
DLFGVPTEYPGQEFHI

SEQ ID NO: 193 - protein, 11 aa, Artificial Sequence
  Synthetic Construct
VQPLYKSDGVY

SEQ ID NO: 194 - protein, 11 aa, Artificial Sequence
  Synthetic Construct
VPPLYKRDGVY

SEQ ID NO: 195 - protein, 6 aa, Artificial Sequence
  Synthetic Construct
DGRGDE

SEQ ID NO: 196 - protein, 11 aa, Artificial Sequence
  Synthetic Construct
  VARIANT 2, 6, 10: Xaa = Ala, Asp, Gly, His, Ile, Leu, Asn, Pro, Arg, Ser, Thr, or Val
PXPAPXPAPXP

SEQ ID NO: 197 - protein, 15 aa, Artificial Sequence
  Synthetic Construct
  VARIANT 2: Xaa = Ala, Asp, Gly, His, Ile, Leu, Asn, Pro, Arg, Ser, Thr, or Val
  VARIANT 6: Xaa = Ala, Asp, Gly, His, Ile, Leu, Asn, Pro, Arg, Ser, Thr, or Val
  VARIANT 10: Xaa = Ala, Gly, Pro, or Arg
  VARIANT 14: Xaa = Ala, Asp, Gly, His, Ile, Leu, Asn, Pro, Arg, Ser, Thr, or Val
PXPAPXPAPXPAPXP

SEQ ID NO: 198 - protein, 19 aa, Artificial Sequence
  Synthetic Construct
  VARIANT 2: Xaa = Ala, Asp, Gly, His, Ile, Leu, Asn, Pro, Arg, Ser, Thr, or Val
  VARIANT 6, 14: Xaa = Ala, Gly, Pro, or Arg
  VARIANT 10: Xaa = Ala, Gly, Pro, Arg, Ser, or Thr
  VARIANT 18: Xaa = Ala, Asp, Gly, His, Ile, Leu, Asn, Pro, Arg, Ser, Thr, or Val
PXPAPXPAPXPAPXPAPXP

SEQ ID NO: 199 - protein, 23 aa, Artificial Sequence
  Synthetic Construct
  VARIANT 2, 10, 18: Xaa = Ala, Gly, Pro, Arg, Ser, or Thr
  VARIANT 22: Xaa = Ala, Asp, Gly, His, Ile, Leu, Asn, Pro, Arg, Ser, Thr, or Val
PXPAPAPAPXPAPAPAPXPAPXP

SEQ ID NO: 200 - protein, 27 aa, Artificial Sequence
  Synthetic Construct
  VARIANT 2, 10, 18: Xaa = Ala, Gly, Pro, Arg, Ser, or Thr
  VARIANT 26: Xaa = Ala, Asp, Gly, His, Ile, Leu, Asn, Pro, Arg, Ser, Thr, or Val
PXPAPAPAPXPAPAPAPXPAPAPAPXP

SEQ ID NO: 201 - protein, 31 aa, Artificial Sequence
  Synthetic Construct
  VARIANT 2, 10, 18, 26: Xaa = Ala, Gly, Ser, or Thr
  VARIANT 30: Xaa = Ala, Asp, Gly, His, Ile, Leu, Asn, Pro, Arg, Ser, Thr, or Val
PXPAPAPAPXPAPAPAPXPAPAPAPXPAPXP

SEQ ID NO: 202 - protein, 24 aa, Artificial Sequence
  Synthetic Construct
  VARIANT 2, 3: Xaa = Ala, Gly, Ser, or Thr
  VARIANT 13, 21: Xaa = Ala, Gly, Pro, Arg, Ser, or Thr
  VARIANT 23: Xaa = Ala, Asp, Gly, His, Ile, Leu, Asn, Pro, Arg, Ser, Thr, or Val
PXXPTPPPPAPPXPPPPAPPXPXP

SEQ ID NO: 203 - protein, 28 aa, Artificial Sequence
  Synthetic Construct
  VARIANT 2, 3: Xaa = Ala, Gly, Ser, or Thr
  VARIANT 13, 23: Xaa = Ala, Gly, Pro, Arg, Ser, or Thr
  VARIANT 27: Xaa = Ala, Asp, Gly, His, Ile, Leu, Asn, Pro, Arg, Ser, Thr, or Val
PXXPTPPPPAPPXPTPPPPAPPXPTPXP

SEQ ID NO: 204 - protein, 16 aa, Artificial Sequence
  Synthetic Construct
  VARIANT 2, 13: Xaa = Ala, Gly, Pro, Arg, Ser, or Thr
  VARIANT 3: Xaa = Ala, Gly, Ser, or Thr
  VARIANT 15: Xaa = Ala, Asp, Gly, His, Ile, Leu, Asn, Pro, Arg, Ser, Thr, or Val
PXXPTPPPPAPPXPXP

SEQ ID NO: 205 - protein, 20 aa, Artificial Sequence
  Synthetic Construct
  VARIANT 2, 13: Xaa = Ala, Gly, Pro, Arg, Ser, or Thr
  VARIANT 3: Xaa = Ala, Gly, Ser, or Thr
  VARIANT 19: Xaa = Ala, Asp, Gly, His, Ile, Leu, Asn, Pro, Arg, Ser, Thr, or Val
PXXPTPPPPAPPXPTPPPXP

SEQ ID NO: 206 - protein, 31 aa, Artificial Sequence
  Synthetic Construct
  VARIANT 2, 3, 13, 23: Xaa = Ala, Gly, Ser, or Thr
  VARIANT 28: Xaa = Ala, Gly, Pro, or Arg
  VARIANT 30: Xaa = Ala, Asp, Gly, His, Ile, Leu, Asn, Pro, Arg, Ser, Thr, or Val
PXXPTPPPPAPPXPTPPPPAPPXPTPPXPXP

SEQ ID NO: 207 - protein, 34 aa, Artificial Sequence
  Synthetic Construct
  VARIANT 2, 3, 13, 23: Xaa = Ala, Gly, Ser, or Thr
  VARIANT 33: Xaa = Ala, Asp, Gly, His, Ile, Leu, Asn, Pro, Arg, Ser, Thr, or Val
PXXPTPPPPAPPXPTPPPPAPPXPTPPPPAPPXP

SEQ ID NO: 208 - protein, 21 aa, Artificial Sequence
  Synthetic Construct
  VARIANT 1: Xaa = Ala, Gly, Ser, or Thr
  VARIANT 2: Xaa = Ala, Asp, or Gly
  VARIANT 20: Xaa = Ala, Asp, Phe, His, Ile, Leu, Asn, Pro, Ser, Thr, Val, or Tyr
  VARIANT 21: Xaa = Ala, Asp, Gly, His, Ile, Leu, Asn, Pro, Arg, Ser, Thr, or Val
XXPTPPTPPNPPTPPSPPPXX

SEQ ID NO: 209 - protein, 18 aa, Artificial Sequence
  Synthetic Construct
  VARIANT 1: Xaa = Ala, Gly, Ser, or Thr
  VARIANT 2: Xaa = Ala, Asp, or Gly
  VARIANT 17: Xaa = Ala, Asp, Phe, His, Ile, Leu, Asn, Pro, Ser, Thr, Val, or Tyr
  VARIANT 18: Xaa = Ala, Asp, Gly, His, Ile, Leu, Asn, Pro, Arg, Ser, Thr, or Val
XXPTPPTPPNPPSPPPXX

SEQ ID NO: 210 - protein, 12 aa, Artificial Sequence
  Synthetic Construct
  VARIANT 1: Xaa = Ala, Gly, Ser, or Thr
  VARIANT 2: Xaa = Ala, Asp, or Gly
  VARIANT 11: Xaa = Ala, Asp, Phe, His, Ile, Leu, Asn, Pro, Ser, Thr, Val, or Tyr
  VARIANT 12: Xaa = Ala, Asp, Gly, His, Ile, Leu, Asn, Pro, Arg, Ser, Thr, or Val
XXPTPPSPPPXX

SEQ ID NO: 211 - protein, 15 aa, Artificial Sequence
  Synthetic Construct
  VARIANT 1: Xaa = Ala, Gly, Ser, or Thr
  VARIANT 2: Xaa = Ala, Asp, or Gly
  VARIANT 14: Xaa = Ala, Asp, Phe, His, Ile, Leu, Asn, Pro, Ser, Thr, Val, or Tyr
  VARIANT 15: Xaa = Ala, Asp, Gly, His, Ile, Leu, Asn, Pro, Arg, Ser, Thr, or Val
XXPTPPTPPNPPTXX

SEQ ID NO: 212 - protein, 23 aa, Artificial Sequence
  Synthetic Construct
  VARIANT 1: Xaa = Ala, Gly, Ser, or Thr
  VARIANT 2: Xaa = Ala, Asp, or Gly
  VARIANT 22: Xaa = Ala, Asp, Phe, His, Ile, Leu, Asn, Pro, Ser, Thr, Val, or Tyr
  VARIANT 23: Xaa = Ala, Asp, Gly, His, Ile, Leu, Asn, Pro, Arg, Ser, Thr, or Val
XXPTPPTPPNPPTPPSPPTPPXX

SEQ ID NO: 213 - protein, 26 aa, Artificial Sequence
  Synthetic Construct
  VARIANT 1: Xaa = Ala, Gly, Ser, or Thr
  VARIANT 2: Xaa = Ala, Asp, or Gly
  VARIANT 25: Xaa = Ala, Asp, Phe, His, Ile, Leu, Asn, Pro, Ser, Thr, Val, or Tyr
  VARIANT 26: Xaa = Ala, Asp, Gly, His, Ile, Leu, Asn, Pro, Arg, Ser, Thr, or Val
XXPTPPTPPNPPTPPSPTPPSPPPXX

SEQ ID NO: 214 - protein, 29 aa, Artificial Sequence
  Synthetic Construct
  VARIANT 1: Xaa = Ala, Gly, Ser, or Thr
  VARIANT 2: Xaa = Ala, Asp, or Gly
  VARIANT 28: Xaa = Ala, Asp, Phe, His, Ile, Leu, Asn, Pro, Ser, Thr, Val, or Tyr
  VARIANT 29: Xaa = Ala, Asp, Gly, His, Ile, Leu, Asn, Pro, Arg, Ser, Thr, or Val
XXPTPPTPPNPPTPPSPTPPNPPSPPPXX

SEQ ID NO: 215 - protein, 32 aa, Artificial Sequence
  Synthetic Construct
  VARIANT 1: Xaa = Ala, Gly, Ser, or Thr
  VARIANT 2: Xaa = Ala, Asp, or Gly
  VARIANT 31: Xaa = Ala, Asp, Phe, His, Ile, Leu, Asn, Pro, Ser, Thr, Val, or Tyr
  VARIANT 32: Xaa = Ala, Asp, Gly, His, Ile, Leu, Asn, Pro, Arg, Ser, Thr, or Val
XXPTPPTPPNPPTPPSPTPPTPPNPPSPPPXX

SEQ ID NO: 216 - protein, 6 aa, Artificial Sequence
  Synthetic Construct
  VARIANT 5: Xaa = Ala, Asp, Phe, His, Ile, Leu, Asn, Pro, Ser, Thr, Val, or Tyr
  VARIANT 6: Xaa = Ala, Asp, Gly, His, Ile, Leu, Asn, Pro, Arg, Ser, Thr, or Val
GGGSXX

SEQ ID NO: 217 - protein, 7 aa, Artificial Sequence
  Synthetic Construct
  VARIANT 6: Xaa = Ala, Asp, Phe, His, Ile, Leu, Asn, Pro, Ser, Thr, Val, or Tyr
  VARIANT 7: Xaa = Ala, Asp, Gly, His, Ile, Leu, Asn, Pro, Arg, Ser, Thr, or Val
GGGGSXX

SEQ ID NO: 218 - protein, 8 aa, Artificial Sequence
  Synthetic Construct
  VARIANT 7: Xaa = Ala, Asp, Phe, His, Ile, Leu, Asn, Pro, Ser, Thr, Val, or Tyr
  VARIANT 8: Xaa = Ala, Asp, Gly, His, Ile, Leu, Asn, Pro, Arg, Ser, Thr, or Val
GGSGGSXX

SEQ ID NO: 219 - protein, 9 aa, Artificial Sequence
  Synthetic Construct
  VARIANT 8: Xaa = Ala, Asp, Phe, His, Ile, Leu, Asn, Pro, Ser, Thr, Val, or Tyr
  VARIANT 9: Xaa = Ala, Asp, Gly, His, Ile, Leu, Asn, Pro, Arg, Ser, Thr, or Val
GGGSGGSXX

SEQ ID NO: 220 - protein, 10 aa, Artificial Sequence
  Synthetic Construct
  VARIANT 9: Xaa = Ala, Asp, Phe, His, Ile, Leu, Asn, Pro, Ser, Thr, Val, or Tyr
  VARIANT 10: Xaa = Ala, Asp, Gly, His, Ile, Leu, Asn, Pro, Arg, Ser, Thr, or Val
GGGSGGGSXX

SEQ ID NO: 221 - protein, 11 aa, Artificial Sequence
  Synthetic Construct
  VARIANT 10: Xaa = Ala, Asp, Phe, His, Ile, Leu, Asn, Pro, Ser, Thr, Val, or Tyr
  VARIANT 11: Xaa = Ala, Asp, Gly, His, Ile, Leu, Asn, Pro, Arg, Ser, Thr, or Val
GGSGGSGGSXX

SEQ ID NO: 222 - protein, 12 aa, Artificial Sequence
  Synthetic Construct
  VARIANT 11: Xaa = Ala, Asp, Phe, His, Ile, Leu, Asn, Pro, Ser, Thr, Val, or Tyr
  VARIANT 12: Xaa = Ala, Asp, Gly, His, Ile, Leu, Asn, Pro, Arg, Ser, Thr, or Val
GGGGSGGGGSXX

SEQ ID NO: 223 - protein, 13 aa, Artificial Sequence
  Synthetic Construct
  VARIANT 12: Xaa = Ala, Asp, Phe, His, Ile, Leu, Asn, Pro, Ser, Thr, Val, or Tyr
  VARIANT 13: Xaa = Ala, Asp, Gly, His, Ile, Leu, Asn, Pro, Arg, Ser, Thr, or Val
GGGSGGGSGGSXX

SEQ ID NO: 224 - protein, 14 aa, Artificial Sequence
  Synthetic Construct
  VARIANT 13: Xaa = Ala, Asp, Phe, His, Ile, Leu, Asn, Pro, Ser, Thr, Val, or Tyr
  VARIANT 14: Xaa = Ala, Asp, Gly, His, Ile, Leu, Asn, Pro, Arg, Ser, Thr, or Val
GGGSGGGSGGGSXX

SEQ ID NO: 225 - protein, 15 aa, Artificial Sequence
  Synthetic Construct
  VARIANT 14: Xaa = Ala, Asp, Phe, His, Ile, Leu, Asn, Pro, Ser, Thr, Val, or Tyr
  VARIANT 15: Xaa = Ala, Asp, Gly, His, Ile, Leu, Asn, Pro, Arg, Ser, Thr, or Val
GGGGSGGGSGGGSXX

SEQ ID NO: 226 - protein, 16 aa, Artificial Sequence
  Synthetic Construct
  VARIANT 15: Xaa = Ala, Asp, Phe, His, Ile, Leu, Asn, Pro, Ser, Thr, Val, or Tyr
  VARIANT 16: Xaa = Ala, Asp, Gly, His, Ile, Leu, Asn, Pro, Arg, Ser, Thr, or Val
GGGGSGGGGSGGGSXX

SEQ ID NO: 227 - protein, 17 aa, Artificial Sequence
  Synthetic Construct
  VARIANT 16: Xaa = Ala, Asp, Phe, His, Ile, Leu, Asn, Pro, Ser, Thr, Val, or Tyr
  VARIANT 17: Xaa = Ala, Asp, Gly, His, Ile, Leu, Asn, Pro, Arg, Ser, Thr, or Val
GGGGSGGGGSGGGGSXX

SEQ ID NO: 228 - protein, 18 aa, Artificial Sequence
  Synthetic Construct
  VARIANT 17: Xaa = Ala, Asp, Phe, His, Ile, Leu, Asn, Pro, Ser, Thr, Val, or Tyr
  VARIANT 18: Xaa = Ala, Asp, Gly, His, Ile, Leu, Asn, Pro, Arg, Ser, Thr, or Val
GGGSGGGSGGGSGGGSXX

SEQ ID NO: 229 - protein, 19 aa, Artificial Sequence
  Synthetic Construct
  VARIANT 18: Xaa = Ala, Asp, Phe, His, Ile, Leu, Asn, Pro, Ser, Thr, Val, or Tyr
  VARIANT 19: Xaa = Ala, Asp, Gly, His, Ile, Leu, Asn, Pro, Arg, Ser, Thr, or Val
GGGGSGGGSGGGSGGGSXX

SEQ ID NO: 230 - protein, 20 aa, Artificial Sequence
  Synthetic Construct
  VARIANT 19: Xaa = Ala, Asp, Phe, His, Ile, Leu, Asn, Pro, Ser, Thr, Val, or Tyr
  VARIANT 20: Xaa = Ala, Asp, Gly, His, Ile, Leu, Asn, Pro, Arg, Ser, Thr, or Val
GGSGGSGGSGGSGGSGGSXX

SEQ ID NO: 231 - protein, 21 aa, Artificial Sequence
  Synthetic Construct
  VARIANT 20: Xaa = Ala, Asp, Phe, His, Ile, Leu, Asn, Pro, Ser, Thr, Val, or Tyr
  VARIANT 21: Xaa = Ala, Asp, Gly, His, Ile, Leu, Asn, Pro, Arg, Ser, Thr, or Val
GGGGSGGGGSGGGGSGGGSXX

SEQ ID NO: 232 - protein, 22 aa, Artificial Sequence
  Synthetic Construct
  VARIANT 21: Xaa = Ala, Asp, Phe, His, Ile, Leu, Asn, Pro, Ser, Thr, Val, or Tyr
  VARIANT 22: Xaa = Ala, Asp, Gly, His, Ile, Leu, Asn, Pro, Arg, Ser, Thr, or Val
GGGSGGGSGGGSGGGSGGGSXX

SEQ ID NO: 233 - protein, 22 aa, Artificial Sequence
  Synthetic Construct
  VARIANT 21: Xaa = Ala, Asp, Phe, His, Ile, Leu, Asn, Pro, Ser, Thr, Val, or Tyr
  VARIANT 22: Xaa = Ala, Asp, Gly, His, Ile, Leu, Asn, Pro, Arg, Ser, Thr, or Val
GGGGSGGGGSGGGGSGGGGSXX

SEQ ID NO: 234 - protein, 23 aa, Artificial Sequence
  Synthetic Construct
  VARIANT 22: Xaa = Ala, Asp, Phe, His, Ile, Leu, Asn, Pro, Ser, Thr, Val, or Tyr
  VARIANT 23: Xaa = Ala, Asp, Gly, His, Ile, Leu, Asn, Pro, Arg, Ser, Thr, or Val
GGSGGSGGSGGSGGSGGSGGSXX

SEQ ID NO: 235 - protein, 24 aa, Artificial Sequence
  Synthetic Construct
  VARIANT 23: Xaa = Ala, Asp, Phe, His, Ile, Leu, Asn, Pro, Ser, Thr, Val, or Tyr
  VARIANT 24: Xaa = Ala, Asp, Gly, His, Ile, Leu, Asn, Pro, Arg, Ser, Thr, or Val
GGGGSGGGGSGGGSGGGSGGGSXX

SEQ ID NO: 236 - protein, 25 aa, Artificial Sequence
  Synthetic Construct
  VARIANT 24: Xaa = Ala, Asp, Phe, His, Ile, Leu, Asn, Pro, Ser, Thr, Val, or Tyr
  VARIANT 25: Xaa = Ala, Asp, Gly, His, Ile, Leu, Asn, Pro, Arg, Ser, Thr, or Val
GGGGSGGGGSGGGGSGGGSGGGSXX

SEQ ID NO: 237 - protein, 26 aa, Artificial Sequence
  Synthetic Construct
  VARIANT 25: Xaa = Ala, Asp, Phe, His, Ile, Leu, Asn, Pro, Ser, Thr, Val, or Tyr
  VARIANT 26: Xaa = Ala, Asp, Gly, His, Ile, Leu, Asn, Pro, Arg, Ser, Thr, or Val
GGGSGGGSGGGSGGGSGGGSGGGSXX

SEQ ID NO: 238 - protein, 27 aa, Artificial Sequence
  Synthetic Construct
  VARIANT 26: Xaa = Ala, Asp, Phe, His, Ile, Leu, Asn, Pro, Ser, Thr, Val, or Tyr
  VARIANT 27: Xaa = Ala, Asp, Gly, His, Ile, Leu, Asn, Pro, Arg, Ser, Thr, or Val
GGGGSGGGGSGGGGSGGGGSGGGGSXX

SEQ ID NO: 239 - protein, 28 aa, Artificial Sequence
  Synthetic Construct
  VARIANT 27: Xaa = Ala, Asp, Phe, His, Ile, Leu, Asn, Pro, Ser, Thr, Val, or Tyr
  VARIANT 28: Xaa = Ala, Asp, Gly, His, Ile, Leu, Asn, Pro, Arg, Ser, Thr, or Val
GGGGSGGGGSGGGSGGGSGGGSGGGSXX

SEQ ID NO: 240 - protein, 29 aa, Artificial Sequence
  Synthetic Construct
  VARIANT 28: Xaa = Ala, Asp, Phe, His, Ile, Leu, Asn, Pro, Ser, Thr, Val, or Tyr
  VARIANT 29: Xaa = Ala, Asp, Gly, His, Ile, Leu, Asn, Pro, Arg, Ser, Thr, or Val
GGSGGSGGSGGSGGSGGSGGSGGSGGSXX

SEQ ID NO: 241 - protein, 30 aa, Artificial Sequence
  Synthetic Construct
  VARIANT 29: Xaa = Ala, Asp, Phe, His, Ile, Leu, Asn, Pro, Ser, Thr, Val, or Tyr
  VARIANT 30: Xaa = Ala, Asp, Gly, His, Ile, Leu, Asn, Pro, Arg, Ser, Thr, or Val
GGGSGGGSGGGSGGGSGGGSGGGSGGGSXX

SEQ ID NO: 242 - protein, 31 aa, Artificial Sequence
  Synthetic Construct
  VARIANT 30: Xaa = Ala, Asp, Phe, His, Ile, Leu, Asn, Pro, Ser, Thr, Val, or Tyr
  VARIANT 31: Xaa = Ala, Asp, Gly, His, Ile, Leu, Asn, Pro, Arg, Ser, Thr, or Val
GGGGSGGGSGGGSGGGSGGGSGGGSGGGSXX

SEQ ID NO: 243 - protein, 32 aa, Artificial Sequence
  Synthetic Construct
  VARIANT 31: Xaa = Ala, Asp, Phe, His, Ile, Leu, Asn, Pro, Ser, Thr, Val, or Tyr
  VARIANT 32: Xaa = Ala, Asp, Gly, His, Ile, Leu, Asn, Pro, Arg, Ser, Thr, or Val
GGGGSGGGGSGGGGSGGGGSGGGGSGGGGSXX

SEQ ID NO: 244 - protein, 33 aa, Artificial Sequence
  Synthetic Construct
  VARIANT 32: Xaa = Ala, Asp, Phe, His, Ile, Leu, Asn, Pro, Ser, Thr, Val, or Tyr
  VARIANT 33: Xaa = Ala, Asp, Gly, His, Ile, Leu, Asn, Pro, Arg, Ser, Thr, or Val
GGGGSGGGGSGGGGSGGGSGGGSGGGSGGGSXX

SEQ ID NO: 245 - protein, 34 aa, Artificial Sequence
  Synthetic Construct
  VARIANT 33: Xaa = Ala, Asp, Phe, His, Ile, Leu, Asn, Pro, Ser, Thr, Val, or Tyr
  VARIANT 34: Xaa = Ala, Asp, Gly, His, Ile, Leu, Asn, Pro, Arg, Ser, Thr, or Val
GGGSGGGSGGGSGGGSGGGSGGGSGGGSGGGSXX

SEQ ID NO: 246 - protein, 10 aa, Artificial Sequence
  Synthetic Construct
AGAFISDTVY

SEQ ID NO: 247 - protein, 10 aa, Artificial Sequence
  Synthetic Construct
AGAFIGDTVY

SEQ ID NO: 248 - protein, 10 aa, Artificial Sequence
  Synthetic Construct
GIGFSSNTMG

SEQ ID NO: 249 - protein, 16 aa, Artificial Sequence
  Synthetic Construct
SITNIGDAHYAASAKG

SEQ ID NO: 250 - protein, 16 aa, Artificial Sequence
  Synthetic Construct
SITNIGDAHYAASVKG

SEQ ID NO: 251 - protein, 16 aa, Artificial Sequence
  Synthetic Construct
SISNIGDAHYAASVKG

SEQ ID NO: 252 - protein, 16 aa, Artificial Sequence
  Synthetic Construct
FISSNGRQNYADSVKG

SEQ ID NO: 253 - protein, 5 aa, Artificial Sequence
  Synthetic Construct
WLVDY

SEQ ID NO: 254 - protein, 15 aa, Artificial Sequence
  Synthetic Construct
AGPTPPTPPNPPTAG

SEQ ID NO: 255 - protein, 12 aa, Artificial Sequence
  Synthetic Construct
GGPTPPSPPPSP

SEQ ID NO: 256 - protein, 28 aa, Artificial Sequence
  Synthetic Construct
TPSPAPAPAPGPAPAPAPRPAPAPAPVP

SEQ ID NO: 257 - protein, 24 aa, Artificial Sequence
  Synthetic Construct
GPAPAPAPAPRPAPAPTPGPAPGP

SEQ ID NO: 258 - protein, 24 aa, Artificial Sequence
  Synthetic Construct
SPAPAPAPAPRPAPAPAPGPAPGP

SEQ ID NO: 259 - protein, 28 aa, Artificial Sequence
  Synthetic Construct
APRPAPAPAPGPAPAPAPPPAPAPAPSP

SEQ ID NO: 260 - protein, 29 aa, Artificial Sequence
  Synthetic Construct
SPSSPTPPPPAPPSPTPPPPAPPGPTPDP

SEQ ID NO: 261 - protein, 24 aa, Artificial Sequence
  Synthetic Construct
GPGPAPAPAPGPAPAPAPAPAPIP

SEQ ID NO: 262 - protein, 26 aa, Artificial Sequence
  Synthetic Construct
GDPTPPTPPNPPTPPSPTPPSPPPPR

SEQ ID NO: 263 - protein, 10 aa, Artificial Sequence
  Synthetic Construct
RSAWSIINMA

SEQ ID NO: 264 - protein, 10 aa, Artificial Sequence
  Synthetic Construct
GIIFSHYTMG

SEQ ID NO: 265 - protein, 10 aa, Artificial Sequence
  Synthetic Construct
GTILRFITMD

SEQ ID NO: 266 - protein, 9 aa, Artificial Sequence
  Synthetic Construct
RSLSANGVS

SEQ ID NO: 267 - protein, 10 aa, Artificial Sequence
  Synthetic Construct
GSIFSGNAMD

SEQ ID NO: 268 - protein, 10 aa, Artificial Sequence
  Synthetic Construct
ETIFSGNAMD

SEQ ID NO: 269 - protein, 10 aa, Artificial Sequence
  Synthetic Construct
AGAFISDTVY

SEQ ID NO: 270 - protein, 10 aa, Artificial Sequence
  Synthetic Construct
GSIFSTNPIQ

SEQ ID NO: 271 - protein, 10 aa, Artificial Sequence
  Synthetic Construct
GFTFGSLTMK

SEQ ID NO: 272 - protein, 16 aa, Artificial Sequence
  Synthetic Construct
RLTPGGSAVYGEFVKG

SEQ ID NO: 273 - protein, 16 aa, Artificial Sequence
  Synthetic Construct
LITPGDGTYYADSVKG

SEQ ID NO: 274 - protein, 16 aa, Artificial Sequence
  Synthetic Construct
RITGGGTTLYANSVKD

SEQ ID NO: 275 - protein, 16 aa, Artificial Sequence
  Synthetic Construct
TITGSGFGHYADSVKG

SEQ ID NO: 276 - protein, 16 aa, Artificial Sequence
  Synthetic Construct
LITSDGSTTYADSVKG

SEQ ID NO: 277 - protein, 16 aa, Artificial Sequence
  Synthetic Construct
TITGGGGTNYLDSVKG

SEQ ID NO: 278 - protein, 17 aa, Artificial Sequence
  Synthetic Construct
SISSGGGTAAYSDSVKG

SEQ ID NO: 279 - protein, 10 aa, Artificial Sequence
  Synthetic Construct
DTPPYGGPDY

SEQ ID NO: 280 - protein, 10 aa, Artificial Sequence
  Synthetic Construct
KSRSIVNRDY

SEQ ID NO: 281 - protein, 7 aa, Artificial Sequence
  Synthetic Construct
LAPGRDY

SEQ ID NO: 282 - protein, 6 aa, Artificial Sequence
  Synthetic Construct
VPPTPY

SEQ ID NO: 283 - protein, 6 aa, Artificial Sequence
  Synthetic Construct
VPPLGR

SEQ ID NO: 284 - protein, 6 aa, Artificial Sequence
  Synthetic Construct
VPPLPR

SEQ ID NO: 285 - protein, 5 aa, Artificial Sequence
  Synthetic Construct
YSYPY

SEQ ID NO: 286 - protein, 11 aa, Artificial Sequence
  Synthetic Construct
AASPANAYGIY

SEQ ID NO: 287 - protein, 10 aa, Artificial Sequence
  Synthetic Construct
GFTFTDYKIH

SEQ ID NO: 288 - protein, 17 aa, Artificial Sequence
  Synthetic Construct
YFNPNSGYSTYAQKFQG

SEQ ID NO: 289 - protein, 11 aa, Artificial Sequence
  Synthetic Construct
LSPGGYYVMDA

SEQ ID NO: 290 - protein, 15 aa, Artificial Sequence
  Synthetic Construct
GGGGSSGGGSGGGGS

SEQ ID NO: 291 - protein, 11 aa, Artificial Sequence
  Synthetic Construct
RASQGINNYLN

SEQ ID NO: 292 - protein, 7 aa, Artificial Sequence
  Synthetic Construct
NTNNLQT

SEQ ID NO: 293 - protein, 8 aa, Artificial Sequence
  Synthetic Construct
LQHNSFPT

(19) United States
(12) Patent Application Publication
(10) Pub. No.: US 2025/0199010 A1
(43) Pub. Date: Jun. 19, 2025

(54) BINDER MOLECULES WITH HIGH AFFINITY AND/ OR SPECIFICITY AND METHODS OF MAKING AND USE THEREOF

(71) Applicant: Alamar Biosciences, Inc., Fremont, CA (US)

(72) Inventors: Yiyuan YIN, Fremont, CA (US); Yuling LUO, Fremont, CA (US); Adrian GRZYBOWSKI, Fremont, CA (US)

(73) Assignee: Alamar Biosciences, Inc., Fremont, CA (US)

(21) Appl. No.: 18/268,936

(22) PCT Filed: Dec. 29, 2021
(86) PCT No.: PCT/US2021/073173

§ 371 Date: Jun. 21, 2023

Related U.S. Application Data
(60) Provisional application No. 63/133,005, filed on Dec. 31, 2020.
(60) Provisional application No. 63/133,020, filed on Dec. 31, 2020.

(51) Int. Cl. G01N 33/68; C07K 16/10; C07K 16/28

(52) U.S. Cl. CPC G01N 33/6845; C07K 16/1054; C07K 16/2863; C07K 2317/33; C07K 2317/569; C07K 2317/92

(57) ABSTRACT

Provided herein, in some aspects, are binding molecules including co-binders having high affinity and/or high specificity to a target. In other aspects, provided herein are compositions, methods of making, and methods of using the binding molecules taught herein, such as diagnostic and therapeutic methods of use involving the co-binders taught herein

### CROSS-REFERENCE TO RELATED APPLICATIONS

This application claims priority to and the benefit of U.S. Provisional Patent Application No. 63/133,005, filed on Dec. 31, 2020, and U.S. Provisional Patent Application No. 63/133,020, filed on Dec. 31, 2020, each of which is incorporated herein by reference in its entirety.

### TECHNICAL FIELD

The present application is directed to, in some aspects, binder molecules, such as co-binders, having high affinity and/or high specificity to a target molecule. In other aspects, also provided are methods of making, methods of using, such as diagnostic and therapeutic methods, and compositions comprising a binder molecule, such as co-binders.

### BACKGROUND

Antibodies and other binding molecules are useful in numerous fields, including those involving molecular detection, diagnosis, and methods of treatment. Producing such binding molecules with desired characteristics, such as size and immunogenicity, much less a desired binding affinity and specificity, remains a challenge in the field.

### BRIEF SUMMARY

In some aspects, provided is a co-binder comprising a first binding moiety specifically recognizing a first target site and a second binding moiety specifically recognizing a second target site, wherein, optionally, the second binding moiety is a second antibody moiety comprising an antibody variable domain having an N-terminal truncation (“N-terminal truncated antibody variable domain”), and wherein the first binding moiety is connected to the second binding moiety through N-terminus of the N-terminal truncated antibody variable domain, optionally via a linker. In some embodiments, the second binding moiety is a second antibody moiety comprising an antibody variable domain having an N-terminal truncation (“N-terminal truncated antibody variable domain”). In some embodiments, the co-binder comprises a linker. In some embodiments, the co-binder comprises a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), and wherein the first binding moiety is connected to the second binding moiety through N-terminus of the N-terminal truncated antibody variable domain via a linker.

In some embodiments, the co-binder binds to the second target site with an affinity of at least about 3 fold of that of a control co-binder. In some embodiments, the control co-binder comprises an antibody variable domain not having the N-terminal truncation (e.g., an N-terminal truncated antibody variable domain of a second binding moiety of a co-binder described herein).

In some embodiments, the first target site and the second target site are non-overlapping binding sites on a target molecule. In some embodiments, the co-binder binds to the target molecule with an affinity of at least about 3 fold of that of a control co-binder comprising an antibody variable domain not having the N-terminal truncation.

In some embodiments, the first binding moiety is a first antibody moiety. In some embodiments, the first antibody moiety is selected from the group consisting of a Fab, an Fv, an scFv, a dsFv, a Fab′, or a (Fab′)2 fragment. In some embodiments, the first antibody moiety is a single domain antibody.

In some embodiments, the second antibody moiety is selected from the group consisting of a Fab, Fv, scFv, dsFv, Fab′, or (Fab′)2 fragment. In some embodiments, the N-terminal truncated antibody variable domain is a truncated VH or truncated VL domain. In some embodiments, the second antibody moiety is a single domain antibody. In some embodiments, the N-terminal truncated antibody variable domain is a truncated V_{H}H domain.

In some embodiments, the first binding moiety comprises a first V_{H}H domain; wherein the second binding moiety comprises a second V_{H}H domain having an N-terminal truncation (“truncated V_{H}H domain”), wherein the C-terminus of the first V_{H}H domain is connected to the N-terminus of the second V_{H}H domain via a linker.

In some embodiments, the N-terminal truncation of the N-terminal truncated antibody variable domain is about 1 to about 25 amino acids. In some embodiments, the N-terminal truncation of the N-terminal truncated antibody variable domain is 1 amino acid.

In some embodiments, the linker is a peptide linker. In some embodiments, the C-terminal amino acid of the peptide linker immediately connected to the N-terminal truncated antibody variable domain is G.

In some embodiments, the C-terminal three amino acids of the peptide linker immediately connected to the N-terminal truncated antibody variable domain are X₁-X₂-X₃, wherein X₁ is V, L, W, P, S, G, K, D, F, M, T, N, or R; X₂ is V, A, L, S, G, R, K, M, C, F, T, P, or E; and X₃ is G. In some embodiments, the three C-terminal amino acids of the peptide linker immediately connected to the N-terminal truncated antibody variable domain is selected from the group consisting of: GVG, DSG, LLG, VSG, PPG, SCG, TLG, and NPG.

In some embodiments, the linker comprises (GₓS_{y})ₙ, wherein x is 1 to 5, y is 0 to 5, and n is 1 or more. In some embodiments, the linker comprises [EAAAK]ₙ, wherein n is 1 or more. In some embodiments, the linker is no more than about 40 amino acids long. In some embodiments, the linker comprises [EEEEKKKK]ₙ, wherein n is 1 or more. In some embodiments, the linker comprises [AP]ₙ, wherein n is 1 or more.

In some embodiments, the truncated variable domain is from an antibody variable domain of any of IgG, IgA, IgE, IgM, or IgD type.

In some embodiments, the co-binder further comprises a third binding moiety specifically recognizing a third target site. In some embodiments, the third binding moiety is a third antibody moiety. In some embodiments, the third antibody moiety comprises an antibody variable domain having an N-terminal truncation (“N-terminal truncated antibody variable domain”). In some embodiments, the third antibody moiety is connected to the second antibody moiety through the N-terminus of the N-terminal truncated antibody variable domain of the third antibody moiety via a linker.

In some embodiments, the third antibody moiety is connected to a fourth binding moiety through the N-terminus of the N-terminal truncated antibody variable domain of the third antibody moiety via a linker.

In some embodiments, the co-binder is an antibody comprising an Fc region.

In some embodiments, the co-binder is a chimeric antigen receptor (“CAR”).

In other aspects, provided is a co-binder comprising a first binding moiety specifically recognizing a first target site and a second binding moiety specifically recognizing a second target site, wherein the second binding moiety is a second antibody moiety comprising an antibody variable domain; wherein the first binding moiety is connected to the second binding moiety through N-terminus of the N-terminal truncated antibody variable domain via a peptide linker; wherein the C-terminal three amino acids of the peptide linker immediately connected to the antibody variable domain of the second binding moiety are X₁-X₂-X₃, wherein X₁ is any amino acid; X₂ is K, R, Y, M, G, or N; and X₃ is R, G, Y, or P.

In some embodiments, the co-binder binds to the second target site with an affinity of at least about 3 fold of linker control co-binder.

In some embodiments, the first target site and the second target site are non-overlapping binding sites on a target molecule.

In some embodiments, the co-binder binds to the target molecule with an affinity of at least about 3 fold of that of linker control co-binder.

In some embodiments, the first binding moiety is a first antibody moiety. In some embodiments, the first antibody moiety is selected from the group consisting of a Fab, an Fv, an scFv, a dsFv, a Fab′, or a (Fab′)2 fragment. In some embodiments, the first antibody moiety is a single domain antibody.

In some embodiments, the second antibody moiety is selected from the group consisting of a Fab, an Fv, an scFv, a dsFv, a Fab′, or a (Fab′)2 fragment. In some embodiments, the antibody variable domain is a VH or VL domain. In some embodiments, the second antibody moiety is a single domain antibody. In some embodiments, the antibody variable domain is a V_{H}H domain.

In some embodiments, the first binding moiety comprises a first V_{H}H domain; wherein the second binding moiety comprises a second V_{H}H domain, wherein the C-terminus of the first V_{H}H domain is connected to the N-terminus of the second V_{H}H domain via the peptide linker.

In some embodiments, the three C-terminal amino acids of the peptide linker immediately connected to the N-terminal truncated antibody variable domain is selected from the group consisting of: GVG, DSG, LLG, VSG, PPG, SCG, TLG, and NPG.

In some embodiments, the linker comprises (GₓS_{y})ₙ, wherein x is 1 to 5, y is 0 to 5, and n is 1 or more. In some embodiments, the linker comprises [EAAAK]ₙ, wherein n is 1 or more. In some embodiments, the linker is no more than about 40 amino acids long. In some embodiments, the linker comprises [EEEEKKKK]ₙ, wherein n is 1 or more. In some embodiments, the linker comprises [AP]ₙ, wherein n is 1 or more.

In some embodiments, the co-binder further comprises a third binding moiety specifically recognizing a third target site. In some embodiments, the third binding moiety is a third antibody moiety. In some embodiments, the third antibody moiety comprises an antibody variable domain having an N-terminal truncation (“N-terminal truncated antibody variable domain”). In some embodiments, the third antibody moiety is connected to the second antibody moiety through the N-terminus of the N-terminal truncated antibody variable domain of the third antibody moiety via a linker. In some embodiments, the third antibody moiety is connected to a fourth binding moiety through the N-terminus of the N-terminal truncated antibody variable domain of the third antibody moiety via a linker.

In some embodiments, the co-binder is an antibody comprising an Fc region.

In some embodiments, the co-binder is a chimeric antigen receptor (“CAR”).

In other aspects, provided is a library comprising a plurality of co-binders or a plurality of polynucleotides encoding a plurality of co-binders, each co-binder comprising a first binding moiety specifically recognizing a first target site and a second binding moiety specifically recognizing a second target site, wherein the second binding moiety is a second antibody moiety comprising an antibody variable domain, wherein the first binding moiety is connected to the second binding moiety through N-terminus of the antibody variable domain via a peptide linker, wherein at least two co-binders in the library differ from each other in the peptide linker sequence.

In some embodiments, the first target site and the second target site are non-overlapping binding sites on a target molecule.

In some embodiments, the antibody variable domain has an N-terminal truncation (“N-terminal truncated antibody variable domain”). In some embodiments, at least two co-binders in the library differ from each other in the N-terminal truncation of the antibody variable domain.

In some embodiments, the diversity of the library is at least about 5000.

In some embodiments, substantially all of the plurality of co-binders comprise the same first binding moiety and second binding moiety.

In some embodiments, at least two of the plurality of co-binders comprise a different first binding moiety and/or second binding moiety.

In other aspects, provided is a method of screening for a co-binder specifically binding to a second target site at a desired affinity, the method comprising: (1) contacting a library described herein with a target molecule comprising the second target site to form complexes between the co-binders that specifically bind to the target molecule and the target molecule, and (2) identifying a co-binder that binds to the second target site with the desired affinity.

In other aspects, provided is a method of screening for a co-binder specifically binding to a target molecule at a desired affinity, the method comprising: (1) contacting a library described herein with the target molecule to form complexes between the co-binders that specifically bind to the target molecule and the target molecule, and (2) identifying a co-binder that binds to the target molecule with the desired affinity.

In other aspects, provided is a method of increasing binding affinity of a control co-binder specifically binding to a target molecule, wherein the control co-binder comprise a first binding moiety specifically recognizing a first target site and a second binding moiety specifically recognizing a second binding target site, wherein the second binding moiety is a second antibody moiety comprising an antibody variable domain, wherein the first binding moiety is connected to the second binding moiety through N-terminus of the antibody variable domain via a linker, wherein the control co-binder comprises a full length antibody variable domain, wherein the binding affinity of the control co-binder to the second target site is lower than that of a second antibody moiety in free state, the method comprising obtaining a co-binder having an N-terminal truncation at the antibody variable domain of the second antibody moiety as compared to the control co-binder.

In some embodiments, the first target site and the second target site are non-overlapping binding sites on a target molecule.

All applications, publications, patents and other references, GenBank citations cited herein are incorporated by reference in their entirety. In case of conflict, the specification, including definitions, will control.

### BRIEF DESCRIPTION OF THE DRAWINGS

FIG. 1 depicts an exemplary algorithm for determining the truncation or deletion of N-terminal residues in an antibody variable region.

FIG. 2 depicts another exemplary algorithm for determining the truncation or deletion of N-terminal residues in an antibody variable region.

FIGS. 3A-3D depict exemplary sources of binding energy loss when linking two binding moieties together. FIG. 3E depicts the crystal structure of 7D12 and 9G8 V_{H}Hs bound to EGFR, in which the cetuximab crystal structure overlaid for comparison.

FIG. 4A depicts the strategies for improving binding characteristics of co-binders by modifying linker attachment point between linker and antigen. FIGS. 4B-4C depict SDS-PAGE gel from purified proteins of (4B) HuL6-7D12 variants with truncations at the N-terminus of 7D12 and (4C) HuL6-9G8 variants with truncations at the N-terminus of 9G8. All variants in FIGS. 4B and 4C except non-truncated co-binders were expressed and purified in the same manner.

FIG. 5 depicts the co-binder library design with 3 amino acids randomization at C-terminus of the linker with or without first amino-acid of the second binder.

FIG. 6A depicts the consensus sequence for each library as described in Table 15 and accompanying text, the top 20 most enriched sequences were subjected to motif analysis using WebLogo software (Crooks et al., Genome Res. 2004 June; 14 (6): 1188-90). FIG. 6B depicts the yeast display and SPR measurements of affinities (K_{D}) between selected constructs having linker terminal modifications and human EGFR.

FIG. 7A depicts co-binder library design with 3 amino acids randomization at N-terminus and 2 amino acids randomization at C-terminus of the linker with last C-terminal amino-acid of the linker being a glycine. Library utilizes 4 different linker motifs: EAAAK and E4K4 repeats, AP repeat, and G3-4S repeat. FIG. 7B depicts the consensus sequence for each library as described in Table 16 and accompanying text, the top 20 most enriched sequences were subjected to motif analysis using WebLogo software (Crooks et al., Genome Res. 2004 June; 14 (6): 1188-90). FIG. 7C depicts linker length enrichment from the screening as described in Table 16 and accompanying text.

FIG. 8 depicts SPR affinity measurement of engineered co-binders toward murine EGFR-Fc and human EGFR-Fc mutant (L325V, S340A).

FIG. 9 depicts a schematic representation of a method for the discovery of co-binders with synergistic co-binding.

FIG. 10A shows that an anti-EGFR VHH yeast surface display library SB0 was constructed and single binder selection was done with FACS. FIG. 10B depicts selection of high-affinity co-binders from the CB0 co-binder library using FACS.

FIG. 11 shows down regulation of EGF-induced EGFR signaling by co-binders.

FIG. 12A shows sensogram of 81 nM 1E10 EGFR binder injected over immobilized EGFR-Fc, followed by injection of 81 nM 15E2 EGFR binder. FIG. 12B shows sensogram of 81 nM 7D12-9G8 EGFR binder injected over immobilized EGFR-Fc, followed by injection of 81 nM 15E2 EGFR binder. FIG. 12C shows sensogram of 81 nM 7D12-9G8 EGFR co-binder injected over immobilized EGFR-Fc, followed by injection of 81 nM 1E10 EGFR binder.

FIG. 13 shows Plot of the distances between the N-terminus of VHH and Fab domains to the antigenic surface. Each individual dot represents a unique structure selected from the PDB.

FIG. 14 shows a plot of the affinities of anti-EGFR (filled circle) and anti-HIV p24 (empty square) co-binders and single binders.

FIG. 15 shows a plot of the affinities of co-binders for 14 different targets and regular antibodies for said targets.

### DETAILED DESCRIPTION

Provided herein are binder molecules comprising a second binding moiety specifically recognizing a target site, wherein the second binding moiety is a second antibody moiety comprising an antibody variable domain having an N-terminal truncation (“N-terminal truncated antibody variable domain”). The disclosure of the application is based on the inventors' unexpected findings that such binder molecules, such as a co-binder, comprising a second binding moiety having an N-terminal truncated antibody variable domain provided a platform technology for binder molecules having high affinity and specificity. Moreover, the second binding moiety having an N-terminal truncated antibody variable domain can be combined with various other features, including a linker, a first binding moiety, a label, and/or drug, to produce desired binder molecules. Moreover, the design of the binder molecules encompassed herein enable production, such as via polypeptide expression, without post-production synthetic steps that often lead to loss of yield and contamination.

Thus, in some aspects, provided herein is a binder molecule comprising a second binding moiety specifically recognizing a target site, such as a target polypeptide, wherein the second binding moiety is a second antibody moiety comprising an antibody variable domain having an N-terminal truncation (“N-terminal truncated antibody variable domain”).

In other aspects, provided herein is a co-binder comprising a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), wherein the first binding moiety is connected to the second binding moiety through N-terminus of the N-terminal truncated antibody variable domain via a linker.

In other aspects, provided herein is a co-binder comprising a first binding moiety specifically recognizing a first target site and a second binding moiety specifically recognizing a second target site, wherein the second binding moiety is a second antibody moiety comprising an antibody variable domain; wherein the first binding moiety is connected to the second binding moiety through N-terminus of the N-terminal truncated antibody variable domain via a peptide linker; wherein the C-terminal three amino acids of the peptide linker immediately connected to the antibody variable domain of the second binding moiety are X₁-X₂-X₃, wherein X₁ is any amino acid; X₂ is K, R, Y, M, G, or N; and X₃ is R, G, Y, or P. In some embodiments, X₃ of X₁-X₂-X₃ is G.

In other aspects, provided herein is a library comprising a plurality of co-binders or a plurality of polynucleotides encoding a plurality of co-binders, each co-binder comprising a first binding moiety specifically recognizing a first target site and a second binding moiety specifically recognizing a second target site, wherein the second binding moiety is a second antibody moiety comprising an antibody variable domain, wherein the first binding moiety is connected to the second binding moiety through N-terminus of the antibody variable domain via a peptide linker, wherein at least two co-binders in the library differ from each other in the peptide linker sequence.

In other aspects, provided herein is a method of screening for a co-binder specifically binding to a second target site at a desired affinity, the method comprising: (1) contacting a library described herein with a target molecule comprising the second target site to form complexes between the co-binders that specifically bind to the target molecule and the target molecule, and (2) identifying a co-binder that binds to the second target site with the desired affinity.

In other aspects, provided herein is a method of screening for a co-binder specifically binding to a target molecule at a desired affinity, the method comprising: (1) contacting a library described herein with the target molecule to form complexes between the co-binders that specifically bind to the target molecule and the target molecule, and (2) identifying a co-binder that binds to the target molecule with the desired affinity.

In other aspects, provided herein is a method of increasing binding affinity of a control co-binder specifically binding to a target molecule, wherein the control co-binder comprise a first binding moiety specifically recognizing a first target site and a second binding moiety specifically recognizing a second binding target site, wherein the second binding moiety is a second antibody moiety comprising an antibody variable domain, wherein the first binding moiety is connected to the second binding moiety through N-terminus of the antibody variable domain via a linker, wherein the control co-binder comprises a full length antibody variable domain, wherein the binding affinity of the control co-binder to the second target site is lower than that of a second antibody moiety in free state, the method comprising obtaining a co-binder having an N-terminal truncation at the antibody variable domain of the second antibody moiety as compared to the control co-binder.

### I. Definitions

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

The terms “co-binder,” “co-binders,” “cobinder,” and “cobinders” are intended to mean a molecule that has at least two binding moieties (i.e., a first binding moiety comprising a first paratope and a second binding moiety comprising a second paratope) that bind non-overlapping epitopes of one target molecule or one target complex (e.g., protein complex). In some embodiments, the first and the second binding moieties simultaneously bind non-overlapping epitopes of one target molecule or one target complex (e.g., protein complex). In some embodiments, the at least two binding moieties simultaneously bind non-overlapping epitopes of one target molecule or one target complex (e.g. protein complex). The co-binders described herein comprise at least two binding moieties, such as any of 2, 3, 4, 5, 6, or 7 or more binding moieties. In some embodiments, the two or more binding moieties on one binding molecule are the same. In some embodiments, the two or more binding moieties on one binding molecule are different. In some embodiments, a co-binder has two binding moieties and the two epitopes recognized by a co-binder are non-overlapping and distinct. In some embodiments, a co-binder has two binding moieties and the two epitopes recognized by the co-binder are located close to each other, but still allow sufficient space to accommodate the linker of the co-binder. In some embodiments, a co-binder has two binding moieties and the first and second epitopes have a distance of no more than 150 angstroms. In some embodiments, a co-binder has two binding moieties and the first and second epitopes have a distance of no more than 100 angstroms, no more than 50 angstroms, no more than 40 angstroms, no more than 30 angstroms, no more than 20 angstroms, no more than 15 angstroms, no more than 10 angstroms, or no more than 5 angstroms. For linear epitopes on a target peptide or target protein, the distance between the any two epitopes can be within 200 amino acids of each other. In some embodiments, a co-binder has two binding moieties and the distance between the two epitopes can be within 200 amino acids, 150 amino acids, within 100 amino acids, within 50 amino acids, within 40 amino acids, within 30 amino acids, within 20 amino acids, within 15 amino acids, or within 10 amino acids of each other. In some embodiments, a co-binder has two binding moieties and the two epitopes recognized by the co-binder are selected such that the two binding interactions are cooperative and synergistic, and do not interfere with each other. A co-binder has both higher binding affinity and higher binding specificity than a typical bivalent antibody because of, for example, the additive effect of the two paratope-epitope binding interactions.

As used herein, the term “binding moiety” refers to a molecule or a portion of a molecule which binds a specific target molecule. A binding moiety can comprise a protein, peptide, nucleic acid, carbohydrate, lipid, or small molecular weight compound. In some embodiments, a binding moiety comprises an antibody. In some embodiments, a binding moiety comprises an antigen-binding fragment of an antibody. In some embodiments, a binding moiety comprises an antibody or an antigen-binding fragment thereof. In some embodiments, a binding moiety comprises a heavy chain variable region of an antibody. In some embodiments, a binding moiety comprises a light chain variable region of an antibody. In some embodiments, a binding moiety comprises a variable region of an antibody. In some embodiments, a binding moiety comprises an antibody mimetic. In some embodiments, a binding moiety comprises a small molecular weight component. In some embodiments, a binding molecule has only one binding moiety. In some embodiments, a binding molecule has two binding moieties. In some embodiments, a binding molecule has three or more binding moieties. In some embodiments, the two or more binding moieties on one binding molecule are the same. In some embodiments, the two or more binding moieties on one binding molecule are different. For example, a binding molecule can have two binding moieties, both being antigen binding fragments, such as VHHs. For another example, a binding molecule can also have two binding moieties, one being a VHH, and the other being scFv.

As used herein, the term “paratope,” is part of a binding moiety that recognizes and binds to a target molecule. A paratope of an antibody is also referred to as “an antigen-binding site.” The epitope and paratope for a given target molecule/binding molecule (e.g., Ag/Ab) pair can be identified by routine methods. For example, the target molecule and binding molecule can be combined to form a complex, which can be crystallized. The crystal structure of the complex can be determined by, for example, X-ray diffraction, and used to identify specific sites of interaction between the target molecule/binding molecule, namely, the epitope/paratope.

An “epitope” is the site on the surface of an antigen molecule to which a single antibody molecule binds, such as a localized region on the surface of an antigen (e.g. EGFR), that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. Generally an antigen has several or many different epitopes and may react with many different antibodies.

The term “binding protein” refers to a protein comprising a portion (e.g., one or more binding regions such as CDRs) that binds to a target antigen (e.g. EGFR) and, optionally, a scaffold or framework portion (e.g., one or more scaffold or framework regions) that allows the binding portion to adopt a conformation that promotes binding of the binding protein to a target polypeptide, fragment, or epitope thereof. Examples of such binding proteins include antibodies, such as a human antibody, a humanized antibody, a chimeric antibody, a recombinant antibody, a single chain antibody, a diabody, a triabody, a tetrabody, a Fab fragment, a F(ab′)₂ fragment, an IgD antibody, an IgE antibody, an IgM antibody, an IgG1 antibody, an IgG2 antibody, an IgG3 antibody, or an IgG4 antibody, and fragments thereof. The binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the binding protein as well as wholly synthetic scaffolds comprising, for example, a biocompatible polymer. See, e.g., Korndorfer et al., 2003, Proteins: Structure, Function, and Bioinformatics 53 (1): 121-29; and Roque et al., 2004, Biotechnol. Prog. 20:639-54. In addition, peptide antibody mimetics (“PAMs”) can be used, as well as scaffolds based on antibody mimetics utilizing fibronectin components as a scaffold. In the context of the present disclosure, a binding protein is said to specifically bind or selectively bind to a target, for example, when the dissociation constant (K_{D}) is ≤10⁻⁵ M. In some embodiments, the binding proteins (e.g., co-binders and antibodies) may specifically bind to a target with a K_{D} of from about 10⁻⁷ M to about 10⁻¹² M. In certain embodiments, the binding protein (e.g., co-binders and antibodies) may specifically bind to a target with high affinity when the K_{D} is ≤10⁻⁸ M or KD is ≤10⁻⁹ M. In one embodiment, the binding proteins (e.g., co-binders and antibodies) may specifically bind to purified human a target with a KD of from 1×10⁻⁹ M to 10×10⁻⁹ M as measured by Biacore®. In another embodiment, the binding proteins (e.g., co-binders and antibodies) may specifically bind to purified human a target with a KD of from 0.1×10⁻⁹ M to 1×10⁻⁹ M as measured by KinExA™ (Sapidyne, Boise, ID). In yet another embodiment, the binding proteins (e.g., co-binders and antibodies) specifically bind to a target expressed on cells with a K_{D} of from 0.1×10⁻⁹ M to 10×10⁻⁹ M. In certain embodiments, the binding proteins (e.g., co-binders and antibodies) specifically bind to a target expressed on cells with a K_{D} of from 0.1×10⁻⁹ M to 1×10⁻⁹ M. In some embodiments, the binding proteins (e.g., co-binders and antibodies) specifically bind to a target expressed on cells with a K_{D} of 1×10⁻⁹ M to 10×10⁻⁹ M. In certain embodiments, the binding proteins (e.g., co-binders and antibodies) specifically bind to a target expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof.

The term “antibody,” “immunoglobulin,” or “Ig” is used interchangeably herein, and is used in the broadest sense and specifically covers, for example, individual monoclonal antibodies (including agonist, antagonist, neutralizing antibodies, full length or intact monoclonal antibodies), antibody compositions with polyepitopic or monoepitopic specificity, polyclonal antibodies, monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. Thus, the term “antibody” encompasses various antibody structures, including but not limited to, polyclonal antibodies, recombinant antibodies, monoclonal antibodies, chimeric antibodies, humanized antibodies, human antibodies, biparatopic antibodies, bispecific antibodies, multispecific antibodies, diabodies, tribodies, tetrabodies, single chain Fv (scFv) antibodies, and antibody fragments as long as they exhibit the desired antigen-binding activity. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). The term “intact antibody” or “full-length antibody” refers to an antibody having a structure substantially similar to a native antibody structure. This includes, for example, an antibody comprising two light chains each comprising a variable region and a light chain constant region (CL) and two heavy chains each comprising a variable region and at least heavy chain constant regions CH1, CH2, and CH3. In specific embodiments, the specific molecular antigen can be bound by an antibody provided herein. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)2 fragments, disulfide-linked Fvs (dsFv), disulfide-linked scFv (dsscFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; P1ückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. An antibody may be an agonistic antibody or antagonistic antibody. Provided herein are antagonistic antibodies to a target antigen such as EGFR.

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” “antibody fragment,” and similar terms refer to that portion of an antibody, which comprises the amino acid residues that interact with an antigen and confer on the binding agent its specificity and affinity for the antigen (e.g., the CDRs). Examples of antigen-binding fragments include, but are not limited to, Fab, Fab′, F(ab′)2, Fv, single chain antibody molecules (e.g., scFv), disulfide-linked Fvs (dsFv), disulfide-linked scFv (dsscFv), Fd fragments, diabodies, tribodies, tetrabodies, minibodies, dual variable domain antibodies (DVD), single variable domain antibodies (e.g., camelid antibodies, alpaca antibodies), single variable domain of heavy chain antibodies (VHH), and multispecific antibodies formed from antibody fragments.

Unless otherwise specifically indicated herein, light chain variable region (VLAb) as used herein encompasses all the light chain variable region subtypes, including for example kappa (κ) light chain variable region (KVLAb) and/or lambda (λ) light chain variable region (λVLAb). Unless otherwise specifically indicated herein, heavy chain variable region (VHAb) as used herein encompasses all the heavy chain variable region subtypes, including for example y, 8, a, u and/or & heavy chain variable regions. In some embodiments, VLAb is followed by a Arabic numeral to label the different VLAb. In some embodiments, VHAb is followed by a Arabic numeral to label the different VHAb.

As used herein, the term “antibody mimetic” refers molecules that, like antibodies, can specifically bind antigens, but that are not structurally related to antibodies. The antibody mimetics are usually artificial peptides with in a molar mass of about 2 to 20 kDa. Nucleic acids and small molecules are sometimes considered antibody mimetics as well. Antibody mimetics known in the art including affibodies, affilins, affimers, affitins, alphabodies, anticalins, aptamers, avimers, DARPins, Fynomers, Kunitz domain peptides, monobodies, and nanoCLAMPs.

As used herein, the term “antagonist,” when used in reference to a function of an antigen, is intended to mean a molecule that is capable of inhibiting, decreasing, attenuating, reducing, or otherwise completely blocking one or more of the biological activities or functions of the antigen. An antagonist of a function of an antigen includes a molecule that can block, inhibit, attenuate, or reduce the antigen-mediated or antigen-dependent signaling in a cell expressing the antigen. An antagonist of a function of an antigen also includes a molecule that can block, inhibit, attenuate, or reduce antigen signaling, including downstream signaling induced by ligation or engagement between the antigen and its ligand. In some examples, an antagonist of an antigen further includes molecules that can block, inhibit, attenuate, or reduce the antigen binding to a natural antigen-binding molecule. In other examples, an antagonist of an antigen additionally includes molecules that can block, inhibit, or reduce the antigen binding to a ligand of the antigen. An “antagonist” of an antigen is “antagonistic” to the antigen function. In some embodiments, provided herein are antagonistic co-binders. In some embodiments, provided herein are co-binders that are EGFR antagonist.

A “blocking” co-binder, a “neutralizing” co-binder, or an “antagonist” co-binder when used in reference to a function of an antigen, is intended to mean a co-binder that binds to the antigen and act as an antagonist to the activities or functions of the antigen. For example, blocking co-binders or antagonist co-binders may substantially or completely inhibit the biological activity of an antigen or the binding of the antigen to its ligand. In some embodiments, provided herein are blocking co-binders. In some embodiments, provided herein are EGFR blocking co-binders.

The terms “binds” or “binding” refer to an interaction between molecules including, for example, a binding molecule (e.g. a co-binder or a binding moiety) and a target molecule to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single binding molecule and a single epitope of a target molecule is the affinity of the binding molecule or binding moiety for that epitope. The ratio of dissociation rate (k_{off}) to association rate (kₒₙ) of a binding molecule to a monovalent antigen (k_{off}/kₒₙ) is the dissociation constant K_{D}, which is inversely related to affinity. The lower the K_{D} value, the higher the affinity of the antibody. The value of K_{D} varies for different complexes of the binding molecule and the target molecule and depends on both kₒₙ and k_{off}. The dissociation constant K_{D} for a binding molecule provided herein can be determined using any method provided herein or any other method well known to those skilled in the art. The affinity at one binding site does not always reflect the true strength of the interaction between a binding molecule and a target molecule. When a target molecule containing multiple epitopes come in contact with a binding molecule containing multiple binding moieties that bind the target molecule, the interaction of the binding molecule with the target molecule at one site will increase the probability of a reaction at a second site. The strength of such multiple interactions between a multivalent binding molecule and a target molecule is called the avidity. The avidity of a binding molecule can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The term “specifically binds” as used herein refers to a binding molecule or a binding moiety that interacts more frequently, interacts more rapidly, interacts with longer duration, interacts with greater affinity, interacts with greater strength, dissociate less frequently, dissociation less rapidly, or dissociate for shorter duration, or some combination or permutation of the above to a particular epitope or target molecule than with alternative substances. A binding molecule (e.g. a co-binder, a binding moiety, an antibody or antigen binding fragments thereof) that specifically binds a target molecule (e.g. antigen) can be identified, for example, by immunoassays, radioimmunoassays (RIA), enzyme linked immunosorbent assays (ELISAs), SPR (e.g., Biacore), or other techniques known to those of skill in the art. Typically a specific reaction will be at least twice background signal or noise and can be more than 10 times background. See, e.g., Paul, ed., 1989, Fundamental Immunology Second Edition, Raven Press, New York at pages 332-336 for a discussion regarding antibody specificity. A binding molecule (e.g. a co-binder, a binding moiety, an antibody or antigen binding fragments thereof) that specifically binds a target molecule can bind the target molecule at a higher affinity than its affinity for a different molecule. In some embodiments, a binding molecule (e.g. a co-binder, a binding moiety, an antibody or antigen binding fragments thereof) that specifically binds a target molecule can bind the target molecule with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different molecule. In some embodiments, a binding agent that specifically binds a particular target molecule binds a different molecule at such a low affinity that binding cannot be detected using an assay described herein or otherwise known in the art. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular target molecule or an epitope on a particular target molecule as used herein can be exhibited, for example, by a molecule having a K_{D} for the target of at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, alternatively at least about 10⁻¹³ M, alternatively at least about 10⁻¹⁴ M, alternatively at least about 10⁻¹⁵ M or lower. In one embodiment, the term “specific binding” refers to binding where a binding molecule binds to a particular target molecule or epitope on a particular target molecule without substantially binding to any other polypeptide or polypeptide epitope.

As used herein, the term “bispecific antibody” refers to an antibody that is at least bispecific, namely, capable of binding to two different antigens or target molecules. A bispecific antibody has at least two different antigen binding sites, wherein the first antigen binding site binds to a first antigen or target molecule, and the second antigen binding site binds to a second antigen or target molecule. Among other things, bispecific antibodies can bind to different surface molecules of two different cells, bringing these cells into close proximity. For example, bispecific antibodies that recognize both an antigen on target cells (e.g. FLT3 or CD19 on leukemia cells, the CSPG4-antigen on melanoma cells or EGFR on glioblastoma cells) and the antigen specific T cell receptor (TCR)/CD3-complex, can target the tumor cell for T cell mediated lysis.

As used herein, the term “linker” refers to a molecule that connects two binding moieties through either a covalent bond or noncovalent binding. As such, a peptide linker is an intervening peptide sequence that does not include amino acid residues from either the C-terminus of the variable region (e.g. variable light chain or variable heavy chain) of the first binding moiety or the N-terminus of the variable region (e.g. variable light chain or variable heavy chain) of the second binding moiety. As a linker “links” two binding moieties, the linkage with each binding moiety can be either a covalent bond or noncovalent binding. Specifically, the two linkages of a linker with two binding moieties can be covalent and covalent, covalent and non-covalent, or non-covalent and non-covalent. In some embodiments, the linker of a co-binder facilitates the co-binder to achieve binding interaction to its target molecule. In some embodiment, the linker does not interfere with the binding interaction of the first and the second binding moieties to their respective epitopes in an antigen. In some embodiments, the length of the linker is minimized to reduce or minimize the entropy loss upon binding. In some embodiments, the rigidity of the linker is enhanced or maximized to reduce or minimize the entropy loss upon binding. The linker can be a “non-cleavable” linker. The linker can be a “cleavable linker,” which can be cleaved under various physiological or nonphysiological conditions. Such cleavable linkers include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37). The linker can be made of different composition or chemistry. In some embodiments, the linker is a polypeptide linker, nucleic acid linker and/or chemical linker. In some embodiments, linkers are not antigenic and do not elicit an immune response. The linkers can connect the variable region of the first antibody that is part of the first binding moiety and the variable region of the second antibody that is part of a second binding moiety through covalent bonds. The linkers can also connect the variable region of the first antibody that is part of the first binding moiety and the variable region of the second antibody that is part of a second binding moiety through noncovalent binding. Some examples of polypeptide linkers are described in Chen et al., Adv Drug Deliv Rev. 2013 Oct. 15; 65 (10): 1357-1369, which is incorporated herein by reference in its entirety.

An “isolated” antibody is substantially free of cellular material or other contaminating proteins from the cell or tissue source and/or other contaminant components from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 25%, 20%, 15%, 10%, 5%, or 1% (by dry weight) of heterologous protein (also referred to herein as a “contaminating protein”). In certain embodiments, when the antibody is recombinantly produced, it is substantially free of culture medium, e.g., culture medium represents less than about 20%, 15%, 10%, 5%, or 1% of the volume of the protein preparation. In certain embodiments, when the antibody is produced by chemical synthesis, it is substantially free of chemical precursors or other chemicals, for example, it is separated from chemical precursors or other chemicals that are involved in the synthesis of the protein. Accordingly such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and may include enzymes, hormones, and other proteinaceous or nonproteinaceous solutes. In certain embodiments, the antibody will be purified (1) to greater than 95% by weight of antibody as determined by the Lowry method (Lowry et al., 1951, J. Bio. Chem. 193:265-75), such as 96%, 97%, 98%, or 99%, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step. In specific embodiments, antibodies provided herein are isolated.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994).

The term “variable region,” “variable domain,” “V region,” or “V domain” refers to a portion of the light or heavy chains of an antibody that is generally located at the amino-terminal of the light or heavy chain and has a length of about 110 to 140 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHo.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Antibody fragments” comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50:98-101; and Streltsov et al., 2004, Proc Natl Acad Sci USA. 101:12444-49); and multispecific antibodies formed from antibody fragments.

A “functional fragment,” “binding fragment,” or “antigen-binding fragment” of a therapeutic antibody will exhibit at least one if not some or all of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only an epitope of a target as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002). Exemplary methods of producing monoclonal antibodies are provided in the Examples herein.

The term “native” when used in connection with biological materials such as nucleic acid molecules, polypeptides, host cells, and the like, refers to those which are found in nature and not manipulated, modified, and/or changed (e.g., isolated, purified, selected) by a human being.

The antibodies provided herein can include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

A “human antibody” is one that possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1:755-68). Also available for the preparation of human monoclonal antibodies are methods described in Cole et al., Monoclonal Antibodies and Cancer Therapy 77 (1985); Boerner et al., 1991, J. Immunol. 147 (1): 86-95; and van Dijk and van de Winkel, 2001, Curr. Opin. Pharmacol. 5:368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6 (5): 561-66; Brüggemann and Taussing, 1997, Curr. Opin. Biotechnol. 8 (4): 455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., 1997, J. Biol. Chem. 252:6609-16; Kabat, 1978, Adv. Prot. Chem. 32:1-75). CDR region sequences also have been defined structurally by Chothia as those residues that are not part of the conserved β-sheet framework, and thus are able to adapt different conformations (Chothia and Lesk, 1987, J. Mol. Biol. 196:901-17). Both terminologies are well recognized in the art. CDR region sequences have also been defined by AbM, Contact, and IMGT. The positions of CDRs within a canonical antibody variable region have been determined by comparison of numerous structures (A1-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (A1-Lazikani et al., supra). Such nomenclature is similarly well known to those skilled in the art.

The term “hypervariable region,” “HVR,” or “HV,” when used herein refers to the regions of an antibody variable region that are hypervariable in sequence and/or form structurally defined loops. Generally, antibodies comprise six hypervariable regions, three in the VH (H1, H2, H3) and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. The residues from each of these hypervariable regions or CDRs are noted below.

Recently, a universal numbering system has been developed and widely adopted, ImMunoGeneTics (IMGT) Information System® (Lafranc et al., 2003, Dev. Comp. Immunol. 27 (1): 55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHo) has been developed by Honegger and P1ückthun, 2001, J. Mol. Biol. 309:657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra).

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 53-55 | 47-58 |
| V_{H} CDR3 | 105-117 | 95-102 | 95-102 | 96-101 | 93-101 |
| V_{L} CDR1 | 27-38 | 24-34 | 24-34 | 26-32 | 30-36 |
| V_{L} CDR2 | 56-65 | 50-56 | 50-56 | 50-52 | 46-55 |
| V_{L} CDR3 | 105-117 | 89-97 | 89-97 | 91-96 | 89-96 |

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH. As used herein, the terms “HVR” and “CDR” are used interchangeably.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

An “affinity matured” antibody is one with one or more alterations (e.g., amino acid sequence variations, including changes, additions, and/or deletions) in one or more HVRs thereof which result in an improvement in the affinity of the antibody for antigen, compared to a parent antibody which does not possess those alteration(s). Affinity matured antibodies can have nanomolar or even picomolar affinities for the target antigen. Affinity matured antibodies are produced by procedures known in the art. For review, see Hudson and Souriau, 2003, Nature Medicine 9:129-34; Hoogenboom, 2005, Nature Biotechnol. 23:1105-16; Quiroz and Sinclair, 2010, Revista Ingeneria Biomedia 4:39-51.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using surface plasmon resonance assays by Biacore®, using, for example, a Biacore®™-2000 or a Biacore®™-3000, or by biolayer interferometry using, for example, the Octet® QK384 system. An “on-rate” or “rate of association” or “association rate” or “kₒₙ” may also be determined with the same surface plasmon resonance or biolayer interferometry techniques described above using, for example, a Biacore®™-2000 or a Biacore®™-3000, or the Octet® QK384 system. An “off-rate” or “rate of dissociation” or “dissociation rate” or “k_{off}” may also be determined with the same surface plasmon resonance or biolayer interferometry techniques described above using, for example, a Biacore®™-2000 or a Biacore®™-3000, or the Octet® QK384 system.

The term “effective amount” as used herein refers to the amount of a co-binder or pharmaceutical composition provided herein which is sufficient to result in beneficial or desired outcome. An effective amount can be administered in one or more administrations, applications or dosages. Such delivery is dependent on a number of variables including the time period for which the individual dosage unit is to be used, the bioavailability of the agent, the route of administration, etc.

As used herein, the term “therapeutically effective amount” refers to the amount of a therapeutic agent (e.g., a co-binder as provided herein) which is sufficient to reduce and/or ameliorate the severity and/or duration of a given disease and/or a symptom related thereto. A therapeutically effective amount of a therapeutic agent can be an amount necessary for the reduction or amelioration of the advancement or progression of a given disease, reduction or amelioration of the recurrence, development or onset of a given disease, and/or to improve or enhance the prophylactic or therapeutic effect of another therapy (e.g., a therapy other than the administration of the co-binders provided herein).

The term “variant” when used in relation to polypeptide refers to a polypeptide comprising one or more (such as, for example, about 1 to about 50, about 1 to about 45, about 1 to about 40, about 1 to about 35, about 1 to about 30, about 1 to about 25, about 1 to about 20, about 1 to about 18, about 1 to about 15, about 1 to about 10, or about 1 to about 5) amino acid sequence substitutions, deletions, and/or additions as compared to a native or unmodified sequence of the polypeptide. For example, a variant of co-binder may results from one or more (such as, for example, about 1 to about 25, about 1 to about 20, about 1 to about 18, about 1 to about 15, about 1 to about 10, or about 1 to about 5) changes to an amino acid sequence of a native or previously unmodified co-binder. A variant may be constructed by molecular cloning technologies known to a person of ordinary skill in the art, for example, random mutagenesis or site directed mutagenesis. A variant may be prepared from the corresponding nucleic acid molecules encoding the variants. In specific embodiments, the variants of a co-binder retains the functional properties or activities of the co-binder (e.g. binding, agonist, antagonist, blocking, neutralizing, and/or activating activities/properties). In specific embodiments, a variant is encoded by a nucleic acid molecule including one or more single nucleotide polymorphism (SNP) in one or more regions or subregions of the co-binder, such as one or more CDRs.

The term “vector” refers to a substance that is used to carry or include a nucleic acid sequence, including for example, a nucleic acid sequence encoding a co-binder as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a co-binder as described herein), and it is further understood that expression levels can be optimized to obtain sufficient expression using methods well known in the art.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions can be made in accordance with those set forth in Table 1 and description below. In a conservative amino acid substitution, an amino acid residue is replaced with an amino acid residue comprising a side chain with a similar charge or a side chain with similar property. Families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acids can also be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also may be substituted, for example, with another amino acid, such as alanine or serine, to improve the oxidative stability of the molecule and to prevent aberrant crosslinking. In certain embodiments, conservative substitutions include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine(S) for threonine (T) and vice versa. Other substitutions can also be considered conservative, depending on the environment of the particular amino acid and its role in the three-dimensional structure of the protein. For example, glycine (G) and alanine (A) can be interchangeable, as can alanine (A) and valine (V). Methionine (M), which is relatively hydrophobic, can be interchanged with leucine and isoleucine, and sometimes with valine. Lysine (K) and arginine (R) can be interchangeable in locations in which the significant feature of the amino acid residue is its charge and the differing pK's of these two amino acid residues are not significant. Still other changes can be considered “conservative” in particular environments (see, e.g. Table 1 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270 (20): 11882-11886). Other substitutions are also permissible and may be determined empirically or in accord with known conservative substitutions.

TABLE 1
Amino Acid Substitution or Similarity Matrix
Adapted from the GCG Software 9.0 BLOSUM62 amino acid
substitution matrix (block substitution matrix). The higher the value,
the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence aligned for purposes of comparison. If a given position of two polypeptide sequences is not identical, the similarity or conservativeness of that position can be determined by assessing the similarity of the amino acid of the position, for example, according to Table 1, according to the similarity in the charges of the side chain as described above, or according to the similarity in the properties of the side chain as described above. A degree of similarity between sequences is a function of the number of matching (identical) or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “truncation” when used in the context of a polypeptide/protein refers to a shortening in the amino acid sequence of a polypeptide from either end of the polypeptide sequence, the algorithm for determining which is provided further below and in the several paragraphs following the paragraph starting with the sentence “[i]n certain embodiments of the co-binders provided herein, the disclosure provides that the truncation or deletion in the VR2, VLAb2, VHAb2, or the second binding moiety is determined, for example, by the following exemplary process”. Similarly, the term “truncation” when used in the context of a nucleic acid refers to a shortening in the nucleotide sequence of a nucleic acid from either 5 prime end or 3 prime end of the nucleotide sequence. An N-terminal truncation or a truncation from the N-terminus of a polypeptide/protein truncation refers to the shortening of the polypeptide/protein sequences from the N-terminal end, i.e. N terminus, of the polypeptide/protein. Similarly, a C-terminal truncation or a truncation from the C-terminus of a polypeptide/protein truncation refers to the shortening of the polypeptide/protein sequences from the C-terminal end, i.e. C-terminus, of the polypeptide/protein. A truncation can be a shortening of one or a plurality of amino acids from either end or both ends of the polypeptide/protein. For example, a truncation can be a shortening of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids from either the N-terminal end or the C-terminal end of the polypeptide/protein. For example, a truncation can be a shortening of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids from both the N-terminal end and the C-terminal end of the polypeptide/protein. A protein truncation can be the result of a truncation in the nucleic acid sequence encoding the protein, a substitution or other mutation that creates a premature stop codon without shortening the nucleic acid sequence, or from alternate splicing of RNA in which a substitution or other mutation that does not itself cause a truncation results in aberrant RNA processing. A “truncation mutant” or a “truncation mutation” refers a variant that have a truncation of one or more amino acids in the context of polypeptides/proteins or a truncation of one or more nucleotides in the context of nucleic acids.

As used herein, the term “deletion” when used in the context of a polypeptide/protein refers to a removal of one or more amino acids from the sequence of the polypeptide/protein. The removed one or more amino acids can be a continuous sequence, i.e. a continuous part, of the polypeptide/protein, or can be interspersed in the sequence of the polypeptide/protein. A deletion can be an internal deletion, in which none removed one or more amino acids is the N-terminal or the C-terminal amino acid of the sequence of the polypeptide/protein. A deletion can also be a deletion from the N-terminal end (N-terminal deletion) or a deletion from the C-terminal end (C-terminal deletion), in which a sequence of one or more amino acids continuous from the N-terminal end or the C-terminal end of the polypeptide/protein are removed. A deletion can also be a deletion including an internal deletion, a N-terminal deletion, and/or a C-terminal deletion. As is clear from the description, a N-terminal deletion is also an N-terminal truncation and a C-terminal deletion is also an C-terminal truncation. A sequence meeting the definition of an internal deletion may also be considered as an N-terminal truncation described herein, if the criteria for N-terminal truncation is satisfied by applying the algorithm described herein.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain a co-binder, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a co-binder of the present disclosure may include a parent hybridoma cell, as well as bacterial and eukaryotic host cells into which nucleic acids encoding the antibodies have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “recombinant antibody,” “recombinant co-binder,” or “recombinant polypeptide/protein,” refers to an antibody, a co-binder, a polypeptide/protein, that is prepared, expressed, created, or isolated by recombinant means. For example, recombinant co-binders can be co-binders expressed using a recombinant expression vector transfected into a host cell, co-binders isolated from a recombinant, combinatorial library, or co-binders prepared, expressed, created, or isolated by any other means that involves splicing of immunoglobulin gene sequences to other DNA sequences. For a further example, recombinant polypeptides/proteins can be polypeptides/proteins expressed using a recombinant expression vector transfected into a host cell, polypeptides/proteins isolated from a recombinant, combinatorial library, or polypeptides/proteins prepared, expressed, created, or isolated by any other means that involves splicing of immunoglobulin gene sequences to other DNA sequences. For another example, recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see, e.g., Taylor et al., 1992, Nucl. Acids Res. 20:6287-95), or antibodies prepared, expressed, created, or isolated by any other means that involves splicing of immunoglobulin gene sequences to other DNA sequences. Such recombinant antibodies can have variable and constant regions, including those derived from human germline immunoglobulin sequences (See Kabat et al., supra). In certain embodiments, however, such recombinant antibodies may be subjected to in vitro mutagenesis (or, when an animal transgenic for human Ig sequences is used, in vivo somatic mutagenesis), thus the amino acid sequences of the VH and VL regions of the recombinant antibodies are sequences that, while derived from and related to human germline VH and VL sequences, may not naturally exist within the human antibody germline repertoire in vivo.

As used herein, the term “therapeutic agent” refers to an agent that can be used in the treatment, management or amelioration of a disease and/or a symptom related thereto. In certain embodiments, a therapeutic agent comprises the co-binder as described herein.

As used herein, the term “diagnostic agent” refers to a substance that aids in the diagnosis of a disease. A diagnostic agent can be used in vitro or in vivo. In some embodiments, a diagnostic agent is used in in vitro assays. In some embodiments, a diagnostic agent is administered to a subject. Such agents can be used to reveal, pinpoint, and/or define the localization of a disease causing process. In some embodiments, a diagnostic agent when administered to a subject or contacted to a sample from a subject aids in the diagnosis of cancer or tumor formation. In certain embodiments, a diagnostic agent comprises the co-binders as described here.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human.

“Substantially all” refers to at least about 60%, at least about 65%, at least about 70%, at least about 75%, at least about 80%, at least about 85%, at least about 90%, at least about 95%, at least about 98%, at least about 99%, or about 100%.

The terms “detectable agent” or “detectable molecule” are used interchangeably herein and refers to a substance that can be used to ascertain the existence or presence of a desired molecule, such as a co-binder as described herein, in a sample or subject. A detectable agent can be a substance that is capable of being visualized or a substance that is otherwise able to be determined and/or measured (e.g., by quantitation).

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

As used herein, the term “compound” encompasses small organic molecules and inorganic chemicals, which have a molecular weight of less than about 5 kD, less than about 4 kD, less than about 3 kD, less than about 2 kD, less than about 1 kD, or less than about 0.5 kD, including without limitation, all analogs, derivatives, salts, and solvates (for example, hydrates) thereof. In some examples, the compound can include, nucleic acids, peptides, peptidomimetics, peptoids, other small organic compounds or drugs, and the like. Libraries of chemical and/or biological mixtures, such as fungal, bacterial, or algal extracts, are known in the art and can be screened with any of the assays provided herein. Examples of methods for the synthesis of compound libraries can be found in: (Carell et al., 1994a; Carell et al., 1994b; Cho et al., 1993; DeWitt et al., 1993; Gallop et al., 1994; Zuckermann et al., 1994).

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence.

The terms “about” and “approximately” mean within 20%, within 15%, within 10%, within 9%, within 8%, within 7%, within 6%, within 5%, within 4%, within 3%, within 2%, within 1%, or less of a given value or range.

“Administer” or “administration” refers to the act of injecting or otherwise physically delivering a substance as it exists outside the body (e.g., a co-binder as described herein) into a patient, such as by mucosal, intradermal, intravenous, intramuscular delivery, and/or any other method of physical delivery described herein or known in the art.

The term “composition” is intended to encompass a product containing the specified ingredients (e.g., an antibody provided herein) in, optionally, the specified amounts.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the singular forms “a,” “and,” and “the” include plural referents unless the context clearly indicates otherwise. Thus, for example, reference to “a peptide sequence” includes a plurality of such sequences and so forth.

As used herein, numerical values are often presented in a range format throughout this document. The use of a range format is merely for convenience and brevity and should not be construed as an inflexible limitation on the scope of the disclosure unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

### II. Binder Molecules—Components and Configurations Thereof

In some aspects, provided herein is a binder molecule comprising a second binding moiety specifically recognizing a target site, such as a target polypeptide, wherein the second binding moiety is a second antibody moiety comprising an antibody variable domain having an N-terminal truncation (“N-terminal truncated antibody variable domain”). As described herein, the second binding moieties of the binder molecules described herein enable a high affinity binding platform that can include various other components to provide numerous configurations useful for a diverse array of applications. It is to be understood that the term “second binding moiety” does not imply the existence of a separate first binding moiety. In other words, the binder molecule may comprise: 1) a single binding moiety which is the second binding moiety, 2) a first moiety which is not a binding moiety and a second binding moiety; or it may comprise a first binding moiety and a second binding moiety. Similar reasoning applies across other aspects of the description provided herein, e.g., the description of a co-binder as comprising a second antibody moiety does not imply the existence of a separate first antibody moiety.

For example, in some embodiments, the binder molecule comprises a co-binder comprising a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), wherein the first binding moiety is connected to the second binding moiety through N-terminus of the N-terminal truncated antibody variable domain via a linker. In some embodiments, the first binding moiety comprises a first V_{H}H domain, wherein the second binding moiety comprises a second V_{H}H domain having an N-terminal truncation (“truncated V_{H}H domain”), and wherein the C-terminus of the first V_{H}H domain is connected to the N-terminus of the second V_{H}H domain via a linker.

In some embodiments, the binder molecule comprises a first moiety, such as an enzyme, drug, or toxin, wherein the first moiety is connected to the second binding moiety through N-terminus of the N-terminal truncated antibody variable domain via a linker.

In some embodiments, the binder molecule comprises a linker, wherein the second binding moiety is connected to a linker through the N-terminus of the N-terminal truncated antibody variable domain. In some embodiments, the binder molecule does not comprise a linker.

In the following sections, additional description of the various aspects of the binder molecules are provided. Such description in a modular fashion is not intended to limit the scope of the disclosure and based on the teachings provided herein one of ordinary skill in the art will readily appreciate that certain modules can be integrated, at least in part. The section heading used herein are for organizational purposes only and are not to be construed as limiting the subject matter described.

In some embodiments, one or more features of a binder molecule, such as one or more of a FR1, CDR1, VH, or VL are determined according to IMGT numbering scheme or to Kabat numbering scheme.

### A. Second Binding Moieties

The binder molecules provided herein, e.g., a co-binder, comprise a second binding moiety that is a second antibody moiety comprising an antibody variable domain having an N-terminal truncation (“N-terminal truncated antibody variable domain”). As provided herein, the second antibody moiety can take many forms, and description is included to determine N-terminal truncation thereof. In some embodiments, the second binding moiety further comprises another moiety, such as a conjugated label or drug.

### 1. Antibody Moieties of the Second Binding Moiety

Provided herein are antibody moieties comprising an antibody variable domain having an N-terminal truncation (“N-terminal truncated antibody variable domain”). The antibody moieties of a second binding moiety specifically recognize a target site, such as a polypeptide epitope.

In some embodiments, the antibody moiety of a second binding moiety is a variable region (in some embodiments, referred to herein as VR, and optionally, with a numerical identification thereof, e.g., VR2). In some embodiments, the antibody moiety of a second binding moiety is a heavy chain variable region (in some embodiments, referred to herein as VHAb or VH domain). In some embodiments, the heavy chain variable region is associated with a light chain variable region. In some embodiments, wherein the heavy chain variable region associated with the light chain variable region is a single chain, such as an scFv. In some embodiments, the heavy chain variable region is connected to at least one constant domain and/or the light chain variable region is connected to at least one constant domain, e.g., a Fab or scFab. In some embodiments, wherein the heavy chain variable region is associated with a light chain variable region, the heavy chain variable region and the light chain variable region are from the same antibody or antigen binding fragment. In some embodiments, the heavy chain variable region associated with a light chain variable region form a stable complex. In some embodiments, the heavy chain variable region and the light chain variable region associate with each other to form an antigen-binding domain.

In some embodiments, the antibody moiety of a second binding moiety is a light chain variable region (in some embodiments, referred to herein as VLAb or VL domain). In some embodiments, the light chain variable region is a light chain variable region of human lambda (λ) light chain. In some embodiments, the light chain variable region is a light chain variable region of human kappa (κ) light chain. In some embodiments, the light chain variable region is associated with a heavy chain variable region. In some embodiments, wherein the light chain variable region associated with the heavy chain variable region is a single chain, such as an scFv. In some embodiments, the light chain variable region is connected to at least one constant domain and/or the heavy chain variable region is connected to at least one constant domain, e.g., a Fab or scFab. In some embodiments, wherein the light chain variable region is associated with a heavy chain variable region, the light chain variable region and the heavy chain variable region are from the same antibody or antigen binding fragment. In some embodiments, the light chain variable region associated with a heavy chain variable region form a stable complex. In some embodiments, the light chain variable region and the heavy chain variable region associate with each other to form an antigen-binding domain.

In some embodiments, the antibody moiety of a second binding moiety further comprises one or more constant domains, such as any one or more of CH1, CH2, CH3, or CL.

In some embodiments, the antibody moiety of a second binding moiety is a V_{H}H domain. In some embodiments, the antibody moiety of a second binding moiety is selected from the group consisting of a Fab, Fv, scFv, dsFv, Fab′, and (Fab′)2 fragment. In some embodiments, the antibody moiety of a second binding moiety is a single domain antibody.

In some embodiments, the N-terminal truncated antibody variable domain of a second binding moiety is a truncated variable region. In some embodiments, the N-terminal truncated antibody variable domain of a second binding moiety is a truncated heavy chain variable region. In some embodiments, the N-terminal truncated antibody variable domain of a second binding moiety is a truncated heavy chain variable region associated with a light chain variable region. In some embodiments, the N-terminal truncated antibody variable domain of a second binding moiety is a truncated light chain variable region. In some embodiments, the N-terminal truncated antibody variable domain of a second binding moiety is a truncated light chain variable region associated with a heavy chain variable region. In some embodiments, the N-terminal truncated antibody variable domain of a second binding moiety is a truncated V_{H}H domain. In some embodiments, the N-terminal truncated antibody variable domain of a second binding moiety is a truncated Fab, Fv, scFv, dsFv, Fab′, or (Fab′)2 fragment. In some embodiments, the N-terminal truncated antibody variable domain of a second binding moiety is a truncated single domain antibody.

The second binding moieties, or at least a portion thereof, provided herein may be obtained or derived from a variety of sources. For example, in some embodiments, the second binding moiety, or at least a portion thereof, is obtained or derived from a camelid, such as a camelid single chain V_{H}H.

In some embodiments, the second binding moiety, or at least a portion thereof, is obtained or derived from an affibody, affilin, affimer, affitin, alphabody, anticalin, aptamer, avimer, DARPin, Fynomer, Kunitz domain peptide, monobody, nanobody (also referred to as a single-domain antibody, sdAb), or nanoCLAMP. In some embodiments, the second binding moiety, or at least a portion thereof, is obtained or derived from an IgG, IgA, IgE, IgM, or IgD.

In some embodiments, the second binding moiety, or at least a portion thereof, is obtained or derived from a mammal, including a camelid, human, non-human primate (such as a monkey), domestic, farm, or zoo animal, such as a dog, horse, rabbit, cow, pig, hamster, gerbil, mouse, ferret, rat, or cat. In some embodiments, the second binding moiety, or at least a portion thereof, is obtained or derived from a synthetic source.

The antibody moieties of a second binding moieties provided herein specifically recognize a target site. Said target sites encompass a diverse array of epitopes, including on polypeptides, nucleic acids, and small molecules.

### 2. Truncations and Determinations Thereof

In certain aspects, the second binding moiety described herein is a second antibody moiety comprising an antibody variable domain having an N-terminal truncation (“N-terminal truncated antibody variable domain”).

In some embodiments, the truncation of a second binding moiety is a truncation of any of 1, 2, 3, 4, 5, 6, 7, 8, 9, 10, 11, 12, 13, 14, 15, 16, 17, 18, 19, or 20 amino acids. In some embodiments, the N-terminal truncation of the second binding moiety is a truncation in the framework region 1 (FR1) of the second binding moiety. In some embodiments, the second binding moiety comprises a V_{H}H comprising a N-terminal truncation in the framework region 1 (FR1) of the second binding moiety of any of 1, 2, 3, 4, 5, 6, 7, 8, 9, 10, 11, 12, 13, 14, 15, 16, 17, 18, 19, or 20 amino acids.

In some embodiments, the X₃ amino acid of a polypeptide linker and the start of the complementarity determining region 1 (CDR1), as characterized by the first amino acid of the CDR1 on the N-terminal amino acid side of the CDR1, of a second antibody moiety (or the N-terminal amino acid of the second antibody moiety) are separated by no more than 25 amino acids, such as no more than any of 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, or 3 amino acids.

An N-terminal truncation or a truncation from the N-terminus of a polypeptide/protein refers to the shortening of the polypeptide/protein sequences from the N-terminal end, i.e. N terminus, of the polypeptide/protein. For an antibody variable domain (e.g., the second antibody moiety) comprised in a binder molecule, the N-terminal truncation of the antibody variable domain is determined based on comparison with a full length antibody variable domain. The FR1 region of an antibody variable domain is very well-conserved, and whether a polypeptide comprises an antibody variable domain with an N-terminal truncation can be readily determined by methods known in the art. For example, the corresponding positions of amino acids (“numbered amino acids”) in a polypeptide comprising an antibody variable domain can first be determined by aligning the polypeptide sequence with a full length antibody variable domain or according to any of the well-established variable region residue numbering systems such as Kabat, IMGT, EU numbering system, AbM, Chothia, Contact, and AHo. A number of computer algorithm have been developed and available from internet to a person of ordinary skill in the art to input the sequence and obtain the sequence numbered according to any one of the specified numbering schemes provided herein. Such exemplary tools include: Antigen receptor Numbering And Receptor ClassificatIon (ANARCI, opig.stats.ox.ac.uk/webapps/newsabdab/sabpred/anarci/; described in Dunbar et al., Bioinformatics. 2016 Jan. 15; 32 (2): 298-300, which is incorporated herein by reference in its entirety), abYsis online or standalone tool developed by Prof. Andrew C. R. Martin (bioinf.org.uk/abs/; abysis.org/), AHo's Amazing Atlas of Antibody Anatomy (AAAAA; bioc.uzh.ch/antibody; described in A. Honegger & A. P1ückthun. J. Mol. Biol, 309 (2001) 657-670, which is incorporated herein by reference in its entirety). Second, each numbered amino acid of the co-binder (which includes the antibody variable domain sequence and possibly a portion of the linker sequence) is compared to amino acids occurring naturally over certain frequency at the corresponding numbered position under the same numbering scheme. If the amino acid at position No. 1 in the numbered amino acids of the co-binder occurs at a frequency of no more than about 3% for naturally occurring antibody variable domains, the antibody variable domain in the co-binder is deemed to have a truncation at the first N-terminal amino acid, and the amino acid at position No. 1 in the numbered amino acid would be deemed to be part of the linker sequence. Similarly, if the amino acids at position Nos. 1 and 2 in the numbered amino acids of the co-binder occur at a frequency of no more than about 3% for naturally occurring antibody variable domains, the antibody variable domain in the co-binder is deemed to have a truncation at the first and second N-terminal amino acids (i.e., the N-terminal truncation of the N-terminal truncated antibody variable domain is 2 amino acids), and the amino acids at position Nos. 1 and 2 in the numbered amino acids would be deemed to be part of the linker sequence. If the amino acids at position Nos. 1, 2, and 3 in the in the numbered amino acids of the co-binder occur at a frequency of no more than about 3% for naturally occurring antibody variable domains, the antibody variable domain in the co-binder is deemed to have a truncation at the first, second, and third N-terminal amino acids (i.e., the N-terminal truncation of the N-terminal truncated antibody variable domain is 3 amino acids), and the amino acids at position Nos. 1, 2, and 3 in the numbered amino acids would be deemed to be part of the linker sequence. This comparison is performed iteratively for N-terminal N positions of amino acids. If the amino acids at position Nos. 1, 2, 3, . . . and N in the in the numbered amino acids of the co-binder occur at a frequency of no more than about 3% for naturally occurring antibody variable domains, the antibody variable domain in the co-binder is deemed to have a truncation at the first, second, third, and Nth N-terminal amino acids (i.e., the N-terminal truncation of the N-terminal truncated antibody variable domain is N amino acids), and the amino acid at position Nos. 1-N in the numbered amino acids would be deemed to be part of the linker sequence. In some embodiments, the N-terminal truncation is determined using the ANARCI program (see Dunbar et al., Nucleic Acids Res, 44, 2016). In some embodiments, the N-terminal truncation is determined using the abYsis program (e.g., version 3.4.1; see also Swindells et al., J Mol Biol, 429, 2017). In some embodiments, the N-terminal truncation is determined using the AAAAA program (see Honegger & Pluckthun, J Mol Biol, 309, 2001). Alternatively or additionally, the N-terminal truncation of an antibody variable domain (e.g., the second antibody moiety) comprised in a binder molecule can be determined (or confirmed) by modeling the tertiary structure of the second binding moiety and optionally neighboring residues. A shortened beta sheet structure relative to a corresponding full length FR1 region in a wildtype antibody moiety (e.g., V_{H}H) is indicative of the existence of an N-terminal truncation. Various computer programs for modeling antibody tertiary structures are well-known in the art, for example Alphafold (see Jumper et al., Nature, 596, 2021).

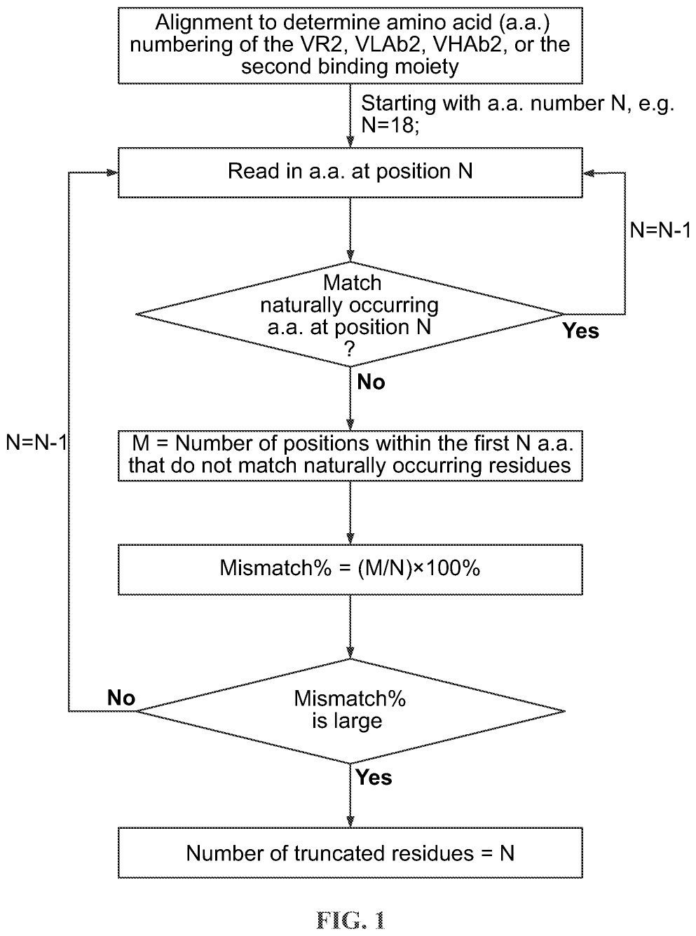

Because the second binding moiety is typically preceded by other amino acid sequences (e.g., linker sequences), the existence of an N-terminal truncation in the second binding moiety may not be readily apparent by visually examining amino acid sequence alignments. Under such circumstances, the truncation in a second binding moiety may be determined, for example, by the following exemplary process. First, the amino acid sequence of the binder molecule (or a portion thereof comprising the second binding moiety and neighboring amino acid residues) is aligned with the amino acid sequence of an immunoglobulin protein (such as an isotype of an immunoglobulin (Ig) family to which the second binding moiety belongs). Second, each amino acid of the sequences of the second binding moiety is then numbered according to the position number of the Ig isotype's amino acid that the second binding moiety aligned to (FIG. 1). Then each numbered amino acid is compared to the amino acids occurring naturally or occurring naturally over certain frequency from the Ig family at that numbered position. In some embodiments, such comparison is made with amino acids occurring naturally at a frequency of over 1%, such as over any of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, at the same numbered position from the Ig family. This comparison is performed iteratively for N-terminal N positions of amino acids in the second binding moiety of a binder molecule (FIG. 1). In some embodiments, N is any of 1, 2, 3, 4, 5, 6, 7, 8, 9, 10, 11, 12, 13, 14, 15, 16, 17, 18, 19, 20, 21, 22, 23, 24, or 25.

Based on the comparison completed for determining truncation, if an amino acid at a numbered position is different from the naturally amino acids of the Ig family in the corresponding position, that numbered position is a mismatch in the second binding moiety of the binder molecule, and the mismatched amino acid is defined as a deleted or missing amino acid (since the naturally occurring amino acid is missing at that position) in the second binding moiety of the binder molecule. The number of mismatches or deletions within the first N amino acids is calculated as M=number of positions within the first N amino acids that do not match naturally occurring residues. The percentage of mismatch (“Mismatch %”) is calculated as (M/N)×100%, which is the percentage converted from the ratio of number of positions within the first N amino acids that do not match naturally occurring amino acids against the number N. When the Mismatch % for the N-terminal N amino acids is over a certain threshold, according to the disclosure provided herein, the N-terminal N amino acids have been truncated. In some embodiments, the certain threshold of the Mismatch % is at least about 50%, such as at least about any of 55%, 60%, 65%, 70%, 75%, 80%, 85%, 90%, 95%, or 99%. In some embodiments, when the Mismatch % for the N-terminal N amino acids is at least 50%, such as at least any of 55%, 60%, 65%, 70%, 75%, 80%, 85%, 90%, 95%, or 99%, the disclosure provides that the N-terminal N amino acids have been truncated. In some embodiments, when the Mismatch % for the N-terminal N amino acids is 100%, the disclosure provides that the N-terminal N amino acids have been truncated. In some embodiments, when the Mismatch % for the N-terminal N amino acids is 50% or more, the N-terminal N amino acids have been truncated. Without being bound by theory, the inventors believe that 50% or more mismatches for the N-terminal N amino acids correlates with a disruption of the beta sheet structure at the N-terminal N-amino acid of the second binding moiety. The presence of an N-terminal truncation can therefore be further confirmed by a shortened beta sheet relative to a corresponding full length FR1 region in a wildtype antibody moiety through structural analysis.

The flow chart in FIG. 1 illustrates the iterative process of determining the total number of amino acids missing, deleted, and/or truncated from the second binding moiety of a binder molecule. In some embodiments, to determine the amino acid truncation, the alignment is performed between the sequence of a binder molecule or a portion thereof, for example the sequence of the second binding moiety, and one or more of the sequences of the framework 1 region (FR1, framework region 1) of an isotype Ig as listed in Table 3, Table 4, and Table 5 (which can be found in the section titled Certain Tables). In some embodiments, to determine the amino acid truncation, the alignment is performed between the sequence of the binder molecule or a portion thereof, for example the sequence of the second binding moiety, and one or more of the sequences of isotype Ig, which sequences are disclosed in the database according to the database identifiers listed in the left column of Table 3, Table 4, and Table 5, and which sequences are incorporated herein by reference. In some embodiments, to determine the amino acid truncation, the alignment is performed between the sequence of a binder molecule or a portion thereof, for example the sequence of the second binding moiety, and one or more of the sequences of the framework 1 region (FR1, framework region 1) of an isotype Ig as listed in Table 3, Table 4, and Table 5 based on the isotype of the binder molecule or the portion thereof.

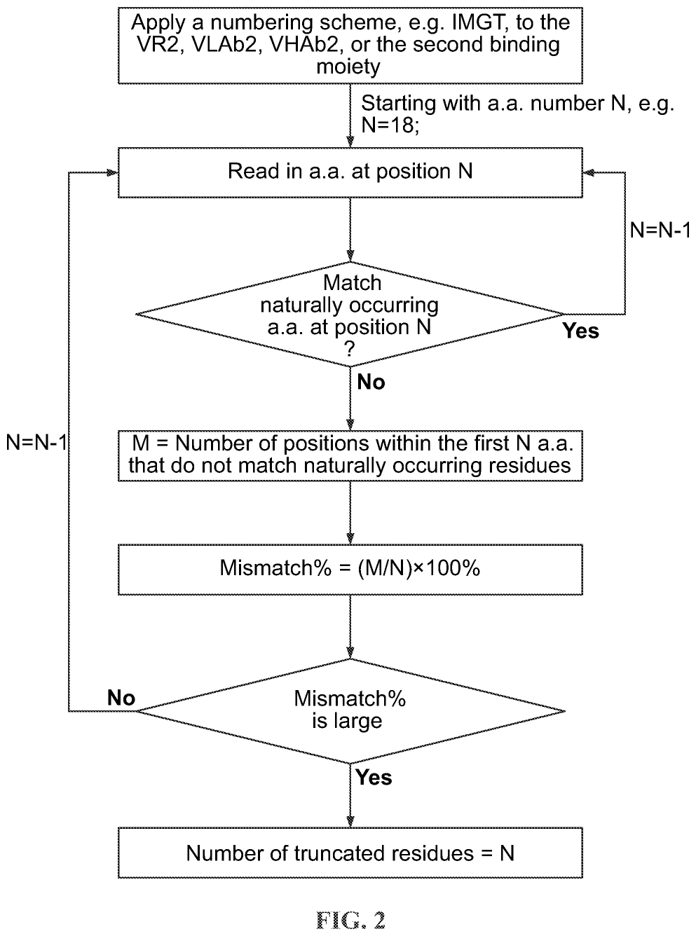

The N-terminal truncation in the second binding moiety can also be determined, for example, by the following additional exemplary process. First, the sequence of the second binding moiety is numbered according to any one of the known antibody numbering scheme, including for example Kabat, Chothia, AbM, Contact, IMGT, or AHo numbering as known to a person of ordinary skill in the art and provided herein (FIG. 2). A number of computer algorithm have been developed and available from internet to a person of ordinary skill in the art to input the sequence and obtain the sequence numbered according to any one of the specified numbering schemes provided herein. Such exemplary tools include: Antigen receptor Numbering And Receptor Classification (ANARCI, opig.stats.ox.ac.uk/webapps/newsabdab/sabpred/anarci/; described in Dunbar et al., Bioinformatics. 2016 Jan. 15; 32 (2): 298-300, which is incorporated herein by reference in its entirety), abYsis online or standalone tool developed by Prof. Andrew C. R. Martin (bioinf.org.uk/abs/; abysis.org/), AHo's Amazing Atlas of Antibody Anatomy (AAAAA; bioc.uzh.ch/antibody; described in A. Honegger & A. Pluckthun. J. Mol. Biol, 309 (2001) 657-670, which is incorporated herein by reference in its entirety). Second, each numbered amino acid of the sequences of the second binding moiety is compared to the amino acids occurring naturally or occurring naturally over certain frequency that numbered position (under the same numbering scheme) from the same Ig family to which the second binding moiety belongs. In some embodiments, such comparison is made with amino acids occurring naturally at a frequency of over any of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% at the same numbered position from the same Ig family to which the second binding moiety belongs. This comparison is performed iteratively for N-terminal N positions of amino acids in the second binding moiety (FIG. 1). In some embodiments, Nis any of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Based on the comparisons described herein, if a second binding moiety at a numbered position is different from the naturally amino acids of the Ig family in the corresponding position, that numbered position is a mismatch in the second binding moiety, and the mismatched amino acid is defined as a deleted or missing amino acid (since the naturally occurring amino acid is missing at that position) in the second binding moiety. The number of mismatches or deletions within the first N amino acids is calculated as M=number of positions within the first N a.a. that do not match naturally occurring residues. The percentage of mismatch (“Mismatch %”) is calculated as (M/N)×100%, which is the percentage converted from the ratio of number of positions within the first N amino acids that do not match naturally occurring amino acids against the number N. When the Mismatch % for the N-terminal N amino acids is over certain threshold, the disclosure provides that the N-terminal N amino acids have been truncated. In one embodiment, when the Mismatch % for the N-terminal N amino acids is at least 20%, such as at least any of 25%, 30%, 35%, 40%, 45%, 50%, 55%, 60%, 65%, 70%, 75%, 80%, 85%, 90%, 95%, or 100%, the disclosure provides that the N-terminal N amino acids have been truncated.

Accordingly, the total number of amino acids missing, deleted, and/or truncated can be determined as described herein. The flow chart in FIG. 2 illustrates the iterative process of for determining the total number of amino acids missing, deleted, and/or truncated from the second binding moiety to classify a second binding moiety as having a “N-terminal truncated antibody variable domain.”

In some embodiments, the naturally occurring frequencies of the amino acids, such as in a second binding moiety, are determined based on the any one or more of the sequences provided in Table 3, Table 4, and Table 5. In some embodiments, the naturally occurring frequencies of the amino acids, such as in a second binding moiety, are determined based on Table 7, Table 9, and/or Table 11.

In some embodiments, the amino acids naturally occurring in a variable heavy chain with over 1% frequency at each position according to an antibody numbering scheme, for example according to the IMGT numbering scheme, are listed in Table 6.

TABLE 6
Naturally occurring amino acids (frequency >1%)
in a variable heavy chain.

| | Naturally occurring amino acids in a |
|---|---|
| IMGT AA # | variable heavy chain (frequency >1%) |
| 1 | E, Q |
| 2 | I, L, M, V |
| 3 | Q, T |
| 4 | L, V |
| 5 | K, L, Q, R, V |
| 6 | E, Q |
| 7 | P, S, W |
| 8 | G |
| 9 | A, G, P, S |
| 10 | |
| 11 | A, E, G, T, V |
| 12 | L, V |
| 13 | I, K, L, R, V |
| 14 | K, Q, R |
| 15 | A, P |
| 16 | G, P, S, T |
| 17 | A, D, E, G, Q, R, S |
| 18 | S, T |

In some embodiments, the amino acids naturally occurring in a variable heavy chain at each position according to an antibody numbering scheme, for example according to the IMGT numbering scheme, and their frequency of occurrence are listed in Table 7.

TABLE 7
Naturally occurring amino acids in a variable heavy chain framework 1 (FR1) region and their frequency of occurrence.

| AA# | A | C | D | E | F | G | H | I | K | L | M | N | P | Q | R | S | T | V | W | Y |
|---|---|---|---|---|---|---|---|---|---|---|---|---|---|---|---|---|---|---|---|---|
| 1 | | | | 0.30 [symbol] | | | | | | | | | | 0.686 | 0.000 | | | | | |
| 2 | | | | | | | | 0.020 | | 0.00 [symbol] | 0.017 | | | | | | | 0.899 | | |
| 3 | | | | | | | 0.003 | | | | | | | 0.910 | 0.003 | | 0.085 | | | |
| 4 | | | | | | | | | | 0.383 | | | | | 0.003 | | | 0.015 | | |
| 5 | | | | | | | | | 0.055 | 0.017 | | | | 0.147 | 0.029 | | | 0. [symbol] 1 | | |
| 6 | | | 0.00 [symbol] | 0.7 [symbol] | | | | | 0.003 | | | | | 0.227 | | | | | | |
| 7 | | | | | | | | | | 0.003 | | | 0.015 | | | 0.945 | 0.005 | | 0.032 | |
| 8 | 0.006 | | | | | 0. [symbol] 88 | | | | | | | | | | | | 0.006 | | |
| 9 | 0.19 [symbol] | | | 0.00 [symbol] | | 0.471 | | | | | | | 0.294 | | | 0.029 | | | | |
| 10 | | | | | | | | | | | | | | | | | | | | |
| 11 | [symbol] .055 | | | 0.189 | | 0.712 | | | | | | | | | | | 0. [symbol] | 0.0 [symbol] | | |
| 12 | | | | | | | | | | 0.727 | | | | | | | | 0.273 | | |
| 13 | | | | | | | | 0.012 | 0.174 | 0.038 | | | | | 0.015 | | | 0.762 | | |
| 14 | | | | | | | | | 0.564 | | | 0.003 | | 0.422 | 0.012 | | | | | |
| 15 | 0.029 | | 0.00 [symbol] | | | | | | | 0.003 | | | 0.953 | | | | 0.009 | | | |
| 16 | | | | | | 0.659 | | | | | | | 0.012 | | | 0.235 | 0.084 | | | |
| 17 | 0.006 | | 0.023 | 0.140 | | 0.387 | | | | | | | 0.003 | 0.180 | 0.102 | 0.058 | 0.009 | 0.003 | | |
| 18 | 0.006 | | | | | | | | | 0.003 | 0.009 | | | | | 0.000 | 0. [symbol] | | | |
| 19 | | | | | | | | | | 0.843 | | | 0.003 | | | | | 0.19 [symbol] | | |
| 20 | | | | | | | | | 0.106 | | | | | | 0.483 | 0.247 | 0.084 | | | |
| 21 | | | | | | | 0.009 | 0.029 | | 0.797 | | | | | | | | 0.165 | | |
| 22 | | | | | | | | | | | | | | | | 0.069 | 0.011 | | | |
| 23 | | 0.991 | | | 0.008 | | | | | | | | | | 0.003 | | | | | |
| 24 | 0.564 | | | | | | | | 0.185 | | | | | | | 0.012 | 0.233 | | | |
| 25 | 0.685 | | | | 0.07 [symbol] | 0.020 | | 0.012 | | 0.003 | | | | | | | | 0.239 | | |
| 26 | | 0.003 | | | | | | | | | | | | | | 0.951 | 0.003 | | | 0.654 |

[symbol]

indicates data missing or illegible when filed

In some embodiments, the amino acids naturally occurring in a variable k light chain with over 2% frequency at each position according to an antibody numbering scheme, for example according to the IMGT numbering scheme, are listed in Table 8.

TABLE 8
Naturally occurring amino acids (frequency >2%)
in a variable κ light chain.

| | Naturally occurring amino acids in a |
|---|---|
| IMGT AA # | variable κ light chain (frequency >2%) |
| 1 | A, D, E, V |
| 2 | I, V |
| 3 | Q, R, V, W |
| 4 | L, M |
| 5 | T |
| 6 | Q |
| 7 | S, T |
| 8 | P |
| 9 | A, D, L, S |
| 10 | A, F, L, S, T |
| 11 | L, M, Q, V |
| 12 | P, S |
| 13 | A, I, L, V |
| 14 | S, T |
| 15 | L, P, T, V |
| 16 | G, K |
| 17 | D, E, Q |
| 18 | K, P, Q, R |

In some embodiments, the amino acids naturally occurring in a variable k light chain at each position according to an antibody numbering scheme, for example according to the IMGT numbering scheme, and their frequency of occurrence are listed in Table 9.

TABLE 9
Naturally occurring amino acids in a variable κ light chain framework 1 (FR1) region and their frequency of occurrence.

| AA# | A | C | D | E | F | G | H | I | K | L | M | N | P | Q | R | S | T | V | W | Y |
|---|---|---|---|---|---|---|---|---|---|---|---|---|---|---|---|---|---|---|---|---|
| 1 | 0.171 | | 0.519 | 0.312 | | | z | | | | | 0.015 | | | | | | 0.030 | | z |
| 2 | | | | | | | | | | | | | | | | | 0.015 | 0.045 | | |
| 3 | | | | | | | | 0.919 | | | | | | 0.409 | 0.050 | | 0.015 | 0.500 | 0.045 | |
| 4 | | | | | | | | | | 0.273 | 0.727 | | | | | | | | | |
| 5 | | | | | | | | | | | | | | | | | 1.000 | | | |
| 6 | | | | | | | | | | | | | | 2.000 | | | | | | |
| 7 | | | | | | | | | | | | | | | | 0. [symbol] 8 | 0.152 | | | |
| 8 | | | | | | | | | | | | | 1.000 | | | | | | | |
| 9 | 0.197 | | 0.076 | | 0.015 | 0.015 | | | | 0.227 | | | | | | 0.470 | | | | |
| 10 | 0.030 | | | | 0.091 | | | | | 0.045 | | | | | | 0.591 | 0.742 | | | |
| 11 | | | | | 0.015 | | | | | 0.803 | 0.045 | | | 0.061 | | 0.015 | | 0.061 | | |
| 12 | 0.015 | | | | | | | | | | | | 0.121 | | | 0.964 | | | | |
| 13 | 0.500 | | | | | | | 0.045 | | 0.152 | | | | | | | | 0.30 [symbol] | | |
| 14 | | | | | | | | | | | | | 0.4 [symbol] 9 | | | 0.897 | 0.307 | | | |
| 15 | | | | | | | | | | 0.076 | | | | | | | 0.061 | 0.424 | | |
| 16 | | | | | | 0.9 [symbol] 5 | | | 0.061 | | | | | 0.121 | | | | | | |
| 17 | | | 0.500 | 0.379 | | | | | | | | | 0.18 [symbol] | 0.045 | 0.857 | | | | | |
| 18 | | | | | | | | | 0.078 | | | | | | | | | | | |
| 19 | 0.439 | | | | | | | | | | | 0.015 | | | | | | 0.561 | | |
| 20 | | | | | | | | 0.773 | | 0.157 | 0.030 | | | | | 0.227 | 0.758 | | | |
| 21 | | | | | | | | 0.013 | | | | 0.015 | | | | | | | | |
| 22 | | | | | | | | | | | | | | | | 0.485 | 0.485 | | | |
| 23 | | 1.000 | | | | | | | | | | | | | | | | | | |
| 24 | | | | | | 0.015 | | | 0.051 | | | | | 0.030 | 0.888 | | | | 0.015 | |
| 25 | 0.7 [symbol] 2 | | | | | | | | | | 0.045 | | | | | 0.242 | | | | |

[symbol]

indicates data missing or illegible when filed

In some embodiments, the amino acids naturally occurring in a variable λ light chain with over 2% frequency at each position according to an antibody numbering scheme, for example according to the IMGT numbering scheme, are listed in Table 10.

TABLE 10
Naturally occurring amino acids (frequency >2%)
in a variable λ light chain.

| | Naturally occurring amino acids in a |
|---|---|
| IMGT AA # | variable λ light chain (frequency >2%) |
| 1 | N, Q, R, S |
| 2 | A, F, L, P, S, T, Y |
| 3 | A, E, G, M, V |
| 4 | L, V |
| 5 | T |
| 6 | Q |
| 7 | E, P, S |
| 8 | A, H, L, P, R, S, T |
| 9 | A, F, S |
| 10 | |
| 11 | A, F, L, V |
| 12 | S, T |
| 13 | A, E, G, K, V |
| 14 | A, G, S, T |
| 15 | L, P, T |
| 16 | A, G, R |
| 17 | A, G, K, Q, S |
| 18 | K, M, R, S, T |

In some embodiments, the amino acids naturally occurring in a variable 2 light chain at each position according to an antibody numbering scheme, for example according to the IMGT numbering scheme, and their frequency of occurrence are listed in Table 11.

TABLE 11
Naturally occurring amino acids in a variable λ light chain framework 1 (FR1) region and their frequency of occurrence.

| AA# | A | C | D | E | F | G | H | I | K | L | M | N | P | Q | R | S | T | V | W | Y |
|---|---|---|---|---|---|---|---|---|---|---|---|---|---|---|---|---|---|---|---|---|
| 1 | | | | | | | | | | 0.013 | | 0.027 | | 0.893 | 0.027 | 0.240 | | | z | |
| 2 | 0.107 | | | | 0.027 | | | | | 0.027 | | | 0.187 | | | 0.400 | 0.040 | | | 0.212 |
| 3 | 0.213 | | | 0.187 | | 0.04 [symbol] | | | | | 0.027 | | | | | | | 0.588 | | |
| 4 | | | | | | | | | | 0.850 | | | 0.013 | | | | | 0.107 | | |
| 5 | | | | | | | | | | | 0.013 | | | | | | 0.987 | | | |
| 6 | | | | | | | | | | | | | | 1.000 | | | | | | |
| 7 | | | 0.013 | 0.083 | | | | | | 0.013 | | | 0.813 | | | 0.067 | | 0.013 | | |
| 8 | 0.090 | | | | | | 0.053 | | | 0.027 | | | 0.667 | | 0.027 | 0.107 | 0.040 | | | |
| 9 | 0.027 | | | | 0.040 | | | | | | | | | | | 0.93 [symbol] | | | | |
| 10 | | | | | | | | | | | | | | | | | | | | |
| 11 | 0.187 | | | | 0.027 | | 0.013 | | | 0.179 | | | | | | 0.013 | | 0.587 | | |
| 12 | | | | | | | | | | | | | | | | 0.963 | 0.040 | | | |
| 13 | 0.300 | | | 0.040 | | 0.307 | | | 0.027 | | | | | | 0.013 | | | 0.307 | | |
| 14 | 0.287 | | | | | 0.027 | | | | 0.013 | | | | | | 0.653 | 0.040 | | | |
| 15 | | | | | | | | | | 0.213 | | | 0.747 | | | 0.013 | 0.027 | | | |
| 16 | 0.027 | | | | | 0.933 | | | | | | | | | 0.040 | | | | | |
| 17 | 0.213 | | | 0.013 | | 0.080 | | | 0.053 | | | | | 0.500 | | 0.040 | | | | |
| 18 | 0.013 | | | | | | | | 0.040 | | 0.053 | | | | 0.107 | 0.459 | 0.3 [symbol] | | | |
| 19 | 0.387 | | | | | | | 0.090 | | | | | | | | | | 0.533 | | |
| 20 | | | | | | | | | 0.080 | | | | | | 0.320 | 0.057 | 0.533 | | | |
| 21 | | | | | 0.027 | | | 0.827 | | 0.347 | | | | | | | | | | |
| 22 | | | | | | | | | | | | | 0.027 | | | 0.587 | 0.587 | | | |
| 23 | | 1.000 | | | | | | | | | | | | | | | | | | |
| 24 | 0.027 | | | | | 0.147 | | | | | 0.013 | | | | | 0.213 | 0.573 | | | |
| 25 | | | | | | 0.640 | | | | 0.307 | | | | | 0.013 | 0.040 | | | | |

[symbol]

indicates data missing or illegible when filed

In some embodiments, the second binding moiety may be deemed as comprising an “internal” deletion and/or insertion. Such internal deletion and/or insertions may also be deemed as being N-terminal truncated based on the N-terminal truncation determination process described herein. Under this circumstances, the sequence N-terminal to the “internal” deletion and/or truncation would be considered to be a part of a linker sequence instead of part of the second binding moiety. The presence of an N-terminal truncation in the second binding moiety may be further confirmed by modeling the tertiary structure of the binder molecule.

In some embodiments, the N-terminal 1st amino acid of the truncated second binding moiety, e.g., VHAb2, is not E or Q. In some embodiments, the N-terminal 1st amino acid of the truncated second binding moiety, e.g., VHAb2, is not E, Q, or R. In some embodiments, the N-terminal 2nd amino acid of the truncated second binding moiety, e.g., VHAb2, is not I, L, M, or V. In some embodiments, the N-terminal 3rd amino acid of the truncated second binding moiety, e.g., VHAb2, is not Q or T. In some embodiments, the N-terminal 3rd amino acid of the truncated second binding moiety, e.g., VHAb2, is not Q, T, H, or R. In some embodiments, the N-terminal 4th amino acid of the truncated second binding moiety, e.g., VHAb2, is not L or V. In some embodiments, the N-terminal 4th amino acid of the truncated second binding moiety, e.g., VHAb2, is not L, V, or R. In some embodiments, the N-terminal 5th amino acid of the truncated second binding moiety, e.g., VHAb2, is not K, L, Q, R, or V. In some embodiments, the N-terminal 6th amino acid of the truncated second binding moiety, e.g., VHAb2, is not E or Q. In some embodiments, the N-terminal 6th amino acid of the truncated second binding moiety, e.g., VHAb2, is not E, K, Q, or D. In some embodiments, the N-terminal 7th amino acid of the truncated second binding moiety, e.g., VHAb2, is not P, S, or W. In some embodiments, the N-terminal 7th amino acid of the truncated second binding moiety, e.g., VHAb2, is not P, S, W, L or T. In some embodiments, the N-terminal 8th amino acid of the truncated second binding moiety, e.g., VHAb2, is not G. In some embodiments, the N-terminal 8th amino acid of the truncated second binding moiety, e.g., VHAb2, is not G, A, or V. In some embodiments, the N-terminal 9th amino acid of the truncated second binding moiety, e.g., VHAb2, is not A, E, G, P, or S. In some embodiments, the N-terminal 11th amino acid of the truncated second binding moiety, e.g., VHAb2, is not A, E, G, T, or V. In some embodiments, the N-terminal 12th amino acid of the truncated second binding moiety, e.g., VHAb2, is not L or V. In some embodiments, the N-terminal 13th amino acid of the truncated second binding moiety, e.g., VHAb2, is not I, K, L, R, or V. In some embodiments, the N-terminal 14th amino acid of the truncated second binding moiety, e.g., VHAb2, is not K, Q, or R. In some embodiments, the N-terminal 14th amino acid of the truncated second binding moiety, e.g., VHAb2, is not K, Q, R, or N. In some embodiments, the N-terminal 15th amino acid of the truncated second binding moiety, e.g., VHAb2, is not A or P. In some embodiments, the N-terminal 15th amino acid of the truncated second binding moiety, e.g., VHAb2, is not A, P, D, L, or T. In some embodiments, the N-terminal 16th amino acid of the truncated second binding moiety, e.g., VHAb2, is not G, P, S, or T. In some embodiments, the N-terminal 17th amino acid of the truncated second binding moiety, e.g., VHAb2, is not A, D, E, G, Q, R, or S. In some embodiments, the N-terminal 17th amino acid of the truncated second binding moiety, e.g., VHAb2, is not A, D, E, G, Q, R, S, P, T, or V. In some embodiments, the N-terminal 18th amino acid of the truncated second binding moiety, e.g., VHAb2, is not S or T. In some embodiments, the N-terminal 18th amino acid of the truncated second binding moiety, e.g., VHAb2, is not S, T, A, L, or M.

In some embodiments, the N-terminal truncated antibody variable domain of the second binding moiety further comprises from 1 to 18 amino acid substitutions, such as in the framework 1 (FR1) region.

In some embodiments, the binder molecule comprising the second binding moiety comprises N-terminal amino acid A₁, wherein A₁ is any amino acids other than E or Q. In some embodiments, the binder molecule comprising the second binding moiety comprises N-terminal amino acids A₁-A₂, wherein A₁ is any amino acids other than E or Q, and wherein A₂ is any amino acid other than I, L, M, or V. In some embodiments, the binder molecule comprising the second binding moiety comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the binder molecule comprising the second binding moiety comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the binder molecule comprising the second binding moiety comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V.

3. Other Features Associated with a Second Binding Moiety

In some embodiments, the second binding moiety is associated with another feature useful for the description provided herein. In some embodiments, the second binding moiety is associated with a drug, such a second binding moiety covalently conjugated to a drug. In some embodiments, the second binding moiety is associated with a label, such as a second binding moiety covalently conjugated to an affinity label (e.g., biotin) or a visual label (such as a fluorescent label). In some embodiments, the second binding moiety is associated with an enzyme, such as a second binding moiety covalently conjugated to an enzyme. In some embodiments, the second binding moiety is associated with a toxin, such as a second binding moiety covalently conjugated to a toxin. In some embodiments, the second binding moiety is associated with a nucleic acid, such as a second binding moiety covalently conjugated to a nucleic acid. In some embodiments, the second binding moiety is associated with an albumin, such as human serum albumin.

### B. Co-Binders

In certain aspects, provided herein is a co-binder comprising a first binding moiety specifically recognizing a first target site and a second binding moiety specifically recognizing a second target site, wherein, optionally, the second binding moiety is a second antibody moiety comprising an antibody variable domain having an N-terminal truncation (“N-terminal truncated antibody variable domain”), and wherein the first binding moiety is connected to the second binding moiety through N-terminus of the N-terminal truncated antibody variable domain optionally via a linker. In some embodiments, the second binding moiety is a second antibody moiety comprising an antibody variable domain having an N-terminal truncation (“N-terminal truncated antibody variable domain”). In some embodiments, the first binding moiety is connected to the second binding moiety through N-terminus of the N-terminal truncated antibody variable domain via a linker, such as a polypeptide linker. In some embodiments, the co-binder comprises a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), and wherein the first binding moiety is connected to the second binding moiety through N-terminus of the N-terminal truncated antibody variable domain via a linker. In some embodiments, the co-binder is a single amino acid chain.

In some embodiments, the co-binder specifically recognizes two target sites (epitopes), on a single target antigen, such as a polypeptide. As discussed herein, the co-binder is configured to increase affinity and specificity to the target antigen via specifically recognizing two target sites (epitopes). In some embodiments, the co-binder is a multispecific co-binder, such as a bispecific co-binder. In some embodiments, the bispecific co-binder recognizes two target antigens in spatial proximity, such as in a complex. In some embodiments, the bispecific co-binder recognizes two of the same target antigen, such as present in a homodimer.

In some embodiments, provided is a co-binder comprising a first binding moiety specifically recognizing a first target site and a second binding moiety specifically recognizing a second target site, wherein the second binding moiety comprises a second V_{H}H domain comprising an N-terminal truncation (“N-terminal truncated V_{H}H domain”), wherein the first binding moiety comprises a first V_{H}H domain, wherein the first binding moiety is connected to the second binding moiety through N-terminus of the N-terminal truncated antibody variable domain via a linker. In some embodiments, the N-terminal truncated V_{H}H domain comprises a truncations in the FR1 region of the V_{H}H domain. In some embodiments, the N-terminal truncated V_{H}H domain comprises a truncation of any of 1, 2, 3, 4, 5, 6, 7, 8, 9, 10, 11, 12, 13, 14, 15, 16, 17, 18, 19, or 20 amino acids. In some embodiments, the N-terminal truncated V_{H}H domain comprises N-terminal amino acid A₁, wherein A₁ is any amino acids other than E or Q. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker. In some embodiments, the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In some embodiments, provided is a co-binder comprising a first binding moiety specifically recognizing a first target site and a second binding moiety specifically recognizing a second target site, wherein the second binding moiety comprises a second V_{H}H domain comprising an N-terminal truncation (“N-terminal truncated V_{H}H domain”) in the FR1 region of the V_{H}H domain, wherein the first binding moiety comprises a first V_{H}H domain, wherein the first binding moiety is connected to the second binding moiety through N-terminus of the N-terminal truncated antibody variable domain via a linker. In some embodiments, the N-terminal truncated V_{H}H domain comprises a truncation of any of 1, 2, 3, 4, 5, 6, 7, 8, 9, 10, 11, 12, 13, 14, 15, 16, 17, 18, 19, or 20 amino acids. In some embodiments, the N-terminal truncated V_{H}H domain comprises N-terminal amino acid A₁, wherein A₁ is any amino acids other than E or Q. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker. In some embodiments, the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In some embodiments, provided is a co-binder comprising a first binding moiety specifically recognizing a first target site and a second binding moiety specifically recognizing a second target site, wherein the second binding moiety comprises a second V_{H}H domain comprising an N-terminal truncation (“N-terminal truncated V_{H}H domain”) of any of 1, 2, 3, 4, 5, 6, 7, 8, 9, 10, 11, 12, 13, 14, 15, 16, 17, 18, 19, or 20 amino acids in the FR1 region of the V_{H}H domain, wherein the first binding moiety comprises a first V_{H}H domain, wherein the first binding moiety is connected to the second binding moiety through N-terminus of the N-terminal truncated antibody variable domain via a linker. In some embodiments, the N-terminal truncated V_{H}H domain comprises N-terminal amino acid A₁, wherein A₁ is any amino acids other than E or Q. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker. In some embodiments, the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In some embodiments, provided is a co-binder comprising a first binding moiety specifically recognizing a first target site and a second binding moiety specifically recognizing a second target site, wherein the second binding moiety comprises a second V_{H}H domain comprising an N-terminal truncation (“N-terminal truncated V_{H}H domain”) of any of 1, 2, 3, 4, 5, 6, 7, 8, 9, 10, 11, 12, 13, 14, 15, 16, 17, 18, 19, or 20 amino acids in the FR1 region of the V_{H}H domain, wherein the first binding moiety comprises a first V_{H}H domain, wherein the N-terminal truncated V_{H}H domain comprises N-terminal amino acid A₁, wherein A₁ is any amino acids other than E or Q, and wherein the first binding moiety is connected to the second binding moiety through N-terminus of the N-terminal truncated antibody variable domain via a linker. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker. In some embodiments, the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In some embodiments, provided is a co-binder comprising a first binding moiety specifically recognizing a first target site and a second binding moiety specifically recognizing a second target site, wherein the second binding moiety comprises a second V_{H}H domain comprising an N-terminal truncation (“N-terminal truncated V_{H}H domain”) of any of 1, 2, 3, 4, 5, 6, 7, 8, 9, 10, 11, 12, 13, 14, 15, 16, 17, 18, 19, or 20 amino acids in the FR1 region of the V_{H}H domain, wherein the first binding moiety comprises a first V_{H}H domain, wherein the N-terminal truncated V_{H}H domain comprises N-terminal amino acid A₁, wherein A₁ is any amino acids other than E or Q, wherein the first binding moiety is connected to the second binding moiety through N-terminus of the N-terminal truncated antibody variable domain via a linker, and wherein the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker.

In some embodiments, provided is a co-binder comprising a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), wherein the first binding moiety is connected to the second binding moiety through N-terminus of the N-terminal truncated antibody variable domain optionally via a linker. In some embodiments, the co-binder binds to the second target site with an affinity of at least about 3 fold of that of a control co-binder comprising an antibody variable domain not having the N-terminal truncation. In some embodiments, the first target site and the second target site are non-overlapping binding sites on a target molecule. In some embodiments, the co-binder binds to the target molecule with an affinity of at least about 3 fold of that of a control co-binder comprising an antibody variable domain not having the N-terminal truncation. In some embodiments, the first antibody moiety is selected from the group consisting of a Fab, an Fv, an scFv, a dsFv, a Fab′, or a (Fab′)2 fragment. In some embodiments, the N-terminal truncated antibody variable domain is a truncated VH or truncated VL domain. In some embodiments, the second antibody moiety is a single domain antibody. In some embodiments, the N-terminal truncation of the N-terminal truncated antibody variable domain is about 1 to about 25 amino acids. In some embodiments, the N-terminal truncation of the N-terminal truncated antibody variable domain is 1 amino acid.

In some embodiments, provided is a co-binder comprising a first binding moiety specifically recognizing a first target site and a second binding moiety specifically recognizing a second target site, wherein the second binding moiety is a second antibody moiety comprising an antibody variable domain having an N-terminal truncation (“N-terminal truncated antibody variable domain”), wherein the first binding moiety is a first antibody moiety, and wherein the first binding moiety is connected to the second binding moiety through N-terminus of the N-terminal truncated antibody variable domain optionally via a linker. In some embodiments, the co-binder binds to the second target site with an affinity of at least about 3 fold of that of a control co-binder comprising an antibody variable domain not having the N-terminal truncation. In some embodiments, the first target site and the second target site are non-overlapping binding sites on a target molecule. In some embodiments, the co-binder binds to the target molecule with an affinity of at least about 3 fold of that of a control co-binder comprising an antibody variable domain not having the N-terminal truncation. In some embodiments, the first antibody moiety is selected from the group consisting of a Fab, an Fv, an scFv, a dsFv, a Fab′, or a (Fab′)2 fragment. In some embodiments, the N-terminal truncated antibody variable domain is a truncated VH or truncated VL domain. In some embodiments, the second antibody moiety is a single domain antibody. In some embodiments, the N-terminal truncation of the N-terminal truncated antibody variable domain is about 1 to about 25 amino acids. In some embodiments, the N-terminal truncation of the N-terminal truncated antibody variable domain is 1 amino acid.

In some embodiments, provided is a co-binder comprising a first binding moiety specifically recognizing a first target site and a second binding moiety specifically recognizing a second target site, wherein the second binding moiety comprises a second V_{H}H domain having an N-terminal truncation (“N-terminal truncated V_{H}H domain”), wherein the first binding moiety comprises a first V_{H}H domain, and wherein the C-terminus of the first V_{H}H domain is connected to the N-terminus of the second V_{H}H domain via a linker. In some embodiments, the co-binder binds to the second target site with an affinity of at least about 3 fold of that of a control co-binder comprising an antibody variable domain not having the N-terminal truncation. In some embodiments, the first target site and the second target site are non-overlapping binding sites on a target molecule. In some embodiments, the co-binder binds to the target molecule with an affinity of at least about 3 fold of that of a control co-binder comprising an antibody variable domain not having the N-terminal truncation. In some embodiments, the first antibody moiety is selected from the group consisting of a Fab, an Fv, an scFv, a dsFv, a Fab′, or a (Fab′)2 fragment. In some embodiments, the N-terminal truncation of the N-terminal truncated antibody variable domain is about 1 to about 25 amino acids.

In some embodiments, provided is a co-binder comprising a first binding moiety specifically recognizing a first target site and a second binding moiety specifically recognizing a second target site, wherein the second binding moiety comprises a second V_{H}H domain having an N-terminal truncation (“N-terminal truncated V_{H}H domain”), wherein the N-terminal truncation of the N-terminal truncated V_{H}H is 1 amino acid, wherein the first binding moiety comprises a first V_{H}H domain, and wherein the C-terminus of the first V_{H}H domain is connected to the N-terminus of the second V_{H}H domain via a linker. In some embodiments, the co-binder binds to the second target site with an affinity of at least about 3 fold of that of a control co-binder comprising an antibody variable domain not having the N-terminal truncation. In some embodiments, the first target site and the second target site are non-overlapping binding sites on a target molecule. In some embodiments, the co-binder binds to the target molecule with an affinity of at least about 3 fold of that of a control co-binder comprising an antibody variable domain not having the N-terminal truncation. In some embodiments, the first antibody moiety is selected from the group consisting of a Fab, an Fv, an scFv, a dsFv, a Fab′, or a (Fab′)2 fragment. In some embodiments, the C-terminal amino acid of the peptide linker immediately connected to the N-terminal truncated antibody variable domain is G. In some embodiments, the C-terminal three amino acids of the peptide linker immediately connected to the N-terminal truncated antibody variable domain are X₁-X₂-X₃, wherein X₁ is V, L, W, P, S, G, K, D, F, M, T, N, or R; X₂ is V, A, L, S, G, R, K, M, C, F, T, P, or E; and X₃ is G.

In some embodiments, provided is a co-binder comprising a first binding moiety specifically recognizing a first target site and a second binding moiety specifically recognizing a second target site, wherein the second binding moiety is a second antibody moiety comprising an antibody variable domain; wherein the first binding moiety is connected to the second binding moiety through N-terminus of the N-terminal truncated antibody variable domain via a peptide linker; wherein the C-terminal three amino acids of the peptide linker immediately connected to the antibody variable domain of the second binding moiety are X₁-X₂-X₃, wherein X₁ is any amino acid; X₂ is K, R, Y, M, G, or N; and X₃ is R, G, Y, or P. In some embodiments, the co-binder binds to the second target site with an affinity of at least about 3 fold of linker control co-binder. In some embodiments, the first target site and the second target site are non-overlapping binding sites on a target molecule. In some embodiments, the co-binder binds to the target molecule with an affinity of at least about 3 fold of that of linker control co-binder.

In some embodiments, provided is a co-binder comprising a first binding moiety specifically recognizing a first target site and a second binding moiety specifically recognizing a second target site, wherein the second binding moiety is a second antibody moiety comprising an antibody variable domain; wherein the first binding moiety is connected to the second binding moiety through N-terminus of the N-terminal truncated antibody variable domain via a peptide linker; wherein the C-terminal three amino acids of the peptide linker immediately connected to the antibody variable domain of the second binding moiety are X₁-X₂-X₃, wherein X₁ is any amino acid; X₂ is K, R, Y, M, G, or N; and X₃ is R, G, Y, or P, and wherein the first binding moiety is a first antibody moiety. In some embodiments, the co-binder binds to the second target site with an affinity of at least about 3 fold of linker control co-binder. In some embodiments, the first target site and the second target site are non-overlapping binding sites on a target molecule. In some embodiments, the co-binder binds to the target molecule with an affinity of at least about 3 fold of that of linker control co-binder. In some embodiments, the antibody variable domain is a VH or VL domain. In some embodiments, the second antibody moiety is a single domain antibody.

In some embodiments, provided is a co-binder comprising a first binding moiety specifically recognizing a first target site and a second binding moiety specifically recognizing a second target site, wherein the first binding moiety comprises a first V_{H}H domain; wherein the second binding moiety comprises a second V_{H}H domain, wherein the C-terminus of the first V_{H}H domain is connected to the N-terminus of the second V_{H}H domain via the peptide linker wherein the C-terminal three amino acids of the peptide linker immediately connected to the antibody variable domain of the second binding moiety are X₁-X₂-X₃, wherein X₁ is any amino acid; X₂ is K, R, Y, M, G, or N; and X₃ is R, G, Y, or P. In some embodiments, the co-binder binds to the second target site with an affinity of at least about 3 fold of linker control co-binder. In some embodiments, the first target site and the second target site are non-overlapping binding sites on a target molecule. In some embodiments, the co-binder binds to the target molecule with an affinity of at least about 3 fold of that of linker control co-binder.

In some embodiments, provided is a co-binder comprising a first binding moiety specifically recognizing a first target site and a second binding moiety specifically recognizing a second target site, wherein the second binding moiety comprises a second V_{H}H domain not comprising an N-terminal truncation, wherein the first binding moiety comprises a first V_{H}H domain, wherein the first binding moiety is connected to the second binding moiety through N-terminus of the N-terminal truncated antibody variable domain via a linker, and wherein the three N-terminal amino acids of the second binding moiety are selected from the group consisting of HKR, FKR, MKR, CKR, QKR, VKR, RKR, LKR, KKR, WKR, SKR, KRG, EKR, YKR, IKR, TKR, NKR, FRR, YRR, AKR, ZLE, ZHQ, MZL, AMV, EHY, TYP, WAP, YMY, IYK, YTY, YYP, QNY, DKR, and SGY.

In some embodiments, the N-terminal truncated V_{H}H domain comprises a truncations in the FR1 region of the V_{H}H domain. In some embodiments, the N-terminal truncated V_{H}H domain comprises a truncation of any of 1, 2, 3, 4, 5, 6, 7, 8, 9, 10, 11, 12, 13, 14, 15, 16, 17, 18, 19, or 20 amino acids. In some embodiments, the N-terminal truncated V_{H}H domain comprises N-terminal amino acid A₁, wherein A₁ is any amino acids other than E or Q. In some embodiments, the N-terminal truncated V_{H}H domain comprises N-terminal amino acids A₁-A₂, wherein A₁ is any amino acids other than E or Q, and wherein A₂ is any amino acid other than I, L, M, or V. In some embodiments, the N-terminal truncated V_{H}H domain comprises N-terminal amino acids A₁-A₂-A₃, wherein A₁ is any amino acids other than E or Q, wherein A₂ is any amino acid other than I, L, M, or V, and wherein A₃ is any amino acid other than Q or T. In some embodiments, the N-terminal truncated V_{H}H domain comprises N-terminal amino acids A₁-A₂-A₃-A₄, wherein A₁ is any amino acids other than E or Q, wherein A₂ is any amino acid other than I, L, M, or V, wherein A₃ is any amino acid other than Q or T, and wherein A₄ is any amino acid other than L or V. In some embodiments, the N-terminal truncated V_{H}H domain comprises N-terminal amino acids A₁-A₂-A₃-A₄-A₅, wherein A₁ is any amino acids other than E or Q, wherein A₂ is any amino acid other than I, L, M, or V, wherein A₃ is any amino acid other than Q or T, wherein A₄ is any amino acid other than L or V, and wherein A₅ is any amino acid other than K, L, Q, R, or V. In some embodiments, the linker is a polypeptide linker. In some embodiments, the linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In some embodiments, the co-binder binds to the second target site with an affinity of at least about 3 fold, such as at least about any of 4 fold, 5 fold, 6 fold, 7 fold, 8 fold, 9 fold, 10 fold, 25 fold, or 50 fold, of that of a control co-binder comprising an antibody variable domain not having the N-terminal truncation. In some embodiments, the first target site and the second target site are non-overlapping binding sites on a target molecule. In some embodiments, the first antibody moiety and the second antibody moiety specifically bind to different targets, such as the first antibody moiety specifically binding to a first polypeptide target and the second antibody moiety specifically binding to a second polypeptide target different from the first polypeptide target. In some embodiments, the first target site and the second target site are on different target molecules, including homo- and hetero-target complexes. In some embodiments, the co-binder binds to the target molecule with an affinity of at least about 3 fold, such as at least about any of 4 fold, 5 fold, 6 fold, 7 fold, 8 fold, 9 fold, 10 fold, 25 fold, or 50 fold, of that of a control co-binder comprising an antibody variable domain not having the N-terminal truncation.

In some aspects, provided herein are co-binders (such as high affinity and/or high specificity co-binders) that specifically bind to a target, and complexes thereof with the target. In some embodiments, the co-binder has a first binding moiety, a second binding moiety, and a linker that connects the first binding moiety and the second binding moiety. In some embodiments, the complex comprises a co-binder and a target, such as a target molecule, wherein the co-binder comprises a first binding moiety, a second binding moiety, and a linker that connects the first binding moiety and the second binding moiety. In some embodiments, the first binding moiety and second binding moiety bind to non-overlapping epitopes on a target, such as a polypeptide or a polypeptide complex. In some embodiments, the first and second binding moieties simultaneously bind to non-overlapping epitopes on a target, such as a polypeptide or a polypeptide complex. In some embodiments, the co-binder has an affinity to a target that is at least 50 fold greater, such as at least any of 100 fold greater, 200 fold greater, 500 fold greater, 1,000 fold greater, 2,000 fold greater, 5,000 fold greater, or 10,000 fold greater, than that of the first binding moiety and/or the second binding moiety. In some embodiments, the linker is a polypeptide linker. In some embodiments, the linker is a nucleic acid linker. In some embodiments, the linker is a chemical linker.

### 1. First Binding Moieties

The co-binders provided herein comprise a first binding moiety specifically recognizing a first target site and a second binding moiety specifically recognizing a second target site. Details of the second binding moiety are provided in the section above.

In some embodiments, the first binding moiety is a first antibody moiety. In some embodiments, the first binding moiety is a non-truncated antibody moiety, such as a non-truncated form of a second binding moiety having an N-terminal truncation described herein. In some embodiments, the first binding moiety is a first antibody moiety comprising an antibody variable domain having an N-terminal truncation (“N-terminal truncated antibody variable domain”). In some embodiments, the first binding moiety is a first antibody moiety comprising an antibody variable domain having a C-terminal truncation. In some embodiments, the first binding moiety is another molecule providing affinity to a target site. For example, in some embodiments, the first binding moiety is a ligand recognizing a receptor or a portion thereof. In some embodiments, the first binding moiety is a receptor or a portion thereof, such as an extracellular domain of a receptor, recognizing a ligand. In some embodiments, the first binding moiety is an aptamer. In some embodiments, the first binding moiety is a non-protein binding moiety, such as biotin or a nucleic acid. In some embodiments, the first binding moiety is a non-immunoglobulin binding agent.

In some embodiments, the antibody moiety of a first binding moiety comprises a variable region (in some embodiments, referred to herein as VR, and optionally, with a numerical identification thereof, e.g., VR1 or VR2). In some embodiments, the antibody moiety of a first binding moiety comprises a heavy chain variable region (in some embodiments, referred to herein as VHAb or VH domain). In some embodiments, the heavy chain variable region is associated with a light chain variable region. In some embodiments, wherein the heavy chain variable region is associated with a light chain variable region, the heavy chain variable region and the light chain variable region are from the same antibody or antigen binding fragment. In some embodiments, the heavy chain variable region associated with a light chain variable region form a stable complex.

In some embodiments, the antibody moiety of a first binding moiety comprises a light chain variable region (in some embodiments, referred to herein as VLAb or VL domain). In some embodiments, the light chain variable region is a light chain variable region of human lambda (λ) light chain. In some embodiments, the light chain variable region is a light chain variable region of human kappa (κ) light chain. In some embodiments, the light chain variable region is associated with a heavy chain variable region. In some embodiments, wherein the light chain variable region is associated with a heavy chain variable region, the light chain variable region and the heavy chain variable region are from the same antibody or antigen binding fragment. In some embodiments, the light chain variable region associated with a heavy chain variable region form a stable complex.

In some embodiments, the antibody moiety of a first binding moiety further comprises one or more constant domains, such as any one or more of CH1, CH2, CH3, or CL.

In some embodiments, the antibody moiety of a first binding moiety comprises a V_{H}H domain. In some embodiments, the antibody moiety of a first binding moiety is selected from the group consisting of a Fab, Fv, scFv, dsFv, Fab′, and (Fab′)2 fragment. In some embodiments, the antibody moiety of a first binding moiety is a single domain antibody.

In some embodiments, the first binding moiety is a truncated first binding moiety, e.g., comprising an N-terminal and/or C-terminal truncation. In some embodiments, the truncated antibody variable domain of a first binding moiety is a truncated variable region. In some embodiments, the truncated antibody variable domain of a first binding moiety is a truncated heavy chain variable region. In some embodiments, the truncated antibody variable domain of a first binding moiety is a truncated heavy chain variable region associated with a light chain variable region. In some embodiments, the truncated antibody variable domain of a first binding moiety is a truncated light chain variable region. In some embodiments, the truncated antibody variable domain of a first binding moiety is a truncated light chain variable region associated with a heavy chain variable region. In some embodiments, the truncated antibody variable domain of a first binding moiety is a truncated VAH domain. In some embodiments, the truncated antibody variable domain of a first binding moiety is a truncated Fab, Fv, scFv, dsFv, Fab′, or (Fab′)2 fragment. In some embodiments, the truncated antibody variable domain of a first binding moiety is a truncated single domain antibody.

The first binding moieties, or at least a portion thereof, provided herein may be obtained or derived from a variety of sources. For example, in some embodiments, the first binding moiety, or at least a portion thereof, is obtained or derived from a camelid, such as a camelid single chain V_{H}H.

In some embodiments, the first binding moiety, or at least a portion thereof, is obtained or derived from an affibody, affilin, affimer, affitin, alphabody, anticalin, aptamer, avimer, DARPin, Fynomer, Kunitz domain peptide, monobody, nanobody (also referred to as a single-domain antibody, sdAb), or nanoCLAMP. In some embodiments, the first binding moiety, or at least a portion thereof, is obtained or derived from an IgG, IgA, IgE, IgM, or IgD.

In some embodiments, the truncation, such as the N-terminal truncation of the first binding moiety, is a truncation of any of 1, 2, 3, 4, 5, 6, 7, 8, 9, 10, 11, 12, 13, 14, 15, 16, 17, 18, 19, or 20 amino acids. In some embodiments, the N-terminal truncation of the first binding moiety is a truncation in the framework region 1 (FR1) of the second binding moiety. In some embodiments, the first binding moiety comprises a V_{H}H comprising a N-terminal truncation in the framework region 1 (FR1) of the first binding moiety of any of 1, 2, 3, 4, 5, 6, 7, 8, 9, 10, 11, 12, 13, 14, 15, 16, 17, 18, 19, or 20 amino acids.

In some embodiments, the first binding moiety, or at least a portion thereof, is obtained or derived from a mammal, including a camelid, human, non-human primate (such as a monkey), domestic, farm, or zoo animal, such as a dog, horse, rabbit, cow, pig, hamster, gerbil, mouse, ferret, rat, or cat. In some embodiments, the first binding moiety, or at least a portion thereof, is obtained or derived from a synthetic source.

The antibody moieties of the first binding moieties provided herein specifically recognize a target site. Said target sites encompass a diverse array of epitopes, including on polypeptides, nucleic acids, and small molecules.

### 2. Certain Co-Binder Configurations

In certain aspects, the co-binders described herein comprises a first antibody moiety of a first binding moiety and a second antibody moiety of a second binding moiety, wherein the first antibody moiety and the second antibody moiety independently comprise one of the following: a variable region (VR), a heavy chain variable region (VH or VHAb), or a light chain variable region (VL or VLAb). One of ordinary skill in the art will readily appreciate that many combinations of pairings of a first binding moiety and a second antibody moiety are possible, including, but not limited to, any of the following first antibody and second antibody moiety pairings: (i) VR1 and VR2; (ii) VHAb1 and VHAb2; (iii) VHAb1 and VLAb2; (iv) VLAb1 and VHAb2; (v) VLAb1 and VLAb2; (vi) VR1 and VHAb2; (vii) VHAb1 and VR2; (viii) VR1 and VLAb2; and (vii) VLAb1 and VR2. In some embodiments, the heavy chain variable region (e.g., VHAb1 or VHAb2) is associated with a light chain variable region. In some embodiments, the light chain variable region (e.g., VLAb1 or VLAb2) is associated with a heavy chain variable region.

In one aspect, provided herein is a co-binder that specifically binds to a target, wherein the co-binder comprises: (i) a first binding moiety comprising a first variable region of a first antibody (VR1); (ii) a second binding moiety comprising a second variable region of a second antibody (VR2) that comprises an N-terminal truncation; and (iii) a polypeptide linker that links the VR1 C-terminal amino acid with the N-terminal amino acid of the truncated VR2.

In one aspect, provided herein is a co-binder that specifically binds to a target, wherein the co-binder comprises: (i) a first binding moiety comprising a first variable region of a first antibody (VR1); (ii) a second binding moiety comprising a second variable region of a second antibody (VR2) that comprises an N-terminal truncation of from 1 to 18 amino acids; and (iii) a polypeptide linker that links the VR1 C-terminal amino acid with the N-terminal amino acid of the truncated VR2; wherein VR1 and VR2 bind to non-overlapping epitopes on the target.

In one aspect, provided herein is a co-binder that specifically binds to a target, wherein the co-binder comprises: (i) a first binding moiety comprising a first variable region of a first antibody (VR1); (ii) a second binding moiety comprising a second variable region of a second antibody (VR2) that comprises a truncation of from 1 to 18 amino acids in the framework 1 (FR1) region; and (iii) a polypeptide linker that links the VR1 C-terminal amino acid with the N-terminal amino acid of the truncated VR2; wherein VR1 and VR2 bind to non-overlapping epitopes on the target.

In some aspect, provided herein is a co-binder that specifically binds to a target, wherein the co-binder comprises: (i) a first binding moiety comprising a first variable region of a first antibody (VR1); (ii) a second binding moiety comprising a second variable region of a second antibody (VR2) that comprises an N-terminal truncation in the framework 1 (FR1) region; and (iii) a polypeptide linker that links the VR1 C-terminal amino acid with the N-terminal amino acid of the truncated VR2; wherein VR1 and VR2 bind to non-overlapping epitopes on the target.

In some embodiments, provided herein is a co-binder comprising: (i) a first binding moiety comprising a first antibody moiety specifically recognizing a first target site; (ii) a second binding moiety comprising a second antibody moiety specifically recognizing a second target site, wherein the second antibody moiety comprises an antibody variable domain having an N-terminal truncation of 1 to 18 amino acids; and (iii) a polypeptide linker that links the C-terminal amino acid of the first antibody moiety with the N-terminal amino acid of the second antibody moiety.

In some embodiments, the N-terminal truncation of from 1 to 18 amino acids of the second antibody moiety is in the framework 1 (FR1) region of the second antibody moiety. In some embodiments, the X₃ amino acid of the polypeptide linker and the start of the complementarity determining region 1 (CDR1), as characterized by the first amino acid of the CDR1 on the N-terminal amino acid side of the CDR1, of the second antibody moiety are separated by 5 to 25 amino acids. In some embodiments, the X₃ amino acid of the polypeptide linker and the start of the complementarity determining region 1 (CDR1) of the second antibody moiety are separated by no more than 25 amino acids.

Linkers are described in more detail in a section title “Linkers” provided herein. In some embodiments, the polypeptide linker comprises a consecutive series of three amino acids forming the C-terminal end of the polypeptide linker of X₁-X₂-X₃, from N- to C-terminal direction, wherein X₁ is V, L, W, P, S, G, K, D, F, M, T, N, or R; X₂ is V, A, L, S, G, R, K, M, C, F, T, P, or E; and X₃ is G.

In one aspect, provided herein is a co-binder that specifically binds to a target, wherein the co-binder comprises: (i) a first variable region of a first antibody (VR1); (ii) a second variable region of a second antibody (VR2); and (iii) a polypeptide linker that links the VR1 C-terminal amino acid with the N-terminal amino acid of the VR2. In some embodiments, the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G. In some embodiments, VR1 and VR2 bind to non-overlapping epitopes on the target. In some embodiments, the VR2 comprises an N-terminal truncation of from 1 to 18 amino acids.

In one aspect, provided herein is a co-binder that specifically binds to a target, wherein the co-binder comprises: (i) a first variable region of a first antibody (VR1); (ii) a second variable region of a second antibody (VR2) comprising an N-terminal truncation of from 1 to 18 amino acids; and (iii) a polypeptide linker that links the VR1 C-terminal amino acid with the N-terminal amino acid of the truncated VR2. In some embodiments, the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G. In some embodiments, VR1 and VR2 bind to non-overlapping epitopes on the target.

In some aspect, provided herein is a co-binder that specifically binds to a target, wherein the co-binder comprises: (i) a first variable region of a first antibody (VR1); (ii) a second variable region of a second antibody (VR2) comprising an N-terminal truncation of from 1 to 18 amino acids in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VR1 and VR2 bind to non-overlapping epitopes on the target.

In some aspect, provided herein is a co-binder that specifically binds to a target, wherein the co-binder comprises: (i) a first variable region of a first antibody (VR1); (ii) a second variable region of a second antibody (VR2) comprising a truncation of from 1 to 18 amino acids in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the VR1 and VR2 bind to non-overlapping epitopes on the target.

In another aspect, provided herein is a co-binder that specifically binds to a target, wherein the co-binder comprises: (i) a first variable region of a first antibody (VR1); (ii) a second variable region of a second antibody (VR2) comprising a truncation in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; wherein the X₃ amino acid of the polypeptide linker and the VR2 complementarity determining region 1 (CDR1) are separated by from 5 to 25 amino acids; and wherein VR1 and VR2 bind to non-overlapping epitopes on the target.

In another aspect, provided herein is a co-binder that specifically binds to a target, wherein the co-binder comprises: (i) a first variable region of a first antibody (VR1); (ii) a second variable region of a second antibody (VR2) comprising a truncation in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; wherein the X₃ amino acid of the polypeptide linker and the VR2 complementarity determining region 1 (CDR1) are separated by no more than 25 amino acids; and wherein VR1 and VR2 bind to non-overlapping epitopes on the target.

In another aspect, provided herein is a co-binder that specifically binds to a target, wherein the co-binder comprises: (i) a first variable region of a first antibody (VR1); (ii) a second variable region of a second antibody (VR2) comprising a truncation of from 1 to 18 amino acids in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; wherein the X₃ amino acid of the polypeptide linker and the VR2 complementarity determining region 1 (CDR1) are separated by from 5 to 25 amino acids; and wherein VR1 and VR2 bind to non-overlapping epitopes on the target.

In another aspect, provided herein is a co-binder that specifically binds to a target, wherein the co-binder comprises: (i) a first variable region of a first antibody (VR1); (ii) a second variable region of a second antibody (VR2) comprising a truncation of from 1 to 18 amino acids in the framework 1 (FR1) region; and (iii) a polypeptide linker that links the VR1 C-terminal amino acid with the N-terminal amino acid of the truncated VR2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; wherein the X₃ amino acid of the polypeptide linker and the VR2 complementarity determining region 1 (CDR1) are separated by no more than 25 amino acids; and wherein VR1 and VR2 bind to non-overlapping epitopes on the target.

In some embodiments, the VR1 is a light chain variable region. In some embodiments, the VR1 is a heavy chain variable region. In some embodiments, the VR2 is a light chain variable region. In some embodiments, the VR2 is a heavy chain variable region. In some embodiments, the VR1 is a light chain variable region and the VR2 is a light chain variable region. In some embodiments, the VR1 is a light chain variable region and the VR2 is a heavy chain variable region. In some embodiments, the VR1 is a heavy chain variable region and the VR2 is a light chain variable region. In some embodiments, the VR1 is a heavy chain variable region and the VR2 is a heavy chain variable region. In some embodiments, the VR1 is a V_{H}H. In some embodiments, the VR2 is a V_{H}H. In some embodiments, the VR1 is a V_{H}H and the VR2 is a V_{H}H.

In some embodiments, the N-terminal truncation of the second binding moiety is a truncation of any of 1, 2, 3, 4, 5, 6, 7, 8, 9, 10, 11, 12, 13, 14, 15, 16, 17, 18, 19, or 20 amino acids. In some embodiments, the X₃ amino acid of the polypeptide linker and the CDR1 of the second binding moiety are separated by no more than 25 amino acids, such as no more than any of 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, or 3 amino acids. In some embodiments, the X₃ amino acid of the polypeptide linker and the CDR1 of the second binding moiety are separated by any of 5 amino acids, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, or 25 amino acids. In some embodiments, the N-terminal truncation of the second binding moiety is a truncation in the framework region 1 (FR1) of the second binding moiety.

In some embodiments, the variable region, such as an N-terminal truncated antibody variable domain of a second binding moiety, is a V_{H}H. In some embodiments, the first binding moiety comprises a first V_{H}H domain; wherein the second binding moiety comprises a second V_{H}H domain having an N-terminal truncation (“truncated V_{H}H domain”), wherein the C-terminus of the first V_{H}H domain is connected to the N-terminus of the second V_{H}H domain via a linker.

Thus, in some aspects, provided herein is a co-binder comprising a first binding moiety specifically recognizing a first target site and a second binding moiety specifically recognizing a second target site, wherein the second binding moiety is a second V_{H}H domain having an N-terminal truncation (“truncated V_{H}H domain”), wherein the first binding moiety comprises a first V_{H}H domain, and wherein the first binding moiety is connected to the second binding moiety through N-terminus of the N-terminal truncated antibody variable domain via a linker. In some embodiments, the truncated V_{H}H domain of the second binding moiety is a truncation of any of 1, 2, 3, 4, 5, 6, 7, 8, 9, 10, 11, 12, 13, 14, 15, 16, 17, 18, 19, or 20 amino acids. In some embodiments, the X₃ amino acid of the polypeptide linker and the CDR1 of the second binding moiety are separated by no more than 25 amino acids, such as no more than any of 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, 5 amino acids, 4 amino acids, or 3 amino acids. In some embodiments, the X₃ amino acid of the polypeptide linker and the CDR1 of the second binding moiety are separated by any of 5 amino acids, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, or 25 amino acids. In some embodiments, the N-terminal truncation of the second binding moiety is a truncation in the framework region 1 (FR1) of the second binding moiety. In some embodiments, the linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G.

In some embodiments, the co-binder only has the first and second binding moieties that bind to nonoverlapping and distinct epitopes on a target molecule. In some embodiments, the co-binder can also have a third binding moiety that binds to a third nonoverlapping and distinct epitope on the target molecule. In some embodiments, the co-binder can also have a third binding moiety and a fourth binding moiety that each binds to a third and a fourth nonoverlapping and distinct epitopes on the target molecule. These third and/or fourth binding moieties may or may not be N-terminal truncated as described for the second binding moiety.

The co-binder can be a monomeric molecule or a multimeric complex. In some embodiments, the co-binder is a monomeric molecule that has one set of the binding moieties. In some embodiments, the co-binder is a monomeric molecule that has one set of the first and second binding moieties. In some embodiments, the co-binder is a monomeric molecule that has one set of the first, second and third binding moieties. In some embodiments, the co-binder is a monomeric molecule that has one set of the first, second, third and fourth binding moieties.

In some embodiments, the co-binder is a multimeric complex that has at least two sets of the binding moieties. In some embodiments, the co-binder is a multimeric complex that has at least three sets of the binding moieties. In some embodiments, the co-binder is a multimeric complex that has at least four sets of the binding moieties. In some embodiments, the co-binder is a multimeric complex that has two sets of the binding moieties. In some embodiments, the co-binder is a multimeric complex that has two sets of the first and second binding moieties. In some embodiments, the co-binder is a multimeric complex that has two sets of the first, second and third binding moieties. In some embodiments, the co-binder is a multimeric complex that has two sets of the first, second, third and fourth binding moieties. In some embodiments, the co-binder is a multimeric complex that has three sets of the binding moieties. In some embodiments, the co-binder is a multimeric complex that has three sets of the first and second binding moieties. In some embodiments, the co-binder is a multimeric complex that has three sets of the first, second and third binding moieties. In some embodiments, the co-binder is a multimeric complex that has three sets of the first, second, third and fourth binding moieties. In some embodiments, the co-binder is a multimeric complex that has four sets of the binding moieties. In some embodiments, the co-binder is a multimeric complex that has four sets of the first and second binding moieties. In some embodiments, the co-binder is a multimeric complex that has four sets of the first, second and third binding moieties. In some embodiments, the co-binder is a multimeric complex that has four sets of the first, second, third and fourth binding moieties.

In some embodiments, the co-binder that is a multimeric complex can have different orientations of the sets of binding moieties. In some embodiments, the sets of binding moieties are arranged sequentially. For example, the two sets of the first binding moieties (containing paratope P1) and the second binding moieties (containing paratope P2) can be arranged as P1-P2-P1-P2. For another example, the two sets of the first binding moieties (containing paratope P1), the second binding moieties (containing paratope P2), and their binding moieties (containing paratope P3) can be arranged as P1-P2-P3-P1-P2-P3. In some embodiments, the sets of binding moieties are arranged inversely. For example, the two sets of the first binding moieties (containing paratope P1) and the second binding moieties (containing paratope P2) can be arranged as P1-P2-P2-P1. For another example, the two sets of the first binding moieties (containing paratope P1), the second binding moieties (containing paratope P2), and their binding moieties (containing paratope P3) can be arranged as P1-P2-P3-P3-P2-P1. In some embodiments, the sets of binding moieties are arranged in a staggered manner. For example, the two sets of the first binding moieties (containing paratope P1) and the second binding moieties (containing paratope P2) can be arranged as P1-P1-P2-P2. For another example, the two sets of the first binding moieties (containing paratope P1), the second binding moieties (containing paratope P2), and their binding moieties (containing paratope P3) can be arranged as P1-P1-P2-P2-P3-P3. As a person of ordinary skill in the art would understand, the binding moieties of a multimeric co-binder described herein can be arranged in any order. In some embodiments, the order of arrangement of the binding moieties of a multimeric co-binder is optimized to maximize the binding affinity to the target molecule and/or to minimize any nonspecific binding.

In some embodiments, the co-binders disclosed herein have a first binding moiety and a second binding moiety, which bind to two distinct and nonoverlapping epitopes in a target molecule. The two distinct and nonoverlapping epitopes in a target molecule can be relatively close to each other. In some embodiments, the two epitopes recognized by the co-binder are located close to each other, but still allow sufficient space to accommodate the linker of the co-binder. In some embodiments, the first and second epitopes have a distance of no more than 150 angstroms, such as no more than about any of 120 angstroms, 100 angstroms, 80 angstroms, 50 angstroms, 40 angstroms, 30 angstroms, 15 angstroms, 10 angstroms, or 5 angstroms.

For linear epitopes on a target peptide or target protein, the distance between the two epitopes can be within 200 amino acids of each other, such as within about any of 150 amino acids of each other, 120 amino acids of each other, 100 amino acids of each other, 80 amino acids of each other, 50 amino acids of each other, 40 amino acids of each other, 30 amino acids of each other, 20 amino acids of each other, 15 amino acids of each other, 10 amino acids of each other, or 5 amino acids of each other. In some embodiments, the two epitopes recognized by the co-binder are selected such that the two binding interactions are cooperative and synergistic, and do not interfere with each other.

In some embodiments, the co-binder comprises a first antibody moiety that is a variable region (VR1) and a second antibody moiety that is a variable region (VR2). In some embodiments, VR1 binds to an epitope of the target producing desired biological effect on the target but binds to the target with insufficient affinity for therapeutic or diagnostic use by VR1 itself; VR2 binds to a different epitope of the target with sufficient affinity; and the resulting co-binder binds to the target with sufficient affinity and produces desired biological efficacy. In some embodiments of the co-binders provided herein, VR2 binds to an epitope of the target producing desired biological effect on the target but binds to the target with insufficient affinity for therapeutic or diagnostic use by VR2 itself; VR1 binds to a different epitope of the target with sufficient affinity; and the resulting co-binder binds to the target with sufficient affinity and produces desired biological efficacy. In some embodiments of the co-binders provided herein, the first binding moiety binds to an epitope of the target producing desired biological effect on the target but binds to the target with insufficient affinity for therapeutic or diagnostic use by the first binding moiety itself, the second binding moiety binds to a different epitope of the target with sufficient affinity; and the resulting co-binder binds to the target with sufficient affinity and produces desired biological efficacy. In some embodiments of the co-binders provided herein, the second binding moiety binds to an epitope of the target producing desired biological effect on the target but binds to the target with insufficient affinity for therapeutic or diagnostic use by the second binding moiety itself; the first binding moiety binds to a different epitope of the target with sufficient affinity; and the resulting co-binder binds to the target with sufficient affinity and produces desired biological efficacy.

In some embodiments, the co-binder comprises a first antibody moiety that is a heavy chain variable region of a first antibody (VHAb1) and a second antibody moiety that is a heavy chain variable region of a second antibody (VHAb2). In some embodiments, VHAb1 binds to an epitope of the target producing desired biological effect on the target but binds to the target with insufficient affinity for therapeutic or diagnostic use by VHAb1 itself, VHAb2 binds to a different epitope of the target with sufficient affinity; and the resulting co-binder binds to the target with sufficient affinity and produces desired biological efficacy. In some embodiments of the co-binders provided herein, VHAb2 binds to an epitope of the target producing desired biological effect on the target but binds to the target with insufficient affinity for therapeutic or diagnostic use by VHAb2 itself; VHAb1 binds to a different epitope of the target with sufficient affinity; and the resulting co-binder binds to the target with sufficient affinity and produces desired biological efficacy.

In some embodiments, the co-binder comprises a first antibody moiety that is a light chain variable region of a first antibody (VLAb1) and a second antibody moiety that is a heavy chain variable region of a second antibody (VHAb2). In some embodiments, VLAb1 binds to an epitope of the target producing desired biological effect on the target but binds to the target with insufficient affinity for therapeutic or diagnostic use by VLAb1 itself; VHAb2 binds to a different epitope of the target with sufficient affinity; and the resulting co-binder binds to the target with sufficient affinity and produces desired biological efficacy. In some embodiments of the co-binders provided herein, VHAb2 binds to an epitope of the target producing desired biological effect on the target but binds to the target with insufficient affinity for therapeutic or diagnostic use by VHAb2 itself; VLAb1 binds to a different epitope of the target with sufficient affinity; and the resulting co-binder binds to the target with sufficient affinity and produces desired biological efficacy.

In some embodiments, the co-binder comprises a first antibody moiety that is a heavy chain variable region of a first antibody (VHAb1) and a second antibody moiety that is a light chain variable region of a second antibody (VLAb2). In some, VHAb1 binds to an epitope of the target producing desired biological effect on the target but binds to the target with insufficient affinity for therapeutic or diagnostic use by VHAb1 itself; VLAb2 binds to a different epitope of the target with sufficient affinity; and the resulting co-binder binds to the target with sufficient affinity and produces desired biological efficacy. In some embodiments of the co-binders provided herein, VLAb2 binds to an epitope of the target producing desired biological effect on the target but binds to the target with insufficient affinity for therapeutic or diagnostic use by VLAb2 itself; VHAb1 binds to a different epitope of the target with sufficient affinity; and the resulting co-binder binds to the target with sufficient affinity and produces desired biological efficacy.

In some embodiments, the co-binder comprises a first antibody moiety that is a light chain variable region of a first antibody (VLAb1) and a second antibody moiety that is a light chain variable region of a second antibody (VLAb2). In some embodiments of the co-binders provided herein, VLAb1 binds to an epitope of the target producing desired biological effect on the target but binds to the target with insufficient affinity for therapeutic or diagnostic use by VLAb1 itself, VLAb2 binds to a different epitope of the target with sufficient affinity; and the resulting co-binder binds to the target with sufficient affinity and produces desired biological efficacy. In some embodiments of the co-binders provided herein, VLAb2 binds to an epitope of the target producing desired biological effect on the target but binds to the target with insufficient affinity for therapeutic or diagnostic use by VLAb2 itself; VLAb1 binds to a different epitope of the target with sufficient affinity; and the resulting co-binder binds to the target with sufficient affinity and produces desired biological efficacy.

In some embodiments of the co-binders provided herein, VR1 binds to an epitope of the target producing desired biological effect on the target but also binds to an epitope of non-target generating undesired side-effect; VR2 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, VR2 binds to an epitope of the target producing desired biological effect on the target but also binds to an epitope of non-target generating undesired side-effect; VR1 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

In some embodiments of the co-binders provided herein, the first binding moiety binds to an epitope of the target producing desired biological effect on the target but also binds to an epitope of non-target generating undesired side-effect; the second binding moiety binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, the second binding moiety binds to an epitope of the target producing desired biological effect on the target but also binds to an epitope of non-target generating undesired side-effect; the first binding moiety binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

In some embodiments of the co-binders provided herein, VHAb1 binds to an epitope of the target producing desired biological effect on the target but also binds to an epitope of non-target generating undesired side-effect; VHAb2 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, VHAb2 binds to an epitope of the target producing desired biological effect on the target but also binds to an epitope of non-target generating undesired side-effect; VHAb1 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

In some embodiments of the co-binders provided herein, VLAb1 binds to an epitope of the target producing desired biological effect on the target but also binds to an epitope of non-target generating undesired side-effect; VHAb2 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, VHAb2 binds to an epitope of the target producing desired biological effect on the target but also binds to an epitope of non-target generating undesired side-effect; VLAb1 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

In some embodiments of the co-binders provided herein, VHAb1 binds to an epitope of the target producing desired biological effect on the target but also binds to an epitope of non-target generating undesired side-effect; VLAb2 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, VLAb2 binds to an epitope of the target producing desired biological effect on the target but also binds to an epitope of non-target generating undesired side-effect; VHAb1 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

In some embodiments of the co-binders provided herein, VLAb1 binds to an epitope of the target producing desired biological effect on the target but also binds to an epitope of non-target generating undesired side-effect; VLAb2 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect. In some embodiments of the co-binders provided herein, VLAb2 binds to an epitope of the target producing desired biological effect on the target but also binds to an epitope of non-target generating undesired side-effect; VLAb1 binds to a different epitope of the target with high affinity but does not bind to the non-target with sufficient affinity; and the resulting co-binder binds to the target with sufficient affinity and produces desired biological efficacy, but cannot bind to the non-target with sufficient affinity to generate the undesired side effect.

The disclosure provides that various VHs or VLs can be used to construct the co-binders provided herein and obtain the affinity and/or specificity improvement provided for such co-binders. In one embodiment, the antigen binding fragments (e.g. VHs and/or VLs) contained in the second binding moiety used for the co-binders provided herein can be selected based on the close proximity of the paratope of the antigen binding fragment with the N-terminus of the antigen binding fragment. In certain embodiments, co-binders can be constructed with a VH and produce the affinity and/or specificity improvement provided for the co-binders of the disclosure, wherein the paratope of the VH is in close proximity to the N-terminus of the VH. In some embodiments, co-binders can be constructed with a VH as a part of the second binding moiety and produce the affinity and/or specificity improvement provided for the co-binders of the disclosure, wherein the paratope of the VH is in close proximity to the N-terminus of the VH. In other embodiments, co-binders can be constructed with a VHAb2 and produce the affinity and/or specificity improvement provided for the co-binders of the disclosure, wherein the paratope of the VHAb2 is in close proximity to the N-terminus of the VHAb2. In certain embodiments, co-binders can be constructed with a VL and produce the affinity and/or specificity improvement provided for the co-binders of the disclosure, wherein the paratope of the VL is in close proximity to the N-terminus of the VL. In some embodiments, co-binders can be constructed with a VL as a part of the second binding moiety and produce the affinity and/or specificity improvement provided for the co-binders of the disclosure, wherein the paratope of the VL is in close proximity to the N-terminus of the VL. In further embodiments, co-binders can be constructed with a VLAb2 and produce the affinity and/or specificity improvement provided for the co-binders of the disclosure, wherein the paratope of the VLAb2 is in close proximity to the N-terminus of the VLAb2. In some embodiments, co-binders can be constructed with a variable region (VR) and produce the affinity and/or specificity improvement provided for the co-binders of the disclosure, wherein the paratope of the VR is in close proximity to the N-terminus of the VR. In certain embodiments, co-binders can be constructed with a variable region as part of the second binding moiety (VR2) and produce the affinity and/or specificity improvement provided for the co-binders of the disclosure, wherein the paratope of the VR2 is in close proximity to the N-terminus of the VR2. The VH, VH as a part of the second binding moiety, VHAb, VHAb2, VL, VL as a part of the second binding moiety, VLAb, VLAb2, VR, and VR2 for this paragraph can be any of the corresponding embodiments described herein.

The proximity between the paratope of an antigen binding fragment (e.g. VH, VL, VHAb, VHAb2, VLAb, VLAb2, VR, and VR2 as described herein, and the N-terminus of such antigen binding fragment can be determined based on the structure of the antigen binding fragment and/or structure of the complex of such antigen binding fragment and its target antigen. In some embodiments, the nearest non-hydrogen atom on the antigen surface in the structure of the complex of such antigen binding fragment and its target antigen can be used as a proxy for the paratope for determining the proximity of the paratope and the N-terminus of the antigen binding fragment. Accordingly, the proximity can be determined based on the distance between the first Ca atom (N-terminus) of the antigen binding fragment to the nearest non-hydrogen atom on the antigen surface in the structure of the complex of such antigen binding fragment and its target antigen. In one embodiment, an antigen binding fragment (e.g. VH, VL, VHAb, VHAb2, VLAb, VLAb2, VR, and VR2 as described herein is suitable to be used to construct a co-binder provided herein and produce the affinity and/or specificity improvement provided for the co-binders of the disclosure, if the proximity as determined by the distance between the first Ca atom (N-terminus) of the antigen binding fragment to the nearest non-hydrogen atom on the antigen surface is no more than or about a certain threshold, wherein such threshold is needed to provide sufficient space for linking the two binding moieties of the co-binders without interfering with the binding to the target antigen. In another embodiment, an antigen binding fragment (e.g. VH, VL, VHAb, VHAb2, VLAb, VLAb2, VR, and VR2 as described herein is suitable to be used as the second binding moiety or part of the second binding moiety to construct a co-binder provided herein and produce the affinity and/or specificity improvement provided for the co-binders of the disclosure, if the proximity as determined by the distance between the first Ca atom (N-terminus) of the antigen binding fragment to the nearest non-hydrogen atom on the antigen surface is no more than or about a certain threshold, wherein such threshold is needed to provide sufficient space for linking the two binding moieties of the co-binders without interfering with the binding to the target antigen. In one specific embodiment, the proximity referred to herein is no more than about 15 Å, such as no more than about 14 Å, 13 Å, 12 Å, 11 Å, 10 Å, 9 Å, 8 Å, 7 Å, 6 Å, or 5 Å.

The disclosure further provides that the proximity between the N-terminus of the antigen binding fragment and the paratope of the antigen binding fragment (e.g. using the nearest non-hydrogen atom on the surface of the bound antigen) can be determined by looking at such from the existing structures in databases (e.g. PDB). Such proximity can also be determined via homology structure modeling using the numerous structures available in the structure databases, as practiced by a person skilled in the art. Additionally, such proximity can be determined via other structures determined by other structure simulation software or methods, such as Molecular Dynamics or Molecular Mechanics (e.g. CHARMM, AMBER, and NAMD) and ab initio protein modelling (e.g. Rosetta), as practiced by a person skilled in the art. Accordingly, the disclosure provides and a person of ordinary skill in the art would understand that the structure of antigen bound to the antigen binding fragment and the proximity between the N-terminus of the antigen binding fragment and the paratope (e.g. using the nearest non-hydrogen atom on the surface of the bound antigen as the proxy) can be determined without having to experimentally determine any structure.

Alternatively, the proximity between the N-terminus of the antigen binding fragment and the paratope (e.g. using the nearest non-hydrogen atom on the surface of the bound antigen as the proxy) can be determined using the functional effect of placing a linkage at the N-terminus of the antigen binding fragment as a reporter for such proximity. As provided above, the proximity between the N-terminus of the antigen binding fragment and the paratope serves to determine whether there is sufficient space for linking the two binding moieties of the co-binders without interfering with the binding to the target antigen. The disclosure further provides that the affinity of the antigen binding fragment would be negatively affected upon inserting or linking a linker to the N-terminus of the antigen binding fragment, if such proximity between the N-terminus of the antigen binding fragment and the paratope is below a certain threshold that is needed to provide sufficient space for linking the two binding moieties of the co-binders without interfering with the binding to the target antigen. Therefore, the disclosure provides that the affinity changes upon inserting or linking a linker to the N-terminus of the antigen binding fragment can be correlated with the determination whether the proximity between the N-terminus of the antigen binding fragment and the paratope is below a certain threshold sufficient for linking the two binding moieties of the co-binders without interfering with the binding to the target antigen.

Accordingly, the disclosure provides that an antigen binding fragment (e.g. VH, VL, VHAb, VHAb2, VLAb, VLAb2, VR, and VR2 as described herein is suitable to be used to construct a co-binder provided herein and produce the affinity and/or specificity improvement provided for the co-binders of the disclosure, if the affinity of the antigen binding fragment to the antigen changes above certain threshold upon inserting or linking a linker to the N-terminus of the antigen binding fragment. Briefly, in some embodiments, the antigen binding fragment (“ABF”) for a target can be fused with a reference immunoglobulin domain (refIg) that does not specifically bind to the target via a (GGGS)₄ linker to create a refIg-GS-ABF construct. Such ABF is suitable to be used to construct a co-binder provided herein and produce the affinity and/or specificity improvement provided for the co-binders of the disclosure, if the affinity of the fusion construct refIg-GS-ABF to the target is weaker by a certain threshold than the affinity of ABF to the target. In one specific embodiment, the affinity of the fusion construct refIg-GS-ABF to the target is at least 2 fold weaker, such as at least any of the following fold weaker-3, 4, 5, 6, 7, 8, 9, 10, 15, of 20, than the affinity of ABF to the target.

Similarly, as affinity can be measured by dissociation equilibrium constant (K_{D}), the disclosure provides that an antigen binding fragment (e.g. VH, VL, VHAb, VHAb2, VLAb, VLAb2, VR, and VR2 as described herein is suitable to be used to construct a co-binder provided herein and produce the affinity and/or specificity improvement provided for the co-binders of the disclosure, if the K_{D} of the antigen binding fragment to the antigen changes above certain threshold upon inserting or linking a linker to the N-terminus of the antigen binding fragment. Briefly, in some embodiments, the antigen binding fragment (“ABF”) for a target can be fused with a reference immunoglobulin domain (refIg) that does not specifically bind to the target via a (GGGS)₄ linker to create a refIg-GS-ABF construct. Such ABF is suitable to be used to construct a co-binder provided herein and produce the affinity and/or specificity improvement provided for the co-binders of the disclosure, if the K_{D} of the fusion construct refIg-GS-ABF to the target is larger by a certain threshold than the K_{D} of ABF to the target. In one specific embodiment, the K_{D} of the fusion construct refIg-GS-ABF to the target is at least 2 fold larger, such as at least any of the following fold larger—3, 4, 5, 6, 7, 8, 9, 10, 15, or 20, than the K_{D} of ABF to the target.

Additionally, in some specific embodiments provided herein, the antigen binding fragment (“ABF”) for a target can be fused with a reference immunoglobulin domain (refIg) that does not specifically bind to the target via a (GGGS)x linker to create the refIg-GS-ABF construct, wherein x can be 1, 2, 3, 4, 5, 6, 7, or 8. In one embodiment, the ABF for a target can be fused with a refIg that does not specifically bind to the target via a GGGS linker to create the refIg-GS-ABF construct described herein. In another embodiment, the ABF for a target can be fused with a refIg that does not specifically bind to the target via a (GGGS)2 linker to create the refIg-GS-ABF construct described herein. In a further embodiment, the ABF for a target can be fused with a refIg that does not specifically bind to the target via a (GGGS)3 linker to create the refIg-GS-ABF construct described herein. In yet another embodiment, the ABF for a target can be fused with a refIg that does not specifically bind to the target via a (GGGS)4 linker to create the refIg-GS-ABF construct described herein. In one embodiment, the ABF for a target can be fused with a refIg that does not specifically bind to the target via a (GGGS)5 linker to create the refIg-GS-ABF construct described herein. In another embodiment, the ABF for a target can be fused with a refIg that does not specifically bind to the target via a (GGGS)6 linker to create the refIg-GS-ABF construct described herein. In a further embodiment, the ABF for a target can be fused with a refIg that does not specifically bind to the target via a (GGGS)7 linker to create the refIg-GS-ABF construct described herein. In yet another embodiment, the ABF for a target can be fused with a refIg that does not specifically bind to the target via a (GGGS)8 linker to create the refIg-GS-ABF construct described herein.

The disclosure provides that the refIg described herein can be any immunoglobulin domain (e.g. VH, VL, scFv, VHH) as long as the refIg does not specifically bind to the same antigen that the co-binder is specifically constructed to bind to. For example and as demonstrated herein, an anti-human lysozyme VHH, HuL6, can be used as such refIg, when the co-binders were constructed to bind to EGFR. Similarly, in some embodiments, the refIg can be an antigen binding domain or fragment (e.g. VH, VL, scFv, or VHH) of an isotype immunoglobulin. In certain embodiments, the refIg can be an antigen binding domain or fragment (e.g. VH, VL, scFv, or VHH) that binds to an antigen different from the antigen that the co-binder is specifically constructed to bind to.

### C. Linkers

In certain aspects of the binder molecules described herein, a binder molecule comprises a linker. Generally, the linkers described herein are associated, such as covalently, with one or more components of the binder molecules described herein. For example, in some embodiments, the co-binder comprises a second binding moiety and a linker, wherein the linker is attached to the second binding moiety via the N-terminus of the second binding moiety. In some embodiments, the co-binder comprises a linker connecting a first binding moiety and a second binding moiety, wherein the linker is attached to the second binding moiety via the N-terminus of the second binding moiety, and wherein the linker is attached to the first binding moiety via the C-terminus of the first binding moiety. In some embodiments, the second binding moiety is an N-terminal truncated antibody variable domain. In some embodiments, the linker connects a first binding moiety and a second binding moiety via covalent bonds. In some embodiments, the linker connects a first binding moiety and a second binding moiety via a combination of a covalent bond and non-covalent bonds, e.g., the linker is covalently bound to the second binding moiety or the first binding moiety and non-covalently bound to the other binding moiety. In some embodiments, the linker of a co-binder facilitates the co-binder to achieve cooperative and/or synergistic binding interaction to its target molecule. As described herein, linkers may take many forms and can be selected based on a variety of characteristics.

In some embodiments, the linker comprises a polypeptide. In some embodiments, the linker is a polypeptide. In some embodiments, the linker comprises a polypeptide complex, such as comprising two or more polypeptide subunits. In some embodiments, the linker comprises a polynucleotide. In some embodiments, the linker is a polynucleotide. In some embodiments, the linker is a polynucleotide complex, such as a first polynucleotide strand and a second polynucleotide strand having a complementary region. In some embodiments, the linker is a chemical or synthetic linker, such as a polymer-based linker.

In some embodiments, the linker is covalently attached to a binding moiety of a binder molecule. For example, in some embodiments, the binder molecule comprises a second binding moiety and a linker, wherein the second binding moiety and the linker are a single polypeptide. In some embodiments, the linker is non-covalently associated with a binding moiety of a binder molecule.

In some embodiments, the linker is associated with, such as covalently attached to, the N-terminus of a second binding moiety. In some embodiments, the terminal portion of the linker associated with, such as covalently attached to, the N-terminus of a second binding moiety comprises three amino acids, X₁-X₂-X₃. For example, in some embodiments, the linker comprises a polypeptide, wherein the C-terminal portion of the linker associated with, such as covalently attached to, the N-terminus of a second binding moiety comprises three amino acids, X₁-X₂-X₃, in the N- to C-terminal direction. In some embodiments, X₃ is covalently attached to the N-terminal residue of a second binding moiety, such as via a peptide bond.

In some embodiments, the X₃ of X₁-X₂-X₃ of the C-terminal portion of a linker is G, R, or Y. In some embodiments, the X₃ of X₁-X₂-X₃ of the C-terminal portion of a linker is G or R. In some embodiments, the X₃ of X₁-X₂-X₃ of the C-terminal portion of a linker is G or Y. In some embodiments, the X₃ of X₁-X₂-X₃ of the C-terminal portion of a linker is R or Y. In some embodiments, the X₃ of X₁-X₂-X₃ of the C-terminal portion of a linker is G. In some embodiments, the X₃ of X₁-X₂-X₃ of the C-terminal portion of a linker is R. In some embodiments, the X₃ of X₁-X₂-X₃ of the C-terminal portion of a linker is Y.

In some embodiments, the X₁ of X₁-X₂-X₃ of the C-terminal portion of a linker is V, L, W, P, S, G, K, D, F, M, T, N, or R. In some embodiments, the X₁ of X₁-X₂-X₃ of the C-terminal portion of a linker is V, L, W, P, S, G, K, D, F, M, T, N, or R; and the X₃ of X₁-X₂-X₃ of the C-terminal portion of the linker is G, R, or Y. In some embodiments, the X₃ of X₁-X₂-X₃ of the C-terminal portion of a linker is G or R. In some embodiments, the X₃ of X₁-X₂-X₃ of the C-terminal portion of a linker is G or Y. In some embodiments, the X₃ of X₁-X₂-X₃ of the C-terminal portion of a linker is R or Y. In some embodiments, the X₃ of X₁-X₂-X₃ of the C-terminal portion of a linker is G. In some embodiments, the X₃ of X₁-X₂-X₃ of the C-terminal portion of a linker is R. In some embodiments, the X₃ of X₁-X₂-X₃ of the C-terminal portion of a linker is Y.

In certain embodiments, the X₂ of X₁-X₂-X₃ of the C-terminal portion of a linker is V, A, L, S, G, R, K, M, C, F, T, P, or E; and the X₃ of X₁-X₂-X₃ of the C-terminal portion of the linker is G, R, or Y. In some embodiments, the X₃ of X₁-X₂-X₃ of the C-terminal portion of a linker is G or R. In some embodiments, the X₃ of X₁-X₂-X₃ of the C-terminal portion of a linker is G or Y. In some embodiments, the X₃ of X₁-X₂-X₃ of the C-terminal portion of a linker is R or Y. In some embodiments, the X₃ of X₁-X₂-X₃ of the C-terminal portion of a linker is G. In some embodiments, the X₃ of X₁-X₂-X₃ of the C-terminal portion of a linker is R. In some embodiments, the X₃ of X₁-X₂-X₃ of the C-terminal portion of a linker is Y.

In some embodiments, the X₁ of X₁-X₂-X₃ of the C-terminal portion of a linker is V, L, W, P, S, G, K, D, F, M, T, N, or R; the X₂ of X₁-X₂-X₃ of the C-terminal portion of the linker is V, A, L, S, G, R, K, M, C, F, T, P, or E; and the X₃ of X₁-X₂-X₃ of the C-terminal portion of the linker is G, R, or Y. In some embodiments, the X₃ of X₁-X₂-X₃ of the C-terminal portion of a linker is G or R. In some embodiments, the X₃ of X₁-X₂-X₃ of the C-terminal portion of a linker is G or Y. In some embodiments, the X₃ of X₁-X₂-X₃ of the C-terminal portion of a linker is R or Y. In some embodiments, the X₃ of X₁-X₂-X₃ of the C-terminal portion of a linker is G. In some embodiments, the X₃ of X₁-X₂-X₃ of the C-terminal portion of a linker is R. In some embodiments, the X₃ of X₁-X₂-X₃ of the C-terminal portion of a linker is Y.

In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is G, R, or Y. In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is G or R. In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is G or Y. In some embodiments, the X₁ of X₁-X₂-X₃ s of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is R or Y. In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the inker is G. In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is R. In some embodiments, the X₁ of X₁-X₂-X₃ of the C-terminal portion of a linker is any amino acid; the X₂ of X₁-X₂-X₃ of the C-terminal portion of the linker is any amino acid; and the X₃ of X₁-X₂-X₃ of the C-terminal portion of the linker is Y.

In some embodiments, the C-terminal portion of a linker (X₁-X₂-X₃) is VVG, VAG, VLG, VSG, VGG, VRG, VKG, VMG, VCG, VFG, VTG, VPG, or VEG. In some embodiments, the C-terminal portion of a linker (X₁-X₂-X₃) is LVG, LAG, LLG, LSG, LGG, LRG, LKG, LMG, LCG, LFG, LTG, LPG, or LEG. In some embodiments, the C-terminal portion of a linker (X₁-X₂-X₃) is WVG, WAG, WLG, WSG, WGG, WRG, WKG, WMG, WCG, WFG, WTG, WPG, or WEG. In some embodiments, the C-terminal portion of a linker (X₁-X₂-X₃) is PVG, PAG, PLG, PSG, PGG, PRG, PKG, PMG, PCG, PFG, PTG, PPG, or PEG. In some embodiments, the C-terminal portion of a linker (X₁-X₂-X₃) is SVG, SAG, SLG, SSG, SGG, SRG, SKG, SMG, SCG, SFG, STG, SPG, or SEG. In some embodiments, the C-terminal portion of a linker (X₁-X₂-X₃) is GVG, GAG, GLG, GSG, GGG, GRG, GKG, GMG, GCG, GFG, GTG, GPG, or GEG. In some embodiments, the C-terminal portion of a linker (X₁-X₂-X₃) is KVG, KAG, KLG, KSG, KGG, KRG, KKG, KMG, KCG, KFG, KTG, KPG, or KEG. In some embodiments, the C-terminal portion of a linker (X₁-X₂-X₃) is DVG, DAG, DLG, DSG, DGG, DRG, DKG, DMG, DCG, DFG, DTG, DPG, or DEG. In some embodiments, the C-terminal portion of a linker (X₁-X₂-X₃) is FVG, FAG, FLG, FSG, FGG, FRG, FKG, FMG, FCG, FFG, FTG, FPG, or FEG. In some embodiments, the C-terminal portion of a linker (X₁-X₂-X₃) is MVG, MAG, MLG, MSG, MGG, MRG, MKG, MMG, MCG, MFG, MTG, MPG, or MEG. In some embodiments, the C-terminal portion of a linker (X₁-X₂-X₃) is TVG, TAG, TLG, TSG, TGG, TRG, TKG, TMG, TCG, TFG, TTG, TPG, or TEG. In some embodiments, the C-terminal portion of a linker (X₁-X₂-X₃) is NVG, NAG, NLG, NSG, NGG, NRG, NKG, NMG, NCG, NFG, NTG, NPG, or NEG. In some embodiments, the C-terminal portion of a linker (X₁-X₂-X₃) is RVG, RAG, RLG, RSG, RGG, RRG, RKG, RMG, RCG, RFG, RTG, RPG, or REG.

In some embodiments, the C-terminal portion of a linker (X₁-X₂-X₃) is VVR, VAR, VLR, VSR, VGR, VRR, VKR, VMR, VCR, VFR, VTR, VPR, or VER. In some embodiments, the C-terminal portion of a linker (X₁-X₂-X₃) is LVR, LAR, LLR, LSR, LGR, LRR, LKR, LMR, LCR, LFR, LTR, LPR, or LER. In some embodiments, the C-terminal portion of a linker (X₁-X₂-X₃) is WVR, WAR, WLR, WSR, WGR, WRR, WKR, WMR, WCR, WFR, WTR, WPR, or WER. In some embodiments, the C-terminal portion of a linker (X₁-X₂-X₃) is PVR, PAR, PLR, PSR, PGR, PRR, PKR, PMR, PCR, PFR, PTR, PPR, or PER. In some embodiments, the C-terminal portion of a linker (X₁-X₂-X₃) is SVR, SAR, SLR, SSR, SGR, SRR, SKR, SMR, SCR, SFR, STR, SPR, or SER. In some embodiments, the C-terminal portion of a linker (X₁-X₂-X₃) is GVR, GAR, GLR, GSR, GGR, GRR, GKR, GMR, GCR, GFR, GTR, GPR, or GER. In some embodiments, the C-terminal portion of a linker (X₁-X₂-X₃) is KVR, KAR, KLR, KSR, KGR, KRR, KKR, KMR, KCR, KFR, KTR, KPR, or KER. In some embodiments, the C-terminal portion of a linker (X₁-X₂-X₃) is DVR, DAR, DLR, DSR, DGR, DRR, DKR, DMR, DCR, DFR, DTR, DPR, or DER. In some embodiments, the C-terminal portion of a linker (X₁-X₂-X₃) is FVR, FAR, FLR, FSR, FGR, FRR, FKR, FMR, FCR, FFR, FTR, FPR, or FER. In some embodiments, the C-terminal portion of a linker (X₁-X₂-X₃) is MVR, MAR, MLR, MSR, MGR, MRR, MKR, MMR, MCR, MFR, MTR, MPR, or MER. In some embodiments, the C-terminal portion of a linker (X₁-X₂-X₃) is TVR, TAR, TLR, TSR, TGR, TRR, TKR, TMR, TCR, TFR, TTR, TPR, or TER. In some embodiments, the C-terminal portion of a linker (X₁-X₂-X₃) is NVR, NAR, NLR, NSR, NGR, NRR, NKR, NMR, NCR, NFR, NTR, NPR, or NER. In some embodiments, the C-terminal portion of a linker (X₁-X₂-X₃) is RVR, RAR, RLR, RSR, RGR, RRR, RKR, RMR, RCR, RFR, RTR, RPR, or RER.

In some embodiments, the C-terminal portion of a linker (X₁-X₂-X₃) is VVY, VAY, VLY, VSY, VGY, VRY, VKY, VMY, VCY, VFY, VTY, VPY, or VEY. In some embodiments, the C-terminal portion of a linker (X₁-X₂-X₃) is LVY, LAY, LLY, LSY, LGY, LRY, LKY, LMY, LCY, LFY, LTY, LPY, or LEY. In some embodiments, the C-terminal portion of a linker (X₁-X₂-X₃) is WVY, WAY, WLY, WSY, WGY, WRY, WKY, WMY, WCY, WFY, WTY, WPY, or WEY. In some embodiments, the C-terminal portion of a linker (X₁-X₂-X₃) is PVY, PAY, PLY, PSY, PGY, PRY, PKY, PMY, PCY, PFY, PTY, PPY, or PEY. In some embodiments, the C-terminal portion of a linker (X₁-X₂-X₃) is SVY, SAY, SLY, SSY, SGY, SRY, SKY, SMY, SCY, SFY, STY, SPY, or SEY. In some embodiments, the C-terminal portion of a linker (X₁-X₂-X₃) is GVY, GAY, GLY, GSY, GGY, GRY, GKY, GMY, GCY, GFY, GTY, GPY, or GEY. In some embodiments, the C-terminal portion of a linker (X₁-X₂-X₃) is KVY, KAY, KLY, KSY, KGY, KRY, KKY, KMY, KCY, KFY, KTY, KPY, or KEY. In some embodiments, the C-terminal portion of a linker (X₁-X₂-X₃) is DVY, DAY, DLY, DSY, DGY, DRY, DKY, DMY, DCY, DFY, DTY, DPY, or DEY. In some embodiments, the C-terminal portion of a linker (X₁-X₂-X₃) is FVY, FAY, FLY, FSY, FGY, FRY, FKY, FMY, FCY, FFY, FTY, FPY, or FEY. In some embodiments, the C-terminal portion of a linker (X₁-X₂-X₃) is MVY, MAY, MLY, MSY, MGY, MRY, MKY, MMY, MCY, MFY, MTY, MPY, or MEY. In some embodiments, the C-terminal portion of a linker (X₁-X₂-X₃) is TVY, TAY, TLY, TSY, TGY, TRY, TKY, TMY, TCY, TFY, TTY, TPY, or TEY. In some embodiments, the C-terminal portion of a linker (X₁-X₂-X₃) is NVY, NAY, NLY, NSY, NGY, NRY, NKY, NMY, NCY, NFY, NTY, NPY, or NEY. In some embodiments, the C-terminal portion of a linker (X₁-X₂-X₃) is RVY, RAY, RLY, RSY, RGY, RRY, RKY, RMY, RCY, RFY, RTY, RPY, or REY.

In some embodiments, the C-terminal portion of a linker (X₁-X₂-X₃) is any one selected from Table 2.

TABLE 2
Exemplary sequences of the C-terminal three amino acids of a linker.

| | | | | | | | | | | |
|---|---|---|---|---|---|---|---|---|---|---|
| HKR | FKR | FRG | GPG | FKR | CCG | YSG | GVG | LLG | PGG | TLG |
| FKR | YKR | TYG | FNG | EKR | FWG | WTG | DSG | VSG | SCG | NPG |
| MKR | EKR | SYG | FSG | YKR | FLG | FWG | LRG | WLG | VMG | VRG |
| CKR | WKR | WRG | FPG | WKR | FTG | YTG | FAG | VSG | GFG | WPG |
| QKR | QKR | YYG | FTG | QKR | FQG | YGG | MVG | LLG | KMG | PAG |
| VKR | VKR | RYG | YAG | VKR | FVG | FTG | SVG | VSG | LVG | VSG |
| RKR | ZLE | TFG | FAG | HKR | FIG | FSG | TKG | VSG | VKG | RGG |
| LKR | ZHQ | VYG | WPG | EHY | YGG | YNG | KMG | WLG | VSG | RAG |
| KKR | HKR | NZV | HPG | CKR | FMG | HTG | DGG | LLG | VGG | VEG |
| WKR | MZL | CCF | CTZ | FRR | YTG | HPG | VSG | LLG | VTG | GTG |
| SKR | AMV | FYG | YPG | IKR | FSG | IAG | SVG | TAG | FTG | HGG |
| KRG | EHY | HYG | SPG | AKR | YLG | WVG | KLG | GIG | GDG | ALG |
| EKR | TYP | QYG | YTG | YRR | YIG | WIG | LGG | WEG | NSG | QRG |
| YKR | WAP | KYG | FVG | MKR | ITG | LTG | VLG | VSG | ALG | SHG |
| IKR | YMY | IYG | FLG | QNY | VVG | WSG | LSG | VYG | YSG | LVG |
| TKR | CKR | CCY | IPG | RKR | FGG | FYG | MAG | WLG | FSG | SQG |
| NKR | IYK | FFG | YLG | LKR | YQG | ATG | GAG | LLG | LMG | RRG |
| FRR | YTY | QYH | FIG | DKR | IIG | YVG | AVG | IGG | YSG | MZG |
| YRR | YYP | GYG | MPG | SKR | YVG | MAG | EAG | IGG | HQG | CMG |
| AKR | FRR | MYG | YVG | SGY | YNG | HVG | VLG | TAG | MAG | ALG |

In some embodiments, the linker comprises a peptide sequence comprising (EAAAK)ₙ, wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (XP)ₙ, (XPP)ₙ, or (XPPP)ₙ, wherein X is any amino acid, and wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (XP)ₙ, (XPP)ₙ, or (XPPP)ₙ, wherein each X is G, A, P, or S, and wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (AP)ₙ or (APAP)ₙ, wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (EEEEKKKK)ₙ, wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (GₓS_{y})ₙ, wherein x is 1 to 5, wherein y is 1 to 5, and wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence comprising (GGGGS)ₙ, wherein n is an integer number from 1 to 25 (e.g., 1 to 20 or 1 to 10), including any of 1, 2, 3, 4, 5, 6, 7, 8, 9, 10, 11, 12, 13, 14, 15, 16, 17, 18, 19, 20, 21, 22, 23, 24, or 25. In some embodiments, the linker comprises a peptide sequence disclosed herein and X₁-X₂-X₃ of the C-terminal portion of the linker disclosed herein.

In some embodiments, the linker is a rigid linker. In some embodiments, the linker is a flexible linker. In some embodiments, the linker is a cleavable linker. In some embodiments, the linker is a non-cleavable linker.

In some embodiments, the rigidity of a rigid linker is maximized to increase the affinity of the co-binding moieties. In some embodiments, the rigid linker can only bend or flex no more than 90 degrees, such as no more than any of 75 degrees, 60 degrees, 45 degrees, 30 degrees, 15 degrees, or 5 degrees. In some embodiments, the rigid linker can only bend or flex no more than 45 degrees and can twist no more than 30 degrees. In some embodiments, the linker can only twist less than 360 degrees, such less than any of 300 degrees, 240 degrees, 180 degrees, 150 degrees, 120 degrees, 90 degrees, 60 degrees, 30 degrees, 15 degrees, or 5 degrees. In some embodiments, a linker can only twist less than 5 degrees. In some embodiments, the rigid linker can only bend or flex no more than 90 degrees, such as no more than any of 75 degrees, 60 degrees, 45 degrees, 30 degrees, 15 degrees, or 5 degrees, and can twist less than any of 360 degrees, 300 degrees, 240 degrees, 180 degrees, 150 degrees, 120 degrees, 90 degrees, 60 degrees, 30 degrees, 15 degrees, or 5 degrees.

In some embodiments, the rigid linker has a rigid middle portion and less rigid tips on one or more ends that connect to binding moieties. In some embodiments, the rigid linker has a rigid middle portion and less rigid tips on one or more ends that connect to binding moieties. For example, such rigid linkers may facilitate simultaneous binding of binding moieties to non-overlapping epitopes on a target molecule.

In some embodiments, the linker associates a first binding moiety and a second binding moiety via a non-covalent interaction. In such embodiments, the first binding moiety and/or second binding moiety comprises a moiety involved in a non-covalent interaction. For example, in some embodiments, the linker comprises a leucine zipper, wherein the first binding moiety comprises a first portion of the leucine zipper, and wherein the second binding moiety comprises a second portion of the leucine zipper. In some embodiments, the linker comprises a double-strand nucleic acid comprising two strands having a complementary region, wherein the first binding moiety comprises a nucleic acid strand, and wherein the second binding moiety comprises a second nucleic acid strand.

In some embodiments, the nucleic acid linker comprises a polynucleotide, such as an oligonucleotide, a double-stranded DNA, a single-stranded DNA, a double-stranded RNA or a single-stranded RNA. In some embodiments, the nucleic acid linker comprises 200 nucleotides or fewer, such as any of 180 nucleotides or fewer, 160 nucleotides or fewer, 140 nucleotides or fewer, 120 nucleotides or fewer, 100 nucleotides or fewer, 80 nucleotides or fewer, 60 nucleotides or fewer, 40 nucleotides or fewer, 20 nucleotides or fewer, or 10 nucleotides or fewer.

In some embodiments, the linker has a length, such as based on the primary structure of the linker, e.g., a linear chain of amino acids. In some embodiments, the length of the linker is assessed based on a primary structure, e.g., a linear chain of amino acids. In some embodiments, the linker has a length of no more than 250 angstroms, such as no more than any of 240 angstroms, 230 angstroms, 220 angstroms, 210 angstroms, 200 angstroms, 190 angstroms, 180 angstroms, 170 angstroms, 160 angstroms, 150 angstroms, 140 angstroms, 130 angstroms, 120 angstroms, 110 angstroms, 100 angstroms, 90 angstroms, 80 angstroms, 70 angstroms, 60 angstroms, 50 angstroms, 40 angstroms, 30 angstroms, 20 angstroms, 15 angstroms, 10 angstroms, or 5 angstroms. In some embodiments, the linker has a length of about any of 250 angstroms, 240 angstroms, 230 angstroms, 220 angstroms, 210 angstroms, 200 angstroms, 190 angstroms, 180 angstroms, 170 angstroms, 160 angstroms, 150 angstroms, 140 angstroms, 130 angstroms, 120 angstroms, 110 angstroms, 100 angstroms, 90 angstroms, 80 angstroms, 70 angstroms, 60 angstroms, 50 angstroms, 40 angstroms, 30 angstroms, 20 angstroms, 15 angstroms, 10 angstroms, or 5 angstroms. In some embodiments, the length of a linker is reduced to the minimum length required to provide linkage of at least a first binding moiety and second binding moiety without interfering with the binding of a respective binding molecule to a target molecule. In some embodiments, the length of a linker is configured to achieve both minimum entropy loss and least interference to the binding of a binding molecule to a target molecule.

In some embodiments, the linker has a length of no more than 120 amino acids, such as no more than any of 115 amino acids, 110 amino acids, 105 amino acids, 100 amino acids, 95 amino acids, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids. In some embodiments, the linker has a length of about any of 120 amino acids, 115 amino acids, 110 amino acids, 105 amino acids, 100 amino acids, 95 amino acids, 90 amino acids, 85 amino acids, 80 amino acids, 75 amino acids, 70 amino acids, 65 amino acids, 60 amino acids, 55 amino acids, 50 amino acids, 45 amino acids, 40 amino acids, 35 amino acids, 30 amino acids, 25 amino acids, 20 amino acids, 15 amino acids, 10 amino acids, or 5 amino acids.

In some embodiments, the linker is a chemical linker, such as a synthetic chemical structure or a polymer. In some embodiments, the linker comprises a plurality of polyethylene glycol subunits. In some embodiments, the linker is a non-peptidyl polymer.

### D. Other Configurations, Alternatives, and Variants of the Binder Molecules

Provided herein are various configurations of binder molecules comprising a second binding moiety specifically recognizing a target site, wherein the second binding moiety is a second antibody moiety comprising an antibody variable domain having an N-terminal truncation (“N-terminal truncated antibody variable domain”). In some embodiments, the binder molecule comprises a linker. In some embodiments, the binder molecule does not comprises a linker. In some embodiments, the binder molecule comprises a first moiety that is not a binding moiety, such as an enzyme, drug, or toxin. In some embodiments, the binder molecule is a multispecific binder molecule, such as a bispecific co-binder. In some embodiments, the binder molecule, such as a co-binder, is a multimeric binder molecule comprising at least a third binding moiety. In some embodiments, the binder molecule is a CAR, including multispecific CAR, such as a bispecific CAR. In some embodiments, the binder molecule is a conjugate, such as a co-binder conjugated to a drug or label, including a bispecific conjugate.

In some embodiments, the binder molecule comprises a first moiety that is a non-immunoglobulin binder molecules that specifically bind to a target. These alternative binder molecules may include, for example, any of the engineered protein scaffolds known in the art. Such scaffolds may comprise one or more CDRs of an antibody against a target. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352:95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

In some embodiments, the disclosure encompasses amino acid sequence modification(s) of the binder molecules, such as co-binders. In some embodiments, the antibody or antigen binding fragments thereof comprise amino acid sequence modification(s). For example, it may be desirable to improve the binding affinity and/or other biological properties of the antibody, including but not limited to specificity, thermostability, expression level, effector functions, glycosylation, reduced immunogenicity, or solubility. Thus, it is contemplated that binder molecule, such as co-binder, variants can be prepared. For example, co-binder variants can be prepared by introducing appropriate nucleotide changes into the encoding DNA, and/or by synthesis of the desired antibody or polypeptide. Those skilled in the art who appreciate that amino acid changes may alter post-translational processes of the co-binder, or the antibody or antigen binding fragments thereof that are part of the co-binder, such as changing the number or position of glycosylation sites or altering the membrane anchoring characteristics.

In some embodiments, binder molecules, such as co-binders, provided herein are chemically modified, for example, by the covalent attachment of any type of molecule to the binder molecule, such as a co-binder, or the antibody or antigen binding fragments thereof. Such derivatives may include, e.g., co-binders that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the polypeptide (of the co-binder, or the antibody or antigen binding fragments thereof that are part of the co-binder) that results in a change in the amino acid sequence as compared to the native sequence of the polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion, or insertion includes no more than 25 amino acid substitutions, such as no more than any of 20 amino acid substitutions, 18 amino acid substitutions, 15 amino acid substitutions, 10 amino acid substitutions, 5 amino acid substitutions, 4 amino acid substitutions, 3 amino acid substitutions, or 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the binder molecule, such as a co-binder, or the antibody or antigen binding fragments thereof, are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe

Non-conservative substitutions entail exchanging a member of one of these classes for another class. Such substituted residues also may be introduced into the conservative substitution sites or, into the remaining (non-conserved) sites. The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the co-binder variant DNA.

Any cysteine residue not involved in maintaining the proper conformation of the binder molecule, such as a co-binder, or the antibody or antigen binding fragments thereof, also may be substituted, for example, with another amino acid, such as alanine or serine, to improve the oxidative stability of the molecule and to prevent aberrant crosslinking. Conversely, e.g., cysteine bond(s) may be added to the co-binder, or the antibody or antigen binding fragments thereof that are part of the co-binder, to improve its stability (e.g., where the antibody is an antibody fragment such as an Fv fragment).

In some embodiments, the binder molecule, such as a co-binder, or the antibody or antigen binding fragments thereof are “de-immunized”. In some embodiments, a “de-immunized” binder molecule, such as a co-binder, comprises a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, for example, by in vitro determination of T-cell epitopes or in silico prediction of such epitopes, as known in the art. Once the critical residues for T-cell epitope function have been identified, mutations can be made to remove immunogenicity and retain antibody activity. For review, see, for example, Jones et al., 2009, Methods in Molecular Biology 525:405-23.

In certain aspects, covalent modifications of binder molecules, such as co-binders, are included within the scope of the present disclosure. Covalent modifications include reacting targeted amino acid residues of a binder molecule, such as a co-binder, with an organic derivatizing agent that is capable of reacting with selected side chains or the N- or C-terminal residues of the binder molecule. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the binder molecules, such as co-binders, included within the scope of this present disclosure include altering the native glycosylation pattern (see, e.g., Beck et al., 2008, Curr. Pharm. Biotechnol. 9:482-501; and Walsh, 2010, Drug Discov. Today 15:773-80), and linking the binder molecule to one of a variety of nonproteinaceous polymers, e.g., polyethylene glycol, polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In some embodiments, the binder molecule, such as a co-binder, of the present disclosure may also be modified to form chimeric molecules comprising a co-binder fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

In some embodiments, also provided herein are fusion proteins comprising a binder molecule, such as a co-binder, provided herein and a heterologous polypeptide. In some embodiments, the heterologous polypeptide to which the binder molecule, such as a co-binder, is fused is useful for targeting the binder molecule to specific cells.

The present disclosure also provides conjugates comprising a binder molecule, such as a co-binder, of the present disclosure covalently bound, such as by linker (e.g., a synthetic linker) to one or more agents.

In some embodiments, the binder molecule, such as a co-binder, provided herein is conjugated or recombinantly fused, e.g., to a detectable molecule.

Such detection can be accomplished, for example, by coupling the co-binders to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵ Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also provided herein are binder molecules, such as co-binders, that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising a co-binder provided herein and a heterologous protein, polypeptide, or peptide. In one embodiment, the heterologous protein, polypeptide, or peptide that the antibody is fused to is useful for targeting the co-binders to a particular cell type. For example, an co-binder that binds to a cell surface receptor expressed by a particular cell type may be fused or conjugated to a cytotoxic antibody or peptide.

Moreover, binder molecules, such as co-binders, provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to binder molecules, such as co-binders, are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88:10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of co-binders as provided herein, including, for example, co-binders with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16 (2): 76-82; Hansson et al., 1999, J. Mol. Biol. 287:265-76; and Lorenzo and Blasco, 1998, Biotechniques 24 (2): 308-13). Co-binders, or the antibodies provided herein for the co-binders, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

A binder molecule, such as a co-binder, provided herein can also be conjugated to a second antibody to form an antibody heteroconjugate as described, for example, in U.S. Pat. No. 4,676,980.

Binder molecule, such as a co-binder, as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of co-binders and agents may be prepared using any suitable methods as disclosed in the art (see, e.g., Bioconjugate Techniques (Hermanson ed., 2d ed. 2008)).

Conventional conjugation strategies for binder molecules, such as co-binders, and agents have been based on random conjugation chemistries involving the s-amino group of Lys residues or the thiol group of Cys residues, which results in heterogenous conjugates. Recently developed techniques allow site-specific conjugation to polypeptides, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332:41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48 (50): 12047-57).

### 1. Chimeric Antigen Receptor (CAR)

In some aspects, the disclosure provides a chimeric antigen receptor (CAR) comprising a binder molecule, such as a co-binder provided, herein. In some aspects, the disclosure provides a cell that expresses a CAR provided herein, such as a CAR effector cell. In some embodiments, the cell is an immune cell, e.g., a T cell. The CAR provided here comprise (a) an extracellular domain comprising a binder molecule described herein, and (b) an intracellular signaling domain. In some embodiments, the CAR comprises a transmembrane domain present between the extracellular domain and the intracellular domain.

In some embodiments, between the extracellular domain and the transmembrane domain or between the intracellular domain and the transmembrane domain there may be a spacer domain. The spacer domain can be any oligo- or polypeptide that functions to link the transmembrane domain to the extracellular domain or the intracellular domain in the polypeptide chain. A spacer domain may comprise up to about 300 amino acids, including for example about 10 to about 100, or about 25 to about 50 amino acids.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the α, β, δ, or γ chain of the T-cell receptor, CD28, CD3ε, CD3ζ, CD45, CD4, CD5, CD8, CD9, CD16, CD22, CD33, CD37, CD64, CD80, CD86, CD134, CD137, or CD154. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of the CAR. In some embodiments, the linker is a glycine-serine doublet.

In some embodiments, the transmembrane domain that naturally is associated with one of the sequences in the intracellular domain of the CAR is used. In some embodiments, the transmembrane domain can be selected or modified by amino acid substitution to avoid binding of such domains to the transmembrane domains of the same or different surface membrane proteins to minimize interactions with other members of the receptor complex.

The intracellular signaling domain of the CAR is responsible for activation of at least one of the normal effector functions of the immune cell in which the CAR has been placed in. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. The CAR constructs in some embodiments comprise one or more ITAMs.

Examples of ITAM containing primary signaling sequences that are of particular use in the invention include those derived from TCRζ, FcRγ, FcRβ, CD3γ, CD3δ, CD3ε, CD5, CD22, CD79a, CD79b, and CD66d.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR described herein. For example, the intracellular domain of the CAR can comprise a CD3ζ intracellular signaling sequence and a costimulatory signaling sequence. The costimulatory signaling sequence can be a portion of the intracellular domain of a costimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like.

In some embodiments, the intracellular signaling domain of the CAR comprises the intracellular signaling sequence of CD3ζ and the intracellular signaling sequence of CD28. In some embodiments, the intracellular signaling domain of the CAR comprises the intracellular signaling sequence of CD3ζ and the intracellular signaling sequence of 4-1BB. In some embodiments, the intracellular signaling domain of the CAR comprises the intracellular signaling sequence of CD3ζ and the intracellular signaling sequences of CD28 and 4-1BB.

Also provided herein are effector cells (such as lymphocytes, e.g., T cells) expressing a CAR described herein.

Also provided is a method of producing an effector cell expressing a CAR described herein, the method comprising introducing a vector comprising a nucleic acid encoding the CAR into the effector cell. In some embodiments, introducing the vector into the effector cell comprises transducing the effector cell with the vector. In some embodiments, introducing the vector into the effector cell comprises transfecting the effector cell with the vector. Transduction or transfection of the vector into the effector cell can be carried about using any method known in the art.

### 2. Immunoconjugates

The binder molecules, in some embodiments, comprise an immunoconjugate comprising a binder molecule, such as a co-binder, attached to an effector molecule (also referred to herein as an “immunoconjugate”). In some embodiments the effector molecule is a therapeutic agent, such as a cancer therapeutic agent, which is either cytotoxic, cytostatic or otherwise provides some therapeutic benefit. In some embodiments, the effector molecule is a label, which can generate a detectable signal, either directly or indirectly.

In some embodiments, there is provided an immunoconjugate comprising a binder molecule and a therapeutic agent (also referred to herein as an “antibody-drug conjugate”, or “ADC”). In some embodiments, the therapeutic agent is a toxin that is either cytotoxic, cytostatic or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos, Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986): 603-605 (1986); Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought thereby.

Therapeutic agents used in immunoconjugates include, for example, daunomycin, doxorubicin, methotrexate, and vindesine (Rowland et al., Cancer Immunol. Immunother. 21:183-187 (1986)). Toxins used in immunoconjugates include bacterial toxins such as diphtheria toxin, plant toxins such as ricin, small molecule toxins such as geldanamycin (Mandler et al., J. Nat. Cancer Inst. 92 (19): 1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Immunoconjugates of a binder molecule and one or more small molecule toxins, such as a calicheamicin, maytansinoids, dolastatins, aurostatins, a trichothecene, and CC1065, and the derivatives of these toxins that have toxin activity, are also contemplated herein.

In some embodiments, there is provided an immunoconjugate comprising a therapeutic agent that has an intracellular activity. In some embodiments, the immunoconjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the anti-AMC immunoconjugate must be internalized upon binding to the target cell in order for the protein to be cytotoxic to the cells.

In some embodiments, there is provided an immunoconjugate comprising a therapeutic agent that acts to disrupt DNA. In some embodiments, the therapeutic agent that acts to disrupt DNA is, for example, selected from the group consisting of enediyne (e.g., calicheamicin and esperamicin) and non-enediyne small molecule agents (e.g., bleomycin, methidiumpropyl-EDTA-Fe(II)). Other cancer therapeutic agents useful in accordance with the present application include, without limitation, daunorubicin, doxorubicin, distamycin A, cisplatin, mitomycin C, ecteinascidins, duocarmycin/CC-1065, and bleomycin/pepleomycin.

The present invention further contemplates an immunoconjugate formed between a binder molecule and a compound with nucleolytic activity (e.g., a ribonuclease or a DNA endonuclease such as a deoxyribonuclease; DNase).

In some embodiments, the immunoconjugate comprises an agent that acts to disrupt tubulin. Such agents may include, for example, rhizoxin/maytansine, paclitaxel, vincristine and vinblastine, colchicine, auristatin dolastatin 10 MMAE, and peloruside A.

In some embodiments, the immunoconjugate comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the cancer therapeutic agent portion of the immunoconjugate of the present application may comprise an antimitotic agent including, without limitation, allocolchicine NSC 406042, Halichondrin B NSC 609395, colchicine NSC 757, colchicine derivative NSC 33410, dolastatin 10 NSC 376128 (NG-auristatin derived), maytansine NSC 153858, rhizoxin NSC 332598, taxol NSC 125973, taxol derivative NSC 608832, thiocolchicine NSC 361792, trityl cysteine NSC 83265, vinblastine sulfate NSC 49842, and vincristine sulfate NSC 67574.

In some embodiments, the immunoconjugate comprises a topoisomerase I inhibitor including, without limitation, camptothecin NSC 94600, camptothecin, Na salt NSC 100880, aminocamptothecin NSC 603071, camptothecin derivative NSC 95382, camptothecin derivative NSC 107124, camptothecin derivative NSC 643833, camptothecin derivative NSC 629971, camptothecin derivative NSC 295500, camptothecin derivative NSC 249910, camptothecin derivative NSC 606985, camptothecin derivative NSC 374028, camptothecin derivative NSC 176323, camptothecin derivative NSC 295501, camptothecin derivative NSC 606172, camptothecin derivative NSC 606173, camptothecin derivative NSC 610458, camptothecin derivative NSC 618939, camptothecin derivative NSC 610457, camptothecin derivative NSC 610459, camptothecin derivative NSC 606499, camptothecin derivative NSC 610456, camptothecin derivative NSC 364830, camptothecin derivative NSC 606497, and morpholinodoxorubicin NSC 354646.

In some embodiments, the immunoconjugate comprises a topoisomerase II inhibitor including, without limitation, doxorubicin NSC 123127, amonafide NSC 308847, m-AMSA NSC 249992, anthrapyrazole derivative NSC 355644, pyrazoloacridine NSC 366140, bisantrene HCL NSC 337766, daunorubicin NSC 82151, deoxydoxorubicin NSC 267469, mitoxantrone NSC 301739, menogaril NSC 269148, N,N-dibenzyl daunomycin NSC 268242, oxanthrazole NSC 349174, rubidazone NSC 164011, VM-26 NSC 122819, and VP-16 NSC 141540.

In some embodiments, the immunoconjugate comprises an RNA or DNA antimetabolite including, without limitation, L-alanosine NSC 153353, 5-azacytidine NSC 102816, 5-fluorouracil NSC 19893, acivicin NSC 163501, aminopterin derivative NSC 132483, aminopterin derivative NSC 184692, aminopterin derivative NSC 134033, an antifol NSC 633713, an antifol NSC 623017, Baker's soluble antifol NSC 139105, dichlorallyl lawsone NSC 126771, brequinar NSC 368390, ftorafur (pro-drug) NSC 148958, 5,6-dihydro-5-azacytidine NSC 264880, methotrexate NSC 740, methotrexate derivative NSC 174121, N-(phosphonoacetyl)-L-aspartate (PALA) NSC 224131, pyrazofurin NSC 143095, trimetrexate NSC 352122, 3-HP NSC 95678, 2′-deoxy-5-fluorouridine NSC 27640, 5-HP NSC 107392, α-TGDR NSC 71851, aphidicolin glycinate NSC 303812, ara-C NSC 63878, 5-aza-2′-deoxycytidine NSC 127716, β-TGDR NSC 71261, cyclocytidine NSC 145668, guanazole NSC 1895, hydroxyurea NSC 32065, inosine glycodialdehyde NSC 118994, macbecin Il NSC 330500, pyrazoloimidazole NSC 51143, thioguanine NSC 752, and thiopurine NSC 755.

In some embodiments, the immunoconjugate comprises a highly radioactive atom. A variety of radioactive isotopes are available for the production of radioconjugated antibodies. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu.

In some embodiments, the binder molecule can be conjugated to a “receptor” (such as streptavidin) for utilization in tumor pre-targeting wherein the binder molecule-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, the immunoconjugate may comprise a binder molecule conjugated to a prodrug-activating enzyme. In some such embodiments, the prodrug-activating enzyme converts a prodrug (e.g., a peptidyl chemotherapeutic agent, see WO 81/01145) to an active drug, such as an anti-cancer drug. Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, the therapeutic portion of the immunoconjugates may be a nucleic acid. Nucleic acids that may be used include, but are not limited to, anti-sense RNA, genes or other polynucleotides, including nucleic acid analogs such as thioguanine and thiopurine.

The present application further provides immunoconjugates comprising a binder molecule attached to an effector molecule, wherein the effector molecule is a label, which can generate a detectable signal, indirectly or directly. These immunoconjugates can be used for research or diagnostic applications, such as for the in vivo detection of cancer. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example ⁹⁹Tc or ¹²³I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

### 3. Nucleic Acids

Nucleic acid molecules encoding a binder molecule, including constructs thereof, are also contemplated. In some embodiments, there is provided a nucleic acid (or a set of nucleic acids) encoding a full-length binder molecule, such as a co-binder. In some embodiments, there is provided a nucleic acid (or a set of nucleic acids) encoding a multi-specific binder molecule (e.g., a multi-specific binder molecule, a bispecific binder molecule, or a bispecific T-cell engager), or polypeptide portion thereof. In some embodiments, there is provided a nucleic acid (or a set of nucleic acids) encoding a CAR. In some embodiments, there is provided a nucleic acid (or a set of nucleic acids) encoding an immunoconjugate, or polypeptide portion thereof.

The present application also includes variants to these nucleic acid sequences. For example, the variants include nucleotide sequences that hybridize to the nucleic acid sequences encoding the binder molecules, including constructs thereof, of the present application under at least moderately stringent hybridization conditions.

The present invention also provides vectors in which a nucleic acid of the present invention is inserted.

In brief summary, the expression of a binder molecule, including a construct thereof (e.g., a CAR), or polypeptide portion thereof by a natural or synthetic nucleic acid encoding the binder molecule or polypeptide portion thereof can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### E. Additional Characteristics and Details Regarding the Binder Molecules and Portions Thereof

In some embodiments, the second binding moiety and/or first binding moiety of the binder molecule, such as a co-binder or a component thereof, is derived from a monoclonal antibody, antibody fragment, humanized antibody or human antibody. Monoclonal antibodies are well known in the art, and methods of making and screening are exemplified in, e.g., Kohler et al., 1975, Nature 256:495-97; U.S. Pat. No. 4,816,567; Munson et al., 1980, Anal. Biochem. 107:220-39; Skerra et al., 1993, Curr. Opinion in Immunol. 5:256-62; Pluckthun, 1992, Immunol. Revs. 130:151-88; Bass et al., 1990, Proteins 8:309-14; WO 92/09690; and Bowers et al., 2011, Proc Natl Acad Sci USA. 108:20455-60. Antibody fragments are well known in the art, and methods of making and screening are exemplified in, e.g., Hudson et al., 2003, Nature Med. 9:129-34; Morimoto et al., 1992, J. Biochem. Biophys. Methods 24:107-17; Brennan et al., 1985, Science 229:81-83; Carter et al., 1992, Bio/Technology 10:163-67; U.S. Pat. No. 5,869,046; WO 93/16185; U.S. Pat. Nos. 5,571,894; 5,587,458; Woolven et al., 1999, Immunogenetics 50:98-101; and Streltsov et al., 2004, Proc Natl Acad Sci USA. 101:12444-49. Humanized and human antibodies are well known in the art, and methods of making and screening are exemplified in, e.g., Jones et al., 1986, Nature 321:522-25; Riechmann et al., 1988, Nature 332:323-27; Verhoeyen et al., 1988, Science 239:1534-36; Padlan et al., 1995, FASEB J. 9:133-39; Sims et al., 1993, J. Immunol. 151:2296-308; Chothia et al., 1987, J. Mol. Biol. 196:901-17; Carter et al., 1992, Proc. Natl. Acad. Sci. USA 89:4285-89; Presta et al., 1993, J. Immunol. 151:2623-32; Tan et al., 2002, J. Immunol. 169:1119-25; Lazar et al., 2007, Mol. Immunol. 44:1986-98; Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; Schlapschy et al., 2004, Protein Eng. Des. Sel. 17:847-60; Foote and Winter, 1992, J. Mol. Biol. 224:487-99); Dall'Acqua et al., 2005, Methods 36:43-60; Studnicka et al., 1994, Protein Engineering 7:805-14; U.S. Pat. Nos. 5,766,886; 5,770,196; 5,821,123; and 5,869,619; PCT Publication WO 93/11794; Kozbor, 1984, J. Immunol. 133:3001-05; Brodeur et al., Monoclonal Antibody Production Techniques and Applications 51-63 (1987); and Boerner et al., 1991, J. Immunol. 147:86-95; Jakobovits, A., 1995, Curr. Opin. Biotechnol. 6 (5): 561-66; Brüggemann and Taussing, 1997, Curr. Opin. Biotechnol. 8 (4): 455-58; U.S. Pat. Nos. 6,075,181 and 6,150,584.

### III. Targets

The binder molecules provided herein comprise elements, such as a first binding moiety and a second binding moiety, that specifically recognize one or more targets comprising a target site (e.g., an epitope). In some embodiments, the first binding moiety and the second binding moiety specifically recognize different epitopes on the same target, e.g., the first binding moiety recognizes a first epitope on a target, such as a polypeptide, and the second binding moiety recognizes a second epitope on the target. In some embodiments, the first binding moiety and the second binding moiety specifically recognize the same epitope on the same target, e.g., the first binding moiety and the second binding moiety specifically recognize a homodimer. In some embodiments, the first binding moiety and the second binding moiety specifically recognize different epitopes on different targets, e.g., the first binding moiety recognizes a first epitope on a first target, such as a polypeptide, and the second binding moiety recognizes a second epitope on a second target, such as a polypeptide. In such an embodiment, the first target and the second target are in proximity to one another. In some embodiments, the first target and the second target form a single complex, such as a protein complex.

In some embodiments, the target is a polypeptide, a multi-protein complex, a nucleic acid, a carbohydrate, a glycan, a lipid molecule, a physiological metabolite, or a small molecule compound. In some embodiments, the target molecule is a polypeptide. In some embodiments, the target molecule is a protein. In some embodiments, the target molecule is a multiprotein complex. In some embodiments, the target molecule is a nucleic acid. In some embodiments, the target molecule is a DNA molecule. In some embodiments, the target molecule is a RNA molecule. In some embodiments, the target molecule is a lipid molecule. In some embodiments, the target molecule is a sugar. In some embodiments, the target molecule is a carbohydrate. In some embodiments, the target molecule is a glycan. In some embodiments, the target molecule is a physiological metabolite. In some embodiments, the target molecule is a small molecule compound.

In some embodiments, the target is a polypeptide, a multi-protein complex, a nucleic acid, a carbohydrate, a glycan, a lipid molecule, a physiological metabolite, or a small molecule compound. In some embodiments, the target is an intracellular molecule, a disease marker, a neoantigen, or a cell surface molecule. In some embodiments, the target molecule is a cancer antigen or cancer marker. In some embodiments, the target is EGFR. In some embodiments, the target is expressed at below 1×10⁶, below 1×10⁵, below 1×10⁴, below 1×10³, or below 1×10² per cell.

Binding moieties with high binding affinity (e.g. K_{D} of 1×10⁻¹² M) for a target molecule are more likely to have high binding affinity for a nonspecific target (e.g. K_{D} of 1×10⁻⁹ M) as compared to binding moieties with low binding affinity (e.g. K_{D} of 1×10⁻⁹ M) for the target molecule. In some embodiments, binding moieties with low binding affinity to a target molecule are selected to make co-binders. In some embodiments, co-binders having high binding affinity (e.g. K_{D} of 1×10⁻¹² M or less) comprise first and second binding moieties either or both having a K_{D} of at least 1×10⁻¹⁰ M, at least 1×10⁻⁹, at least 1×10⁻⁸, at least 1×10⁻⁷, or at least 1×10⁻⁶ M so that the nonspecific binding can be reduced and minimized.

In some embodiments, the binding of a binder molecule is reported relative to that of a control binder molecule, such as a control co-binder. As described herein, in some embodiments, the control binder molecule (such as a control co-binder) is comprising an antibody variable domain not having an N-terminal truncation in the second binding moiety. In some embodiments, the binder molecule, such as a co-binder, comprises a second antibody moiety comprising an N-terminal truncated antibody variable domain that binds to a second target site with an affinity of at least about 3 fold, such as at least about any of 5 fold, 10 fold, 15 fold, 20 fold, 25 fold, 50 fold, 75 fold, 100 fold, 250 fold, 500 fold, or 1000 fold, of that of a control binder molecule, such as a control co-binder, comprising an antibody variable domain not having the N-terminal truncation of the second antibody moiety. In some embodiments, the binder molecule and the control binder molecule both comprise an identical linker, such as having the same amino acid sequence. In some embodiments, the binder molecule and the control binder molecule both comprise an identical first binding moiety. In some embodiments, provided herein is a linker control binder molecule, such as a co-binder, wherein the linker binder molecule is the same as a test binder molecule except that the last three C-terminal amino acids in a linker of the control linker binder molecule, e.g., the last three C-terminal amino acid in a linker of the control linker binder molecule are GGG.

In some embodiments, the first binding moiety or the second binding moiety of the co-binder binds a target with a K_{D} of at least 1×10⁻¹⁰ M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the first binding moiety or the second binding moiety of the co-binder binds a target with a K_{D} of at least 1×10⁻⁹M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶M. In some embodiments, the first binding moiety or the second binding moiety of the co-binder binds a target with a K_{D} of at least 1×10⁻⁸M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the first binding moiety or the second binding moiety of the co-binder binds a target with a K_{D} of at least 1×10⁻⁷M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the first binding moiety or the second binding moiety of the co-binder binds a target with a K_{D} of at least 1×10⁻⁶ M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the first binding moiety or the second binding moiety of the co-binder binds a target with a K_{D} of at least 1×10⁻⁵ M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M.

In some embodiments, the first binding moiety of the co-binder has a relatively high affinity and binds the target with a K_{D} of less than 1×10⁻¹⁰ M. In some embodiments, the first binding moiety of the co-binder binds the target with a K_{D} of less than 1×10⁻¹¹M. In some embodiments, the first binding moiety of the co-binder binds the target with a K_{D} of less than 1×10⁻¹² M. These co-binders can have a second binding moiety with lower binding affinity. In some embodiments, the second binding moiety of the co-binder binds a target with a K_{D} of at least 1×10⁻⁹M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the second binding moiety of the co-binder binds a target with a K_{D} of at least 1×10⁻⁸M; and the co-binder binds the target with a K_{D} of less than 1×10⁻¹⁰M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the second binding moiety of the co-binder binds a target with a K_{D} of at least 1×10⁻⁷M; and the co-binder binds the target with a K_{D} of less than 1×10⁻¹⁰M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the second binding moiety of the co-binder binds a target with a K_{D} of at least 1×10⁶ M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M. In some embodiments, the second binding moiety of the co-binder binds a target with a K_{D} of at least 1×10⁻⁵ M; and the co-binder binds the target with a K_{D} of less than 1×10⁻¹⁰ M, less than 1×10⁻¹¹ M, less than 1×10⁻¹², less than 1×10⁻¹³ M, less than 1×10⁻¹⁴ M, less than 1×10⁻¹⁵ M, or less than 1×10⁻¹⁶ M.

In some embodiments, the first binding moiety and the second binding moiety of the co-binder both bind a target with a K_{D} of at least 1×10⁻¹⁰M; and the co-binder binds the target with a K_{D} of less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, or less than 1×10⁻¹⁶ M. In some embodiments, the first binding moiety and the second binding moiety of the co-binder both bind a target with a K_{D} of at least 1×10⁻⁹M; and the co-binder binds the target with a K_{D} of less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, or less than 1×10⁻¹⁶ M. In some embodiments, the first binding moiety and the second binding moiety of the co-binder both bind a target with a K_{D} of at least 1×10⁻⁸ M; and the co-binder binds the target with a K_{D} of less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, or less than 1×10⁻¹⁶M. In some embodiments, the first binding moiety and the second binding moiety of the co-binder both bind a target with a K_{D} of at least 1×10⁻⁷M; and the co-binder binds the target with a K_{D} of less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, or less than 1×10⁻¹⁶ M. In some embodiments, the first binding moiety and the second binding moiety of the co-binder both bind a target with a K_{D} of at least 1×10⁻⁶M; and the co-binder binds the target with a K_{D} of less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, or less than 1×10⁻¹⁶ M. In some embodiments, the first binding moiety and the second binding moiety of the co-binder both bind a target with a K_{D} of at least 1×10⁻⁵ M; and the co-binder binds the target with a K_{D} of less than 1×10⁻⁹M, less than 1×10⁻¹⁰M, less than 1×10⁻¹¹M, less than 1×10⁻¹² M, less than 1×10⁻¹³ M, less than 1×10⁻¹⁴ M, less than 1×10⁻¹⁵ M, or less than 1×10⁻¹⁶ M.

The first binding moiety or the second binding moiety can have a high binding affinity to a nonspecific molecule, which can be reduced or minimized in the co-binder. In some embodiments, the first binding moiety or the second binding moiety of the co-binder binds a nonspecific molecule with a K_{D} of less than 1×10⁻¹⁰M; and the co-binder binds the nonspecific molecule with a K_{D} of at least 1×10⁻¹⁰M, at least 1×10⁻⁹M, at least 1×10⁻⁸ M, at least 1×10⁻⁷M, at least 1×10⁻⁶M, at least 1×10⁻⁵ M, at least 1×10⁴M, or at least 1×10⁻³ M. In some embodiments, the first binding moiety or the second binding moiety of the co-binder binds a nonspecific molecule with a K_{D} of less than 1×10⁻⁹M; and the co-binder binds the nonspecific molecule with a K_{D} of at least 1×10⁻⁹M, at least 1×10⁻⁸ M, at least 1×10⁻⁷M, at least 1×10⁻⁶ M, at least 1×10⁻⁵ M, at least 1×10⁻⁴M, or at least 1×10³ M. In some embodiments, the first binding moiety or the second binding moiety of the co-binder binds a nonspecific molecule with a K_{D} of less than 1×10⁻⁸ M; and the co-binder binds the nonspecific molecule with a K_{D} of at least 1×10⁻⁸M, at least 1×10⁻⁷M, at least 1×10⁻⁶M, at least 1×10⁻⁵ M, at least 1×10⁻⁴M, or at least 1×10⁻³ M.

### IV. Compositions and Kits

In some aspects, the disclosure provides a composition comprising a binder molecule, such as a co-binder, provided herein. In some aspects, the disclosure provides a pharmaceutical composition comprising a binder molecule, such as a co-binder, provided herein and a pharmaceutically acceptable carrier. In some aspects, the disclosure provides a detection agent comprising a binder molecule, such as a co-binder, provided herein. In one aspect, the disclosure provides a diagnostic agent comprising a binder molecule, such as a co-binder, provided herein. In one aspect, the disclosure provides a therapeutic agent comprising a binder molecule, such as a co-binder, provided herein.

In some aspects, the disclosure provides a cell that expresses a binder molecule, such as a co-binder, provided herein. In some embodiments, the cell is an immune cell.

In some embodiments, the disclosure provides a composition comprising a binder molecule, such as a co-binder, provided herein. In some embodiments, the composition further comprising a second agent. In some embodiments, the second agent is a therapeutic agent. In some embodiments, the second agent is a therapeutic antibody. In some embodiments, the second agent is a therapeutic compound. In some embodiments, the second agent is a therapeutic small molecule compound.

In some embodiments, the disclosure provides a pharmaceutical composition comprising a binder molecule, such as a co-binder, provided herein. In some embodiments, the disclosure provides a pharmaceutical composition comprising a binder molecule, such as a co-binder, provided herein and a pharmaceutically acceptable carrier. A pharmaceutically acceptable carrier that can be used in the pharmaceutical compositions include any of the standard pharmaceutical carriers known in the art, such as phosphate buffered saline solution, water and emulsions such as an oil and water emulsion, and various types of wetting agents. These pharmaceutical compositions can be prepared in liquid unit dose forms or any other dosing form that is sufficient for delivery of the co-binder of present disclosure to the target area of the subject in need of treatment. For example, the pharmaceutical compositions can be prepared in any manner appropriate for the chosen mode of administration, e.g., intravascular, intramuscular, sub-cutaneous, or intraperitoneal. Other optional components, e.g., pharmaceutical grade stabilizers, buffers, preservatives, excipients and the like can be readily selected by one of skill in the art. The preparation of a pharmaceutically composition, having due regard to pH, isotonicity, stability and the like, is within the level of skill in the art.

Pharmaceutical compositions comprising a co-binder are prepared for storage by mixing the co-binder having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol.

The binder molecules, such as a co-binder, of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

The binder molecules, such as a co-binder, provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with a binder molecule, such as a co-binder, as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra, Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., a co-binder as described herein) or a composition of the disclosure (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more co-binders as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

In some embodiments, the disclosure provides a detection agent comprising a binder molecule, such as a co-binder, provided herein.

In some embodiments, the disclosure provides a diagnostic agent comprising a binder molecule, such as a co-binder, provided herein.

In some embodiments, the disclosure provides a diagnostic agent comprising a therapeutic agent comprising a binder molecule, such as a co-binder, provided herein.

The binder molecules, such as a co-binder, provided herein can form a function domain of a molecule. For example, in some embodiments, provided herein are also antibodies having a binder molecule, such as a co-binder, of the instant disclosure such as an antigen-recognition domain. In some embodiments, provided herein are multispecific antibodies having a binder molecule, such as a co-binder, of the instant disclosure such as one of its antigen-recognition domains. In some embodiments, provided herein are also bispecific antibodies having a co-binder of the instant disclosure such as one of its antigen-recognizing domains. In some embodiments, provided herein are chimeric antigen receptors having a binder molecule, such as a co-binder, of the instant disclosure such as its antigen-recognizing domain.

In some embodiments, the disclosure provides a chimeric antigen receptor (CAR) comprising a binder molecule, such as a co-binder, provided herein. In some embodiments, the CAR is expressed in a cell. In some embodiments, the cell is an immune cell. In some embodiments, the cell is a T cell, a T cell precursor, a natural killer (NK) cell, or an antigen presenting cell (APC).

In some embodiments, the binder molecule, such as a co-binder, is a peptide or a protein. Provided herein are also nucleic acid molecules encoding a peptide or protein binder molecule, such as a co-binder, and vectors that include nucleic acid that encodes the peptide or protein. Accordingly, “nucleic acids” include those that encode the binder molecules disclosed herein, as well as those encoding their functional subsequences, sequence variants and modified forms, so long as the foregoing retain at least detectable or measurable activity or function. Nucleic acid, which can also be referred to herein as a gene, polynucleotide, nucleotide sequence, primer, oligonucleotide or probe refers to natural or modified purine- and pyrimidine-containing polymers of any length, either polyribonucleotides or polydeoxyribonucleotides or mixed polyribo-polydeoxyribo nucleotides and α-anomeric forms thereof. The two or more purine- and pyrimidine-containing polymers are typically linked by a phosphoester bond or analog thereof. The terms can be used interchangeably to refer to all forms of nucleic acid, including deoxyribonucleic acid (DNA) and ribonucleic acid (RNA). The nucleic acids can be single strand, double, or triplex, linear or circular. Nucleic acids include genomic DNA and cDNA. RNA nucleic acid can be spliced or unspliced mRNA, RNA, tRNA or antisense. Nucleic acids include naturally occurring, synthetic, as well as nucleotide analogues and derivatives.

As a result of the degeneracy of the genetic code, nucleic acid molecules include sequences degenerate with respect to nucleic acid molecules encoding the binder molecules of the instant disclosure. The term “complementary,” when used in reference to a nucleic acid sequence, means the referenced regions are 100% complementary, i.e., exhibit 100% base pairing with no mismatches. Nucleic acid can be produced using any of a variety of known standard cloning and chemical synthesis methods, and can be altered intentionally by site-directed mutagenesis or other recombinant techniques known to one skilled in the art. Purity of polynucleotides can be determined through sequencing, gel electrophoresis, UV spectrometry.

Nucleic acids can be inserted into a nucleic acid construct in which expression of the nucleic acid is influenced or regulated by an “expression control element,” referred to herein as an “expression cassette.” The term “expression control element” refers to one or more nucleic acid sequence elements that regulate or influence expression of a nucleic acid sequence to which it is operatively linked. An expression control element can include, as appropriate, promoters, enhancers, transcription terminators, gene silencers, a start codon (e.g., ATG) in front of a protein-encoding gene, etc.

An expression control element operatively linked to a nucleic acid sequence controls transcription and, as appropriate, translation of the nucleic acid sequence. The term “operatively linked” refers to a juxtaposition wherein the referenced components are in a relationship permitting them to function in their intended manner. Typically, expression control elements are juxtaposed at the 5′ or the 3′ ends of the genes but can also be intronic.

Expression control elements include elements that activate transcription constitutively, that are inducible (i.e., require an external signal or stimuli for activation), or derepressible (i.e., require a signal to turn transcription off; when the signal is no longer present, transcription is activated or “derepressed”). Also included in the expression cassettes of the disclosure are control elements sufficient to render gene expression controllable for specific cell-types or tissues (i.e., tissue-specific control elements). Typically, such elements are located upstream or downstream (i.e., 5′ and 3′) of the coding sequence. Promoters are generally positioned 5′ of the coding sequence. Promoters, produced by recombinant DNA or synthetic techniques, can be used to provide for transcription of the polynucleotides of the disclosure. A “promoter” typically means a minimal sequence element sufficient to direct transcription.

Nucleic acids can be inserted into a plasmid for transformation into a host cell and for subsequent expression and/or genetic manipulation. A plasmid is a nucleic acid that can be stably propagated in a host cell; plasmids may optionally contain expression control elements in order to drive expression of the nucleic acid. As used herein, a vector is synonymous with a plasmid. Plasmids and vectors generally contain at least an origin of replication for propagation in a cell and a promoter. Plasmids and vectors may also include an expression control element for expression in a host cell, and are therefore useful for expression and/or genetic manipulation of nucleic acids encoding peptide sequences, expressing peptide sequences in host cells and organisms (e.g., a subject in need of treatment), or producing peptide sequences, for example.

As used herein, the term “transgene” means a polynucleotide that has been introduced into a cell or organism by artifice. For example, a cell having a transgene, the transgene has been introduced by genetic manipulation or “transformation” of the cell. A cell or progeny thereof into which the transgene has been introduced is referred to as a “transformed cell” or “transformant.” Typically, the transgene is included in progeny of the transformant or becomes a part of the organism that develops from the cell. Transgenes may be inserted into the chromosomal DNA or maintained as a self-replicating plasmid, YAC, minichromosome, or the like.

Bacterial system promoters include T7 and inducible promoters such as pL of bacteriophage λ, plac, ptrp, ptac (ptrp-lac hybrid promoter) and tetracycline responsive promoters. Insect cell system promoters include constitutive or inducible promoters (e.g., ecdysone). Mammalian cell constitutive promoters include SV40, RSV, bovine papilloma virus (BPV) and other virus promoters, or inducible promoters derived from the genome of mammalian cells (e.g., metallothionein IIA promoter; heat shock promoter) or from mammalian viruses (e.g., the adenovirus late promoter; the inducible mouse mammary tumor virus long terminal repeat). Alternatively, a retroviral genome can be genetically modified for introducing and directing expression of a peptide sequence in appropriate host cells.

Provided herein are also are also vectors designed for in vivo use include in vivo delivery and its expression systems. Particular non-limiting examples include adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979), retroviral vectors (U.S. Pat. Nos. 5,624,820, 5,693,508 and 5,674,703), BPV vectors (U.S. Pat. No. 5,719,054), CMV vectors (U.S. Pat. No. 5,561,063) and parvovirus, rotavirus, Norwalk virus and lentiviral vectors (see, e.g., U.S. Pat. No. 6,013,516). Vectors include those that deliver genes to cells of the intestinal tract, including the stem cells (Croyle et al., Gene Ther. 5:645 (1998); S. J. Henning, Adv. Drug Deliv. Rev. 17:341 (1997), U.S. Pat. Nos. 5,821,235 and 6,110,456). Many of these vectors have been approved for human studies.

Yeast vectors include constitutive and inducible promoters (see, e.g., Ausubel et al., In: Current Protocols in Molecular Biology, Vol. 2, Ch. 13, ed., Greene Publish. Assoc. & Wiley Interscience, 1988; Grant et al. Methods in Enzymology, 153:516 (1987), eds. Wu & Grossman; Bitter Methods in Enzymology, 152:673 (1987), eds. Berger & Kimmel, Acad. Press, N.Y.; and, Strathern et al., The Molecular Biology of the Yeast Saccharomyces (1982) eds. Cold Spring Harbor Press, Vols. I and II). A constitutive yeast promoter such as ADH or LEU2 or an inducible promoter such as GAL may be used (R. Rothstein In: DNA Cloning, A Practical Approach, Vol. 11, Ch. 3, ed. D. M. Glover, IRL Press, Wash., D.C., 1986). Vectors that facilitate integration of foreign nucleic acid sequences into a yeast chromosome, via homologous recombination for example, are known in the art. Yeast artificial chromosomes (YAC) are typically used when the inserted polynucleotides are too large for more conventional vectors (e.g., greater than about 12 Kb).

Expression vectors also can contain a selectable marker conferring resistance to a selective pressure or identifiable marker (e.g., beta-galactosidase), thereby allowing cells having the vector to be selected for, grown and expanded. Alternatively, a selectable marker can be on a second vector that is co-transfected into a host cell with a first vector containing a nucleic acid encoding a peptide sequence. Selection systems include but are not limited to herpes simplex virus thymidine kinase gene (Wigler et al., Cell 11:223 (1977)), hypoxanthine-guanine phosphoribosyltransferase gene (Szybalska et al., Proc. Natl. Acad. Sci. USA 48:2026 (1962)), and adenine phosphoribosyltransferase (Lowy et al., Cell 22:817 (1980)) genes that can be employed in tk-, hgprt_or aprt_cells, respectively. Additionally, antimetabolite resistance can be used as the basis of selection for dhfr, which confers resistance to methotrexate (O'Hare et al., Proc. Natl. Acad. Sci. USA 78:1527 (1981)); the gpt gene, which confers resistance to mycophenolic acid (Mulligan et al., Proc. Natl. Acad. Sci. USA 78:2072 (1981)); neomycin gene, which confers resistance to aminoglycoside G-418 (Colberre-Garapin et al., J. Mol. Biol. 150: 1 (1981)); puromycin; and hygromycin gene, which confers resistance to hygromycin (Santerre et al., Gene 30:147 (1984)). Additional selectable genes include trpB, which allows cells to utilize indole in place of tryptophan; hisD, which allows cells to utilize histinol in place of histidine (Hartman et al., Proc. Natl. Acad. Sci. USA 85:8047 (1988)); and ODC (ornithine decarboxylase), which confers resistance to the ornithine decarboxylase inhibitor, 2-(difluoromethyl)-DL-ornithine, DFMO (McConlogue (1987) In: Current Communications in Molecular Biology, Cold Spring Harbor Laboratory).

Accordingly, provided herein are also transformed or host cell(s) (in vitro, ex vivo and in vivo) that produce a binder molecule, such as a co-binder, disclosed herein, where expression of the binder molecule is conferred by a nucleic acid encoding the co-binder. Transformed and host cells that express a binder molecule, such as a co-binder, typically include a nucleic acid that encodes the binder molecule. In some embodiments, a transformed or host cell is a prokaryotic cell. In another embodiment, a transformed or host cell is a eukaryotic cell. In various aspects, the eukaryotic cell is a yeast or mammalian (e.g., human, primate, etc.) cell.

As used herein, a “transformed” or “host” cell is a cell into which a nucleic acid is introduced that can be propagated and/or transcribed for expression of an encoded peptide sequence. The term also includes any progeny or subclones of the host cell. Transformed and host cells include but are not limited to microorganisms such as bacteria and yeast; and plant, insect and mammalian cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for transient or stable propagation or expression.

In some embodiments, the disclosure provides a cell that expresses a binder molecule, such as a co-binder, provided herein. In some embodiments, the cell expressing the binder molecule is an immune cell. In some embodiments, the cell expressing the binder molecule is a T cell, a T cell precursor, a natural killer (NK) cell, or an antigen presenting cell (APC). In some embodiments, the disclosure provides a host cell that expresses a binder molecule, such as a co-binder, provided herein. In some embodiments, the host cell expressing the binder molecule is an immune cell. In some embodiments, the host cell expressing the binder molecule is a T cell, a T cell precursor, a natural killer (NK) cell, or an antigen presenting cell (APC).

In some embodiments, the disclosure provides a complex comprising a binder molecule, such as a co-binder, provided herein and the target.

Also provided herein are kits comprising a binder molecule, such as a co-binder, provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain binder molecules, such as co-binders, provided herein, or cells that contain nucleic acids encoding the binder molecules, such as co-binders, provided herein. The cells in the kit can be maintained under appropriate storage conditions until ready to use.

### V. Methods of Making, Libraries, and Screening Techniques

The binder molecules, such as a co-binder, described herein can be produced by any method known in the art for the synthesis of peptides, nucleic acids, or other molecules, in particular, by chemical synthesis or by recombinant expression techniques. The practice of the disclosure employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press; Borrebaeck (ed.) (1995) Antibody Engineering, Second Edition, Oxford University Press; Lo (ed.) (2006) Antibody Engineering: Methods and Protocols (Methods in Molecular Biology); Vol. 248, Humana Press, Inc; each of which is incorporated herein by reference in its entirety.

Peptides and peptidomimetics can be produced and isolated using methods known in the art. Peptides can be synthesized, in whole or in part, using chemical methods (see, e.g., Caruthers (1980). Nucleic Acids Res. Symp. Ser. 215; Horn (1980); and Banga, A. K., Therapeutic Peptides and Proteins, Formulation, Processing and Delivery Systems (1995) Technomic Publishing Co., Lancaster, PA). Peptide synthesis can be performed using various solid-phase techniques (see, e.g., Roberge Science 269:202 (1995); Merrifield, Methods Enzymol. 289:3 (1997)) and automated synthesis may be achieved, e.g., using the ABI 431A Peptide Synthesizer (Perkin Elmer) in accordance with the manufacturer's instructions. Peptides and peptide mimetics can also be synthesized using combinatorial methodologies. Synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)). Peptide sequence variations, derivatives, substitutions and modifications can also be made using methods such as oligonucleotide-mediated (site-directed) mutagenesis, alanine scanning, and PCR based mutagenesis. Site-directed mutagenesis (Carter et al., Nucl. Acids Res., 13:4331 (1986); Zoller et al., Nucl. Acids Res. 10:6487 (1987)), cassette mutagenesis (Wells et al., Gene 34:315 (1985)), restriction selection mutagenesis (Wells et al., Philos. Trans. R. Soc. London SerA 317:415 (1986)) and other techniques can be performed on cloned DNA to produce peptide sequences, variants, fusions and chimeras, and variations, derivatives, substitutions and modifications thereof.

The binder molecules, such as a co-binder, described herein that include antigen binding fragment of an antibody can be prepared using a wide variety of techniques known in the art including the use of hybridoma and recombinant technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981), each of which is incorporated herein by reference in its entirety. Other methods of producing the co-binders are also known in the art.

In some embodiments, the co-binders and the antibody provided herein for the co-binders may be produced by culturing cells transformed or transfected with a vector containing co-binder-encoding or antibody encoding nucleic acids. Polynucleotide sequences encoding polypeptide components of the co-binder or the antibody of the present disclosure can be obtained using standard recombinant techniques. Desired polynucleotide sequences may be isolated and sequenced from co-binder or antibody producing cells such as hybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Co-binders produced by the host cells are purified using standard protein purification methods as known in the art.

Methods for the co-binder production including vector construction, expression, and purification are further described in P1ückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherischia coli, in Antibody Expression and Production (A₁-Rubeai ed., 2011); and Therapeutic Monoclonal Antibodies: From Bench to Clinic (An ed., 2009).

It is, of course, contemplated that alternative methods, which are well known in the art, may be employed to prepare co-binders. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the co-binders may be chemically synthesized separately and combined using chemical or enzymatic methods to produce the desired co-binders.

In certain aspects, provided herein is a combinatorial library (e.g., a co-binder library) useful for developing a binder molecule, such as a co-binder, described herein specifically recognizing a target.

In some embodiments, provided is a library comprising a plurality of co-binders or a plurality of polynucleotides encoding a plurality of co-binders, each co-binder comprising a first binding moiety specifically recognizing a first target site and a second binding moiety specifically recognizing a second target site, wherein the second binding moiety is a second antibody moiety comprising an antibody variable domain, wherein the first binding moiety is connected to the second binding moiety through N-terminus of the antibody variable domain via a peptide linker, wherein at least two co-binders in the library differ from each other in the peptide linker sequence. In some embodiments, the first target site and the second target site are non-overlapping binding sites on a target molecule. In some embodiments, the antibody variable domain has an N-terminal truncation (“N-terminal truncated antibody variable domain”). In some embodiments, at least two co-binders in the library differ from each other in the N-terminal truncation of the antibody variable domain of the second antibody moiety.

In some embodiments, the diversity of the library is at least about 5000, e.g., the library contains at least about 5000 unique co-binder sequences.

In some embodiments, substantially all of the plurality of co-binders comprise the same first binding moiety and second binding moiety. In such an embodiments, the library comprises co-binders comprising unique linker sequences.

In some embodiments, at least two, such as at least about any of 10, 25, 50, 100, 250, 500, and 1,000, of the plurality of co-binders comprise a different first binding moiety and/or second binding moiety.

In some embodiments, provided is a method of screening for a co-binder specifically binding to a second target site at a desired affinity, the method comprising: (1) contacting a library described herein with a target molecule comprising the second target site to form complexes between the co-binders that specifically bind to the target molecule and the target molecule, and (2) identifying a co-binder that binds to the second target site with the desired affinity.

In some embodiments, provided is a method of screening for a co-binder specifically binding to a target molecule at a desired affinity, the method comprising: (1) contacting a library described herein with the target molecule to form complexes between the co-binders that specifically bind to the target molecule and the target molecule, and (2) identifying a co-binder that binds to the target molecule with the desired affinity.

In some embodiments, the combinatorial library comprises a collection of any one or more of the following: (a) second binding moiety; (b) first binding moiety; (c) linker; and/or (d) another feature described herein, such as a label.

In some embodiments, the library comprises at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ variable regions of a second binding moiety described herein from a plurality of antibodies, wherein each variable region comprises an N-terminal truncation of from 1 to 18 amino acids. In some embodiments, the library comprises about 2, about 5, about 10, about 15, about 20, about 25, about 30, about 35, about 40, about 45, about 50, about 60, about 70, about 80, about 90, about 100, about 200, about 300, about 400, about 500, about 600, about 700, about 800, about 900, about 1×10³, about 1×10⁴, about 1×10⁵, about 1×10⁶, about 1×10⁷, about 1×10⁸, about 1×10⁹, about 1×10¹⁰, or about 1×10¹¹ variable regions of a second binding moiety described herein from a plurality of antibodies, wherein each variable region comprises an N-terminal truncation of from 1 to 18 amino acids. In some embodiments, the truncation is in the FR1 region of the variable region.

In some embodiments, the library comprises at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ variable regions of a first binding moiety described herein from a plurality of antibodies. In some embodiments, the library comprises about 2, about 5, about 10, about 15, about 20, about 25, about 30, about 35, about 40, about 45, about 50, about 60, about 70, about 80, about 90, about 100, about 200, about 300, about 400, about 500, about 600, about 700, about 800, about 900, about 1×10³, about 1×10⁴, about 1×10⁵, about 1×10⁶, about 1×10⁷, about 1×10⁸, about 1×10⁹, about 1×10¹⁰, or about 1×10¹¹ variable regions of a first binding moiety described herein from a plurality of antibodies.

In some embodiments, the library comprises at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ linkers. In some embodiments, the library comprises about 2, about 5, about 10, about 15, about 20, about 25, about 30, about 35, about 40, about 45, about 50, about 60, about 70, about 80, about 90, about 100, about 200, about 300, about 400, about 500, about 600, about 700, about 800, about 900, about 1×10³, about 1×10⁴, about 1×10⁵, about 1×10⁶, about 1×10⁷, about 1×10⁸, about 1×10⁹, about 1×10¹⁰, or about 1×10¹¹ linkers described herein.

In some embodiments, the library comprises partial forms of a binder molecule described herein, for example, a second binding moiety covalently attached to a linker. Each of a plurality members in the library can comprises a different second binding moiety (e.g., second binding moieties having different CDR sequences or different N-terminal truncations) and/or a different linker sequence. In such embodiments, such library features are useful for efficient identification of a binder molecule, such as a co-binder. In some embodiments, the N-terminal amino acid of the linker in each member of the library is further lined to the C-terminal amino acid of a first binding moiety.

In some embodiments, a single second binding moiety covalently attached to a linker can be fused to a different first binding moiety form a library for the purpose of identifying suitable co-binders.

In some embodiments, the library comprises a first variable region of a first binding moiety, wherein the N-terminal amino acids of the linkers are linked with the C-terminal amino acid of the first variable region. In some embodiments, the library comprises a plurality of first variable regions of a first binding moiety, wherein the N-terminal amino acids of the linkers are linked with the C-terminal amino acids of the first variable regions. In some embodiments, the library comprises a plurality of first variable regions of a plurality of first binding moieties, wherein the N-terminal amino acids of the linkers are linked with the C-terminal amino acid of the first variable regions.

As such, a person of ordinary skill in the art would understand based on the above description, the present disclosure provides libraries of binder molecules, such as a co-binder, comprising: (i) any first subsection of the library selected from the group consisting of: a second heavy chain variable region of a second antibody moiety comprising an N-terminal truncation of from 1 to 18 amino acids; a plurality of second heavy chain variable regions of a second antibody moiety, wherein each of the second heavy chain variable region comprises an N-terminal truncation of from 1 to 18 amino acids; a plurality of second heavy chain variable regions of a plurality of second antibody moieties, wherein each of the second heavy chain variable region comprises an N-terminal truncation of from 1 to 18 amino acids; a second light chain variable region of a second antibody moiety comprising an N-terminal truncation of from 1 to 18 amino acids; a plurality of second light chain variable regions of a second antibody moiety, wherein each of the second light chain variable region comprises an N-terminal truncation of from 1 to 18 amino acids; and a plurality of second light chain variable regions of a plurality of second antibody moieties, wherein each of the second light chain variable region comprises an N-terminal truncation of from 1 to 18 amino acids; (ii) any linker subsection of the library selected from the group consisting of: a polypeptide linker; and a plurality of polypeptide linkers; and (iii) any second subsection of the library selected from the group consisting of: a first heavy chain variable region of a first antibody moiety; a plurality of first heavy chain variable regions of a first antibody moiety; a plurality of first heavy chain variable regions of a plurality of first antibody moieties; a first light chain variable region of a first antibody moiety; a plurality of first light chain variable regions of a first antibody moiety; and a plurality of first light chain variable regions of a plurality of first antibody moieties, wherein the N-terminal amino acids of the linkers are linked with the C-terminal amino acid of the first light chain variable regions and wherein C-terminal amino acids of the linkers are linked with the N-terminal amino acids of the truncated first light chain variable regions. Accordingly, a person of ordinary skill in the art would understand that the co-binder libraries provided herein encompasses any and all combinations or permutations of the subsection of the library as provided for (i), (ii), and (iii) in the present disclosure and specifically in this paragraph.

In some embodiments, the library comprises (i) a first variable region and a second variable region that binds to nonoverlapping epitopes on the same target, (ii) a first variable region and a second variable region that does not bind to the same target, (iii) a first variable region and a second variable region that binds to nonoverlapping epitopes on the same target, or (iv) a first variable region and a second variable region that does not bind to the same target.

In some embodiments, a library of the first binding moieties (paratopes P1) and a library of the second binding moieties (paratopes P2) can be constructed independently that binds to the epitopes of a target antigen. The library of the first binding moieties (paratopes P1) or the library of the second binding moieties (paratopes P2) can be constructed in a gene expression vector that allows the transcription of the cloned genes and translation into recombinant proteins.

The library of the first binding moieties (paratopes P1) can be sequences coding for camelid VHH, scFv, Fab, affibodies, affilins, affimers, affitins, alphabodies, anticalins, aptamers, avimers, DARPins, Fynomers, Kunitz domain peptides, monobodies, or nanoCLAMPs, etc. Similarly, the library of the second binding moieties (paratopes P2) can be sequences coding for camelid VHH, scFv, Fab, affibodies, affilins, affimers, affitins, alphabodies, anticalins, aptamers, avimers, DARPins, Fynomers, Kunitz domain peptides, monobodies, or nanoCLAMPs, etc. Any combinations of different libraries of the first binding moieties (paratopes P1) and the second binding moieties (paratopes P2) are contemplated herein.

In some embodiments, the library of the first binding moieties (paratopes P1) and the library of the second binding moieties (paratopes P2) in the expression vector can both be sequences coding for camelid VHH. In some embodiments, the library of the first binding moieties (paratopes P1) and the library of the second binding moieties (paratopes P2) in the expression vector can both be sequences coding for scFv. In still another preferred embodiment, the library of the first binding moieties (paratopes P1) and the library of the second binding moieties (paratopes P2) in the expression vector can be sequences of one coding for camelid VHH and another coding for scFv.

For example, to construct a library of the first binding moieties (paratopes P1) or a library of the second binding moieties (paratopes P2), mRNA coding for the variable domain of heavy-chain antibodies (VHH) can be isolated from an alpaca immunized against the target molecule, transcribed to cDNA, and cloned into a phagemid vector for phage display library construction. Similarly, to construct a library of the first binding moieties (paratopes P1) or a library of the second binding moieties (paratopes P2), mRNA coding for the variable domains of heavy-chain and light-chain antibodies can be isolated from an animal immunized against the target antigen, transcribed to cDNA, and cloned into a phagemid vector as scFv for phage display library construction. While the expression vector can be a part of phage display library construction, it can also be part of the yeast display, bacterial display, mammalian cell display, ribosome display, or mRNA display library construction.

The library of the first binding moieties (paratopes P1) or the library of the second binding moieties (paratopes P2) can be naïve libraries and they can also be immune libraries of primary or secondary responses. The library of the first binding moieties (paratopes P1) or the library of the second binding moieties (paratopes P2) can be synthetic libraries. The library of the first binding moieties (paratopes P1) or the library of the second binding moieties (paratopes P2) can be affinity-enriched naïve, immune or synthetic libraries for binding to a target antigen of interest. For example, the library of the first binding moieties (paratopes P1) or the library of the second binding moieties (paratopes P2) can be cloned into phagemids of phage-display library construct. These phage-display libraries are then allowed to bind to the immobilized target antigen. Phage-displaying proteins that interact with the target antigens will remain attached, while all others are washed away. Attached phage can then be eluted and used to create more phage by infection of suitable bacterial hosts. The new phage constitutes an enriched mixture, containing considerably less non-binding phage than were present in the initial mixture. By this process, the library of the first binding moieties (paratopes P1) or the library of the second binding moieties (paratopes P2) can be enriched for sequences coding for those paratopes that bind to the target antigen. The affinity-enriched library of the first binding moieties (paratopes P1) or the affinity-enriched library of the second binding moieties (paratopes P2) can be more suitable for screening.

In certain aspects, provided herein is a method of screening for a binder molecule, such as a co-binder, to a target, comprising (i) obtaining a library provided herein; and (ii) contacting the library of candidates from step (i) with the target to identify a binder molecule, such as a co-binder, that specifically binds to the target.

In certain aspects, provided herein is a method of screening for a binder molecule, such as a co-binder, to a target, comprising (i) expressing a library of expression vectors encoding the library provided herein; (ii) obtaining the library provided herein; and (iii) contacting the library of candidates from step (ii) with the target to identify a binder molecule, such as a co-binder, that specifically binds to the target.

In yet another aspect, provided herein is a method of screening for a binder molecule, such as a co-binder, to a target, comprising (i) expressing a library of expression vectors encoding the library of co-binders provided herein; (ii) obtaining the library provided herein; (iii) contacting the library of candidates from step (ii) with the target to form complexes between the binder molecules, such as co-binders, that specifically bind to the target; (iv) enriching for the complexes between the binder molecules, such as co-binders, that specifically bind to the target; and (v) identifying the binder molecules, such as co-binders, that specifically bind to the target.

In some embodiments, the screening methods provided herein identify binder molecules, such as co-binders, that specifically bind to a target, wherein the affinity of the binder molecule to the target is no less than 50 fold, no less than 60 fold, no less than 70 fold, no less than 80 fold, no less than 90 fold, no less than 100 fold, no less than 110 fold, no less than 120 fold, no less than 130 fold, no less than 140 fold, no less than 150 fold, no less than 160 fold, no less than 170 fold, no less than 180 fold, no less than 190 fold, no less than 200 fold, no less than 250 fold, no less than 300 fold, no less than 350 fold, no less than 400 fold, no less than 450 fold, no less than 500 fold, no less than 600 fold, no less than 700 fold, no less than 800 fold, no less than 900 fold, no less than 1000 fold, no less than 1100 fold, no less than 1200 fold, no less than 1300 fold, no less than 1400 fold, no less than 1500 fold, no less than 1600 fold, no less than 1700 fold, no less than 1800 fold, no less than 1900 fold, no less than 2000 fold, no less than 3000 fold, no less than 4000 fold, or no less than 5000 fold, or no less than 10000 fold greater than that of the individual variable regions alone or without truncation.

In some embodiments of the screening methods provided herein, the binder molecules, such as co-binders, identified by the methods bind the target with a K_{D} of less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, less than 1×10⁻¹⁶ M, less than 1×10⁻¹⁷ M, or less than 1×10⁻¹⁸ M.

In some embodiments, provided herein are methods of screening for co-binders, in which an expression vector can be constructed that contains a first coding region for the subsection of a library of the first binding moieties (paratopes P1), a second coding region for the subsection of a library of the second binding moieties (paratopes P2), and a third coding region for a subsection of a library of linkers L that links first binding moieties (paratopes P1) and second binding moieties (paratopes P2). In the phage display system, the expression vector is expressed in the form of fusions with a bacteriophage coat protein (e.g. pIII), so that they are displayed on the surface of the viral particle. The fusion protein displayed corresponds to the genetic sequence within the phage. By this method, those displayed proteins, containing the first binding moieties (paratopes P1) and the second binding moieties (paratopes P2) linked by a linker, having high affinity binding to the target antigen can be identified and they are candidates for co-binders. Similarly, candidate co-binders can be screened using yeast display, bacterial display, mammalian cell display, ribosome display, or mRNA display library constructs.

The disclosure provides that the identification of the co-binders that specifically bind to the target can be achieved by a variety of methods available to a person of ordinary skill in the art. For example, the polynucleotides encoding the co-binders that specifically bind to the target can be sequenced from the sorted host cells or the panned phages as described above. The corresponding polypeptide sequences of the co-binders can be identified by translating the sequence of polynucleotide encoding the co-binders using genetic code table well known in the art. Alternative, the co-binders can be identified by applying amino acid sequencing and/or mass spectrometry to the co-binders and/or the protein complexes between the co-binders and the target.

In some embodiments, the first binding moieties (paratopes P1) in the expression vector contains sequences coding for more than one distinct binding moiety. In some embodiments, the first binding moieties (paratopes P1) in the expression vector contains sequences coding for more than 2, more than 5, more than 10, more than 20, more than 50, more than 100, more than 200, more than 500, more than 1000, more than 1×10⁴, more than 1×10⁵, or more than 1×10⁶ distinct binding moieties. In some embodiments, the second binding moieties (paratopes P2) in the expression vector contains sequences coding for more than one distinct paratope. In some embodiments, the second binding moieties (paratopes P2) in the expression vector contains sequences coding for at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ distinct binding moieties.

Linkers L in the expression vector contains sequences coding for more than one linker. In some embodiments, the linkers L in the expression vector contains sequences coding for at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ distinct linkers. In some embodiments, the linkers L in the expression vectors contain sequences coding for at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ distinct recombinant proteins.

In some embodiments, provided is a library of co-binders each of which contains a first binding moiety and a second binding moiety binding to the same target molecule, wherein the library contains at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ distinct linkers linking pairs of the first binding moiety and the second binding moiety.

The disclosure provides that the enrichment for the complexes between the binder molecules, such as co-binders, that specifically bind to the target and the target can be achieved by a variety of methods available to a person of ordinary skill in the art. For example, host cells expressing a co-binder library can be sorted by suitable sorting means (e.g. fluorescence activated cell sorting, “FACS”, or magnetic beads based sorting) to positively select cells expressing co-binders with high affinity, thereby obtaining a population of cells enriched for pluripotent cells. In one specific embodiment, host cells expressing a co-binder library can be sorted based on the amount of staining using labeled target, wherein the enrichment for the high affinity co-binders can be fine-tuned by adjusting the concentration of the labeled target. In such a fine tuning of the enrichment for high affinity co-binders, when low concentration of the labeled target is used, only the co-binders with K_{D} above an ascertainable level can be stably stained and sorted. The lower the concentration, the higher the binding stringency. High-affinity binders can retain good target engagement under the high stringency but not the weaker binders. In certain embodiment, the host cells expressing a co-binder library is stained with a labeled target at a concentration of less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M, less than 1×10⁻¹⁴ M, or less than 1×10⁻¹⁵ M. In certain embodiment, the host cells expressing a co-binder library is stained with a labeled target at a concentration of about 1×10⁻⁸ M, about 1×10⁻⁹ M, about 1×10⁻¹⁰ M, about 1×10⁻¹¹ M, about 1×10⁻¹² M, about 1×10⁻¹³ M, about 1×10⁻¹⁴ M, or about 1×10⁻¹⁵ M.

Alternatively, the host cells expressing a co-binder library can be sorted based on the amount of staining using labeled target, wherein the labeled target bound to the host cells expressing low affinity co-binders have been washed off by washing under various stringency, thereby enriching for the host cells expressing high affinity co-binders. The stringency of the washes in such embodiments can be fine-tuned and controlled by various means known to a person of ordinary skill in the art. For example, the host cells expressing a co-binder library can be washed with unlabeled target molecules that compete with the labeled target. The stringency of the washes can be controlled by adjusting the ratio between the unlabeled target and labeled target, such that only host cells expressing high affinity co-binders will remained stained by the labeled target and positively sorted, thereby enriching for host cells expressing high affinity co-binder. The stringency of the washes can be controlled by adjusting the strength of washing buffers used, for example by washing with different detergent solutions. In certain embodiment, the host cells expressing a co-binder library is washed with a unlabeled target at a concentration of more than 1×10⁻³ M, more than 1×10⁻⁴ M, or more than 1×10⁻⁵ M, 1×10⁻⁶ M, more than 1×10⁻⁶ M, or more than 1×10⁻⁷ M, more than 1×10⁻⁸ M, more than 1×10⁻⁹ M, more than 1×10⁻¹⁰ M, more than 1×10⁻¹¹ M, or more than 1×10⁻¹² M. In certain embodiment, the host cells expressing a co-binder library is washed with a unlabeled target at a concentration of about 1×10⁻³ M, about 1×10⁻⁴ M, or about 1×10⁻⁵ M, 1×10⁻⁶ M, about 1×10⁻⁶ M, or about 1×10⁻⁷ M, about 1×10⁻⁸ M, about 1×10⁻⁹ M, about 1×10⁻¹⁰ M, about 1×10⁻¹¹ M, or about 1×10⁻¹² M.

Additionally, the washing time can be varied. After the library members are incubated with the target protein, unbound library members or proteins need to be washed away. The longer the washing time, the higher the stringency. Weaker binders may dissociate from the target protein during the washing step, but not the high-affinity binders. Similarly, the time for target protein incubation can also be varied. Strong binders tend to bind targets faster than weaker binders. By limiting the incubation time, high-affinity binders get enriched better than low-affinity ones.

One or more rounds of enrichment can be performed to enrich for the co-binders with high affinity for a target. In some embodiments, after 3-4 rounds of selections with increased stringencies, the resulted co-binder library will be enriched with high-affinity co-binders. In some embodiments, co-binder library is enriched for 1, 2, 3, 4, 5, 6, 7, 8, 9, 10, 11, 12, 13, 14, 15, 16, 17, 18, 19, 20, 21, 22, 23, 24, 25, 30, 35, 40, 45, or 50 rounds to obtain a co-binder library with high affinity co-binders.

In some embodiments described herein, the host cells expressing high affinity co-binders can also be enriched by negatively sorting out (removing) the cells unstained by the labeled target.

These strategies described above for enriching for the complexes between the co-binders that specifically bind to the target and the target can also be used and combined with other technologies described herein and known to a person of ordinary skill in the art. For example, the binding measured in SPR, the binding measured in ELISA, panning of phage display, the binding measured in yeast display, the binding measured in mammalian cell display, can be fine-tuned either by lowering the concentration of the labeled target used for the binding or by adjusting the stringency of the washes as described above, so that only high affinity co-binders will stably bound by the labeled target, thereby enriching for the complexes between the co-binders that specifically bind to the target and the target.

In some embodiments, binder molecule, such as co-binder, variants and/or antibody variants provided herein are be prepared by in vitro affinity maturation improved property such as affinity, stability, or expression level as compared to a parent construct. Like the natural prototype, in vitro affinity maturation is based on the principles of mutation and selection. Libraries of binder molecules, such as co-binder, variants and/or antibody variants provided herein are displayed either on the surface of an organism (e.g., phage, bacteria, yeast, or mammalian cell) or in association (e.g., covalently or non-covalently) with their encoding mRNA or DNA. Affinity selection of the displayed binder molecule, such as co-binder, variants and/or antibody variants allows isolation of organisms or complexes carrying the genetic information encoding the antibodies. Two or three rounds of mutation and selection using display methods such as phage display usually results in antibody fragments with affinities in the low nanomolar range. Affinity matured binder molecule, such as co-binder, variants and/or antibody variants can have nanomolar or even picomolar affinities for the target antigen.

Phage display is a widespread method for display and selection of binder molecule, such as co-binder, variants and/or antibody variants provided herein. The binder molecule, such as co-binder, variants and/or antibody variants are displayed on the surface of Fd or M13 bacteriophages as fusions to the bacteriophage coat protein. Selection involves exposure to antigen to allow phage-displayed binder molecule, such as co-binder, variants and/or antibody variants to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody variants provided herein for the binder molecules, such as co-binders, may be displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of binder molecule, such as co-binder, variants or antibody variants is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34: e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In a mammalian cell display system (see, e.g., Bowers et al., 2011, Proc Natl Acad Sci USA. 108:20455-60), a fully human library of IgGs is constructed based on germline sequence V-gene segments joined to prerecombined D (J) regions. Full-length V regions for heavy chain and light chain are assembled with human heavy chain and light chain constant regions and transfected into a mammalian cell line (e.g., HEK293). The transfected library is expanded and subjected to several rounds of negative selection against streptavidin (SA)-coupled magnetic beads, followed by a round of positive selection against SA-coupled magnetic beads coated with biotinylated target protein, peptide fragment, or epitope. Positively selected cells are expanded, and then sorted by rounds of FACS to isolate single cell clones displaying antibodies that specifically bind to the target protein, peptide fragment, or epitope. Heavy and light chain pairs from these single cell clones are retransfected with AID for further maturation. Several rounds of mammalian cell display, coupled with AID-triggered somatic hypermutation, generate high specificity, high affinity antibodies.

Diversity may also be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. In a specific embodiment, somatic hypermutation is performed by AID-triggered somatic hypermutation, e.g., using the SHM-XEL™ platform (AnaptysBio, San Diego, CA). Random mutations can be introduced throughout the whole V gene using E. coli mutator strains, error-prone replication with DNA polymerases (see, e.g., Hawkins et al., 1992, J. Mol. Biol. 226:889-96), or RNA replicases. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, a target can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### VI. Methods of Use

### A. Methods of Detection

The binder molecules, such as a co-binder, described herein having high affinity and/or high specificity can be used as a detection agent for detecting a target. In some embodiments, the target is a disease marker, and the binder molecule, such as a co-binder, described herein can be used as a diagnostic agent for diagnosing a disease by detecting the disease marker as the target molecule. In some aspects, provided herein is a method for detecting a marker, such as a target, in a sample comprising (i) contacting the sample with a binder molecule, such as a co-binder, provided herein under a condition sufficient to form a complex of the binder molecule and the marker, and (ii) detecting the presence of the complex in the sample. In some aspects, provided herein is a method of diagnosing a disease in a subject comprising (i) contacting a sample from the individual with a binder molecule, such as a co-binder, provided herein under a condition sufficient to form a complex of the binder molecule and a marker of the disease, wherein the binder molecule specifically binds to the marker, and (ii) detecting the presence of the complex in the sample.

Early detection of disease is important for a variety of diseases, including cancer, infectious diseases, cardiovascular diseases, brain injuries, and Alzheimer's disease. Often, the earlier a disease is diagnosed, the more likely that it can be cured or successfully managed. Cancer screening tests include mammograms for breast cancer, Pap smears or high risk HPV detection for cervical cancer, and colonoscopy for colon cancer, etc. Early diagnosis of cancer and other diseases can increase chances of survival and ensure patients receiving the most effective treatment at the earliest possible time.

Detecting disease markers in blood or other bodily fluids for early diagnosis is an attractive approach because of its noninvasive nature, ease of test implementation, and cost effectiveness. Using cancer as an example, disease markers can be protein-based (e.g. detection via ELISA) or DNA-based (e.g. detection via PCR or NGS) as in “liquid biopsy.” A major problem in the identification of disease markers is the very low concentrations of the disease markers at the earliest stage of disease progression. For example, HER2 amplified tumor cells are known to express about 2×10⁶ HER2 protein molecules per cell and HER2 gene amplified at about 25 copies per cell (Kallioniemi, et al. PNAS 1992 June, 89 (12) 5321-5325). Therefore, one tumor cell equivalent of HER2 tumor markers circulating in the blood is only about 4×10² protein molecules per ml of blood and about 5×10⁻³ DNA copies per ml of blood. This calculation suggests that detecting DNA markers is much harder than detecting protein markers for the purpose of early diagnosis.

The K_{D} of a typical strong antibody-antigen interaction is estimated to be at about 1×10⁻¹⁰ M (Foote & Eisen, Proc Natl Acad Sci USA. 1995 Feb. 28; 92 (5): 1254-1256). This means that when a protein disease marker is present at less than 1×10⁻¹² M (i.e. less than 6×10⁸ molecules per ml of blood), it is thermodynamically unfavorable for the very high affinity antibody to bind to the disease marker. This calculation therefore suggests that antibody in general doesn't have sufficient binding affinity for early detection of very low concentration of disease markers such as single tumor cell equivalent HER2 marker. Biotin-streptavidin binding are known to be one of the strongest noncovalent binding interactions and the K_{D} is reported to be at about 1×10⁻¹⁵ M (Foote & Eisen, Proc Natl Acad Sci USA. 1995 Feb. 28; 92 (5): 1254-1256). The binder molecules, such as a co-binder, described herein have high binding affinity are particular suited for disease marker detection.

In addition to the issue of insufficient binding affinity, antibody also lacks specificity/selectivity for detecting very low levels of disease markers. The concentration ranges of plasma proteins are known to cover at least 10-logs from basal level of IL-6 at about 6×10⁷ molecules/ml to human serum albumin at about 3×10¹⁷ molecules/ml (e.g. Geyer et al. Mol Syst Biol. 2017 September; 13 (9): 942 doi: 10.15252/msb.20156297). In order to detect HER2 protein at single tumor cell equivalent level of about 4×10² molecules/ml, anti-HER2 antibody not only needs to have high binding affinity, but also needs to have specificity/selectivity against unintended plasma proteins by at least 1×10⁵ fold. For example, anti-HER2 antibody's KD for HER2 should preferably be 1×10⁵ fold lower than its KD for unintended, nonspecific plasma proteins in order to minimize nonspecific background. Such high level of antibody specificity is difficult to achieve as antibodies are known to have relatively limited sequence and structural diversities in the antigen binding sites (e.g. Peng et al. Proc Natl Acad Sci USA. 2014 Jul. 1; 111 (26): E2656-E2665. doi: 10.1073/pnas.1401131111).

As indicated above, the K_{D} of a typical strong antibody-antigen interaction is estimated to be at about 1×10⁻¹⁰ M (Foote & Eisen, Proc Natl Acad Sci USA. 1995 Feb. 28; 92 (5): 1254-1256). It is therefore concluded that even high affinity antibody generally will not have sufficient binding affinity and/or sufficient specificity to detect disease markers in blood at the earliest possible time. It is known that most antibodies do not bind to unique epitopes and they cross-react with unintended proteins, albeit at lower binding affinity. Furthermore, a nonspecific protein unrelated to the intended target can have an epitope similar but not identical to the specific epitope. Then the antibody's binding affinity for the similar epitope is lower than that for the specific epitope, which results in cross-reactivity with a proportionate lower signal, called nonspecific background. It is this nonspecific background that interferes with the specific antibody-antigen binding interaction, particularly when the intended target is present at very low concentration and the unintended nonspecific protein target is present at relatively high concentration, such as in the situation of early detection of disease markers in blood. Thus, to detect a disease marker at the earliest possible time, the binder for the disease marker needs to have very high binding affinity. The binder also needs to be able to minimize the nonspecific binding to unintended targets even when those unintended targets are present at relatively high concentration. In some embodiments, the binder molecules, such as a co-binder, described herein with significantly better binding affinity and better specificity are particular suited for disease marker detection and early disease diagnosis.

In one aspect, provided herein is a method for detecting a marker in a sample comprising (i) contacting the sample with a binder molecule, such as a co-binder, provided herein under a condition sufficient to form a complex of the binder molecule and the marker, and (ii) detecting the complex in the sample.

In some embodiments of the methods provided herein, the complex is detected by measuring a labeled agent conjugated to the complex. In some embodiments of the methods provided herein, the complex is detected by measuring a labeled agent conjugated to a binder molecule, such as a co-binder.

In some specific embodiments of the methods of detecting a marker, the sample is a bodily fluid, a tissue, or a cell. In some specific embodiments of the methods of detecting a marker, the sample is blood, bone marrow, plasma, serum, urine, or cerebrospinal fluid. In some embodiments, the complex is formed in vitro. In some embodiments, the complex is formed in vivo. In some embodiments, the complex is detected in vitro. In some embodiments, the complex is detected in vivo. In some embodiments, the complex is formed in vitro and the complex is detected in vitro. In some embodiments, the complex is formed in vivo and the complex is detected in vivo. In some embodiments, the complex is formed under physiological conditions. In some embodiments, the complex is formed at 37° C. In some embodiments, the complex is formed under physiological vascular shear stress, for example 10-70 dynes/cm² (range of shear stress in the arteries) or 1-6 dynes/cm² (range of shear stress in the veins). In some embodiments, the complex is formed at 37° C. and under physiological vascular shear stress, for example 10-70 dynes/cm² (range of shear stress in the arteries) or 1-6 dynes/cm² (range of shear stress in the veins). In some embodiments, the complex is detected under physiological conditions. In some embodiments, the complex is detected at 37° C. In some embodiments, the complex is detected under physiological vascular shear stress, for example 10-70 dynes/cm² (range of shear stress in the arteries) or 1-6 dynes/cm² (range of shear stress in the veins). In some embodiments, the complex is detected at 37° C. and under physiological vascular shear stress, for example 10-70 dynes/cm² (range of shear stress in the arteries) or 1-6 dynes/cm² (range of shear stress in the veins). In some embodiments, the complex is formed under normal laboratory conditions (e.g. at room temperature or 25° C.). In some embodiments, the complex is detected under normal laboratory conditions (e.g. at room temperature or 25° C.). In some embodiments, the complex is formed under normal laboratory conditions (e.g. at room temperature or 25° C.) and is detected under normal laboratory conditions (e.g. at room temperature or 25° C.).

In some embodiments of the methods provided herein, the complex is detected by measuring a labeled agent conjugated to the complex. In some embodiments, the labeled agent can be a colorimetric reagent, a fluorescent reagent, a chemiluminescent reagent, a radioisotope, a metal ion, an enzyme, a polymer, or an affinity tag. The colorimetric reagent can be, for example, PNPP (p-nitrophenyl phosphate), ABTS (2,2′-azino-bis(3-ethylbenzothiazoline-6-sulphonic acid)) or OPD (o-phenylenediamine). The fluorescent reagent can be, for example, QuantaBlu™ or QuantaRed™ (Thermo Scientific, Waltham, MA). The luminescent reagent can be, for example, luminol or luciferin.

In some embodiments of the methods provided herein, the labeled agent is a fluorescent molecule, a radioisotope, a metal ion, an enzyme, a biotin, a polymer or an antibody.

In some embodiments, the binder molecule, such as a co-binder, can be conjugated to an affinity tag for detection. In some embodiments, the affinity tag can be Glutathione-S-transferase, HA-tag, His-Tag, FLAG-Tag, or biotin.

In some embodiments, the complex having the binder molecule, such as a co-binder, and the target molecule can be detected by a secondary antibody that recognizes the binder molecule. The secondary antibody can be, for example, an anti-human IgA, anti-human IgD, anti-human IgE, anti-human IgG, or anti-human IgM antibody. The secondary antibody can be a monoclonal or polyclonal antibody. The secondary antibody can be derived from any mammalian organism, including mice, rats, hamsters, goats, camels, chicken, rabbit, and others. The secondary antibody can also be recombinant. Secondary antibodies can be conjugated to enzymes (e.g., horseradish peroxidase (HRP), alkaline phosphatase (AP), luciferase, and the like) or dyes (e.g., colorimetric dyes, fluorescent dyes, fluorescence resonance energy transfer (FRET)-dyes, time-resolved (TR)-FRET dyes, and the like). In some embodiment, the secondary antibody can be conjugated to a fluorescein (FITC) based dye, such as fluorescein isothiocyanate. In some embodiment, the secondary antibody can be conjugated to Alexa Fluor® 488 (Life technologies).

The presence or absence of the complex can also be detected by an enzyme-linked immunosorbent assay (ELISA) (including multiplex ELISA), an immunohistochemistry assay (IHC), an immunofluorescence assay (IF), a western blot (WB), flow cytometry, a fluorescent immunosorbent assay (FIA), a chemiluminescence immuno assay (CIA), a radioimmunoassay (RIA), an enzyme multiplied immunoassay, a solid phase radioimmunoassay (SPROA), a fluorescence polarization (FP) assay, a fluorescence resonance energy transfer (FRET) assay, a time-resolved fluorescence resonance energy transfer (TR-FRET) assay, a surface plasmon resonance (SPR) assay, or a Dot-Blot assay. Methods and protocols for conducting immunoassays and biophysical protein-interaction assays are well known in the art. See, e.g., Wild D., The Immunoassay Handbook, Elsevier Science, 4th Edition (2013); Fu H., Protein-Protein Interactions, Humana Press, 4th Edition (2004).

As a person of ordinary skill in the art would understand, a higher level of a disease marker in the human sample can indicate a higher likelihood of the human subject having the disease. Schouwers S, et al., Clin Chem Lab Med. (2014) 52 (4): 547-51. In some embodiments, the detection step can further include determining the level of the target molecule in the sample. Determining the level of the target molecule can include comparing the level of the target molecule in the sample from the subject to a control level of the target molecule in a sample from a healthy individual, wherein an increase in the target molecule level in the sample from the subject compared to the control level indicates that the subject has the disease. Determining the level of the target molecule can include associating the level with the likelihood of the subject having the disease, wherein a higher level indicates a higher likelihood of having the disease.

The level of the target molecule in a human sample over a period of time can indicate the progression of disease that the target molecule is associated with over the period of time. The time period can be a time course of treatment, wherein the changes in the level of the target molecule can indicate the efficacy of the treatment. In some embodiment, the present disclosure provides a method of monitoring the target molecule level in a patient at different time points, including determining the levels of target molecules in the two or more samples taken at different time points from the patient and comparing the levels of target molecule in the two or more samples. A decreased level of the target molecule in a sample obtained at a subsequent time point relative to the level of target molecule in the sample obtained at the first time point can indicate that the condition of the patient is improving or the treatment received by the patient is efficacious. An increase in level of the target molecule in a sample obtained at a subsequent time point relative to the level of target molecule in the sample obtained at the first time point can indicate that the condition of the human subject is deteriorating. In some embodiments, one or more samples were obtained at the beginning of the course of a particular treatment and one or more samples were obtained at later time points throughout the course of the treatment.

In some embodiments, detection and diagnosis can be accomplished, for example, by conjugating a binder molecule, such as a co-binder, disclosed herein to detectable substances including, but not limited to, radioactive materials, such as, but not limited to, zirconium (⁸⁹Zr), iodine (¹³¹I, ¹²⁵I, ¹²⁴I, ¹²³I, and ¹²¹I,), carbon (¹⁴C, ¹¹C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵O, ¹³N, ⁶⁴Cu, ⁹⁴mTc, ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁸⁶Y, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin, and non-radioactive paramagnetic metal ions.

A conjugate having a binder molecule, such as a co-binder, disclosed herein that is detectably labeled as provided herein can be used for diagnostic purposes to detect, diagnose, or monitor a disease, such as cancer, infectious diseases, cardiovascular diseases, brain injuries, and Alzheimer's disease.

Accordingly, in one aspect, provided herein is a method of diagnosing a disease in a subject comprising (i) contacting the sample with a binder molecule, such as a co-binder, provided herein under a condition sufficient to form a complex of the binder molecule and a marker of the disease, wherein the binder molecule specifically binds to the marker, and (ii) detecting the complex in the sample.

In certain embodiments of the methods provided herein, the complex is detected by measuring a labeled agent conjugated to the complex, as described further above in this section.

In certain embodiments of the methods provided herein, the labeled agent is a labeled agent as described further above in this section.

In some specific embodiments of the methods provided herein, the labeled agent is a fluorescent molecule, a radioisotope, a metal ion, an enzyme, a biotin, a polymer or an antibody.

In some specific embodiments of the methods, the sample is a bodily fluid, a tissue, or a cell. In some specific embodiments of the methods, the sample is blood, bone marrow, plasma, serum, urine, or cerebrospinal fluid. In some embodiments, the complex is formed in vitro. In some embodiments, the complex is formed in vivo. In some embodiments, the complex is detected in vitro. In some embodiments, the complex is detected in vivo. In some embodiments, the complex is formed in vitro and the complex is detected in vitro. In some embodiments, the complex is formed in vivo and the complex is detected in vivo. In some embodiments, the complex is formed under physiological conditions. In some embodiments, the complex is formed at 37° C. In some embodiments, the complex is formed under physiological vascular shear stress, for example 10-70 dynes/cm² (range of shear stress in the arteries) or 1-6 dynes/cm² (range of shear stress in the veins). In some embodiments, the complex is formed at 37° C. and under physiological vascular shear stress, for example 10-70 dynes/cm² (range of shear stress in the arteries) or 1-6 dynes/cm² (range of shear stress in the veins). In some embodiments, the complex is detected under physiological conditions. In some embodiments, the complex is detected at 37° C. In some embodiments, the complex is detected under physiological vascular shear stress, for example 10-70 dynes/cm² (range of shear stress in the arteries) or 1-6 dynes/cm² (range of shear stress in the veins). In some embodiments, the complex is detected at 37° C. and under physiological vascular shear stress, for example 10-70 dynes/cm² (range of shear stress in the arteries) or 1-6 dynes/cm² (range of shear stress in the veins). In some embodiments, the complex is formed under normal laboratory conditions (e.g. at room temperature or 25° C.). In some embodiments, the complex is detected under normal laboratory conditions (e.g. at room temperature or 25° C.). In some embodiments, the complex is formed under normal laboratory conditions (e.g. at room temperature or 25° C.) and is detected under normal laboratory conditions (e.g. at room temperature or 25° C.).

In some embodiments of the methods provided herein, the marker is present in the sample at a concentration of no more than 1×10⁻⁸ M, no more than 0.5×10⁻⁸ M, no more than 1×10⁻⁹ M, no more than 0.5×10⁻⁹ M, no more than 1×10⁻¹⁰ M, no more than 0.5×10⁻¹⁰ M, no more than 1×10⁻¹¹ M, no more than 0.5×10⁻¹¹ M, no more than 1×10⁻¹² M, no more than 0.5×10⁻¹² M, no more than 1×10⁻¹³ M, no more than 0.5×10⁻¹³ M, no more than 1×10⁻¹⁴ M, no more than 0.5×10⁻¹⁴ M, no more than 1×10⁻¹⁵ M, no more than 0.5×10⁻¹⁵ M, no more than 1×10⁻¹⁶ M, no more than 0.5×10⁻¹⁶ M, no more than 1×10⁻¹⁷ M, no more than 0.5×10⁻¹⁷ M, no more than 1×10⁻¹⁸ M, no more than 0.5×10⁻¹⁸ M, no more than 1×10⁻¹⁹ M, no more than 0.5×10⁻¹⁹ M, no more than 1×10⁻²⁰ M, no more than 0.5×10⁻²⁰ M, no more than 1×10⁻²¹ M, or no more than 0.5×10⁻²¹ M.

In some embodiments of the methods provided herein, the marker is present in the sample at a concentration of less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×10⁻¹¹ M, less than 0.5×10⁻¹¹ M, less than 1×10⁻¹² M, less than 0.5×10⁻¹² M, less than 1×10⁻¹³ M, less than 0.5×10⁻¹³ M, less than 1×10⁻¹⁴ M, less than 0.5×10⁻¹⁴ M, less than 1×10⁻¹⁵ M, less than 0.5×10⁻¹⁵ M, less than 1×10⁻¹⁶ M, less than 0.5×10⁻¹⁶ M, less than 1×10⁻¹⁷ M, less than 0.5×10⁻¹⁷ M, less than 1×10⁻¹⁸ M, less than 0.5×10⁻¹⁸ M, less than 1×10⁻¹⁹ M, less than 0.5×10⁻¹⁹ M, less than 1×10⁻²⁰ M, less than 0.5×10⁻²⁰ M, less than 1×10⁻²¹ M, or less than 0.5×10⁻²¹ M.

In some embodiments of the methods provided herein, the marker is present in the sample at a concentration of about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, about 0.5×10⁻¹¹ M, about 1×10⁻¹² M, about 0.5×10⁻¹² M, about 1×10⁻¹³ M, about 0.5×10⁻¹³ M, about 1×10⁻¹⁴ M, about 0.5×10⁻¹⁴ M, about 1×10⁻¹⁵ M, about 0.5×10⁻¹⁵ M, about 1×10⁻¹⁶ M, about 0.5×10⁻¹⁶ M, about 1×10⁻¹⁷ M, about 0.5×10⁻¹⁷ M, about 1×10⁻¹⁸ M, about 0.5×10⁻¹⁸ M, about 1×10⁻¹⁹ M, about 0.5×10⁻¹⁹ M, about 1×10⁻²⁰ M, about 0.5×10⁻²⁰ M, about 1×10⁻²¹ M, or about 0.5×10⁻²¹ M.

In some embodiments, the detection method can further include assaying the expression of a disease marker on the cells or a tissue sample of a subject using co-binders disclosed herein; and comparing the level of the disease marker with a control level, e.g., levels in normal tissue samples (e.g., from a subject not having a disease, or from the same subject before disease onset), whereby an increase in the assayed level of the disease marker compared to the control level is indicative of the disease. Such diagnostic methods can allow health professionals to employ preventative measures or aggressive treatment earlier than otherwise possible thereby preventing the development or further progression of the disease.

The binder molecule, such as a co-binder, disclosed herein can also be used to assay the level of the target molecule in a biological sample using classical immunohistological methods as provided herein or as well known to those of skill in the art (e.g., see Jalkanen et al., 1985, J. Cell. Biol. 101:976-985; and Jalkanen et al., 1987, J. Cell. Biol. 105:3087-3096), such as the enzyme linked immunosorbent assay (ELISA) and the radioimmunoassay (RIA). Suitable assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

In one aspect, the disclosure provides for the detection and diagnosis of disease in a human. In some embodiments, diagnosis includes: a) administering (for example, parenterally, subcutaneously, or intraperitoneally) to a subject an effective amount of a conjugate having a binder molecule, such as a co-binder, disclosed herein; b) waiting for a time interval following the administering for permitting the conjugate to preferentially concentrate at sites in the subject where the disease marker is expressed (and, in some aspects, for unbound conjugate or fusion protein to be cleared to background level); c) determining background level; and d) detecting the conjugate in the subject, such that detection of conjugate above the background level indicates that the subject has a disease. Background level can be determined by various methods including, comparing the amount of conjugate detected to a standard value previously determined for a particular system.

It is understood that the size of the subject and the imaging system used will determine the quantity of imaging moiety needed to produce diagnostic images and can be readily determined by one of skill in the art. For example, in the case of a radioisotope conjugated to a binder molecule described herein, for a human subject the quantity of radioactivity injected will normally range from about 5 to 20 millicuries of 99Tc. The conjugate will then preferentially accumulate at the location of cells which express the target molecule. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of detectable agent used and the mode of administration, the time interval following the administration for permitting the conjugate to preferentially concentrate at sites in the subject and for unbound conjugate to be cleared to background level is 6 to 48 hours or 6 to 24 hours or 6 to 12 hours. In another embodiment, the time interval following administration is 5 to 20 days or 5 to 10 days. In some embodiments, monitoring of a disease is carried out by repeating the method for diagnosing as provided herein, for example, one month after initial diagnosis, six months after initial diagnosis, one year after initial diagnosis, or longer.

The presence of the conjugate or fusion protein can be detected in the subject using methods known in the art for in vivo scanning. These methods depend upon the type of detectable agent used. A skilled artisan will be able to determine the appropriate method for detecting a particular detectable agent. Methods and devices that can be used in the diagnostic methods of the disclosure include, but are not limited to, computed tomography (CT), whole body scan such as position emission tomography (PET), magnetic resonance imaging (MRI), and sonography. In some embodiments, the binder molecule, such as a co-binder, disclosed herein is conjugated to a radioisotope and is detected in the subject using a radiation responsive surgical instrument. In another embodiment, the binder molecule, such as a co-binder, disclosed herein is conjugated to a fluorescent compound and is detected in the subject using a fluorescence responsive scanning instrument. In another embodiment, the binder molecule, such as a co-binder, disclosed herein is conjugated to a positron emitting metal, such as zirconium (89Zr) or any other positron emitting metal provided herein or that is well known in the art to be detectable by positron emission-tomography, and is detected in the subject using positron emission-tomography. In yet another embodiment, the binder molecule, such as a co-binder, disclosed herein is conjugated to a paramagnetic label and is detected in a subject using magnetic resonance imaging (MRI).

Contemplated herein are also the uses of the binder molecule, such as a co-binder, disclosed herein in place of an antibody in applications such as an ELISA, IHC, IF, IP, WB, flow cytometry, flow sorting, imaging, multiplex ELISAs, or multiplex antibody arrays. Contemplated herein are also the use the binder molecule, such as a co-binder, disclosed herein in the detection of markers related to food & environment safety detection and monitoring.

In certain embodiments of the various methods provided herein, the subject is a mammal. In some embodiments, the subject is a mammal selected from the group consisting of Caviinae (guinea pig), Sus (pigs), Macaca Fascicularis (monkeys, e.g. cynomolgus monkey), Hominoid apes (gibbons, orangutans, gorillas, chimpanzees, and humans), Canis (dog), Rattus (rat), and Mus musculus (mouse). In a specific embodiment, the subject is a human.

### B. Methods of Treatment

The binder molecules, such as a co-binder, described herein can also have significant advantages over a typical bivalent antibody for therapeutic applications. Many drug targets such as GPCRs, ion channels, tyrosine kinase receptors, cytokine receptors that have much lower level of expression than that of HER2 on cell surface and they need high affinity binders to block their binding to their natural ligand or to serve as antagonists or agonists.

Accordingly, in one aspect, provided herein is a method of treating a disease in a subject, comprising administering a therapeutically effective amount of a binder molecule, such as a co-binder, provided herein to the subject, wherein the disease is treatable by activating or inhibit the target that the binder molecule, such as a co-binder, specifically binds to. As described herein, a binder molecule, such as a co-binder, provided herein has increased binding affinity and/or specificity over the individual binding moiety in the co-binder or a control binder molecule, such as a control co-binder described herein. Accordingly, in one aspect, provided herein is a method of increasing binding affinity, or use thereof, of a first binding moiety for a target, comprising constructing a co-binder of the first binding moiety with a second binding moiety according to any or any combination of the configuration or embodiments provided herein. In some aspect, provided herein is a method of increasing the therapeutic or diagnostic efficacy, or use thereof, of a binding moiety by link the binding moiety to another binding moiety such that the resulting binding molecule binds to a target with sufficient affinity and specificity for therapeutic or diagnostic and produces desired biological efficacy.

In some embodiments, the binder molecule, such as a co-binder, disclosed herein is an agonist of a target, and provided herein are methods to treat a disease treatable by activating the biological function of the target in a subject, which includes administering a therapeutically effective amount of the binder molecule that specifically binds to the target to the subject. In some embodiments, the binder molecule, such as a co-binder, disclosed herein is an antagonist of a target, and provided herein are methods to treat a disease treatable by inhibiting the biological function of the target in a subject, which includes administering a therapeutically effective amount of the binder molecule that specifically binds to the target to the subject.

In some embodiments of the methods provided herein, the target is expressed at below 1×10⁷, below 0.5×10⁷, below 1×10⁶, below 0.5×10⁶, below 1×10⁵, below 0.5×10⁵, below 1×10⁴, below 0.5×10⁴, below 1×10³, below 0.5×10³, below 1×10², or below 0.5×10² per cell. In some embodiments of the methods provided herein, the target is expressed at no more than 1×10⁷, no more than 0.5×10⁷, no more than 1×10⁶, no more than 0.5×10⁶, no more than 1×10⁵, no more than 0.5×10⁵, no more than 1×10⁴, no more than 0.5×10⁴, no more than 1×10³, no more than 0.5×10³, no more than 1×10², or no more than 0.5×10² per cell. In some embodiments of the methods provided herein, the target is expressed at about 1×10⁷, about 0.5×10⁷, about 1×10⁶, about 0.5×10⁶, about 1×10⁵, about 0.5×10⁵, about 1×10⁴, about 0.5×10⁴, about 1×10³, about 0.5×10³, about 1×10², or about 0.5×10² per cell.

A class of drug targets is secreted molecules in bodily fluid (e.g. blood,), such as growth factors, cytokines, chemokines, which can also benefit from the binder molecule, such as a co-binder, disclosed herein having high affinity to inhibit or activate their biological function. In some embodiments of the methods provided herein, the secreted protein target is at less than 1×10¹⁰, less than 0.5×10¹⁰, less than 1×10⁹, less than 0.5×10⁹, less than 1×10⁸, less than 0.5×10⁸, less than 1×10⁷, less than 0.5×10⁷, less than 1×10⁶, less than 0.5×10⁶, less than 1×10⁵, less than 0.5×10⁵, less than 1×10⁴, less than 0.5×10⁴, less than 1×10³, less than 0.5×10³, less than 1×10², or less than 0.5×10² molecules/ml in a bodily fluid sample, for example, blood, serum, plasma, bone marrow, or cerebrospinal fluid. In some embodiments of the methods provided herein, the secreted protein target is at about 1×10¹⁰, about 0.5×10¹⁰, about 1×10⁹, about 0.5×10⁹, about 1×10⁸, about 0.5×10⁸, about 1×10⁷, about 0.5×10⁷, about 1×10⁶, about 0.5×10⁶, about 1×10⁵, about 0.5×10⁵, about 1×10⁴, about 0.5×10⁴, about 1×10³, about 0.5×10³, about 1×10², or about 0.5×10² molecules/ml in a bodily fluid sample, for example, blood, serum, plasma, bone marrow, or cerebrospinal fluid.

Many drug targets belong to protein families whose members share nearly identical sequences in functionally conserved regions such as the region of protein binding interaction or the region responsible for enzymatic activities, therefore it is difficult to generate a therapeutic antibody that can specifically target the functionally conserved region of a particular family member without affecting the function of other members in the protein family. A binder molecule, such as a co-binder, having the first binding moiety that binds to the functionally conserved region at relatively low affinity could still inhibit or activate the function of the protein target, when the second binding moiety in the co-binder enhances the binding affinity and stabilizes the binding of the first binding moiety to the functionally conserved region. If the second binding moiety binds to a unique sequence in the target that is not shared with other member of the protein family, selective inhibition or activation of this particular target molecule's function can be achieved.

Accordingly, in some embodiments, provided herein is a binder molecule, such as a co-binder, that has the first binding moiety binding to a functionally conserved region or binding site of a protein family at low affinity so that it alone can't bind to the protein target or its family members stably, and the second binding moiety binding to a unique sequence or binding site in the target molecule that is distinct from other members of the family. The resulting binder molecule, such as a co-binder, selectively inhibits or activates the function of the target molecule without affecting other members of the family. In some embodiments, providing herein are co-binders having a first binding moiety, a second binding moiety, and a linker that connects the first binding moiety and second binding moiety, wherein the first and second binding moieties bind to non-overlapping epitopes on a target molecule, and wherein the first binding moiety binds to a functional conserved region or binding site of the target molecule at preferably low binding affinity (e.g. with a KD of at least 1×10⁻⁹ M). In some embodiments, providing herein are binder molecules, such as a co-binder, having a first binding moiety, a second binding moiety, and a linker that connects the first binding moiety and second binding moiety, wherein the first and second binding moieties simultaneously bind to non-overlapping epitopes on a target molecule, and wherein the first binding moiety binds to a functional conserved region or binding site of the target molecule at preferably low binding affinity (e.g. with a KD of at least 1×10⁻⁹ M). In some embodiments, the first binding moiety of a co-binder binds the target molecule with a KD of at least 1×10⁻⁹ M, at least 1×10⁻⁸ M, at least 1×10⁻⁷ M, at least 1×10⁻⁶.

Naïve B-cell library or library of primary immune response contain more diverse repertoire of low affinity binding moieties than that of secondary immune response where the binding moieties are more selective but higher affinity due to affinity maturation. However, therapeutic efficacy of low affinity binding moieties having fast dissociation rate is usually not as good as those high affinity binding moieties having slower dissociation rate. The binding affinity of a desirable binding moiety capable of inhibiting or activating the function of a drug target can be improved greatly with a co-binder that contains a second binding moiety that works cooperatively and synergistically with the desirable first binding moiety. In some embodiment, providing herein are co-binders having a first binding moiety and a second binding moiety that simultaneously bind to non-overlapping epitopes on a drug target, wherein the first binding moiety has the capability to inhibit or activate the function of the drug target upon binding, and a relatively low binding affinity with the drug target (e.g. with a KD of at least 1×10⁻⁹ M), and wherein the binding affinity is improved by at least more than 50-fold due to the presence of second binding moiety that simultaneously and synergistically binds to a distinct, nonoverlapping epitope on the drug target. In some embodiment, providing herein are co-binders having a first binding moiety and a second binding moiety that bind to non-overlapping epitopes on a drug target, wherein the first binding moiety has the capability to inhibit or activate the function of the drug target upon binding, and a relatively low binding affinity with the drug target (e.g. with a KD of at least 1×10⁻⁹ M), and wherein the binding affinity is improved by at least more than 50-fold due to the presence of second binding moiety that binds to a distinct, nonoverlapping epitope on the drug target. In some embodiments, the first binding moiety that can inhibit or activate the function of the drug target upon binds the drug target with a KD of at least 1×10⁻⁹ M. In some embodiments, the first binding moiety that can inhibit or activate the function of the drug target upon binds the drug target with a KD of at least 1×10⁻⁸ M. In some embodiments, the first binding moiety that can inhibit or activate the function of the drug target upon binds the drug target with a KD of at least 1×10⁻⁷ M. In some embodiments, the first binding moiety that can inhibit or activate the function of the drug target upon binds the drug target with a KD of at least 1×10⁻⁶ M. In some embodiments, the binding affinity is improved by more than 100-fold. In some embodiments, the binding affinity is improved by more than 200-fold. In some embodiments, the binding affinity is improved by more than 500-fold. In some embodiments, the binding affinity is improved by more than 1000-fold. In some embodiments, the binding affinity is improved by more than 2000-fold. In some embodiments, the binding affinity is improved by more than 5000-fold. In some embodiments, the binding affinity is improved by more than 10,000-fold.

Furthermore, B-cell library of naïve or primary immune response contain more diverse repertoire of low affinity binding moieties and their binding affinity can be greatly improved through a synergistic second binding moiety in a co-binder. Accordingly, co-binders provided herein can bind to any region of interest in a target molecule with high affinity and/or with broad affinity spectrum for different purposes (affinity fine-tuning). In some embodiments, the first binding moiety of a co-binder binds the target molecule with a KD of at least 1×10⁻⁹M, at least 1×10⁻⁸ M, at least 1×10⁻⁷ M, or at least 1×10⁻⁶ M, and the binding affinity of the co-binder is improved by more than 10-fold, more than 20-fold, more than 50-fold, more than 100-fold, more than 200-fold, more than 500-fold, more than 1000-fold, more than 2000-fold, more than 5000-fold, or more than 10,000-fold due to the presence of the second binding moiety that binds to a distinct, nonoverlapping epitope in the same target molecule.

In some embodiments, provided herein is a method for treating a disease in a subject in need thereof. The methods can include administering a therapeutically effective amount of a pharmaceutical composition provided herein to the subject. For example, the pharmaceutical composition can include a binder molecule, such as a co-binder, provided herein. Diseases that can be treated or prevented using the methods of the disclosure include cancer, infectious diseases, cardiovascular diseases, brain injuries, autoimmune diseases, and neurodegenerative diseases such as Alzheimer's disease. In some embodiments, provided herein is a method for treating a cancer in a subject in need thereof, which include administering a therapeutically effective amount of a pharmaceutical composition having a binder molecule, such as a co-binder, provided herein to the subject. In some embodiments of the disclosure, the binder molecule, such as a co-binder, is used as a part of CAR-T construct that binds to target molecule such as a tumor antigen or a neoantigen with higher affinity and specificity.

The binder molecule, such as a co-binder, provided herein can also be used to specifically target a therapeutic agent to a diseased cell, tissue, or organ. In some embodiments, provided herein are therapeutic uses of the binder molecule, such as a co-binder, provided herein conjugated (covalent or non-covalent conjugations) or recombinantly fused to one or more therapeutic agent. In this context, for example, the binder molecule, such as a co-binder, can be conjugated or recombinantly fused to a therapeutic agent, such as a cytotoxin, e.g., a cytostatic or cytocidal agent, or a radioactive metal ion, e.g., alpha-emitters. A cytotoxin or cytotoxic agent includes any agent that is detrimental to cells. A therapeutic agent can be a chemotherapeutic such as, but is not limited to, an anthracycline (e.g., doxorubicin and daunorubicin (formerly daunomycin)); a taxan (e.g., paclitaxel (Taxol) and docetaxel (Taxotere); an antimetabolite (e.g., methotrexate, 6-mercaptopurine, 6-thioguanine, cytarabine, 5-fluorouracil and decarbazine); or an alkylating agent (e.g., mechlorethamine, thioepa chlorambucil, melphalan, carmustine (BCNU), lomustine (CCNU), cyclothosphamide, busulfan, dibromomannitol, streptozotocin, mitomycin C, cisdichlorodiamine platinum (II) (DDP) and cisplatin); an antibiotic (e.g., actinomycin D, bleomycin, mithramycin, and anthramycin (AMC)); an Auristatin molecule (e.g., auristatin PHE, bryostatin 1, solastatin 10, monomethyl auristatin E (MMAE) and monomethylauristatin F (MMAF)); a hormone (e.g., glucocorticoids, progestins, androgens, and estrogens); a nucleoside analoge (e.g. Gemcitabine), a DNA-repair enzyme inhibitor (e.g., etoposide and topotecan), a kinase inhibitor (e.g., compound ST1571, also known as Gleevec or imatinib mesylate); a cytotoxic agent (e.g., maytansine, paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, 1-dehydrotestosterone, glucorticoids, procaine, tetracaine, lidocaine, propranolol, puromycin and analogs or homologs thereof; a farnesyl transferase inhibitor (e.g., R115777, BMS-214662, and those disclosed by, for example, U.S. Pat. No. 6,458,935); a topoisomerase inhibitor (e.g., camptothecin, irinotecan, SN-38, topotecan, 9-aminocamptothecin, GG-211 (GI 147211), DX-8951f, IST-622, rubitecan, pyrazoloacridine, XR-5000, saintopin, UCE6, UCE1022, TAN-1518A, TAN 1518B, KT6006, KT6528, ED-110, NB-506, ED-110, NB-506, fagaronine, coralyne, beta-lapachone and rebeccamycin); a DNA minor groove binder (e.g., Hoescht dye 33342 and Hoechst dye 33258); adenosine deaminase inhibitors (e.g., Fludarabine phosphate and 2-Chlorodeoxyadenosine); or pharmaceutically acceptable salts, solvates, clathrates, or prodrugs thereof. A therapeutic agent can be a immunotherapeutic such as, but is not limited to, cetuximab, bevacizumab, heceptin, rituximab).

In addition, the binder molecule, such as a co-binder, provided herein can be conjugated to a therapeutic agent such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm; or a macrocyclic chelator, such as 1,4,7,10-tetraazacyclododecane-N,N′,N″,N″-tetraacetic acid (DOTA).

Further, the binder molecule, such as a co-binder, provided herein can be conjugated (covalent or non-covalent conjugations) or recombinantly fused to a therapeutic agent that modifies a given biological response. Thus, therapeutic agents are not to be construed as limited to classical chemical therapeutic agents. For example, therapeutic agent can be a protein, peptide, or polypeptide possessing a desired biological activity. Such proteins can include, for example, a toxin (e.g., abrin, ricin A, pseudomonas exotoxin, cholera toxin and diphtheria toxin); a protein such as tumor necrosis factor, γ-interferon, α-interferon, nerve growth factor, platelet derived growth factor, tissue plasminogen activator, an apoptotic agent (e.g., TNF-γ, AIM I, AIM II, Fas Ligand and VEGF), an anti-angiogenic agent (e.g., angiostatin, endostatin and a component of the coagulation pathway such as tissue factor); a biological response modifier (e.g., a cytokine such as interferon gamma, interleukin-1, interleukin-2, interleukin-5, interleukin-6, interleukin-7, interleukin-9, interleukin-10, interleukin-12, interleukin-15, interleukin-23, granulocyte macrophage colony stimulating factor, and granulocyte colony stimulating factor); a growth factor (e.g., growth hormone), or a coagulation agent (e.g., calcium, vitamin K, tissue factors, such as but not limited to, Hageman factor (factor XII), high-molecular-weight kininogen (HMWK), prekallikrein (PK), coagulation proteins-factors II (prothrombin), factor V, XIIa, VIII, XIIIa, XI, XIa, IX, IXa, X, phospholipid, and fibrin monomer).

Therapeutic agent conjugated or recombinantly fused to co-binders provided herein can be chosen to achieve the desired prophylactic or therapeutic effect(s). It is understood that it is within the skill level of a clinician or other medical personnel to consider the following to decide which therapeutic agent to conjugate or recombinantly fuse to co-binders provided herein: the nature of the disease, the severity of the disease, and the condition of the subject.

The co-binders or pharmaceutical compositions described herein can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

In some embodiments, one or more co-binders described herein are in a liquid pharmaceutical formulation as described above in Section 4.4.

Methods for administering a pharmaceutical composition having co-binders described herein are well known in the art. It is understood that the appropriate route of administration of a pharmaceutical composition can be readily determined by a skilled clinician. Exemplary routes of administration include intravenous injection, intramuscular injection, intradermal injection or subcutaneous injection. Moreover, it is understood that the formulation of the pharmaceutical composition can be readily adjusted to accommodate the route of administration.

The methods provided herein for treating a disease is intended to include (1) preventing the disease, i.e., causing the clinical symptoms of the disease not to develop in a subject that can be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. Provided herein are also methods of preventing a disease, which include forestalling of a clinical symptom indicative of disease. Therapeutically effective amount of the pharmaceutical composition used in the methods of the disclosure will vary depending on the pharmaceutical composition used, the disease and its severity and the age, weight, etc., of the subject to be treated, all of which is within the skill of the attending clinician.

### Exemplary Embodiments

Embodiment 1. A co-binder that specifically binds to a target, wherein the co-binder comprises: (i) a heavy chain variable region of a first antibody (VHAb1); (ii) a heavy chain variable region of a second antibody (VHAb2) comprising an N-terminal truncation of from 1 to 18 amino acids; and (iii) a polypeptide linker that links the VHAb1 C-terminal amino acid with the N-terminal amino acid of the truncated VHAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VHAb1 and VHAb2 bind to non-overlapping epitopes on the target.
Embodiment 2. The co-binder of embodiment 1, wherein the N-terminal truncation of the VHAb2 is from 1 to 10 amino acids.
Embodiment 3. A co-binder that specifically binds to a target, wherein the co-binder comprises: (i) a VHAb1; (ii) a VHAb2 comprising a truncation or a deletion in the framework 1 (FR1) region; and (iii) a polypeptide linker that links the VHAb1 C-terminal amino acid with the N-terminal amino acid of the VHAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VHAb2 complementarity determining region 1 (CDR1) are separated by from 8 to 25 amino acids; and wherein VHAb1 and VHAb2 bind to non-overlapping epitopes on the target.
Embodiment 4. A co-binder that specifically binds to a target, wherein the co-binder comprises: (i) a VHAb1; (ii) a VHAb2 comprising a truncation or a deletion in the framework 1 (FR1) region; and (iii) a polypeptide linker that links the VHAb1 C-terminal amino acid with the N-terminal amino acid of the VHAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VHAb2 complementarity determining region 1 (CDR1) are separated by no more than 25 amino acids; and wherein VHAb1 and VHAb2 bind to non-overlapping epitopes on the target.
Embodiment 5. A co-binder that specifically binds to a target, wherein the co-binder comprises: (i) a VHAb1; (ii) a VHAb2 comprising an N-terminal truncation of from 1 to 18 amino acids in the framework 1 (FR1) region; and (iii) a polypeptide linker that links the VHAb1 C-terminal amino acid with the N-terminal amino acid of the truncated VHAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VHAb1 and VHAb2 bind to non-overlapping epitopes on the target.
Embodiment 6. The co-binder of embodiment 5, wherein the N-terminal truncation of the FR1 is from 1 to 10 amino acids.
Embodiment 7. A co-binder that specifically binds to a target, wherein the co-binder comprises: (i) a heavy chain variable region of a first antibody (VHAb1); (ii) a light chain variable region of a second antibody (VLAb2) comprising an N-terminal truncation of from 1 to 18 amino acids; and (iii) a polypeptide linker that links the VHAb1 C-terminal amino acid with the N-terminal amino acid of the truncated VLAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VHAb1 and VLAb2 bind to non-overlapping epitopes on the target.
Embodiment 8. The co-binder of embodiment 7, wherein the N-terminal truncation of the VLAb2 is from 1 to 10 amino acids.
Embodiment 9. A co-binder that specifically binds to a target, wherein the co-binder comprises: (i) a VHAb1; (ii) a VLAb2 comprising a truncation or a deletion in the framework 1 (FR1) region; and (iii) a polypeptide linker that links the VHAb1 C-terminal amino acid with the N-terminal amino acid of the VLAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VLAb2 complementarity determining region 1 (CDR1) are separated by from 5 to 22 amino acids; and wherein VHAb1 and VLAb2 bind to non-overlapping epitopes on the target.
Embodiment 10. A co-binder that specifically binds to a target, wherein the co-binder comprises: (i) a VHAb1; (ii) a VLAb2 comprising a truncation or a deletion in the framework 1 (FR1) region; and (iii) a polypeptide linker that links the VHAb1 C-terminal amino acid with the N-terminal amino acid of the VLAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VLAb2 complementarity determining region 1 (CDR1) are separated by no more than 22 amino acids; and wherein VHAb1 and VLAb2 bind to non-overlapping epitopes on the target.
Embodiment 11. A co-binder that specifically binds to a target, wherein the co-binder comprises: (i) a VHAb1; (ii) a VLAb2 comprising an N-terminal truncation of from 1 to 18 amino acids in the framework 1 (FR1) region; and (iii) a polypeptide linker that links the VHAb1 C-terminal amino acid with the N-terminal amino acid of the truncated VLAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VHAb1 and VLAb2 bind to non-overlapping epitopes on the target.
Embodiment 12. The co-binder of embodiment 11, wherein the N-terminal truncation of the FR1 is from 1 to 10 amino acids.
Embodiment 13. A co-binder that specifically binds to a target, wherein the co-binder comprises: (i) a light chain variable region of a first antibody (VLAb1); (ii) a light chain variable region of a second antibody (VLAb2) comprising an N-terminal truncation of from 1 to 18 amino acids; and (iii) a polypeptide linker that links the VLAb1 C-terminal amino acid with the N-terminal amino acid of the truncated VLAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VLAb1 and VLAb2 bind to non-overlapping epitopes on the target.
Embodiment 14. The co-binder of embodiment 13, wherein the N-terminal truncation of the VLAb2 is from 1 to 10 amino acids.
Embodiment 15. A co-binder that specifically binds to a target, wherein the co-binder comprises: (i) a VLAb1; (ii) a VLAb2 comprising a truncation or a deletion in the framework 1 (FR1) region; and (iii) a polypeptide linker that links the VLAb1 C-terminal amino acid with the N-terminal amino acid of the VLAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VLAb2 complementarity determining region 1 (CDR1) are separated by from 5 to 22 amino acids; and wherein VLAb1 and VLAb2 bind to non-overlapping epitopes on the target.
Embodiment 16. A co-binder that specifically binds to a target, wherein the co-binder comprises: (i) a VLAb1; (ii) a VLAb2 comprising a truncation or a deletion in the framework 1 (FR1) region; and (iii) a polypeptide linker that links the VLAb1 C-terminal amino acid with the N-terminal amino acid of the VLAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VLAb2 complementarity determining region 1 (CDR1) are separated by no more than 22 amino acids; and wherein VLAb1 and VLAb2 bind to non-overlapping epitopes on the target.
Embodiment 17. A co-binder that specifically binds to a target, wherein the co-binder comprises: (i) a VLAb1; (ii) a VLAb2 comprising an N-terminal truncation of from 1 to 18 amino acids in the framework 1 (FR1) region; and (iii) a polypeptide linker that links the VLAb1 C-terminal amino acid with the N-terminal amino acid of the truncated VLAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VLAb1 and VLAb2 bind to non-overlapping epitopes on the target.
Embodiment 18. The co-binder of embodiment 17, wherein the N-terminal truncation of the FR1 is from 1 to 10 amino acids.
Embodiment 19. A co-binder that specifically binds to a target, wherein the co-binder comprises: (i) a light chain variable region of a first antibody (VLAb1); (ii) a heavy chain variable region of a second antibody (VHAb2) comprising an N-terminal truncation of from 1 to 18 amino acids; and (iii) a polypeptide linker that links the VLAb1 C-terminal amino acid with the N-terminal amino acid of the truncated VHAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VLAb1 and VHAb2 bind to non-overlapping epitopes on the target.
Embodiment 20. The co-binder of embodiment 19, wherein the N-terminal truncation of the VHAb2 is from 1 to 10 amino acids.
Embodiment 21. A co-binder that specifically binds to a target, wherein the co-binder comprises: (i) a VLAb1; (ii) a VHAb2 comprising a truncation or a deletion in the framework 1 (FR1) region; and (iii) a polypeptide linker that links the VLAb1 C-terminal amino acid with the N-terminal amino acid of the VHAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VHAb2 complementarity determining region 1 (CDR1) are separated by from 8 to 25 amino acids; and wherein VLAb1 and VHAb2 bind to non-overlapping epitopes on the target.
Embodiment 22. A co-binder that specifically binds to a target, wherein the co-binder comprises: (i) a VLAb1; (ii) a VHAb2 comprising a truncation or a deletion in the framework 1 (FR1) region; and (iii) a polypeptide linker that links the VLAb1 C-terminal amino acid with the N-terminal amino acid of the VHAb2 comprising the truncation or the deletion; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein the X₃ amino acid of the polypeptide linker and the VHAb2 complementarity determining region 1 (CDR1) are separated by no more than 25 amino acids; and wherein VLAb1 and VHAb2 bind to non-overlapping epitopes on the target.
Embodiment 23. A co-binder that specifically binds to a target, wherein the co-binder comprises: (i) a VLAb1; (ii) a VHAb2 comprising an N-terminal truncation of from 1 to 18 amino acids in the framework 1 (FR1) region; and (iii) a polypeptide linker that links the VLAb1 C-terminal amino acid with the N-terminal amino acid of the truncated VHAb2; wherein the polypeptide linker C-terminal three amino acids are X₁-X₂-X₃, wherein X₁ is V, L, W, P, S, G, K, D, F, M, T, N, or R; X₂ is V, A, L, S, G, R, K, M, C, F, T, P, or E; and X₃ is G; and wherein VLAb1 and VHAb2 bind to non-overlapping epitopes on the target.
Embodiment 24. The co-binder of embodiment 23, wherein the N-terminal truncation of the FR1 is from 1 to 10 amino acids.
Embodiment 25. The co-binder of any one of embodiments 1 to 24, wherein the polypeptide linker C-terminal three amino acids are VVG, VAG, VLG, VSG, VGG, VRG, VKG, VMG, VCG, VFG, VTG, VPG or VEG.
Embodiment 26. The co-binder of any one of embodiments 1 to 24, wherein the polypeptide linker C-terminal three amino acids are LVG, LAG, LLG, LSG, LGG, LRG, LKG, LMG, LCG, LFG, LTG, LPG or LEG.
Embodiment 27. The co-binder of any one of embodiments 1 to 24, wherein the polypeptide linker C-terminal three amino acids are WVG, WAG, WLG, WSG, WGG, WRG, WKG, WMG, WCG, WFG, WTG, WPG or WEG.
Embodiment 28. The co-binder of any one of embodiments 1 to 24, wherein the polypeptide linker C-terminal three amino acids are PVG, PAG, PLG, PSG, PGG, PRG, PKG, PMG, PCG, PFG, PTG, PPG or PEG.
Embodiment 29. The co-binder of any one of embodiments 1 to 24, wherein the polypeptide linker C-terminal three amino acids are SVG, SAG, SLG, SSG, SGG, SRG, SKG, SMG, SCG, SFG, STG, SPG or SEG.
Embodiment 30. The co-binder of any one of embodiments 1 to 24, wherein the polypeptide linker C-terminal three amino acids are GVG, GAG, GLG, GSG, GGG, GRG, GKG, GMG, GCG, GFG, GTG, GPG or GEG.
Embodiment 31. The co-binder of any one of embodiments 1 to 24, wherein the polypeptide linker C-terminal three amino acids are KVG, KAG, KLG, KSG, KGG, KRG, KKG, KMG, KCG, KFG, KTG, KPG or KEG.
Embodiment 32. The co-binder of any one of embodiments 1 to 24, wherein the polypeptide linker C-terminal three amino acids are DVG, DAG, DLG, DSG, DGG, DRG, DKG, DMG, DCG, DFG, DTG, DPG or DEG.
Embodiment 33. The co-binder of any one of embodiments 1 to 24, wherein the polypeptide linker C-terminal three amino acids are FVG, FAG, FLG, FSG, FGG, FRG, FKG, FMG, FCG, FFG, FTG, FPG or FEG.
Embodiment 34. The co-binder of any one of embodiments 1 to 24, wherein the polypeptide linker C-terminal three amino acids are MVG, MAG, MLG, MSG, MGG, MRG, MKG, MMG, MCG, MFG, MTG, MPG or MEG.
Embodiment 35. The co-binder of any one of embodiments 1 to 24, wherein the polypeptide linker C-terminal three amino acids are TVG, TAG, TLG, TSG, TGG, TRG, TKG, TMG, TCG, TFG, TTG, TPG or TEG.
Embodiment 36. The co-binder of any one of embodiments 1 to 24, wherein the polypeptide linker C-terminal three amino acids are NVG, NAG, NLG, NSG, NGG, NRG, NKG, NMG, NCG, NFG, NTG, NPG or NEG.
Embodiment 37. The co-binder of any one of embodiments 1 to 24, wherein the polypeptide linker C-terminal three amino acids are RVG, RAG, RLG, RSG, RGG, RRG, RKG, RMG, RCG, RFG, RTG, RPG or REG.
Embodiment 38. A co-binder that specifically binds to a target, wherein the co-binder comprises: (i) a heavy chain variable region of a first antibody (VHAb1); (ii) a heavy chain variable region of a second antibody (VHAb2) comprising an N-terminal truncation of from 1 to 18 amino acids; and (iii) a linker that links the VHAb1 C-terminal amino acid with the N-terminal amino acid of the truncated VHAb2; wherein VHAb1 and VHAb2 bind to non-overlapping epitopes on the target.
Embodiment 39. The co-binder of embodiment 38, wherein the N-terminal truncation of the VHAb2 is from 1 to 10 amino acids.
Embodiment 40. A co-binder that specifically binds to a target, wherein the co-binder comprises: (i) a VHAb1; (ii) a VHAb2 comprising a truncation or a deletion of from 1 to 18 amino acids in the framework 1 (FR1) region; and (iii) a linker that links the VHAb1 C-terminal amino acid with the N-terminal amino acid of the VHAb2 comprising the truncation or the deletion; wherein VHAb1 and VHAb2 bind to non-overlapping epitopes on the target.
Embodiment 41. A co-binder that specifically binds to a target, wherein the co-binder comprises: (i) a VHAb1; (ii) a VHAb2 comprising an N-terminal truncation of from 1 to 18 amino acids in the framework 1 (FR1) region; and (iii) a linker that links the VHAb1 C-terminal amino acid with the N-terminal amino acid of the truncated VHAb2; wherein VHAb1 and VHAb2 bind to non-overlapping epitopes on the target.
Embodiment 42. The co-binder of embodiment 41, wherein the N-terminal truncation of the FR1 is from 1 to 10 amino acids.
Embodiment 43. A co-binder that specifically binds to a target, wherein the co-binder comprises: (i) a heavy chain variable region of a first antibody (VHAb1); (ii) a light chain variable region of a second antibody (VLAb2) comprising an N-terminal truncation of from 1 to 18 amino acids; and (iii) a linker that links the VHAb1 C-terminal amino acid with the N-terminal amino acid of the truncated VLAb2; wherein VHAb1 and VLAb2 bind to non-overlapping epitopes on the target.
Embodiment 44. The co-binder of embodiment 43, wherein the N-terminal truncation of the VLAb2 is from 1 to 10 amino acids.
Embodiment 45. A co-binder that specifically binds to a target, wherein the co-binder comprises: (i) a VHAb1; (ii) a VLAb2 comprising a truncation or a deletion of from 1 to 18 amino acids in the framework 1 (FR1) region; and (iii) a linker that links the VHAb1 C-terminal amino acid with the N-terminal amino acid of the VLAb2 comprising the truncation or the deletion; wherein VHAb1 and VLAb2 bind to non-overlapping epitopes on the target.
Embodiment 46. A co-binder that specifically binds to a target, wherein the co-binder comprises: (i) a VHAb1; (ii) a VLAb2 comprising an N-terminal truncation of from 1 to 18 amino acids in the framework 1 (FR1) region; and (iii) a linker that links the VHAb1 C-terminal amino acid with the N-terminal amino acid of the truncated VLAb2; wherein VHAb1 and VLAb2 bind to non-overlapping epitopes on the target.
Embodiment 47. The co-binder of embodiment 46, wherein the N-terminal truncation of the FR1 is from 1 to 10 amino acids.
Embodiment 48. A co-binder that specifically binds to a target, wherein the co-binder comprises: (i) a light chain variable region of a first antibody (VLAb1); (ii) a light chain variable region of a second antibody (VLAb2) comprising an N-terminal truncation of from 1 to 18 amino acids; and (iii) a linker that links the VLAb1 C-terminal amino acid with the N-terminal amino acid of the truncated VLAb2; wherein VLAb1 and VLAb2 bind to non-overlapping epitopes on the target.
Embodiment 49. The co-binder of embodiment 48, wherein the N-terminal truncation of the VLAb2 is from 1 to 10 amino acids.
Embodiment 50. A co-binder that specifically binds to a target, wherein the co-binder comprises: (i) a VLAb1; (ii) a VLAb2 comprising a truncation or a deletion of from 1 to 18 amino acids in the framework 1 (FR1) region; and (iii) a linker that links the VLAb1 C-terminal amino acid with the N-terminal amino acid of the VLAb2 comprising the truncation or the deletion; wherein VLAb1 and VLAb2 bind to non-overlapping epitopes on the target.
Embodiment 51. A co-binder that specifically binds to a target, wherein the co-binder comprises: (i) a VLAb1; (ii) a VLAb2 comprising an N-terminal truncation of from 1 to 18 amino acids in the framework 1 (FR1) region; and (iii) a linker that links the VLAb1 C-terminal amino acid with the N-terminal amino acid of the truncated VLAb2; wherein VLAb1 and VLAb2 bind to non-overlapping epitopes on the target.
Embodiment 52. The co-binder of embodiment 51, wherein the N-terminal truncation of the FR1 is from 1 to 10 amino acids.
Embodiment 53. A co-binder that specifically binds to a target, wherein the co-binder comprises: (i) a light chain variable region of a first antibody (VLAb1); (ii) a heavy chain variable region of a second antibody (VHAb2) comprising an N-terminal truncation of from 1 to 18 amino acids; and (iii) a linker that links the VLAb1 C-terminal amino acid with the N-terminal amino acid of the truncated VHAb2; wherein VLAb1 and VHAb2 bind to non-overlapping epitopes on the target.
Embodiment 54. The co-binder of embodiment 53, wherein the N-terminal truncation of the VHAb2 is from 1 to 10 amino acids.
Embodiment 55. A co-binder that specifically binds to a target, wherein the co-binder comprises: (i) a VLAb1; (ii) a VHAb2 comprising a truncation or a deletion of from 1 to 18 amino acids in the framework 1 (FR1) region; and (iii) a linker that links the VLAb1 C-terminal amino acid with the N-terminal amino acid of the VHAb2 comprising the truncation or the deletion; wherein VLAb1 and VHAb2 bind to non-overlapping epitopes on the target.
Embodiment 56. A co-binder that specifically binds to a target, wherein the co-binder comprises: (i) a VLAb1; (ii) a VHAb2 comprising an N-terminal truncation of from 1 to 18 amino acids in the framework 1 (FR1) region; and (iii) a linker that links the VLAb1 C-terminal amino acid with the N-terminal amino acid of the truncated VHAb2; wherein VLAb1 and VHAb2 bind to non-overlapping epitopes on the target.
Embodiment 57. The co-binder of embodiment 56, wherein the N-terminal truncation of the FR1 is from 1 to 10 amino acids.
Embodiment 58. The co-binder of any one of embodiments 1 to 12, and 25 to 47, wherein the VHAb1 is further linked to a light chain variable region (VL).
Embodiment 59. The co-binder of any one of embodiments 1 to 6, 19 to 42, and 53 to 57, wherein the VHAb2 is further linked to a VL.
Embodiment 60. The co-binder of any one of embodiments 13 to 37, 48 to 57, and 59, wherein the VLAb1 is further linked to a heavy chain variable region (VH).
Embodiment 61. The co-binder of any one of embodiments 7 to 18, 25 to 37, 43 to 52, 58, and 60, wherein the VLAb2 is further linked to a VH.
Embodiment 62. The co-binder of any one of embodiments 1 to 6, and 25 to 42, wherein the VHAb1 is further linked to a first VL, and the VHAb2 is further linked to a second VL.
Embodiment 63. The co-binder of any one of embodiments 7 to 12, 25 to 37, and 43 to 47, wherein the VHAb1 is further linked to a VL, and the VLAb2 is further linked to a VH.
Embodiment 64. The co-binder of any one of embodiments 13 to 18, 25 to 37, and 48 to 52, wherein the VLAb1 is further linked to a first VH, and the VLAb2 is further linked to a second VH.
Embodiment 65. The co-binder of any one of embodiments 19 to 37, and 53 to 57, wherein the VLAb1 is further linked to a VH, and the VHAb2 is further linked to a VL.
Embodiment 66. The co-binder of any one of embodiments 1 to 6, 19 to 42, 53 to 60, 62, and 65, wherein the truncated VHAb2 further comprises from 1 to 18 additional amino acids substituting the amino acids truncated from the VHAb2.
Embodiment 67. The co-binder of any one of embodiments 1 to 6, 19 to 42, 53 to 60, 62, and 65, wherein the VHAb2 comprising a deletion further comprises from 1 to 18 additional amino acids substituting the amino acids deleted from the VHAb2.
Embodiment 68. The co-binder of any one of embodiments 1 to 6, 19 to 42, 53 to 60, 62, and 65 to 67, wherein the N-terminal 1st amino acid of the truncated VHAb2 is not E or Q.
Embodiment 69. The co-binder of any one of embodiments 1 to 6, 19 to 42, 53 to 60, 62, and 65 to 68, wherein the N-terminal 1st amino acid of the truncated VHAb2 is not E, Q, or R.
Embodiment 70. The co-binder of any one of embodiments 1 to 6, 19 to 42, 53 to 60, 62, and 65 to 69, wherein the N-terminal 2nd amino acid of the truncated VHAb2 is not I, L, M, or V.
Embodiment 71. The co-binder of any one of embodiments 1 to 6, 19 to 42, 53 to 60, 62, and 65 to 70, wherein the N-terminal 3rd amino acid of the truncated VHAb2 is not Q or T.
Embodiment 72. The co-binder of any one of embodiments 1 to 6, 19 to 42, 53 to 60, 62, and 65 to 70, wherein the N-terminal 3rd amino acid of the truncated VHAb2 is not Q, T, H, or R.
Embodiment 73. The co-binder of any one of embodiments 1 to 6, 19 to 42, 53 to 60, 62, and 65 to 72, wherein the N-terminal 4th amino acid of the truncated VHAb2 is not L or V.
Embodiment 74. The co-binder of any one of embodiments 1 to 6, 19 to 42, 53 to 60, 62, and 65 to 72, wherein the N-terminal 4th amino acid of the truncated VHAb2 is not L, V, or R.
Embodiment 75. The co-binder of any one of embodiments 1 to 6, 19 to 42, 53 to 60, 62, and 65 to 74, wherein the N-terminal 5th amino acid of the truncated VHAb2 is not K, L, Q, R, or V.
Embodiment 76. The co-binder of any one of embodiments 1 to 6, 19 to 42, 53 to 60, 62, and 65 to 75, wherein the N-terminal 6th amino acid of the truncated VHAb2 is not E or Q.
Embodiment 77. The co-binder of any one of embodiments 1 to 6, 19 to 42, 53 to 60, 62, and 65 to 75, wherein the N-terminal 6th amino acid of the truncated VHAb2 is not E, K, Q, or D.
Embodiment 78. The co-binder of any one of embodiments 1 to 6, 19 to 42, 53 to 60, 62, and 65 to 77, wherein the N-terminal 7th amino acid of the truncated VHAb2 is not P, S, or W.
Embodiment 79. The co-binder of any one of embodiments 1 to 6, 19 to 42, 53 to 60, 62, and 65 to 77, wherein the N-terminal 7th amino acid of the truncated VHAb2 is not P, S, W, L or T.
Embodiment 80. The co-binder of any one of embodiments 1 to 6, 19 to 42, 53 to 60, 62, and 65 to 79, wherein the N-terminal 8th amino acid of the truncated VHAb2 is not G.
Embodiment 81. The co-binder of any one of embodiments 1 to 6, 19 to 42, 53 to 60, 62, and 65 to 79, wherein the N-terminal 8th amino acid of the truncated VHAb2 is not G, A, or V.
Embodiment 82. The co-binder of any one of embodiments 1 to 6, 19 to 42, 53 to 60, 62, and 65 to 81, wherein the N-terminal 9th amino acid of the truncated VHAb2 is not A, E, G, P, or S.
Embodiment 83. The co-binder of any one of embodiments 1 to 6, 19 to 42, 53 to 60, 62, and 65 to 82, wherein the N-terminal 11th amino acid of the truncated VHAb2 is not A, E, G, T, or V.
Embodiment 84. The co-binder of any one of embodiments 1 to 6, 19 to 42, 53 to 60, 62, and 65 to 83, wherein the N-terminal 12th amino acid of the truncated VHAb2 is not L or V.
Embodiment 85. The co-binder of any one of embodiments 1 to 6, 19 to 42, 53 to 60, 62, and 65 to 84, wherein the N-terminal 13th amino acid of the truncated VHAb2 is not I, K, L, R, or V.
Embodiment 86. The co-binder of any one of embodiments 1 to 6, 19 to 42, 53 to 60, 62, and 65 to 85, wherein the N-terminal 14th amino acid of the truncated VHAb2 is not K, Q, or R.
Embodiment 87. The co-binder of any one of embodiments 1 to 6, 19 to 42, 53 to 60, 62, and 65 to 85, wherein the N-terminal 14th amino acid of the truncated VHAb2 is not K, Q, R, or N.
Embodiment 88. The co-binder of any one of embodiments 1 to 6, 19 to 42, 53 to 60, 62, and 65 to 87, wherein the N-terminal 15th amino acid of the truncated VHAb2 is not A or P.
Embodiment 89. The co-binder of any one of embodiments 1 to 6, 19 to 42, 53 to 60, 62, and 65 to 87, wherein the N-terminal 15th amino acid of the truncated VHAb2 is not A, P, D, L, or T.
Embodiment 90. The co-binder of any one of embodiments 1 to 6, 19 to 42, 53 to 60, 62, and 65 to 89, wherein the N-terminal 16th amino acid of the truncated VHAb2 is not G, P, S, or T.
Embodiment 91. The co-binder of any one of embodiments 1 to 6, 19 to 42, 53 to 60, 62, and 65 to 90, wherein the N-terminal 17th amino acid of the truncated VHAb2 is not A, D, E, G, Q, R, or S.
Embodiment 92. The co-binder of any one of embodiments 1 to 6, 19 to 42, 53 to 60, 62, and 65 to 90, wherein the N-terminal 17th amino acid of the truncated VHAb2 is not A, D, E, G, Q, R, S, P, T, or V.
Embodiment 93. The co-binder of any one of embodiments 1 to 6, 19 to 42, 53 to 60, 62, and 65 to 92, wherein the N-terminal 18th amino acid of the truncated VHAb2 is not S or T.
Embodiment 94. The co-binder of any one of embodiments 1 to 6, 19 to 42, 53 to 60, 62, and 65 to 92, wherein the N-terminal 18th amino acid of the truncated VHAb2 is not S, T, A, L, or M.
Embodiment 95. The co-binder of any one of embodiments 7 to 18, 25 to 37, 43 to 52, 58, 60 to 61, and 63 to 64, wherein the truncated VLAb2 further comprises from 1 to 18 additional amino acids substituting the amino acids truncated from the VLAb2.
Embodiment 96. The co-binder of any one of embodiments 7 to 18, 25 to 37, 43 to 52, 58, 60 to 61, and 63 to 64, wherein the VLAb2 comprising a deletion further comprises from 1 to 18 additional amino acids substituting the amino acids deleted from the VLAb2.
Embodiment 97. The co-binder of any one of embodiments 7 to 18, 25 to 37, 43 to 52, 58, 60 to 61, 63 to 64, and 95 to 96, wherein the VLAb2 is a light chain variable region of human lambda (λ) light chain (AVLAb2).
Embodiment 98. The co-binder of any one of embodiments 7 to 18, 25 to 37, 43 to 52, 58, 60 to 61, 63 to 64, and 95 to 97, wherein the N-terminal 1st amino acid of the truncated AVLAb2 is not N, Q, R, or S.
Embodiment 99. The co-binder of any one of embodiments 7 to 18, 25 to 37, 43 to 52, 58, 60 to 61, 63 to 64, and 95 to 97, wherein the N-terminal 1st amino acid of the truncated AVLAb2 is not N, Q, R, S or L.
Embodiment 100. The co-binder of any one of embodiments 7 to 18, 25 to 37, 43 to 52, 58, 60 to 61, 63 to 64, and 95 to 99, wherein the N-terminal 2nd amino acid of the truncated AVLAb2 is not A, F, L, P, S, T, or Y.
Embodiment 101. The co-binder of any one of embodiments 7 to 18, 25 to 37, 43 to 52, 58, 60 to 61, 63 to 64, and 95 to 100, wherein the N-terminal 3rd amino acid of the truncated AVLAb2 is not A, E, G, M, or V.
Embodiment 102. The co-binder of any one of embodiments 7 to 18, 25 to 37, 43 to 52, 58, 60 to 61, 63 to 64, and 95 to 101, wherein the N-terminal 4th amino acid of the truncated AVLAb2 is not L or V.
Embodiment 103. The co-binder of any one of embodiments 7 to 18, 25 to 37, 43 to 52, 58, 60 to 61, 63 to 64, and 95 to 101, wherein the N-terminal 4th amino acid of the truncated AVLAb2 is not L, V, or P.
Embodiment 104. The co-binder of any one of embodiments 7 to 18, 25 to 37, 43 to 52, 58, 60 to 61, 63 to 64, and 95 to 103, wherein the N-terminal 5th amino acid of the truncated AVLAb2 is not T.
Embodiment 105. The co-binder of any one of embodiments 7 to 18, 25 to 37, 43 to 52, 58, 60 to 61, 63 to 64, and 95 to 103, wherein the N-terminal 5th amino acid of the truncated AVLAb2 is not T or M.
Embodiment 106. The co-binder of any one of embodiments 7 to 18, 25 to 37, 43 to 52, 58, 60 to 61, 63 to 64, and 95 to 105, wherein the N-terminal 6th amino acid of the truncated AVLAb2 is not Q.
Embodiment 107. The co-binder of any one of embodiments 7 to 18, 25 to 37, 43 to 52, 58, 60 to 61, 63 to 64, and 95 to 106, wherein the N-terminal 7th amino acid of the truncated AVLAb2 is not E, P, or S.
Embodiment 108. The co-binder of any one of embodiments 7 to 18, 25 to 37, 43 to 52, 58, 60 to 61, 63 to 64, and 95 to 106, wherein the N-terminal 7th amino acid of the truncated AVLAb2 is not E, P, S, D, L, or V.
Embodiment 109. The co-binder of any one of embodiments 7 to 18, 25 to 37, 43 to 52, 58, 60 to 61, 63 to 64, and 95 to 108, wherein the N-terminal 8th amino acid of the truncated AVLAb2 is not A, H, L, P, R, S, or T.
Embodiment 110. The co-binder of any one of embodiments 7 to 18, 25 to 37, 43 to 52, 58, 60 to 61, 63 to 64, and 95 to 109, wherein the N-terminal 9th amino acid of the truncated AVLAb2 is not A, F, or S.
Embodiment 111. The co-binder of any one of embodiments 7 to 18, 25 to 37, 43 to 52, 58, 60 to 61, 63 to 64, and 95 to 110, wherein the N-terminal 11th amino acid of the truncated AVLAb2 is not A, F, L, or V.
Embodiment 112. The co-binder of any one of embodiments 7 to 18, 25 to 37, 43 to 52, 58, 60 to 61, 63 to 64, and 95 to 110, wherein the N-terminal 11th amino acid of the truncated AVLAb2 is not A, F, L, V, H, or S.
Embodiment 113. The co-binder of any one of embodiments 7 to 18, 25 to 37, 43 to 52, 58, 60 to 61, 63 to 64, and 95 to 112, wherein the N-terminal 12th amino acid of the truncated AVLAb2 is not S or T.
Embodiment 114. The co-binder of any one of embodiments 7 to 18, 25 to 37, 43 to 52, 58, 60 to 61, 63 to 64, and 95 to 113, wherein the N-terminal 13th amino acid of the truncated 2VLAb2 is not A, E, G, K, or V.
Embodiment 115. The co-binder of any one of embodiments 7 to 18, 25 to 37, 43 to 52, 58, 60 to 61, 63 to 64, and 95 to 113, wherein the N-terminal 13th amino acid of the truncated AVLAb2 is not A, E, G, K, V, or R.
Embodiment 116. The co-binder of any one of embodiments 7 to 18, 25 to 37, 43 to 52, 58, 60 to 61, 63 to 64, and 95 to 115, wherein the N-terminal 14th amino acid of the truncated AVLAb2 is not A, G, S, or T.
Embodiment 117. The co-binder of any one of embodiments 7 to 18, 25 to 37, 43 to 52, 58, 60 to 61, 63 to 64, and 95 to 115, wherein the N-terminal 14th amino acid of the truncated AVLAb2 is not A, G, S, T, or L.
Embodiment 118. The co-binder of any one of embodiments 7 to 18, 25 to 37, 43 to 52, 58, 60 to 61, 63 to 64, and 95 to 117, wherein the N-terminal 15th amino acid of the truncated AVLAb2 is not L, P, or T.
Embodiment 119. The co-binder of any one of embodiments 7 to 18, 25 to 37, 43 to 52, 58, 60 to 61, 63 to 64, and 95 to 117, wherein the N-terminal 15th amino acid of the truncated AVLAb2 is not L, P, T or S.
Embodiment 120. The co-binder of any one of embodiments 7 to 18, 25 to 37, 43 to 52, 58, 60 to 61, 63 to 64, and 95 to 119, wherein the N-terminal 16th amino acid of the truncated AVLAb2 is not A, G, or R.
Embodiment 121. The co-binder of any one of embodiments 7 to 18, 25 to 37, 43 to 52, 58, 60 to 61, 63 to 64, and 95 to 120, wherein the N-terminal 17th amino acid of the truncated AVLAb2 is not A, G, K, Q, or S.
Embodiment 122. The co-binder of any one of embodiments 7 to 18, 25 to 37, 43 to 52, 58, 60 to 61, 63 to 64, and 95 to 120, wherein the N-terminal 17th amino acid of the truncated AVLAb2 is not A, G, K, Q, S, or E.
Embodiment 123. The co-binder of any one of embodiments 7 to 18, 25 to 37, 43 to 52, 58, 60 to 61, 63 to 64, and 95 to 122, wherein the N-terminal 18th amino acid of the truncated AVLAb2 is not K, M, R, S, or T.
Embodiment 124. The co-binder of any one of embodiments 7 to 18, 25 to 37, 43 to 52, 58, 60 to 61, 63 to 64, and 95 to 122, wherein the N-terminal 18th amino acid of the truncated AVLAb2 is not K, M, R, S, T, or A.
Embodiment 125. The co-binder of any one of embodiments 7 to 18, 25 to 37, 43 to 52, 58, 60 to 61, 63 to 64, and 95, wherein the VLAb2 is a light chain variable region of human kappa (κ) light chain (KVLAb2).
Embodiment 126. The co-binder of any one of embodiments 7 to 18, 25 to 37, 43 to 52, 58, 60 to 61, 63 to 64, 95, and 125, wherein the N-terminal 1st amino acid of the truncated KVLAb2 is not A, D, E, or V.
Embodiment 127. The co-binder of any one of embodiments 7 to 18, 25 to 37, 43 to 52, 58, 60 to 61, 63 to 64, 95, and 125, wherein the N-terminal 1st amino acid of the truncated KVLAb2 is not A, D, E, V, or N.
Embodiment 128. The co-binder of any one of embodiments 7 to 18, 25 to 37, 43 to 52, 58, 60 to 61, 63 to 64, 95, and 125 to 127, wherein the N-terminal 2nd amino acid of the truncated KVLAb2 is not I or V.
Embodiment 129. The co-binder of any one of embodiments 7 to 18, 25 to 37, 43 to 52, 58, 60 to 61, 63 to 64, 95, and 125 to 127, wherein the N-terminal 2nd amino acid of the truncated KVLAb2 is not I, V or T.
Embodiment 130. The co-binder of any one of embodiments 7 to 18, 25 to 37, 43 to 52, 58, 60 to 61, 63 to 64, 95, and 125 to 129, wherein the N-terminal 3rd amino acid of the truncated KVLAb2 is not Q, R, V, or W.
Embodiment 131. The co-binder of any one of embodiments 7 to 18, 25 to 37, 43 to 52, 58, 60 to 61, 63 to 64, 95, and 125 to 129, wherein the N-terminal 3rd amino acid of the truncated KVLAb2 is not Q, R, V, W, or T.
Embodiment 132. The co-binder of any one of embodiments 7 to 18, 25 to 37, 43 to 52, 58, 60 to 61, 63 to 64, 95, and 125 to 129, wherein the N-terminal 4th amino acid of the truncated KVLAb2 is not L or M.
Embodiment 133. The co-binder of any one of embodiments 7 to 18, 25 to 37, 43 to 52, 58, 60 to 61, 63 to 64, 95, and 125 to 132, wherein the N-terminal 5th amino acid of the truncated KVLAb2 is not T.
Embodiment 134. The co-binder of any one of embodiments 7 to 18, 25 to 37, 43 to 52, 58, 60 to 61, 63 to 64, 95, and 125 to 133, wherein the N-terminal 6th amino acid of the truncated KVLAb2 is not Q.
Embodiment 135. The co-binder of any one of embodiments 7 to 18, 25 to 37, 43 to 52, 58, 60 to 61, 63 to 64, 95, and 125 to 134, wherein the N-terminal 7th amino acid of the truncated KVLAb2 is not S or T.
Embodiment 136. The co-binder of any one of embodiments 7 to 18, 25 to 37, 43 to 52, 58, 60 to 61, 63 to 64, 95, and 125 to 135, wherein the N-terminal 8th amino acid of the truncated KVLAb2 is not P.
Embodiment 137. The co-binder of any one of embodiments 7 to 18, 25 to 37, 43 to 52, 58, 60 to 61, 63 to 64, 95, and 125 to 136, wherein the N-terminal 9th amino acid of the truncated KVLAb2 is not A, D, L, or S.
Embodiment 138. The co-binder of any one of embodiments 7 to 18, 25 to 37, 43 to 52, 58, 60 to 61, 63 to 64, 95, and 125 to 136, wherein the N-terminal 9th amino acid of the truncated KVLAb2 is not A, D, L, S, F, or G.
Embodiment 139. The co-binder of any one of embodiments 7 to 18, 25 to 37, 43 to 52, 58, 60 to 61, 63 to 64, 95, and 125 to 138, wherein the N-terminal 10th amino acid of the truncated KVLAb2 is not A, F, L, S, or T.
Embodiment 140. The co-binder of any one of embodiments 7 to 18, 25 to 37, 43 to 52, 58, 60 to 61, 63 to 64, 95, and 125 to 139, wherein the N-terminal 11th amino acid of the truncated KVLAb2 is not L, M, Q, or V.
Embodiment 141. The co-binder of any one of embodiments 7 to 18, 25 to 37, 43 to 52, 58, 60 to 61, 63 to 64, 95, and 125 to 139, wherein the N-terminal 11th amino acid of the truncated KVLAb2 is not L, M, Q, V, F, or S.
Embodiment 142. The co-binder of any one of embodiments 7 to 18, 25 to 37, 43 to 52, 58, 60 to 61, 63 to 64, 95, and 125 to 141, wherein the N-terminal 12th amino acid of the truncated KVLAb2 is not P or S.
Embodiment 143. The co-binder of any one of embodiments 7 to 18, 25 to 37, 43 to 52, 58, 60 to 61, 63 to 64, 95, and 125 to 141, wherein the N-terminal 12th amino acid of the truncated KVLAb2 is not P, S, or A.
Embodiment 144. The co-binder of any one of embodiments 7 to 18, 25 to 37, 43 to 52, 58, 60 to 61, 63 to 64, 95, and 125 to 143, wherein the N-terminal 13th amino acid of the truncated KVLAb2 is not A, I, L, or V.
Embodiment 145. The co-binder of any one of embodiments 7 to 18, 25 to 37, 43 to 52, 58, 60 to 61, 63 to 64, 95, and 125 to 144, wherein the N-terminal 14th amino acid of the truncated KVLAb2 is not S, or T.
Embodiment 146. The co-binder of any one of embodiments 7 to 18, 25 to 37, 43 to 52, 58, 60 to 61, 63 to 64, 95, and 125 to 145, wherein the N-terminal 15th amino acid of the truncated KVLAb2 is not L, P, T or V.
Embodiment 147. The co-binder of any one of embodiments 7 to 18, 25 to 37, 43 to 52, 58, 60 to 61, 63 to 64, 95, and 125 to 146, wherein the N-terminal 16th amino acid of the truncated KVLAb2 is not G or K.
Embodiment 148. The co-binder of any one of embodiments 7 to 18, 25 to 37, 43 to 52, 58, 60 to 61, 63 to 64, 95, and 125 to 147, wherein the N-terminal 17th amino acid of the truncated KVLAb2 is not D, E, or Q.
Embodiment 149. The co-binder of any one of embodiments 7 to 18, 25 to 37, 43 to 52, 58, 60 to 61, 63 to 64, 95, and 125 to 148, wherein the N-terminal 18th amino acid of the truncated KVLAb2 is not K, P, Q, or R.
Embodiment 150. An antigen binding protein comprising a heavy chain variable region of an antibody (VHAb), wherein the VHAb comprises an N-terminal truncation of from 1 to 18 amino acids, and wherein the VHAb is linked to a second molecule via a linker.
Embodiment 151. An antigen binding protein comprising a light chain variable region of an antibody (VLAb), wherein the VLAb comprises an N-terminal truncation of from 1 to 18 amino acids, and wherein the VLAb is linked to a second molecule via a linker.
Embodiment 152. The antigen binding protein of embodiment 150, wherein the VHAb is further linked to a light chain variable region.
Embodiment 153. The antigen binding protein of embodiment 151, wherein the VLAb is further linked to a heavy chain variable region.
Embodiment 154. The antigen binding protein of any one of embodiments 150 to 153, wherein the second molecule is a DNA molecule or a protein molecule.
Embodiment 155. The antigen binding protein of any one of embodiments 150 to 154, wherein the second molecule is an antibody or antigen binding fragment thereof.
Embodiment 156. The co-binder of any one of embodiments 1 to 149, wherein the FR1, CDR1, or both FR1 and CDR1 are determined according to IMGT numbering scheme.
Embodiment 157. The co-binder of any one of embodiments 1 to 149, wherein the FR1, CDR1, or both FR1 and CDR1 are determined according to Kabat numbering scheme.
Embodiment 158. The co-binder of any one of embodiments 1 to 157, wherein the VH, VL, or both VH and VL are determined according to IMGT numbering scheme.
Embodiment 159. The co-binder of any one of embodiments 1 to 157, wherein the VH, VL, or both VH and VL are determined according to Kabat numbering scheme.
Embodiment 160. The co-binder of any one of embodiments 68 to 159, wherein the amino acid position is determined according to IMGT numbering scheme.
Embodiment 161. The co-binder of any one of embodiments 68 to 159, wherein the amino acid position is determined according to Kabat numbering scheme.
Embodiment 162. The co-binder of any one of embodiments 1 to 161, wherein the VHAb1, VHAb2, or both VHAb1 and VHAb2 are from camelid single chain V_{H}H.
Embodiment 163. The co-binder of any one of embodiments 1 to 161, wherein the VHAb1, VHAb2, or both VHAb1 and VHAb2 are from IgG, IgA, IgE, IgM, or IgD.
Embodiment 164. The co-binder of any one of embodiments 1 to 161, wherein the VHAb1, VHAb2, or both VHAb1 and VHAb2 are camelid single chain V_{H}H.
Embodiment 165. The co-binder of any one of embodiments 1 to 161, wherein the VHAb1, VHAb2, or both VHAb1 and VHAb2 are a part of a scFv.
Embodiment 166. The co-binder of any one of embodiments 1 to 161, wherein the VHAb1, VHAb2, or both VHAb1 and VHAb2 are a part of a Fab.
Embodiment 167. The co-binder of any one of embodiments 7 to 166, wherein the VLAb1, VLAb2, or both VLAb1 and VLAb2 are from IgG, IgA, IgE, IgM, or IgD.
Embodiment 168. The co-binder of any one of embodiments 7 to 166, wherein the VLAb1, VLAb2, or both VLAb1 and VLAb2 are a part of a scFv.
Embodiment 169. The co-binder of any one of embodiments 7 to 166, wherein the VLAb1, VLAb2, or both VLAb1 and VLAb2 are a part of a Fab.
Embodiment 170. The co-binder of any one of embodiments 1 to 169, wherein the linker is a rigid linker.
Embodiment 171. The co-binder of any one of embodiments 1 to 169, wherein the linker is a flexible linker.
Embodiment 172. The co-binder of any one of embodiments 1 to 169, wherein the linker is a cleavable linker.
Embodiment 173. The co-binder of any one of embodiments 1 to 169, wherein the linker is a non-cleavable linker.
Embodiment 174. The co-binder of any one of embodiments 38 to 173, wherein the linker is a peptide, a nucleic acid, or a chemical linker.
Embodiment 175. The co-binder of any one of embodiments 1 to 174, wherein the linker connects the variable region of the first antibody and the variable region of the second antibody through covalent bonds.
Embodiment 176. The co-binder of any one of embodiments 1 to 175, wherein the linker is a peptide linker.
Embodiment 177. The co-binder of embodiment 176, wherein the peptide linker comprises peptide sequences of (EAAAK)ₙ, wherein n is an integer number from 1 to 10.
Embodiment 178. The co-binder of embodiment 176, wherein the peptide linker comprises peptide sequences of (XP)ₙ, (XPP)ₙ, or (XPPP)ₙ wherein X is any amino acid and wherein n is an integer number from 1 to 20.
Embodiment 179. The co-binder of embodiment 176, wherein the peptide linker comprises peptide sequences of (AP)ₙ, wherein n is an integer number from 1 to 20.
Embodiment 180. The co-binder of embodiment 176, wherein the peptide linker comprises peptide sequences of (EEEEKKKK)ₙ, wherein n is an integer number from 1 to 10.
Embodiment 181. The co-binder of embodiment 176, wherein the peptide linker comprises peptide sequences of (GₓS_{y})ₙ, wherein x is 1 to 5, y is 1 to 5, and n is an integer number from 1 to 10.
Embodiment 182. The co-binder of any one of embodiments 38 to 175, wherein the linker is a chemical linker.
Embodiment 183. The co-binder of embodiment 182, wherein the chemical linker comprises polyethylene glycol (“PEG”).
Embodiment 184. The co-binder of any one of embodiments 1 to 174, wherein the linker connects the variable region of the first antibody and the variable region of the second antibody through noncovalent binding.
Embodiment 185. The co-binder of embodiment 184, wherein the linker comprises a leucine zipper.
Embodiment 186. The co-binder of embodiment 184, wherein the linker comprises a double-strand nucleic acid having two complementary strands, each covalently linked to the variable region from the first antibody and the variable region from the second antibody, respectively.
Embodiment 187. The co-binder of any one of embodiments 1 to 186, wherein the linker has a length of no more than 150 angstroms, no more than 140 angstroms, no more than 130 angstroms, no more than 120 angstroms, no more than 110 angstroms, no more than 100 angstroms, no more than 90 angstroms, no more than 80 angstroms, no more than 70 angstroms, no more than 60 angstroms, no more than 50 angstroms, no more than 40 angstroms, no more than 30 angstroms, no more than 20 angstroms, no more than 15 angstroms, no more than 10 angstroms, or no more than 5 angstroms.
Embodiment 188. The co-binder of any one of embodiments 1 to 186, wherein the linker has a length of about 150 angstroms, about 140 angstroms, about 130 angstroms, about 120 angstroms, about 110 angstroms, about 100 angstroms, about 90 angstroms, about 80 angstroms, about 70 angstroms, about 60 angstroms, about 50 angstroms, about 40 angstroms, about 30 angstroms, about 20 angstroms, about 15 angstroms, about 10 angstroms, or about 5 angstroms.
Embodiment 189. The co-binder of any one of embodiments 1 to 181, wherein the linker has a length of no more than 60 amino acids, no more than 55 amino acids, no more than 50 amino acids, no more than 45 amino acids, no more than 40 amino acids, no more than 35 amino acids, no more than 30 amino acids, no more than 25 amino acids, no more than 20 amino acids, no more than 15 amino acids, no more than 10 amino acids, or no more than 5 amino acids.
Embodiment 190. The co-binder of any one of embodiments 1 to 181, wherein the linker has a length of about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, or about 5 amino acids.
Embodiment 191. The co-binder of any one of embodiments 1 to 190, wherein the target is a polypeptide, a multi-protein complex, a nucleic acid, a carbohydrate, a glycan, a lipid molecule, a physiological metabolite, or a small molecule compound.
Embodiment 192. The co-binder of any one of embodiments 1 to 190, wherein the target is a intracellular molecule, a disease marker, a neoantigen, or a cell surface molecule.
Embodiment 193. The co-binder of embodiment 192, wherein the target molecule is a cancer antigen or cancer marker.
Embodiment 194. The co-binder of any one of embodiments 1 to 193, wherein the target is EGFR.
Embodiment 195. The co-binder of any one of embodiments 1 to 194, wherein the target is expressed at below 1×10⁶, below 1×10⁵, below 1×10⁴, below 1×10³, or below 1×10² per cell.
Embodiment 196. The co-binder of any one of embodiments 1 to 193, wherein the target is a secreted protein.
Embodiment 197. The co-binder of embodiment 196, wherein the secreted protein is a growth factor, a cytokine, or a chemokine.
Embodiment 198. The co-binder of embodiment 196 or 197, wherein the secreted protein is at below 1×10¹⁰, below 1×10⁹, below 1×10⁸, below 1×10⁷, below 1×10⁶, below 1×10⁵, below 1×10⁴, below 1×10³, or below 1×10² molecules/ml in the blood.
Embodiment 199. The co-binder of any one of embodiments 1 to 198, wherein the co-binder is an antagonist to the target.
Embodiment 200. The co-binder of any one of embodiments 1 to 198, wherein the co-binder is an agonist to the target.
Embodiment 201. The co-binder of any one of embodiments 1 to 200, wherein the co-binder binds the target with a K_{D} of less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, less than 1×10⁻¹⁶ M, less than 1×10⁻¹⁷ M, or less than 1×10⁻¹⁸ M.
Embodiment 202. The co-binder of any one of embodiments 1 to 200, wherein the co-binder binds the target with a K_{D} of about 1×10⁻⁸ M, about 1×10⁻⁹ M, about 1×10⁻¹⁰ M, about 1×10⁻¹¹ M, about 1×10⁻¹² M, about 1×10⁻¹³ M, about 1×10⁻¹⁴ M, about 1×10⁻¹⁵ M, about 1×10⁻¹⁶ M, about 1×10⁻¹⁷ M, or about 1×10⁻¹⁸ M.
Embodiment 203. The co-binder of any one of embodiments 1 to 200, wherein the affinity of the co-binder to the target is no less than 50 fold, no less than 100 fold, no less than 150 fold, no less than 200 fold, no less than 250 fold, no less than 300 fold, no less than 350 fold, no less than 400 fold, no less than 450 fold, no less than 500 fold, no less than 600 fold, no less than 700 fold, no less than 800 fold, no less than 900 fold, no less than 1000 fold, no less than 1100 fold, no less than 1200 fold, no less than 1300 fold, no less than 1400 fold, no less than 1500 fold, no less than 1600 fold, no less than 1700 fold, no less than 1800 fold, no less than 1900 fold, no less than 2000 fold, no less than 3000 fold, no less than 4000 fold, or no less than 5000 fold, or no less than 10000 fold greater than that of the individual variable regions alone.
Embodiment 204. The co-binder of any one of embodiments 1 to 6, 19 to 42, 53 to 60, 62, 65 to 94, 156 to 166, and 170 to 203, wherein the VHAb2 is such a heavy chain variable region that, if conjugated to a reference Ig domain at the N-terminus of the VHAb2 via a linker comprising (GGGS)4 (refIg-VHAb2), the dissociation constant (KD) between the refIg-VHAb2 and the target is more than 3 times larger than the KD between VHAb2 and the target.
Embodiment 205. The co-binder of any one of embodiments 7 to 18, 25 to 37, 43 to 52, 58, 60, 61, 63, 64, 95 to 149, 156 to 161, and 167 to 203, wherein the VLAb2 is such a light chain variable region that, if conjugated to a reference Ig domain at the N-terminus of the VLAb2 via a linker comprising (GGGS)4 (refIg-VLAb2), the dissociation constant (KD) between the refIg-VLAb2 and the target is more than 3 times larger than the KD between VLAb2 and the target.
Embodiment 206. The co-binder of any one of embodiments 150, 152, 154 to 166, and 170 to 203, wherein the VHAb is such a heavy chain variable region that, if conjugated to a reference Ig domain at the N-terminus of the VHAb via a linker comprising (GGGS)4 (refIg-VHAb), the dissociation constant (KD) between the refIg-VHAb and the target is more than 3 times larger than the KD between VHAb and the target.
Embodiment 207. The co-binder of any one of embodiments 151, 153 to 161, and 167 to 203, wherein the VLAb is such a light chain variable region that, if conjugated to a reference Ig domain at the N-terminus of the VLAb via a linker comprising (GGGS)4 (refIg-VLAb), the dissociation constant (KD) between the refIg-VLAb and the target is more than 3 times larger than the KD between VLAb and the target.
Embodiment 208. A composition comprising the co-binder of any one of embodiments 1 to 207.
Embodiment 209. A pharmaceutical composition comprising the co-binder of any one of embodiments 1 to Embodiment 207 and a pharmaceutically acceptable carrier.
Embodiment 210. A detection agent comprising the co-binder of any one of embodiments 1 to 207.
Embodiment 211. A diagnostic agent comprising the co-binder of any one of embodiments 1 to 207.
Embodiment 212. A therapeutic agent comprising the co-binder of any one of embodiments 1 to 207.
Embodiment 213. A chimeric antigen receptor comprising the co-binder of any one of embodiments 1 to 207.
Embodiment 214. A cell that expresses the co-binder of any one of embodiments 1 to 207.
Embodiment 215. The cell of embodiment 214, wherein the cell is an immune cell.
Embodiment 216. A complex comprising the co-binder of any one of embodiments 1 to 207 and the target.
Embodiment 217. A method for detecting a marker in a sample comprising (i) contacting the sample with the co-binder of any one of embodiments 1 to 207 under a condition sufficient to form a complex of the co-binder and the marker, and (ii) detecting the complex in the sample.
Embodiment 218. The method of embodiment 217, wherein the complex is detected by measuring a labeled agent conjugated to the complex.
Embodiment 219. The method of embodiment 217 or 218, wherein the labeled agent is a fluorescent molecule, a radioisotope, a metal ion, an enzyme, a biotin, a polymer or an antibody.
Embodiment 220. The method of any one of embodiments 217 to 219, wherein the marker is present in the sample at a concentration of no more than 1×10⁻¹⁰ M, no more than 1×10⁻¹¹ M, no more than 1×10⁻¹² M, no more than 1×10⁻¹³ M, no more than 1×10⁻¹⁴ M, no more than 1×10⁻¹⁵ M, no more than 1×10⁻¹⁶ M, no more than 1×10⁻¹⁷ M, no more than 1×10⁻¹⁸ M, no more than 1×10⁻¹⁹ M, no more than 1×10⁻²⁰ M, or no more than 1×10⁻²¹ M.
Embodiment 221. A method of diagnosing a disease in a subject comprising (i) contacting the sample with the co-binder of any one of embodiments 1 to 207 under a condition sufficient to form a complex of the co-binder and a marker of the disease, wherein the co-binder specifically binds to the marker, and (ii) detecting the complex in the sample.
Embodiment 222. The method of embodiment 221, wherein the complex is detected by measuring a labeled agent conjugated to the complex.
Embodiment 223. The method of embodiment 221 or 222, wherein the labeled agent is a fluorescent molecule, a radioisotope, a metal ion, an enzyme, a biotin, a polymer or an antibody.
Embodiment 224. The method of any one of embodiments 221 to 223, wherein the sample is a bodily fluid, a tissue, or a cell.
Embodiment 225. The method of any one of embodiments 221 to 224, wherein the sample is blood, bone marrow, plasma, serum, urine, or cerebrospinal fluid.
Embodiment 226. The method of any one of embodiments 221 to 225, wherein the disease marker is present in the sample at a concentration of no more than 1×10⁻¹⁰ M, no more than 1×10⁻¹¹ M, no more than 1×10⁻¹² M, no more than 1×10⁻¹³ M, no more than 1×10⁻¹⁴ M, no more than 1×10⁻¹⁵ M, no more than 1×10⁻¹⁶ M, no more than 1×10⁻¹⁷ M, no more than 1×10⁻¹⁸ M, no more than 1×10⁻¹⁹ M, no more than 1×10⁻²⁰ M, or no more than 1×10⁻²¹ M.
Embodiment 227. A method of treating a disease in a subject, comprising administering a therapeutically effective amount of the co-binder of any one of embodiments 1 to 207 to the subject, wherein the disease is treatable by activating or inhibit the target molecule
Embodiment 228. A combinatorial library comprising: (i) a first variable region of a first antibody comprising an N-terminal truncation of from 1 to 18 amino acids; and (ii) a plurality of polypeptide linkers; wherein C-terminal amino acids of the linkers are linked with the N-terminal amino acid of the truncated first variable region.
Embodiment 229. A combinatorial library comprising: (i) a plurality of first variable regions of a first antibody, wherein each of the first variable region comprises an N-terminal truncation of from 1 to 18 amino acids; and (ii) a plurality of polypeptide linkers; wherein C-terminal amino acids of the linkers are linked with the N-terminal amino acids of the truncated first variable regions.
Embodiment 230. A combinatorial library comprising: (i) a plurality of first variable regions of a plurality of antibodies, wherein each of the first variable region comprises an N-terminal truncation of from 1 to 18 amino acids; and (ii) a plurality of polypeptide linkers; wherein C-terminal amino acids of the linkers are linked with the N-terminal amino acids of the truncated first variable regions.
Embodiment 231. The combinational library of embodiment 228, wherein the first variable region is a heavy chain variable region.
Embodiment 232. The combinational library of embodiment 229 or 230, wherein the first variable regions are heavy chain variable regions.
Embodiment 233. The combinational library of embodiment 228, wherein the first variable region is a light chain variable region.
Embodiment 234. The combinational library of embodiment 229 or 230, wherein the first variable regions are light chain variable regions.
Embodiment 235. The combinational library of any one of embodiments 229 to 234, wherein the library comprises up to 2, 3, 4, 5, 6, 7, 8, 9, 10, 11, 12, 13, 14, or 15 first variable regions.
Embodiment 236. The combinational library of any one of embodiments 230 to 234, wherein the library comprises at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ first variable regions.
Embodiment 237. The combinational library of any one of embodiments 228 to 236, wherein the linkers comprise from 1 to 18 random amino acids at the C-terminus of the linkers.
Embodiment 238. The combinational library of any one of embodiments 228 to 237, wherein the library comprises at least 2, at least 5, at least 10, at least 15, at least 20, at least 25, at least 30, at least 35, at least 40, at least 45, at least 50, at least 60, at least 70, at least 80, at least 90, at least 100, at least 200, at least 300, at least 400, at least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, at least 1×10¹⁰, or at least 1×10¹¹ linkers.
Embodiment 239. The combinational library of any one of embodiments 228 to 238, further comprising a second variable region of a second antibody, wherein the N-terminal amino acids of the linkers are linked with the C-terminal amino acid of the second variable region.
Embodiment 240. The combinational library of any one of embodiments 228 to 238, further comprising a plurality of second variable regions of a second antibody, wherein the N-terminal amino acids of the linkers are linked with the C-terminal amino acids of the second variable regions.
Embodiment 241. The combinational library of any one of embodiments 228 to 238, further comprising a plurality of second variable regions of a plurality of antibodies, wherein the N-terminal amino acids of the linkers are linked with the C-terminal amino acid of the second variable regions.
Embodiment 242. The combinational library of embodiment 239, wherein the second variable region is a heavy chain variable region
Embodiment 243. The combinational library of embodiment 240 or 241, wherein the second variable regions are heavy chain variable regions.
Embodiment 244. The combinational library of embodiment 239, wherein the second variable region is a light chain variable region
Embodiment 245. The combinational library of embodiment 240 or 241, wherein the second variable regions are light chain variable regions.
Embodiment 246. The combinational library of any one of embodiments 239 to 245, wherein (i) the first variable region and the second variable region bind to nonoverlapping epitopes on the same target, (ii) the first variable region and the second variable region do not bind to the same target, (iii) the first variable regions and the second variable regions bind to nonoverlapping epitopes on the same target, or (iv) the first variable regions and the second variable regions do not bind to the same target.
Embodiment 247. A method of screening for a co-binder to a target, comprising (i) obtaining the library of any one of embodiments 228 to 246; and (ii) contacting the library of co-binder candidates from step (i) with the target to identify a co-binder that specifically binds to the target.
Embodiment 248. A method of screening for a co-binder to a target, comprising (i) expressing a library of expression vectors encoding the library of any one of embodiments 228 to 246; (ii) obtaining the library of any one of embodiments 228 to 246; and (iii) contacting the library of co-binder candidates from step (ii) with the target to identify a co-binder that specifically binds to the target.
Embodiment 249. A method of screening for a co-binder to a target, comprising (i) expressing a library of expression vectors encoding the library of any one of embodiments 228 to 246; (ii) obtaining the library of any one of embodiments 228 to 246; (iii) contacting the library of co-binder candidates from step (ii) with the target to form complexes between the co-binders that specifically bind to the target and the target; (iv) enriching for the complexes between the co-binders that specifically bind to the target and the target; and (v) identifying the co-binders that specifically bind to the target and the target.
Embodiment 250. The method of any one of embodiments 247 to 249, wherein the co-binder binds the target with a K_{D} of less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, less than 1×10⁻¹⁶ M, less than 1×10⁻¹⁷ M, or less than 1×10⁻¹⁸ M.
Embodiment 251. The method of any one of embodiments 247 to 249, wherein the co-binder binds the target with a K_{D} of about 1×10⁻⁸ M, about 1×10⁻⁹ M, about 1×10⁻¹⁰ M, about 1×10⁻¹¹ M, about 1×10⁻¹² M, about 1×10⁻¹³ M, about 1×10⁻¹⁴ M, about 1×10⁻¹⁵ M, about 1×10⁻¹⁶ M, about 1×10⁻¹⁷ M, or about 1×10⁻¹⁸ M.
Embodiment 252. The method of any one of embodiments 247 to 251, wherein the affinity of the co-binder to the target is no less than 50 fold, no less than 100 fold, no less than 150 fold, no less than 200 fold, no less than 250 fold, no less than 300 fold, no less than 350 fold, no less than 400 fold, no less than 450 fold, no less than 500 fold, no less than 600 fold, no less than 700 fold, no less than 800 fold, no less than 900 fold, no less than 1000 fold, no less than 1100 fold, no less than 1200 fold, no less than 1300 fold, no less than 1400 fold, no less than 1500 fold, no less than 1600 fold, no less than 1700 fold, no less than 1800 fold, no less than 1900 fold, no less than 2000 fold, no less than 3000 fold, no less than 4000 fold, or no less than 5000 fold, or no less than 10000 fold greater than that of the individual variable regions alone.

### CERTAIN TABLES

Provided in this section are certain lengthy tables described herein.

TABLE 3
The sequences of the framework 1 region (FR1, framework region 1) of
isotype Ig heavy chain variable regions. The description in the left
column contains database identifiers, based on which the full
length sequences of the immunoglobulin sequence records can be
obtained, which are hereby incorporated in their entireties by reference.

| Description | Sequences |
|---|---|
| Consensus | ZXXLXZXGXXXXXXXXXXXXCXXS (SEQ ID NO: 1) |
| M99641\|IGHV1-18*01\|Homo | QVQLVQSGAEVKKPGASVKVSCKAS (SEQ ID NO: 2) |
| X60503\|IGHV1-18*02\|Homo | QVQLVQSGAEVKKPGASVKVSCKAS (SEQ ID NO: 2) |
| HM855463\|IGHV1-18*03\|Homo | QVQLVQSGAEVKKPGASVKVSCKAS (SEQ ID NO: 2) |
| KC713938\|IGHV1-18*04\|Homo | QVQLVQSGAEVKKPGASVKVSCKAS (SEQ ID NO: 2) |
| X07448\|IGHV1-2*01\|Homo | QVQLVQSGAEVKKPGASVKVSCKAS (SEQ ID NO: 2) |
| X62106\|IGHV1-2*02\|Homo | QVQLVQSGAEVKKPGASVKVSCKAS (SEQ ID NO: 2) |
| KF698733\|IGHV1-2*04\|Homo | QVQLVQSGAEVKKPGASVKVSCKAS (SEQ ID NO: 2) |
| HM855674\|IGHV1-2*05\|Homo | QVQLVQSGAEVKKPGASVKVSCKAS (SEQ ID NO: 2) |
| MH267285\|IGHV1-2*06\|Homo | QVQLVQSGAEVKKPGASVKVSCKAS (SEQ ID NO: 2) |
| X62109\|IGHV1-3*01\|Homo | QVQLVQSGAEVKKPGASVKVSCKAS (SEQ ID NO: 2) |
| MK540645\|IGHV1-3*03\|Homo | QVQLVQSGAEVKKPGASVKVSCKAS (SEQ ID NO: 2) |
| MH779622\|IGHV1-3*04\|Homo | QVQLVQSGAEVKKPGASVKVSCKAS (SEQ ID NO: 2) |
| X92343\|IGHV1-46*01\|Homo | QVQLVQSGAEVKKPGASVKVSCKAS (SEQ ID NO: 2) |
| J00240\|IGHV1-46*02\|Homo | QVQLVQSGAEVKKPGASVKVSCKAS (SEQ ID NO: 2) |
| L06612\|IGHV1-46*03\|Homo | QVQLVQSGAEVKKPGASVKVSCKAS (SEQ ID NO: 2) |
| MK540650\|IGHV1-46*04\|Homo | QVQLVQSGAEVKKPGASVKVSCKAS (SEQ ID NO: 2) |
| M99637\|IGHV1-8*01\|Homo | QVQLVQSGAEVKKPGASVKVSCKAS (SEQ ID NO: 2) |
| HM855457\|IGHV1-8*02\|Homo | QVQLVQSGAEVKKPGASVKVSCKAS (SEQ ID NO: 2) |
| MG719312\|IGHV1-8*03\|Homo | QVQLVQSGAEVKKPGASVKVSCKAS (SEQ ID NO: 2) |
| X92208\|IGHV1-2*03\|Homo | QVQLVQSGAEVKKLGASVKVSCKAS (SEQ ID NO: 3) |
| M99642\|IGHV1-24*01\|Homo | QVQLVQSGAEVKKPGASVKVSCKVS (SEQ ID NO: 4) |
| X92209\|IGHV1-45*01\|Homo | QMQLVQSGAEVKKTGSSVKVSCKAS (SEQ ID NO: 5) |
| AB019438\|IGHV1-45*02\|Homo | QMQLVQSGAEVKKTGSSVKVSCKAS (SEQ ID NO: 5) |
| MG719320\|IGHV1-45*03\|Homo | QMQLVQSGAEVKKTGSSVKVSCKAS (SEQ ID NO: 5) |
| L22582\|IGHV1-69*01\|Homo | QVQLVQSGAEVKKPGSSVKVSCKAS (SEQ ID NO: 6) |
| Z27506\|IGHV1-69*02\|Homo | QVQLVQSGAEVKKPGSSVKVSCKAS (SEQ ID NO: 6) |
| X92340\|IGHV1-69*03\|Homo | QVQLVQSGAEVKKPGSSVKVSCKAS (SEQ ID NO: 6) |
| M83132\|IGHV1-69*04\|Homo | QVQLVQSGAEVKKPGSSVKVSCKAS (SEQ ID NO: 6) |
| X67905\|IGHV1-69*05\|Homo | QVQLVQSGAEVKKPGSSVKVSCKAS (SEQ ID NO: 6) |
| L22583\|IGHV1-69*06\|Homo | QVQLVQSGAEVKKPGSSVKVSCKAS (SEQ ID NO: 6) |
| Z14309\|IGHV1-69*08\|Homo | QVQLVQSGAEVKKPGSSVKVSCKAS (SEQ ID NO: 6) |
| Z14307\|IGHV1-69*09\|Homo | QVQLVQSGAEVKKPGSSVKVSCKAS (SEQ ID NO: 6) |
| Z14300\|IGHV1-69*10\|Homo | QVQLVQSGAEVKKPGSSVKVSCKAS (SEQ ID NO: 6) |
| Z14296\|IGHV1-69*11\|Homo | QVQLVQSGAEVKKPGSSVKVSCKAS (SEQ ID NO: 6) |
| Z14214\|IGHV1-69*13\|Homo | QVQLVQSGAEVKKPGSSVKVSCKAS (SEQ ID NO: 6) |
| KC713948\|IGHV1-69*14\|Homo | QVQLVQSGAEVKKPGSSVKVSCKAS (SEQ ID NO: 6) |
| MG719326\|IGHV1-69*15\|Homo | QVQLVQSGAEVKKPGSSVKVSCKAS (SEQ ID NO: 6) |
| MG719328\|IGHV1-69*16\|Homo | QVQLVQSGAEVKKPGSSVKVSCKAS (SEQ ID NO: 6) |
| MH359407\|IGHV1-69*17\|Homo | QVQLVQSGAEVKKPGSSVKVSCKAS (SEQ ID NO: 6) |
| KC713934\|IGHV1-69D*01\|Homo | QVQLVQSGAEVKKPGSSVKVSCKAS (SEQ ID NO: 6) |
| M29809\|IGHV1-58*01\|Homo | QMQLVQSGPEVKKPGTSVKVSCKAS (SEQ ID NO: 7) |
| AB019438\|IGHV1-58*02\|Homo | QMQLVQSGPEVKKPGTSVKVSCKAS (SEQ ID NO: 7) |
| MK321684\|IGHV1-58*03\|Homo | QMQLVQSGPEVKKPGTSVKVSCKAS (SEQ ID NO: 7) |
| IMGT000035\|IGHV7-4-1*01\|Homo | QVQLVQSGSELKKPGASVKVSCKAS (SEQ ID NO: 8) |
| X62110\|IGHV7-4-1*02\|Homo | QVQLVQSGSELKKPGASVKVSCKAS (SEQ ID NO: 8) |
| X92290\|IGHV7-4-1*03\|Homo | QVQLVQSGSELKKPGASVKVSCKAS (SEQ ID NO: 8) |
| HM855485\|IGHV7-4-1*04\|Homo | QVQLVQSGSELKKPGASVKVSCKAS (SEQ ID NO: 8) |
| HM855361\|IGHV7-4-1*05\|Homo | QVQLVQSGSELKKPGASVKVSCKAS (SEQ ID NO: 8) |
| AM939697\|IGHVIS2*01\|Vicugna | EVQLVQPGAELRKPGASVKVSCKAS (SEQ ID NO: 9) |
| AM939701\|IGHVIS6*01\|Vicugna | EVQLVQPGAELRNPGASVKVSCKAS (SEQ ID NO: 10) |
| AM939698\|IGHVIS3*01\|Vicugna | EVQLVQPGAELRKPGASLKVSCKAS (SEQ ID NO: 11) |
| AM939699\|IGHVIS4*01\|Vicugna | EVQLVQPGAELRKPGASLKVSCKAS (SEQ ID NO: 11) |
| AM773729\|IGHV1-1*01\|Vicugna | QVQLVQPGAELRKPGALLKVSCKAS (SEQ ID NO: 12) |
| X92227\|IGHV5-10-1*01\|Homo | EVQLVQSGAEVKKPGESLRISCKGS (SEQ ID NO: 13) |
| X92279\|IGHV5-10-1*02\|Homo | EVQLVQSGAEVKKPGESLRISCKGS (SEQ ID NO: 13) |
| X56375\|IGHV5-10-1*03\|Homo | EVQLVQSGAEVKKPGESLRISCKGS (SEQ ID NO: 13) |
| X56376\|IGHV5-10-1*04\|Homo | EVQLVQSGAEVKKPGESLRISCKGS (SEQ ID NO: 13) |
| M99686\|IGHV5-51*01\|Homo | EVQLVQSGAEVKKPGESLKISCKGS (SEQ ID NO: 14) |
| M18806\|IGHV5-51*02\|Homo | EVQLVQSGAEVKKPGESLKISCKGS (SEQ ID NO: 14) |
| X56368\|IGHV5-51*03\|Homo | EVQLVQSGAEVKKPGESLKISCKGS (SEQ ID NO: 14) |
| X56367\|IGHV5-51*04\|Homo | EVQLVQSGAEVKKPGESLKISCKGS (SEQ ID NO: 14) |
| MK321694\|IGHV5-51*06\|Homo | EVQLVQSGAEVKKPGESLKISCKGS (SEQ ID NO: 14) |
| KF698734\|IGHV1-69-2*01\|Homo | EVQLVQSGAEVKKPGATVKISCKVS (SEQ ID NO: 15) |
| AM939745\|IGHV3S46*01\|Vicugna | EVQLVESGGGLVQPGGSLRLSCAAS (SEQ ID NO: 16) |
| AM939766\|IGHV3S67*01\|Vicugna | EVQLVESGGGLVQPGGSLRLSCAAS (SEQ ID NO: 16) |
| AM939743\|IGHV3S44*01\|Vicugna | EVQLVESGGGLVQPGGSLRLSCAAS (SEQ ID NO: 16) |
| AM939741\|IGHV3S42*01\|Vicugna | EVQLVESGGGLVQPGGSLRLSCAAS (SEQ ID NO: 16) |
| AM939739\|IGHV3S40*01\|Vicugna | EVQLVESGGGLVQPGGSLRLSCAAS (SEQ ID NO: 16) |
| AM939735\|IGHV3S36*01\|Vicugna | EVQLVESGGGLVQPGGSLRLSCAAS (SEQ ID NO: 16) |
| AM939731\|IGHV3S32*01\|Vicugna | EVQLVESGGGLVQPGGSLRLSCAAS (SEQ ID NO: 16) |
| AM939730\|IGHV3S31*01\|Vicugna | EVQLVESGGGLVQPGGSLRLSCAAS (SEQ ID NO: 16) |
| AM939716\|IGHV3S2*01\|Vicugna | EVQLVESGGGLVQPGGSLRLSCAAS (SEQ ID NO: 16) |
| AM939711\|IGHV3S15*01\|Vicugna | EVQLVESGGGLVQPGGSLRLSCAAS (SEQ ID NO: 16) |
| AM939709\|IGHV3S13*01\|Vicugna | EVQLVESGGGLVQPGGSLRLSCAAS (SEQ ID NO: 16) |
| AM939707\|IGHV3S11*01\|Vicugna | EVQLVESGGGLVQPGGSLRLSCAAS (SEQ ID NO: 16) |
| AM939768\|IGHV3S10*01\|Vicugna | EVQLVESGGGLVQPGGSLRLSCAAS (SEQ ID NO: 16) |
| AM773729\|IGHV3-1*01\|Vicugna | EVQLVESGGGLVQPGGSLRLSCAAS (SEQ ID NO: 16) |
| J00239\|IGHV3-74*03\|Homo | EVQLVESGGGLVQPGGSLRLSCAAS (SEQ ID NO: 16) |
| Z17392\|IGHV3-74*02\|Homo | EVQLVESGGGLVQPGGSLRLSCAAS (SEQ ID NO: 16) |
| X92206\|IGHV3-72*01\|Homo | EVQLVESGGGLVQPGGSLRLSCAAS (SEQ ID NO: 16) |
| L33851\|IGHV3-74*01\|Homo | EVQLVESGGGLVQPGGSLRLSCAAS (SEQ ID NO: 16) |
| HM855666\|IGHV3-7*03\|Homo | EVQLVESGGGLVQPGGSLRLSCAAS (SEQ ID NO: 16) |
| X92288\|IGHV3-7*02\|Homo | EVQLVESGGGLVQPGGSLRLSCAAS (SEQ ID NO: 16) |
| X70208\|IGHV3-66*04\|Homo | EVQLVESGGGLVQPGGSLRLSCAAS (SEQ ID NO: 16) |
| M99649\|IGHV3-7*01\|Homo | EVQLVESGGGLVQPGGSLRLSCAAS (SEQ ID NO: 16) |
| Z27504\|IGHV3-66*02\|Homo | EVQLVESGGGLVQPGGSLRLSCAAS (SEQ ID NO: 16) |
| X92218\|IGHV3-66*01\|Homo | EVQLVESGGGLVQPGGSLRLSCAAS (SEQ ID NO: 16) |
| M99682\|IGHV3-64*01\|Homo | EVQLVESGGGLVQPGGSLRLSCAAS (SEQ ID NO: 16) |
| MK540647\|IGHV3-64*07\|Homo | EVQLVESGGGLVQPGGSLRLSCAAS (SEQ ID NO: 16) |
| HM855453\|IGHV3-53*04\|Homo | EVQLVESGGGLVQPGGSLRLSCAAS (SEQ ID NO: 16) |
| HM855336\|IGHV3-48*04\|Homo | EVQLVESGGGLVQPGGSLRLSCAAS (SEQ ID NO: 16) |
| AB019438\|IGHV3-48*02\|Homo | EVQLVESGGGLVQPGGSLRLSCAAS (SEQ ID NO: 16) |
| U03893\|IGHV3-48*03\|Homo | EVQLVESGGGLVQPGGSLRLSCAAS (SEQ ID NO: 16) |
| M99675\|IGHV3-48*01\|Homo | EVQLVESGGGLVQPGGSLRLSCAAS (SEQ ID NO: 16) |
| AC245166\|IGHV3-23*04\|Homo | EVQLVESGGGLVQPGGSLRLSCAAS (SEQ ID NO: 16) |
| HM855616\|IGHV3-13*04\|Homo | EVQLVESGGGLVQPGGSLRLSCAAS (SEQ ID NO: 16) |
| KC713939\|IGHV3-13*05\|Homo | EVQLVESGGGLVQPGGSLRLSCAAS (SEQ ID NO: 16) |
| X92217\|IGHV3-13*01\|Homo | EVQLVESGGGLVQPGGSLRLSCAAS (SEQ ID NO: 16) |
| U29582\|IGHV3-13*03\|Homo | EVQLVESGGGLVQPGGSLRLSCAAC (SEQ ID NO: 17) |
| AM939712\|IGHV3S16*01\|Vicugna | EVQVVESGGGLVQPGGSLRLSCAAS (SEQ ID NO: 18) |
| AM939734\|IGHV3S35*01\|Vicugna | EVQVVESGGGLVQPGGSLRLSCAAS (SEQ ID NO: 18) |
| AM939760\|IGHV3S64*01\|Vicugna | EVQVVESGGGLVQPGGSLRLSCAAS (SEQ ID NO: 18) |
| AM939753\|IGHV3S59*01\|Vicugna | EVQVVESGGGLVQAGGSLRLSCAAS (SEQ ID NO: 19) |
| AM939762\|IGHV3S66*01\|Vicugna | EVQLVESGGGLVQAGGSLRLSCAAS (SEQ ID NO: 20) |
| MK540647\|IGHV3-62*04\|Homo | EVQLVKSGGGLVQPGGSLRLSCAAS (SEQ ID NO: 21) |
| M99653IGHV3-13*02\|Homo | EVHLVESGGGLVQPGGALRLSCAAS (SEQ ID NO: 22) |
| AB019437\|IGHV3-62*01\|Homo | EVQLVESGEGLVQPGGSLRLSCAAS (SEQ ID NO: 23) |
| IMGT000035\|IGHV3-62*03\|Homo | EVQLVESGEGLVQPGGSLRLSCAAS (SEQ ID NO: 23) |
| AB019437\|IGHV3-64*02\|Homo | EVQLVESGEGLVQPGGSLRLSCAAS (SEQ ID NO: 23) |
| M99679\|IGHV3-53*01\|Homo | EVQLVESGGGLIQPGGSLRLSCAAS (SEQ ID NO: 24) |
| J03617\|IGHV3-53*03\|Homo | EVQLVESGGGLIQPGGSLRLSCAAS (SEQ ID NO: 24) |
| AB019437\|IGHV3-66*03\|Homo | EVQLVESGGGLIQPGGSLRLSCAAS (SEQ ID NO: 24) |
| KF698735\|IGHV3-53*02\|Homo | EVQLVETGGGLIQPGGSLRLSCAAS (SEQ ID NO: 25) |
| X70197\|IGHV3-73*01\|Homo | EVQLVESGGGLVQPGGSLKLSCAAS (SEQ ID NO: 26) |
| AB019437\|IGHV3-73*02\|Homo | EVQLVESGGGLVQPGGSLKLSCAAS (SEQ ID NO: 26) |
| M99660\|IGHV3-23*01\|Homo | EVQLLESGGGLVQPGGSLRLSCAAS (SEQ ID NO: 27) |
| M35415\|IGHV3-23*02\|Homo | EVQLLESGGGLVQPGGSLRLSCAAS (SEQ ID NO: 27) |
| AM940223\|IGHV3-23*03\|Homo | EVQLLESGGGLVQPGGSLRLSCAAS (SEQ ID NO: 27) |
| AY757302\|IGHV3-23*05\|Homo | EVQLLESGGGLVQPGGSLRLSCAAS (SEQ ID NO: 27) |
| AC244492\|IGHV3-23D*01\|Homo | EVQLLESGGGLVQPGGSLRLSCAAS (SEQ ID NO: 27) |
| M99408\|IGHV3-15*03\|Homo | EVQLVESAGALVQPGGSLRLSCAAS (SEQ ID NO: 28) |
| M99400\|IGHV3-15*08\|Homo | EVQLVESAGGLVQPGGSLRLSCAAS (SEQ ID NO: 29) |
| M99676\|IGHV3-49*01\|Homo | EVQLVESGGGLVQPGRSLRLSCTAS (SEQ ID NO: 30) |
| AB019438\|IGHV3-49*03\|Homo | EVQLVESGGGLVQPGRSLRLSCTAS (SEQ ID NO: 30) |
| AM940220\|IGHV3-49*04\|Homo | EVQLVESGGGLVQPGRSLRLSCTAS (SEQ ID NO: 30) |
| AM940221\|IGHV3-49*05\|Homo | EVQLVESGGGLVKPGRSLRLSCTAS (SEQ ID NO: 31) |
| M99401\|IGHV3-49*02\|Homo | EVQLVESGGGLVQPGPSLRLSCTAS (SEQ ID NO: 32) |
| M99651\|IGHV3-9*01\|Homo | EVQLVESGGGLVQPGRSLRLSCAAS (SEQ ID NO: 33) |
| HM855577\|IGHV3-9*02\|Homo | EVQLVESGGGLVQPGRSLRLSCAAS (SEQ ID NO: 33) |
| KC713947\|IGHV3-9*03\|Homo | EVQLVESGGGLVQPGRSLRLSCAAS (SEQ ID NO: 33) |
| M77298\|IGHV3-64*03\|Homo | EVQLVESGGGLVQPGGSLRLSCSAS (SEQ ID NO: 34) |
| M77301\|IGHV3-64*05\|Homo | EVQLVESGGGLVQPGGSLRLSCSAS (SEQ ID NO: 34) |
| KC713941\|IGHV3-64D*06\|Homo | EVQLVESGGGLVQPGGSLRLSCSAS (SEQ ID NO: 34) |
| M77299\|IGHV3-64*04\|Homo | QVQLVESGGGLVQPGGSLRLSCSAS (SEQ ID NO: 35) |
| M99657\|IGHV3-20*01\|Homo | EVQLVESGGGVVRPGGSLRLSCAAS (SEQ ID NO: 36) |
| MK540646\|IGHV3-20*04\|Homo | EVQLVESGGGVVRPGGSLRLSCAAS (SEQ ID NO: 36) |
| KC713937\|IGHV3-20*02\|Homo | EVQLVESGGGVVRPGGSLRLSFAAS (SEQ ID NO: 37) |
| MH332884\|IGHV3-20*03\|Homo | EVQLVESGGGVVRPGGSLRLSFAAS (SEQ ID NO: 37) |
| M99672\|IGHV3-43*01\|Homo | EVQLVESGGVVVQPGGSLRLSCAAS (SEQ ID NO: 38) |
| KC713950\|IGHV3-43D*03\|Homo | EVQLVESGGVVVQPGGSLRLSCAAS (SEQ ID NO: 38) |
| AC242184\|IGHV3-43D*04\|Homo | EVQLVESGGVVVQPGGSLRLSCAAS (SEQ ID NO: 38) |
| HM855392\|IGHV3-43*02\|Homo | EVQLVESGGGVVQPGGSLRLSCAAS (SEQ ID NO: 39) |
| M83134\|IGHV3-30*01\|Homo | QVQLVESGGGVVQPGRSLRLSCAAS (SEQ ID NO: 40) |
| M99663\|IGHV3-30*03\|Homo | QVQLVESGGGVVQPGRSLRLSCAAS (SEQ ID NO: 40) |
| L06615\|IGHV3-30*04\|Homo | QVQLVESGGGVVQPGRSLRLSCAAS (SEQ ID NO: 40) |
| M77323\|IGHV3-30*05\|Homo | QVQLVESGGGVVQPGRSLRLSCAAS (SEQ ID NO: 40) |
| L06617\|IGHV3-30*06\|Homo | QVQLVESGGGVVQPGRSLRLSCAAS (SEQ ID NO: 40) |
| L06614\|IGHV3-30*07\|Homo | QVQLVESGGGVVQPGRSLRLSCAAS (SEQ ID NO: 40) |
| M77300\|IGHV3-30*09\|Homo | QVQLVESGGGVVQPGRSLRLSCAAS (SEQ ID NO: 40) |
| M77326\|IGHV3-30*10\|Homo | QVQLVESGGGVVQPGRSLRLSCAAS (SEQ ID NO: 40) |
| M77331\|IGHV3-30*11\|Homo | QVQLVESGGGVVQPGRSLRLSCAAS (SEQ ID NO: 40) |
| M77338\|IGHV3-30*12\|Homo | QVQLVESGGGVVQPGRSLRLSCAAS (SEQ ID NO: 40) |
| M77339\|IGHV3-30*13\|Homo | QVQLVESGGGVVQPGRSLRLSCAAS (SEQ ID NO: 40) |
| M77324\|IGHV3-30*14\|Homo | QVQLVESGGGVVQPGRSLRLSCAAS (SEQ ID NO: 40) |
| M77327\|IGHV3-30*15\|Homo | QVQLVESGGGVVQPGRSLRLSCAAS (SEQ ID NO: 40) |
| M77328\|IGHV3-30*16\|Homo | QVQLVESGGGVVQPGRSLRLSCAAS (SEQ ID NO: 40) |
| M77329\|IGHV3-30*17\|Homo | QVQLVESGGGVVQPGRSLRLSCAAS (SEQ ID NO: 40) |
| X92214\|IGHV3-30*18\|Homo | QVQLVESGGGVVQPGRSLRLSCAAS (SEQ ID NO: 40) |
| L06616\|IGHV3-30*19\|Homo | QVQLVESGGGVVQPGRSLRLSCAAS (SEQ ID NO: 40) |
| AC244456\|IGHV3-30-3*01\|Homo | QVQLVESGGGVVQPGRSLRLSCAAS (SEQ ID NO: 40) |
| M77302\|IGHV3-30-3*02\|Homo | QVQLVESGGGVVQPGRSLRLSCAAS (SEQ ID NO: 40) |
| KC713945\|IGHV3-30-3*03\|Homo | QVQLVESGGGVVQPGRSLRLSCAAS (SEQ ID NO: 40) |
| AC244456\|IGHV3-30-5*01\|Homo | QVQLVESGGGVVQPGRSLRLSCAAS (SEQ ID NO: 40) |
| AB019439\|IGHV3-33*01\|Homo | QVQLVESGGGVVQPGRSLRLSCAAS (SEQ ID NO: 40) |
| M99665\|IGHV3-33*02\|Homo | QVQLVESGGGVVQPGRSLRLSCAAS (SEQ ID NO: 40) |
| M77305\|IGHV3-33*03\|Homo | QVQLVESGGGVVQPGRSLRLSCAAS (SEQ ID NO: 40) |
| M77335\|IGHV3-33*04\|Homo | QVQLVESGGGVVQPGRSLRLSCAAS (SEQ ID NO: 40) |
| M77334\|IGHV3-33*05\|Homo | QVQLVESGGGVVQPGRSLRLSCAAS (SEQ ID NO: 40) |
| HM855436\|IGHV3-33*06\|Homo | QVQLVESGGGVVQPGRSLRLSCAAS (SEQ ID NO: 40) |
| M62737\|IGHV3-30*08\|Homo | QVQLVDSGGGVVQPGRSLRLSCAAS (SEQ ID NO: 41) |
| L26401\|IGHV3-30*02\|Homo | QVQLVESGGGVVQPGGSLRLSCAAS (SEQ ID NO: 42) |
| AC245243\|IGHV3-30-5*02\|Homo | QVQLVESGGGVVQPGGSLRLSCAAS (SEQ ID NO: 42) |
| HM855939\|IGHV3-NL1*01\|Homo | QVQLVESGGGVVQPGGSLRLSCAAS (SEQ ID NO: 42) |
| AM773729\|IGHV3-2*01\|Vicugna | QVQLVESGGGLVQPGGSLRLSCAAS (SEQ ID NO: 43) |
| AM773548\|IGHV3S1*01\|Vicugna | QVQLVESGGGLVQPGGSLRLSCAAS (SEQ ID NO: 43) |
| AM939708\|IGHV3S12*01\|Vicugna | QVQLVESGGGLVQPGGSLRLSCAAS (SEQ ID NO: 43) |
| AM939724\|IGHV3S26*01\|Vicugna | QVQLVESGGGLVQPGGSLRLSCAAS (SEQ ID NO: 43) |
| AM939732\|IGHV3S33*01\|Vicugna | QVQLVESGGGLVQPGGSLRLSCAAS (SEQ ID NO: 43) |
| AM939733\|IGHV3S34*01\|Vicugna | QVQLVESGGGLVQPGGSLRLSCAAS (SEQ ID NO: 43) |
| AM939736\|IGHV3S37*01\|Vicugna | QVQLVESGGGLVQPGGSLRLSCAAS (SEQ ID NO: 43) |
| AM939717\|IGHV3S38*01\|Vicugna | QVQLVESGGGLVQPGGSLRLSCAAS (SEQ ID NO: 43) |
| AM939737\|IGHV3S39*01\|Vicugna | QVQLVESGGGLVQPGGSLRLSCAAS (SEQ ID NO: 43) |
| AM939742\|IGHV3S43*01\|Vicugna | QVQLVESGGGLVQPGGSLRLSCAAS (SEQ ID NO: 43) |
| AM939748\|IGHV3S5*01\|Vicugna | QVQLVESGGGLVQPGGSLRLSCAAS (SEQ ID NO: 43) |
| AM773548\|IGHV3S53*01\|Vicugna | QVQLVESGGGLVQPGGSLRLSCAAS (SEQ ID NO: 43) |
| AM939756\|IGHV3S54*01\|Vicugna | QVQLVESGGGLVQPGGSLRLSCAAS (SEQ ID NO: 43) |
| AM939749\|IGHV3S6*01\|Vicugna | QVQLVESGGGLVQPGGSLRLSCAAS (SEQ ID NO: 43) |
| AM939754\|IGHV3S60*01\|Vicugna | QVQLVESGGGLVQPGGSLRLSCAAS (SEQ ID NO: 43) |
| AM939757\|IGHV3S61*01\|Vicugna | QVQLVESGGGLVQPGGSLRLSCAAS (SEQ ID NO: 43) |
| AM939758\|IGHV3S62*01\|Vicugna | QVQLVESGGGLVQPGGSLRLSCAAS (SEQ ID NO: 43) |
| AM939767\|IGHV3S9*01\|Vicugna | QVQLVESGGGLVQPGGSLRLSCAAS (SEQ ID NO: 43) |
| AM773729\|IGHV3-3*01\|Vicugna | QVQLVESGGGLVQAGGSLRLSCAAS (SEQ ID NO: 44) |
| AM939765\|IGHV3S57*01\|Vicugna | QVQLVESGGGLVQAGGSLRLSCAAS (SEQ ID NO: 44) |
| AM939759\|IGHV3S63*01\|Vicugna | QVQLVESGGGLVQAGGSLRLSCAAS (SEQ ID NO: 44) |
| AM939764\|IGHV3S56*01\|Vicugna | QVQLVESGGGLVQAGGSLRHSCAAS (SEQ ID NO: 45) |
| AM939738\|IGHV3S4*01\|Vicugna | QVQLVESVGGLVQDGGSLRLSCAAS (SEQ ID NO: 46) |
| AM939713\|IGHV3S17*01\|Vicugna | QLQLVESGGGLVQPGGSLRLSCAAS (SEQ ID NO: 47) |
| AM939723\|IGHV3S25*01\|Vicugna | QLQLVESGGGLVQPGGSLRLSCAAS (SEQ ID NO: 47) |
| AM939729\|IGHV3S30*01\|Vicugna | QLQLVESGGGLVQPGGSLRLSCAAS (SEQ ID NO: 47) |
| AM939761\|IGHV3S65*01\|Vicugna | QLQLVESGGGLVQPGGSLRLSCAAS (SEQ ID NO: 47) |
| AM939726\|IGHV3S28*01\|Vicugna | QLQLVESGGGLVQPGGSLRVSCAAS (SEQ ID NO: 48) |
| AM939727\|IGHV3S29*01\|Vicugna | QLQLVESGGGLVQPGGSLRVSCAAS (SEQ ID NO: 48) |
| AM939750\|IGHV3S7*01\|Vicugna | QVQLVETGGGLVQPGGSLRLSCAAS (SEQ ID NO: 49) |
| AM939718\|IGHV3S20*01\|Vicugna | QVQLVESGGGLVQPGVSLRLSCAAS (SEQ ID NO: 50) |
| X92216\|IGHV3-15*01\|Homo | EVQLVESGGGLVKPGGSLRLSCAAS (SEQ ID NO: 51) |
| M99402\|IGHV3-15*04\|Homo | EVQLVESGGGLVKPGGSLRLSCAAS (SEQ ID NO: 51) |
| M99403\|IGHV3-15*05\|Homo | EVQLVESGGGLVKPGGSLRLSCAAS (SEQ ID NO: 51) |
| M99404\|IGHV3-15*06\|Homo | EVQLVESGGGLVKPGGSLRLSCAAS (SEQ ID NO: 51) |
| M99406\|IGHV3-15*07\|Homo | EVQLVESGGGLVKPGGSLRLSCAAS (SEQ ID NO: 51) |
| AB019439\|IGHV3-21*01\|Homo | EVQLVESGGGLVKPGGSLRLSCAAS (SEQ ID NO: 51) |
| M99658\|IGHV3-21*02\|Homo | EVQLVESGGGLVKPGGSLRLSCAAS (SEQ ID NO: 51) |
| HM855323\|IGHV3-21*03\|Homo | EVQLVESGGGLVKPGGSLRLSCAAS (SEQ ID NO: 51) |
| HM855688\|IGHV3-21*04\|Homo | EVQLVESGGGLVKPGGSLRLSCAAS (SEQ ID NO: 51) |
| M99654\|IGHV3-15*02\|Homo | EVQLVESGGALVKPGGSLRLSCAAS (SEQ ID NO: 52) |
| M99652\|IGHV3-11*01\|Homo | QVQLVESGGGLVKPGGSLRLSCAAS (SEQ ID NO: 53) |
| HM855329\|IGHV3-11*04\|Homo | QVQLVESGGGLVKPGGSLRLSCAAS (SEQ ID NO: 53) |
| HM855583\|IGHV3-11*05\|Homo | QVQLVESGGGLVKPGGSLRLSCAAS (SEQ ID NO: 53) |
| KC713940\|IGHV3-11*06\|Homo | QVQLVESGGGLVKPGGSLRLSCAAS (SEQ ID NO: 53) |
| X92287\|IGHV3-11*03\|Homo | QVQLLESGGGLVKPGGSLRLSCAAS (SEQ ID NO: 54) |
| M99648\|IGHV2-26*01\|Homo | QVTLKESGPVLVKPTETLTLTCTVS (SEQ ID NO: 55) |
| MG719335\|IGHV2-26*02\|Homo | QVTLKESGPVLVKPTETLTLTCTVS (SEQ ID NO: 55) |
| MG719336\|IGHV2-26*03\|Homo | QVTLKESGPVLVKPTETLTLTCTIS (SEQ ID NO: 56) |
| X62111\|IGHV2-5*01\|Homo | QITLKESGPTLVKPTQTLTLTCTFS (SEQ ID NO: 57) |
| KF698731\|IGHV2-5*02\|Homo | QITLKESGPTLVKPTQTLTLTCTFS (SEQ ID NO: 57) |
| L21963\|IGHV2-5*04\|Homo | QITLKESGPTLVKPTQTLTLTCTFS (SEQ ID NO: 57) |
| L21964\|IGHV2-5*05\|Homo | QITLKESGPTLVKPTQTLTLTCTFS (SEQ ID NO: 57) |
| L21966\|IGHV2-5*06\|Homo | QITLKESGPTLVKPTQTLTLTCTFS (SEQ ID NO: 57) |
| L21970\|IGHV2-70*12\|Homo | QITLKESGPTLVKPTQTLTLTCTFS (SEQ ID NO: 57) |
| L21962\|IGHV2-70*09\|Homo | QITLKESGPTLVKPTQTLTLTRTFS (SEQ ID NO: 58) |
| L21972\|IGHV2-5*09\|Homo | QVTLKESGPTLVKPTQTLTLTCTFS (SEQ ID NO: 59) |
| L21971\|IGHV2-5*08\|Homo | QVTLKESGPALVKPTQTLTLTCTFS (SEQ ID NO: 60) |
| X92238\|IGHV2-70*03\|Homo | QVTLKESGPALVKPTQTLTLTCTFS (SEQ ID NO: 60) |
| MG719337\|IGHV2-70*04\|Homo | QVTLKESGPALVKPTQTLTLTCTFS (SEQ ID NO: 60) |
| X92239\|IGHV2-70*06\|Homo | QVTLKESGPALVKPTQTLTLTCTFS (SEQ ID NO: 60) |
| L21965\|IGHV2-70*10\|Homo | QVTLKESGPALVKPTQTLTLTCTFS (SEQ ID NO: 60) |
| KC713935\|IGHV2-70D*04\|Homo | QVTLKESGPALVKPTQTLTLTCTFS (SEQ ID NO: 60) |
| KC713949\|IGHV2-70D*14\|Homo | QVTLKESGPALVKPTQTLTLTCTFS (SEQ ID NO: 60) |
| MG719340\|IGHV2-70*16\|Homo | QVTLKESGPVLVKPTQTLTLTCTFS (SEQ ID NO: 61) |
| L21969\|IGHV2-70*01\|Homo | QVTLRESGPALVKPTQTLTLTCTFS (SEQ ID NO: 62) |
| X92241\|IGHV2-70*02\|Homo | QVTLRESGPALVKPTQTLTLTCTFS (SEQ ID NO: 62) |
| X92243\|IGHV2-70*07\|Homo | QVTLRESGPALVKPTQTLTLTCTFS (SEQ ID NO: 62) |
| AB019437\|IGHV2-70*13\|Homo | QVTLRESGPALVKPTQTLTLTCTFS (SEQ ID NO: 62) |
| MG719338\|IGHV2-70*15\|Homo | QVTLRESGPALVKPTQTLTLTCTFS (SEQ ID NO: 62) |
| MG719342\|IGHV2-70*17\|Homo | QVTLRESGPALVKPTQTLTLTCTFS (SEQ ID NO: 62) |
| MK540648\|IGHV2-70*18\|Homo | QVTLRESGPALVKPTQTLTLTCTFS (SEQ ID NO: 62) |
| MK540649\|IGHV2-70*19\|Homo | QVTLRESGPALVKPTQTLTLTCTFS (SEQ ID NO: 62) |
| L21967\|IGHV2-70*11\|Homo | RVTLRESGPALVKPTQTLTLTCTFS (SEQ ID NO: 63) |
| X92245\|IGHV2-70*08\|Homo | QVTLRESGPALVKPTQTLTLTCAFS (SEQ ID NO: 64) |
| AM939770\|IGHV4S3*01\|Vicugna | EVQVQESGPGLVKPSQALSLTCTAS (SEQ ID NO: 65) |
| AM939771\|IGHV4S4*01\|Vicugna | QVQRQESGPGLVKPSQMLSLTCTAS (SEQ ID NO: 66) |
| AM939702\|IGHV4S10*01\|Vicugna | EVQLQESGPGLVKPSQMLSLTCTLS (SEQ ID NO: 67) |
| AM939705\|IGHV4S8*01\|Vicugna | QVQLQESGPGLVKPSQTLSLTCTAS (SEQ ID NO: 68) |
| X05714\|IGHV4-28*01\|Homo | QVQLQESGPGLVKPSDTLSLTCAVS (SEQ ID NO: 69) |
| X92233\|IGHV4-28*03\|Homo | QVQLQESGPGLVKPSDTLSLTCAVS (SEQ ID NO: 69) |
| X56358\|IGHV4-28*04\|Homo | QVQLQESGPGLVKPSDTLSLTCAVS (SEQ ID NO: 69) |
| HM855339\|IGHV4-28*05\|Homo | QVQLQESGPGLVKPSDTLSLTCAVS (SEQ ID NO: 69) |
| HM855782\|IGHV4-28*06\|Homo | QVQLQESGPGLVKPSDTLSLTCAVS (SEQ ID NO: 69) |
| KC713936\|IGHV4-28*07\|Homo | QVQLQESGPGLVKPSDTLSLTCAVS (SEQ ID NO: 69) |
| Z12367\|IGHV4-38-2*01\|Homo | QVQLQESGPGLVKPSETLSLTCAVS (SEQ ID NO: 70) |
| M83133\|IGHV4-28*02\|Homo | QVQLQESGPGLVKPSQTLSLTCAVS (SEQ ID NO: 71) |
| KC713946\|IGHV4-30-4*07\|Homo | QVQLQESGPGLVKPSQTLSLTCAVS (SEQ ID NO: 71) |
| MK321691\|IGHV4-31*11\|Homo | QVQLQESGPGLVKPSQTLSLTCAVS (SEQ ID NO: 71) |
| Z14238\|IGHV4-30-4*01\|Homo | QVQLQESGPGLVKPSQTLSLTCTVS (SEQ ID NO: 72) |
| X92274\|IGHV4-30-4*03\|Homo | QVQLQESGPGLVKPSQTLSLTCTVS (SEQ ID NO: 72) |
| MH779624\|IGHV4-30-4*08\|Homo | QVQLQESGPGLVKPSQTLSLTCTVS (SEQ ID NO: 72) |
| L10098\|IGHV4-31*01\|Homo | QVQLQESGPGLVKPSQTLSLTCTVS (SEQ ID NO: 72) |
| M99683\|IGHV4-31*02\|Homo | QVQLQESGPGLVKPSQTLSLTCTVS (SEQ ID NO: 72) |
| Z14237\|IGHV4-31*03\|Homo | QVQLQESGPGLVKPSQTLSLTCTVS (SEQ ID NO: 72) |
| M95121\|IGHV4-31*05\|Homo | QVQLQESGPGLVKPSQTLSLTCTVS (SEQ ID NO: 72) |
| X92270\|IGHV4-31*06\|Homo | QVQLQESGPGLVKPSQTLSLTCTVS (SEQ ID NO: 72) |
| X92271\|IGHV4-31*07\|Homo | QVQLQESGPGLVKPSQTLSLTCTVS (SEQ ID NO: 72) |
| X92272\|IGHV4-31*08\|Homo | QVQLQESGPGLVKPSQTLSLTCTVS (SEQ ID NO: 72) |
| X92273\|IGHV4-31*09\|Homo | QVQLQESGPGLVKPSQTLSLTCTVS (SEQ ID NO: 72) |
| L10097\|IGHV4-61*02\|Homo | QVQLQESGPGLVKPSQTLSLTCTVS (SEQ ID NO: 72) |
| MK540647\|IGHV4-61*09\|Homo | QVQLQESGPGLVKPSQTLSLTCTVS (SEQ ID NO: 72) |
| AM773548\|IGHV4S1*01\|Vicugna | QVQLQESGPGLVKPSQTLSLTCTVS (SEQ ID NO: 72) |
| AM939772\|IGHV4S5*01\|Vicugna | QVQLQESGPGLVKPSQTLSLTCTVS (SEQ ID NO: 72) |
| M95120\|IGHV4-31*04\|Homo | QVRLQESGPGLVKPSQTLSLTCTVS (SEQ ID NO: 73) |
| X92275\|IGHV4-30-4*04\|Homo | QVQLQDSGPGLVKPSQTLSLTCTVS (SEQ ID NO: 74) |
| Z14235\|IGHV4-31*10\|Homo | QVQLQESGPGLLKPSQTLSLTCTVS (SEQ ID NO: 75) |
| AM939769\|IGHV4S2*01\|Vicugna | EVQLQESGPGLVKPSQTLSLTCTVS (SEQ ID NO: 76) |
| Z14239\|IGHV4-30-4*02\|Homo | QVQLQESGPGLVKPSDTLSLTCTVS (SEQ ID NO: 77) |
| X56360\|IGHV4-59*07\|Homo | QVQLQESGPGLVKPSDTLSLTCTVS (SEQ ID NO: 77) |
| AC233755\|IGHV4-38-2*02\|Homo | QVQLQESGPGLVKPSETLSLTCTVS (SEQ ID NO: 78) |
| X62112\|IGHV4-4*07\|Homo | QVQLQESGPGLVKPSETLSLTCTVS (SEQ ID NO: 78) |
| KC713942\|IGHV4-4*08\|Homo | QVQLQESGPGLVKPSETLSLTCTVS (SEQ ID NO: 78) |
| AB019438\|IGHV4-59*01\|Homo | QVQLQESGPGLVKPSETLSLTCTVS (SEQ ID NO: 78) |
| M29812\|IGHV4-59*02\|Homo | QVQLQESGPGLVKPSETLSLTCTVS (SEQ ID NO: 78) |
| M95114\|IGHV4-59*03\|Homo | QVQLQESGPGLVKPSETLSLTCTVS (SEQ ID NO: 78) |
| M95117\|IGHV4-59*04\|Homo | QVQLQESGPGLVKPSETLSLTCTVS (SEQ ID NO: 78) |
| M95118\|IGHV4-59*05\|Homo | QVQLQESGPGLVKPSETLSLTCTVS (SEQ ID NO: 78) |
| HM855471\|IGHV4-59*08\|Homo | QVQLQESGPGLVKPSETLSLTCTVS (SEQ ID NO: 78) |
| MK540647\|IGHV4-59*11\|Homo | QVQLQESGPGLVKPSETLSLTCTVS (SEQ ID NO: 78) |
| MK321692\|IGHV4-59*12\|Homo | QVQLQESGPGLVKPSETLSLTCTVS (SEQ ID NO: 78) |
| M29811\|IGHV4-61*01\|Homo | QVQLQESGPGLVKPSETLSLTCTVS (SEQ ID NO: 78) |
| X92230\|IGHV4-61*03\|Homo | QVQLQESGPGLVKPSETLSLTCTVS (SEQ ID NO: 78) |
| X92250\|IGHV4-61*04\|Homo | QVQLQESGPGLVKPSETLSLTCTVS (SEQ ID NO: 78) |
| AB019437\|IGHV4-61*08\|Homo | QVQLQESGPGLVKPSETLSLTCTVS (SEQ ID NO: 78) |
| M95119\|IGHV4-59*06\|Homo | QVQLQESGPGLVKPSETLSLTCTVT (SEQ ID NO: 79) |
| AB019439\|IGHV4-39*01\|Homo | QLQLQESGPGLVKPSETLSLTCTVS (SEQ ID NO: 80) |
| X05715\|IGHV4-39*02\|Homo | QLQLQESGPGLVKPSETLSLTCTVS (SEQ ID NO: 80) |
| X92259\|IGHV4-39*03\|Homo | QLQLQESGPGLVKPSETLSLTCTVS (SEQ ID NO: 80) |
| AM940222\|IGHV4-39*07\|Homo | QLQLQESGPGLVKPSETLSLTCTVS (SEQ ID NO: 80) |
| X56356\|IGHV4-61*05\|Homo | QLQLQESGPGLVKPSETLSLTCTVS (SEQ ID NO: 80) |
| M95116\|IGHV4-39*05\|Homo | QLQLQESGPGLVKPSETPSLTCTVS (SEQ ID NO: 81) |
| Z14236\|IGHV4-39*06\|Homo | RLQLQESGPGLVKPSETLSLTCTVS (SEQ ID NO: 82) |
| AB019439\|IGHV4-34*01\|Homo | QVQLQQWGAGLLKPSETLSLTCAVY (SEQ ID NO: 83) |
| M99684\|IGHV4-34*02\|Homo | QVQLQQWGAGLLKPSETLSLTCAVY (SEQ ID NO: 83) |
| X92255\|IGHV4-34*03\|Homo | QVQLQQWGAGLLKPSETLSLTCAVY (SEQ ID NO: 83) |
| X92236\|IGHV4-34*04\|Homo | QVQLQQWGAGLLKPSETLSLTCAVY (SEQ ID NO: 83) |
| X92237\|IGHV4-34*05\|Homo | QVQLQQWGAGLLKPSETLSLTCAVY (SEQ ID NO: 83) |
| X92256\|IGHV4-34*06\|Homo | QVQLQQWGAGLLKPSETLSLTCAVY (SEQ ID NO: 83) |
| X92258\|IGHV4-34*07\|Homo | QVQLQQWGAGLLKPSETLSLTCAVY (SEQ ID NO: 83) |
| M95113\|IGHV4-34*08\|Homo | QVQLQQWGAGLLKPSETLSLTCAVY (SEQ ID NO: 83) |
| X05716\|IGHV4-34*11\|Homo | QVQLQQWGAGLLKPSETLSLTCAVY (SEQ ID NO: 83) |
| X56591\|IGHV4-34*12\|Homo | QVQLQQWGAGLLKPSETLSLTCAVY (SEQ ID NO: 83) |
| Z14243\|IGHV4-59*10\|Homo | QVQLQQWGAGLLKPSETLSLTCAVY (SEQ ID NO: 83) |
| AM939773\|IGHV4S6*01\|Vicugna | EVQLQESGPGLLKPSQTLSLTCAVY (SEQ ID NO: 84) |
| Z14241\|IGHV4-34*09\|Homo | QVQLQESGPGLVKPSQTLSLTCAVY (SEQ ID NO: 85) |
| AM939704\|IGHV4S7*01\|Vicugna | QVQLQESGPGLVKPSQTLSLTCAVY (SEQ ID NO: 85) |
| Z14242\|IGHV4-34*10\|Homo | QVQLQESGPGLVKPSETLSLTCAVY (SEQ ID NO: 86) |
| J04097\|IGHV6-1*01\|Homo | QVQLQQSGPGLVKPSQTLSLTCAIS (SEQ ID NO: 87) |
| Z14223\|IGHV6-1*02\|Homo | QVQLQQSGPGLVKPSQTLSLTCAIS (SEQ ID NO: 87) |
| L10089\|IGHV4-30-2*01\|Homo | QLQLQESGSGLVKPSQTLSLTCAVS (SEQ ID NO: 88) |
| M95122\|IGHV4-30-2*02\|Homo | QLQLQESGSGLVKPSQTLSLTCAVS (SEQ ID NO: 88) |
| X92229\|IGHV4-30-2*03\|Homo | QLQLQESGSGLVKPSQTLSLTCAVS (SEQ ID NO: 88) |
| HM855593\|IGHV4-30-2*05\|Homo | QLQLQESGSGLVKPSQTLSLTCAVS (SEQ ID NO: 88) |
| KC713944\|IGHV4-30-2*06\|Homo | QLQLQESGSGLVKPSQTLSLTCAVS (SEQ ID NO: 88) |
| X92232\|IGHV4-4*02\|Homo | QVQLQESGPGLVKPSGTLSLTCAVS (SEQ ID NO: 89) |
| X05713\|IGHV4-4*01\|Homo | QVQLQESGPGLVKPPGTLSLTCAVS (SEQ ID NO: 90) |
| MH779623\|IGHV4-4*03\|Homo | QVQLQESGPGLVKPPGTLSLTCAVS (SEQ ID NO: 90) |
| X92254\|IGHV4-4*05\|Homo | QVQLQELGPGLVKPPGTLSLTCAVS (SEQ ID NO: 91) |
| X92253\|IGHV4-4*04\|Homo | QVQLQESGPGLVKPPGTLSLTCAIS (SEQ ID NO: 92) |

TABLE 4
The sequences of the framework 1 region (FR1, framework region 1) of
isotype Ig kappa (κ) light chain variable regions. The description
in the left column contains database identifiers, based on which
the full length sequences of the immunoglobulin sequence records can
be obtained, which are hereby incorporated in their entireties by reference.

| Description | Sequences |
|---|---|
| M64856\|IGKV1-33*01\|Homo | DIQMTQSPSSLSASVGDRVTITCQA (SEQ ID NO: 93) |
| M64855\|IGKVID-33*01\|Homo | DIQMTQSPSSLSASVGDRVTITCQA (SEQ ID NO: 93) |
| V01577\|IGKV1-12*01\|Homo | DIQMTQSPSSVSASVGDRVTITCRA (SEQ ID NO: 94) |
| V01576\|IGKV1-12*02\|Homo | DIQMTQSPSSVSASVGDRVTITCRA (SEQ ID NO: 94) |
| X17263\|IGKVID-12*01\|Homo | DIQMTQSPSSVSASVGDRVTITCRA (SEQ ID NO: 94) |
| V01576\|IGKVID-12*02\|Homo | DIQMTQSPSSVSASVGDRVTITCRA (SEQ ID NO: 94) |
| J00248\|IGKV1-16*01\|Homo | DIQMTQSPSSLSASVGDRVTITCRA (SEQ ID NO: 95) |
| FM164406\|IGKV1-16*02\|Homo | DIQMTQSPSSLSASVGDRVTITCRA (SEQ ID NO: 95) |
| K01323\|IGKVID-16*01\|Homo | DIQMTQSPSSLSASVGDRVTITCRA (SEQ ID NO: 95) |
| V00558\|IGKVID-16*02\|Homo | DIQMTQSPSSLSASVGDRVTITCRA (SEQ ID NO: 95) |
| X72808\|IGKV1-17*01\|Homo | DIQMTQSPSSLSASVGDRVTITCRA (SEQ ID NO: 95) |
| D88255\|IGKV1-17*02\|Homo | DIQMTQSPSSLSASVGDRVTITCRA (SEQ ID NO: 95) |
| M64858\|IGKV1-6*01\|Homo | AIQMTQSPSSLSASVGDRVTITCRA (SEQ ID NO: 96) |
| KM455558\|IGKV1-6*02\|Homo | AIQMTQSPSSLSASVGDRVTITCRA (SEQ ID NO: 96) |
| KM455566\|IGKV1-17*03\|Homo | DIQMTQSPSAMSASVGDRVTITCRA (SEQ ID NO: 97) |
| X63392\|IGKVID-17*01\|Homo | NIQMTQSPSAMSASVGDRVTITCRA (SEQ ID NO: 98) |
| Z00006\|IGKV1-13*02\|Homo | AIQLTQSPSSLSASVGDRVTITCRA (SEQ ID NO: 99) |
| KM455562\|IGKVID-13*02\|Homo | AIQLTQSPSSLSASVGDRVTITCRA (SEQ ID NO: 99) |
| X17262\|IGKVID-13*01\|Homo | AIQLTQSPSSLSASVGDRVTITCRA (SEQ ID NO: 99) |
| Z00001\|IGKV1-5*01\|Homo | DIQMTQSPSTLSASVGDRVTITCRA (SEQ ID NO: 100) |
| M23851\|IGKV1-5*02\|Homo | DIQMTQSPSTLSASVGDRVTIICRA (SEQ ID NO: 101) |
| X72813\|IGKV1-5*03\|Homo | DIQMTQSPSTLSASVGDRVTITCRA (SEQ ID NO: 100) |
| Z00013\|IGKV1-9*01\|Homo | DIQLTQSPSFLSASVGDRVTITCRA (SEQ ID NO: 102) |
| X63398\|IGKV1-27*01\|Homo | DIQMTQSPSSLSASVGDRVTITCRA (SEQ ID NO: 95) |
| Y14865\|IGKV1-NL1*01\|Homo | DIQMTQSPSSLSASVGDRVTITCRA (SEQ ID NO: 95) |
| Z00008\|IGKVID-8*01\|Homo | VIWMTQSPSLLSASTGDRVTISCRM (SEQ ID NO: 103) |
| KM455567\|IGKVID-8*03\|Homo | VIWMTQSPSLLSASTGDRVTISCRM (SEQ ID NO: 103) |
| KM455563\|IGKVID-8*02\|Homo | AIWMTQSPSLLSASTGDRVTISCRM (SEQ ID NO: 104) |
| Z00014\|IGKV1-8*01\|Homo | AIRMTQSPSSFSASTGDRVTITCRA (SEQ ID NO: 105) |
| X72817\|IGKVID-43*01\|Homo | AIRMTQSPFSLSASVGDRVTITCWA (SEQ ID NO: 106) |
| X59315\|IGKV1-39*01\|Homo | DIQMTQSPSSLSASVGDRVTITCRA (SEQ ID NO: 95) |
| X59312\|IGKVID-39*01\|Homo | DIQMTQSPSSLSASVGDRVTITCRA (SEQ ID NO: 95) |
| X63399\|IGKV6-21*01\|Homo | EIVLTQSPDFQSVTPKEKVTITCRA (SEQ ID NO: 107) |
| X12683\|IGKV6D-21*01\|Homo | EIVLTQSPDFQSVTPKEKVTITCRA (SEQ ID NO: 107) |
| KM455568\|IGKV6-21*02\|Homo | EIVLTQSPDFQSVTPKEKVTITCRA (SEQ ID NO: 107) |
| KM455569\|IGKV6D-21*02\|Homo | EIVLTQSPDFQSVTPKEKVTITCRA (SEQ ID NO: 107) |
| X02485\|IGKV5-2*01\|Homo | ETTLTQSPAFMSATPGDKVNISCKA (SEQ ID NO: 108) |
| X01668\|IGKV3-11*01\|Homo | EIVLTQSPATLSLSPGERATLSCRA (SEQ ID NO: 109) |
| K02768\|IGKV3-11*02\|Homo | EIVLTQSPATLSLSPGERATLSCRA (SEQ ID NO: 109) |
| X17264\|IGKV3D-11*01\|Homo | EIVLTQSPATLSLSPGERATLSCRA (SEQ ID NO: 109) |
| L19271\|IGKV3D-11*03\|Homo | EIVLTQSPATLSLSPGERATLSCRA (SEQ ID NO: 109) |
| KM455561\|IGKV3D-11*02\|Homo | EIVLTQSPATLSLSPGERATLSCRA (SEQ ID NO: 109) |
| L37729\|IGKV3-20*02\|Homo | EIVLTQSPATLSLSPGERATLSCRA (SEQ ID NO: 109) |
| L19272\|IGKV3D-20*02\|Homo | EIVLTQSPATLSLSPGERATLSCRA (SEQ ID NO: 109) |
| X12686\|IGKV3-20*01\|Homo | EIVLTQSPGTLSLSPGERATLSCRA (SEQ ID NO: 110) |
| X12687\|IGKV3D-20*01\|Homo | EIVLTQSPATLSLSPGERATLSCGA (SEQ ID NO: 111) |
| M23090\|IGKV3-15*01\|Homo | EIVMTQSPATLSVSPGERATLSCRA (SEQ ID NO: 112) |
| X72815\|IGKV3D-15*01\|Homo | EIVMTQSPATLSVSPGERATLSCRA (SEQ ID NO: 112) |
| KM455564\|IGKV3D-15*03\|Homo | EIVMTQSPATLSVSPGERATLSCRA (SEQ ID NO: 112) |
| X72820\|IGKV3D-7*01\|Homo | EIVMTQSPATLSLSPGERATLSCRA (SEQ ID NO: 113) |
| Z00023\|IGKV4-1*01\|Homo | DIVMTQSPDSLAVSLGERATINCKS (SEQ ID NO: 114) |
| X63403\|IGKV2-30*01\|Homo | DVVMTQSPLSLPVTLGQPASISCRS (SEQ ID NO: 115) |
| X63402\|IGKV2D-30*01\|Homo | DVVMTQSPLSLPVTLGQPASISCRS (SEQ ID NO: 115) |
| FM164408\|IGKV2-30*02\|Homo | DVVMTQSPLSLPVTLGQPASISCRS (SEQ ID NO: 115) |
| X12684\|IGKV2-24*01\|Homo | DIVMTQTPLSSPVTLGQPASISCRS (SEQ ID NO: 116) |
| X63397\|IGKV2-28*01\|Homo | DIVMTQSPLSLPVTPGEPASISCRS (SEQ ID NO: 117) |
| X12691\|IGKV2D-28*01\|Homo | DIVMTQSPLSLPVTPGEPASISCRS (SEQ ID NO: 117) |
| U41645\|IGKV2-29*02\|Homo | DIVMTQTPLSLSVTPGQPASISCKS (SEQ ID NO: 118) |
| AJ783437\|IGKV2-29*03\|Homo | DIVMTQTPLSLSVTPGQPASISCKS (SEQ ID NO: 118) |
| M31952\|IGKV2D-29*01\|Homo | DIVMTQTPLSLSVTPGQPASISCKS (SEQ ID NO: 118) |
| U41644\|IGKV2D-29*02\|Homo | DIVMTQTPLSLSVTPGQPASISCKS (SEQ ID NO: 118) |
| X59314\|IGKV2-40*01\|Homo | DIVMTQTPLSLPVTPGEPASISCRS (SEQ ID NO: 119) |
| X59311\|IGKV2D-40*01\|Homo | DIVMTQTPLSLPVTPGEPASISCRS (SEQ ID NO: 119) |
| Z27499\|IGKV2D-26*02\|Homo | EIVMTQTPLSLSITPGEQASMSCRS (SEQ ID NO: 120) |
| KM455565\|IGKV2D-26*03\|Homo | EIVMTQTPLSLSITPGEQASMSCRS (SEQ ID NO: 120) |
| AP001216\|IGKV2D-26*01\|Homo | EIVMTQTPLSLSITPGEQASISCRS (SEQ ID NO: 121) |

TABLE 5
The sequences of the framework 1 region (FR1, framework region 1) of
isotype Ig lampda (λ) light chain variable regions. The description in
the left column contains database identifiers, based on which the
full length sequences of the immunoglobulin sequence records can be
obtained, which are hereby incorporated in their entireties by reference.

| | |
|---|---|
| D86996\|IGLV11-55*01\|Homo | RPVLTQPPSLSASPGATARLPCTL(SEQ ID NO: 122) |
| KM455555\|IGLV11-55*02\|Homo | RPVLTQPPSLSASPGATARLPCTL(SEQ ID NO: 122) |
| Z73668\|IGLV5-39*01\|Homo | QPVLTQPTSLSASPGASARFTCTL(SEQ ID NO: 123) |
| AF216776\|IGLV5-39*02\|Homo | QPVLTQPTSLSASPGASARFTCTL(SEQ ID NO: 123) |
| Z73671\|IGLV5-45*02\|Homo | QAVLTQPSSLSASPGASASLTCTL(SEQ ID NO: 124) |
| D86999\|IGLV5-45*03\|Homo | QAVLTQPSSLSASPGASASLTCTL(SEQ ID NO: 124) |
| KM455553\|IGLV5-45*04\|Homo | QAVLTQPSSLSASPGASASLTCTL(SEQ ID NO: 124) |
| Z73670\|IGLV5-45*01\|Homo | QAVLTQPASLSASPGASASLTCTL(SEQ ID NO: 125) |
| Z73649\|IGLV5-48*01\|Homo | QPVLTQPTSLSASPGASARLTCTL(SEQ ID NO: 126) |
| Z73672\|IGLV5-37*01\|Homo | QPVLTQPPSSSASPGESARLTCTL(SEQ ID NO: 127) |
| Z73669\|IGLV5-52*01\|Homo | QPVLTQPSSHSASSGASVRLTCML(SEQ ID NO: 128) |
| Z73676\|IGLV10-54*01\|Homo | QAGLTQPPSVSKGLRQTATLTCTG (SEQ ID NO: 129) |
| D86996\|IGLV10-54*02\|Homo | QAGLTQPPSVSKGLRQTATLTCTG (SEQ ID NO: 129) |
| Z73663\|IGLV1-47*01\|Homo | QSVLTQPPSASGTPGQRVTISCSG (SEQ ID NO: 130) |
| D87016\|IGLV1-47*02\|Homo | QSVLTQPPSASGTPGQRVTISCSG (SEQ ID NO: 130) |
| Z73654\|IGLV1-44*01\|Homo | QSVLTQPPSASGTPGQRVTISCSG (SEQ ID NO: 130) |
| Z73653\|IGLV1-36*01\|Homo | QSVLTQPPSVSEAPRQRVTISCSG (SEQ ID NO: 131) |
| X53936\|IGLV1-40*02\|Homo | QSVVTQPPSVSGAPGQRVTISCTG (SEQ ID NO: 132) |
| Z22192\|IGLV1-40*03\|Homo | QSVVTQPPSVSGAPGQRVTISCTG (SEQ ID NO: 132) |
| M94116\|IGLV1-40*01\|Homo | QSVLTQPPSVSGAPGQRVTISCTG (SEQ ID NO: 133) |
| M94112\|IGLV1-50*01\|Homo | QSVLTQPPSVSGAPGQRVTISCTG (SEQ ID NO: 133) |
| Z73661\|IGLV1-51*01\|Homo | QSVLTQPPSVSAAPGQKVTISCSG (SEQ ID NO: 134) |
| M30446\|IGLV1-51*02\|Homo | QSVLTQPPSVSAAPGQKVTISCSG (SEQ ID NO: 134) |
| M94118\|IGLV1-41*01\|Homo | QSVLTQPPSVSAAPGQKVTISCSG (SEQ ID NO: 134) |
| L27697\|IGLV2-18*02\|Homo | QSALTQPPSVSGSPGQSVTISCTG (SEQ ID NO: 135) |
| L27692\|IGLV2-18*04\|Homo | QSALTQPPSVSGSPGQSVTISCTG (SEQ ID NO: 135) |
| L27694\|IGLV2-18*03\|Homo | QSALTQPPSVSGSPGQSVTISCTG (SEQ ID NO: 135) |
| Z73642\|IGLV2-18*01\|Homo | QSALTQPPSVSGSPGQSVTISCTG (SEQ ID NO: 135) |
| Z73657\|IGLV2-11*01\|Homo | QSALTQPRSVSGSPGQSVTISCTG (SEQ ID NO: 136) |
| Z22198\|IGLV2-11*02\|Homo | QSALTQPRSVSGSPGQSVTISCTG (SEQ ID NO: 136) |
| X97462\|IGLV2-8*01\|Homo | QSALTQPPSASGSPGQSVTISCTG (SEQ ID NO: 137) |
| L27695\|IGLV2-8*02\|Homo | QSALTQPPSASRSPGQSVTISCTG (SEQ ID NO: 138) |
| X14616\|IGLV2-23*01\|Homo | QSALTQPASVSGSPGQSITISCTG (SEQ ID NO: 139) |
| D86994\|IGLV2-23*03\|Homo | QSALTQPASVSGSPGQSITISCTG (SEQ ID NO: 139) |
| Z73665\|IGLV2-23*02\|Homo | QSALTQPASVSGSPGQSITISCTG (SEQ ID NO: 139) |
| L27822\|IGLV2-14*02\|Homo | QSALTQPASVSGSPGQSITISCTG (SEQ ID NO: 139) |
| Z73664\|IGLV2-14*01\|Homo | QSALTQPASVSGSPGQSITISCTG (SEQ ID NO: 139) |
| Z73643\|IGLV2-33*01\|Homo | QSALTQPPFVSGAPGQSVTISCTG (SEQ ID NO: 140) |
| L27823\|IGLV2-33*02\|Homo | QSALTQPPFVSGAPGQSVTISCTG (SEQ ID NO: 140) |
| L27691\|IGLV2-33*03\|Homo | QSALTQPPFVSGAPGQSVTISCTG (SEQ ID NO: 140) |
| Z73673\|IGLV6-57*01\|Homo | NFMLTQPHSVSESPGKTVTISCTR (SEQ ID NO: 141) |
| KM455556\|IGLV6-57*02\|Homo | NFMLTQPHSVSESPGKTVTISCTG (SEQ ID NO: 142) |
| Z73658\|IGLV3-12*01\|Homo | SYELTQPHSVSVATAQMARITCGG (SEQ ID NO: 143) |
| D86998\|IGLV3-12*02\|Homo | SYELTQPHSVSVATAQMARITCGG (SEQ ID NO: 143) |
| D87007\|IGLV3-21*02\|Homo | SYVLTQPPSVSVAPGQTARITCGG (SEQ ID NO: 144) |
| M94115\|IGLV3-21*03\|Homo | SYVLTQPPSVSVAPGKTARITCGG (SEQ ID NO: 145) |
| X71966\|IGLV3-21*01\|Homo | SYVLTQPPSVSVAPGKTARITCGG (SEQ ID NO: 145) |
| X97473\|IGLV3-9*01\|Homo | SYELTQPLSVSVALGQTARITCGG (SEQ ID NO: 146) |
| X74288\|IGLV3-9*02\|Homo | SYELTQPLSVSVALGQAARITCGG (SEQ ID NO: 147) |
| X57826\|IGLV3-1*01\|Homo | SYELTQPPSVSVSPGQTASITCSG (SEQ ID NO: 148) |
| X97464\|IGLV3-10*01\|Homo | SYELTQPPSVSVSPGQTARITCSG (SEQ ID NO: 149) |
| L29166\|IGLV3-10*02\|Homo | SYELTQPPSVSVSPGQTARITCSG (SEQ ID NO: 149) |
| X97474\|IGLV3-25*01\|Homo | SYELMQPPSVSVSPGQTARITCSG (SEQ ID NO: 150) |
| D86994\|IGLV3-25*02\|Homo | SYELTQPPSVSVSPGQTARITCSG (SEQ ID NO: 149) |
| L29165\|IGLV3-25*03\|Homo | SYELTQPPSVSVSPGQTARITCSG (SEQ ID NO: 149) |
| X97471\|IGLV3-16*01\|Homo | SYELTQPPSVSVSLGQMARITCSG (SEQ ID NO: 151) |
| D86994\|IGLV3-27*01\|Homo | SYELTQPSSVSVSPGQTARITCSG (SEQ ID NO: 152) |
| Z73666\|IGLV3-22*01\|Homo | SYELTQLPSVSVSPGQTARITCSG (SEQ ID NO: 153) |
| X56178\|IGLV3-19*01\|Homo | SSELTQDPAVSVALGQTVRITCQG (SEQ ID NO: 154) |
| Z73645\|IGLV3-32*01\|Homo | SSGPTQVPAVSVALGQMARITCQG (SEQ ID NO: 155) |
| Z73674\|IGLV7-46*01\|Homo | QAVVTQEPSLTVSPGGTVTLTCGS (SEQ ID NO: 156) |
| D86999\|IGLV7-46*02\|Homo | QAVVTQEPSLTVSPGGTVTLTCGS (SEQ ID NO: 156) |
| X14614\|IGLV7-43*01\|Homo | QTVVTQEPSLTVSPGGTVTLTCAS (SEQ ID NO: 157) |
| Z73650\|IGLV8-61*01\|Homo | QTVVTQEPSFSVSPGGTVTLTCGL(SEQ ID NO: 158) |
| U03637\|IGLV8-61*02\|Homo | QTVVTQEPSFSVSPGGTVTLTCGL(SEQ ID NO: 158) |
| U03636\|IGLV8/OR8-1*02\|Homo | QSVVTQEPSLSGSPGGTVTLTCAL(SEQ ID NO: 159) |
| Z73667\|IGLV4-60*01\|Homo | QPVLTQSSSASASLGSSVKLTCTL(SEQ ID NO: 160) |
| D87000\|IGLV4-60*02\|Homo | QPVLTQSSSASASLGSSVKLTCTL(SEQ ID NO: 160) |
| AF073885\|IGLV4-60*03\|Homo | QPVLTQSSSASASLGSSVKLTCTL(SEQ ID NO: 160) |
| Z73648\|IGLV4-69*01\|Homo | QLVLTQSPSASASLGASVKLTCTL(SEQ ID NO: 161) |
| U03868\|IGLV4-69*02\|Homo | QLVLTQSPSASASLGASVKLTCTL(SEQ ID NO: 161) |
| X57828\|IGLV4-3*01\|Homo | LPVLTQPPSASALLGASIKLTCTL(SEQ ID NO: 162) |
| Z73675\|IGLV9-49*01\|Homo | QPVLTQPPSASASLGASVTLTCTL(SEQ ID NO: 163) |
| D87016\|IGLV9-49*02\|Homo | QPVLTQPPSASASLGASVTLTCTL(SEQ ID NO: 163) |
| U03869\|IGLV9-49*03\|Homo | QPVLTQPPSASASLGASVTLTCTL(SEQ ID NO: 163) |

### EXAMPLES

The examples in this section are offered as illustrations of the inventions taught herein, and are not intended to limit the disclosure of the present application. A number of embodiments have been described. Nevertheless, it will be understood that various modifications may be made without departing from the spirit and scope of the disclosure. Accordingly, the following examples are intended to illustrate but not limit the scope as described in the claims.

### Example 1: Affinity and Specificity Gain in a Co-Binder

To determine the affinity and specificity gain in a co-binder, the affinity of the co-binder is expressed as K_{D}=K_{D1}×K_{D2}×K′, wherein K_{D} is the affinity of the co-binder, K_{D1}, K_{D2} are the respective binding affinities (i.e. dissociation constants) of the two individual binding moieties, and K′ is the factor caused by the loss of binding energy linking the two binders together. Therefore, the affinity gain of a co-binder over the individual single binders is dependent on K′. Such affinity terms can be translated into energy terms according to thermodynamics laws. The binding free energy of a co-binder can be expressed as: ΔG_{CB}=ΔG_{B1}+ΔG_{B2}+ΔG′, wherein ΔG_{CB} is the binding free energy of the co-binder, ΔG_{B1} and ΔG_{B2} are the respective binding free energy of the two connected individual binding moieties, and ΔG′ is the binding energy loss via linking the two binders together. The goal of maximizing for the co-binder affinity (K_{D}) for a co-binder during the linkage is to minimize the binding energy loss ΔG′, or is to minimize linker-related energy loss, i.e. minimizing K′ factor.

The specificity of a binder is expressed as: SP=K_{D,NT}/K_{D,T}, wherein K_{D,NT} is the affinity for the non-target molecule and K_{D,T} is the affinity for the target molecule. Specificity is described as the ratio of binding to non-target over the target, which is a number larger than 1. For co-binders, binding affinity for non-target and target can be further expressed as: K_{D,CB,NT}=K_{D,B1,NT}. K_{D,B2,NT}. K′_{NT}; and K_{D,CB,T}=K_{D,B1,T}. K_{D,B2,T}. K′_{T}, wherein K_{D, CB} is the affinity of the co-binder, K_{D, B1} and K_{D, B2} are the respective binding affinities (i.e. dissociation constants) of the two connected individual binding moieties, K′ is the affinity loss factor by linking the two binders together, T indicates target, and NT indicates non-target. The specificity of a co-binder can then be expressed as: SP_{CB}=SP_{B1}·SP_{B2}·(K′_{NT}/K′_{T}). For a target and a homologous non-target, the affinity loss factors K_{D,NT} and K_{D,T} should be similar due to the structural resemblance. Therefore, SP_{CB}≈SP_{B1}·SP_{B2}, which is a product of the specificity of individual binding moieties. Based on this equation, linking two single binders to form a co-binder results in a specificity gain.

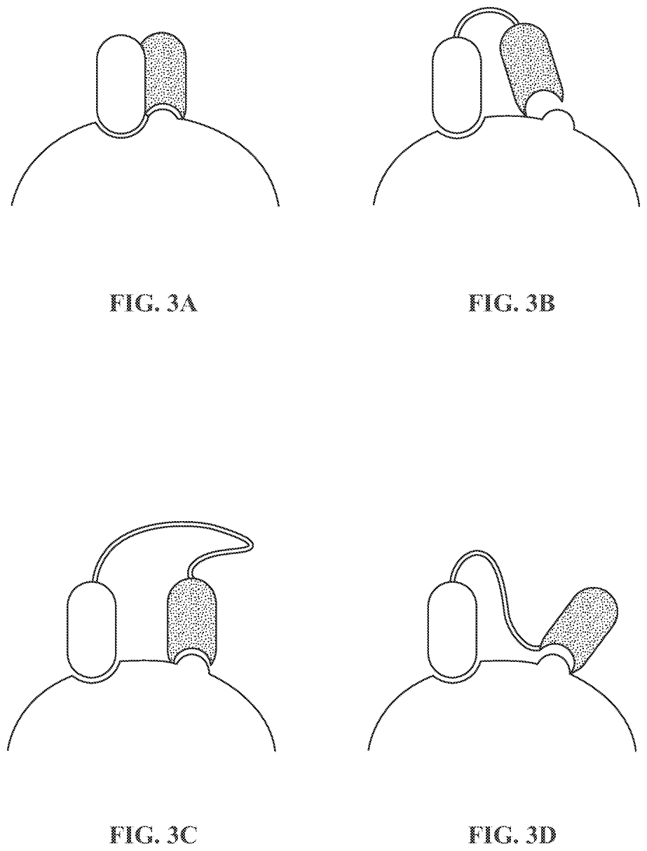

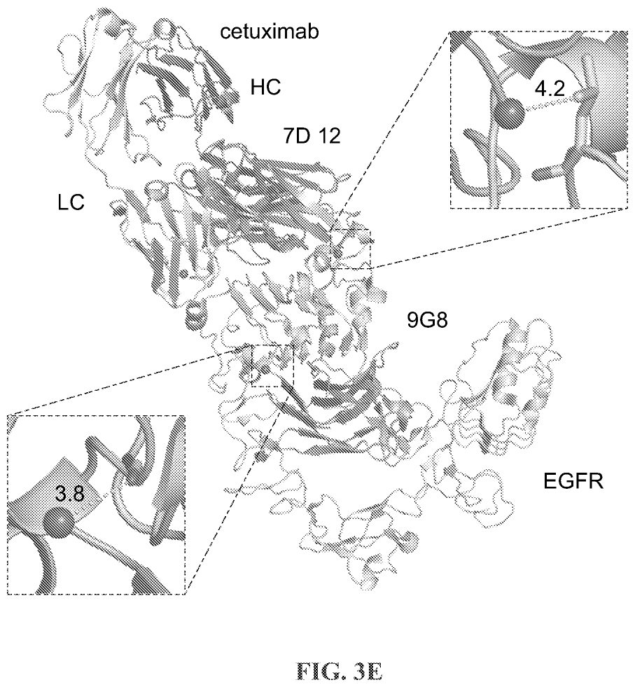

FIGS. 3A-3D illustrates major scenarios where energy losses could occur. In FIG. 3A, the two single binders may interfere with each other's binding even without the linker. This could be due to the sizes of the binding moieties, the relative positions and/or orientation of the two epitopes, etc. In FIG. 3B, the binding of one or more moieties could be disrupted by the linker with improper length and/or rigidity. In FIG. 3C, excessive linker length may add entropy to the system. In FIG. 3D, the linker could intrude into and/or interfere with the binding of one or both binding moieties. The disclosure provides solutions for these and other scenarios.

To confirm the improvement in binding affinity and specificity as taught herein, we selected two anti-EGFR VHH as the single binders as an exemplary demonstration. 7D12 and 9G8 were discovered previously from llama immunization and showed inhibitory activity towards tumor growth (Roovers, Laeremans et al. Cancer Immunol Immunother 56 (3): 303-317; Roovers, Vosjan et al. Int J Cancer. 2011 Oct. 15; 129 (8): 2013-24). From their co-crystal structures with EGFR, the binding epitopes of these two VHHs are not overlapping with each other (PDB code 4KRM and 4KRP) so that the study can focus on the impact of the linkage on binding affinity and specificity gains (FIG. 3E) (Schmitz, Bagchi et al. Structure 21 (7): 1214-1224).

A co-binder 7D12-GS-9G8 was created by inserting a flexible (GGGS)₄ linker in between the C-terminus of 7D12 and N-terminus of 9G8 (referred to as 7D12-GS-9G8 or 7D12-(GGGS)4-9G8). Information regarding 7D12 and 9G8 can be found at <https://www.ebi.ac.uk/pdbe/entry/pdb/4krm/protein/2> and <https://www.ebi.ac.uk/pdbe/entry/pdb/4krp/protein/2>, respectively. The co-binder was cloned, expressed and purified, and its binding affinity for human and mouse EGFR was determined by Biacore as shown in Table 12 below. The co-binder has an affinity (K_{D}) of 0.4 pM for human EGFR, which represents over 1,000-fold improvement over that of single binder 7D12 (KD, 0.5 nM) or 9G8 (KD, 1.1 nM). The K′ factor for human EGFR is 727,000. In contrast, the same co-binder has an affinity of (K_{D}) 120 nM for mouse EGFR, which is weaker than that of 9G8, the stronger of the two single binders. The K′ factor for mouse EGFR is 570,000. This result thus indicates that a co-binder could indeed achieve synergistic co-binding resulting in improved affinity if the linker-related energy loss is smaller than the binding energy gained with an additional binder, as in the case of human EGFR. However, if the energy loss is greater than the binding energy gained with an additional binder, there could be a net loss of binding affinity as in the case of mouse EGFR. The specificity of the co-binder is approximately the same as the multiplication of specificity for 7D12 and 9G8. The specificity improvement is in excess of 10,000-fold when the lower specificity binder 9G8 is used for comparison. If we consider human EGFR as the target and mouse EGFR as the homologous non-target, KD,NT and KD,T are similar as indicated in the above paragraph, which leads to the co-binder specificity of 7D12-GS-9G8 is close to the product of 7D12 and 9G8's specificities. This result thus indicates that, in addition to affinity gain, a co-binder could indeed have improved specificity as well.

We also performed Biacore measurement at 37° C. for the wildtype human EGFR and a mutant human EGFR containing L325A and S340A double substitutions (Table 13). The higher temperature resulted in faster kinetics but weaker affinity for the same co-binder and single binders. The co-binder displayed stronger affinity than the single binders for both WT and mutant EGFR. The K′ factor was measured as 130,000 for the WT and 185,000 for the mutant. Here, the K′ factors for WT and mutant are similar to each other too since there are only 2 mutations between the two. The co-binder has 288-fold specificity for the WT over the mutant EGFR, which is close to the product of the specificities of 7D12 and 9G8. This result again demonstrates the affinity and specificity gain by forming a co-binder.

TABLE 12
Kinetic parameters, K_{D}, K′ and specificity of free 7D12, 9G8 and co-
binder 7D12-(GGGS)₄-9G8 for human and mouse EGFR-Fc measured by SPR at 25° C.

| | Human EGFR-Fc | | | | Mouse EGFR-Fc | | | | Mouse/Human |
|---|---|---|---|---|---|---|---|---|---|
| | kₒₙ (M⁻¹s⁻¹) | k_{off} (s⁻¹) | K_{D} (M) | K′ | kₒₙ (M⁻¹s⁻¹) | k_{off} (s⁻¹) | K_{D} (M) | K′ | Specificity |
| 7D12- | 3.4 × 10⁶ | 1.4 × 10⁻⁶ | 0.4 × 10⁻¹² | 727,000 | 2.3 × 10⁵ | 2.8 × 10⁻² | 1.2 × 10⁻⁷ | 570,000 | 297,500 |
| GS-9G8 | | | | | | | | | |
| 7D12 | 7.3 × 10⁶ | 3.7 × 10⁻³ | 0.5 × 10⁻⁹ | NA | ND | ND | 7.2 × 10⁻⁶ | NA | 14,400 |
| 9G8 | 4.8 × 10⁵ | 5.2 × 10⁻⁴ | 1.1 × 10⁻⁹ | NA | 1.7 × 10⁶ | 5.0 × 10⁻² | 2.9 × 10⁻⁸ | NA | 26 |

TABLE 13
Kinetic parameters, K_{D}, K′ and specificity of 7D12, 9G8 and co-binder 7D12-
GS-9G8 for human and mutant EGFR (L325V, S340A) measured by Biacore at 37° C.

| | | | | | | | | | Mutant/Human |
|---|---|---|---|---|---|---|---|---|---|
| | Human EGFR | | | | Mutant EGFR (L325V, S340A) | | | | Specificity |
| 37° C. | kₒₙ (M⁻¹s⁻¹) | k_{off} (s⁻¹) | K_{D} (M) | K′ | kₒₙ (M⁻¹s⁻¹) | k_{off} (s⁻¹) | K_{D} (M) | K′ | (KD ratio) |
| 7D12- | 1.1 × 10⁷ | 1.8 × 10⁻⁵ | 1.7 × 10⁻¹² | 130,000 | 3.1 × 10⁶ | 1.5 × 10⁻³ | 4.9 × 10⁻¹⁰ | 185,000 | 288 |
| GS-9G8 | | | | | | | | | |
| 7D12 | 7.5 × 10⁶ | 2.5 × 10⁻² | 3.3 × 10⁻⁹ | NA | ND | ND | 2.1 × 10⁻⁷ | NA | 66 |
| 9G8 | 7.7 × 10⁵ | 2.9 × 10⁻³ | 3.8 × 10⁻⁹ | NA | 2.2 × 10⁶ | 2.7 × 10⁻² | 1.3 × 10⁻⁸ | NA | 3.4 |

### Example 2: Factors Affecting Linker-Related Energy Loss in Co-Binders (K′)

Interestingly, we observed that although 7D12-GS-9G8 binding for mouse EGFR is stronger than 7D12 but is 4-fold weaker than 9G8 (Table 12). In this case, linking the two binders together resulted in non-optimal binding of 9G8, which significantly counteracted binding affinity gain by forming a co-binder.

We first examined the crystal structures of 7D12 and 9G8 in details and found that their N-termini are in close contact with EGFR protein surface. The Ca atom of the first residue in 9G8 has a shortest distance of 3.8 Å to EGFR, and that of 7D12 has 4.2 Å. In both cases, the first amino acids are in direct contact with the antigen EGFR. Therefore, addition of a linker to their N-terminus is likely to impair binding. To investigate whether the source of the lost affinity is the linker, we generated 7D12 and 9G8 variants with an anti-human lysozyme VHH, HuL6, attached to their N-termini through a (GGGS)₄ linker. Information regarding HuL6 can be found at <https://www.ebi.ac.uk/pdbe/entry/pdb/1op9/protein/1>. Although the linker is deemed flexible, both HuL6-GS-7D12 and HuL6-GS-9G8 showed significant loss in binding affinity. Compared to the free VHHs, the N-terminal linker attachment caused 26- and 15-fold decreased KD for human EGFR, respectively (Table 14). The construct with a reversed orientation, 7D12-GS-HuL6, showed a similar binding affinity to the free 7D12, suggesting that the affinity loss was not due to any interference from HuL6, but was sourced from the linker at its N-terminus.

TABLE 14
Binding affinities of free 7D12, 9G8 (as individual single binders)
and their HuL6-linked variants as measured by Biacore at 25° C.

| Construct | K_{D} (huEGFR (human EGFR), M) | Fold change |
|---|---|---|
| 7D12 | 5.1 × 10⁻¹⁰ | 26 |
| HuL6-GS-7D12 | 1.3 × 10⁻⁸ | |
| 7D12-GS-HuL6 | 4.4 × 10⁻¹⁰ | 0.86 |
| 9G8 | 1.1 × 10⁻⁹ | 15 |
| HuL6-GS-9G8 | 1.7 × 10⁻⁸ | |

In the case of co-binder 7D12-GS-9G8, since the mouse EGFR binding is much weaker than human EGFR binding, the linker-related affinity loss becomes more discernible (Tables 12 and 15). Similar to the HuL6-fused construct, 9G8 in this co-binder construct also suffers affinity loss due to linker attachment, which led to a weaker affinity than 9G8 alone. In addition, we also constructed a reversed co-binder 9G8-GS-7D12 and measured its affinity for mouse EGFR (Table 15). The binding affinity is 2-fold weaker than 9G8 alone. In this case that 7D12 is placed as the second binder, its contribution to binding is negligible, also likely due to linker-impaired binding, which is consistent to the affinity loss as seen in the HuL6-GS-7D12 measurement.

TABLE 15
Binding measurements of single binders 7D12, 9G8 and the
co-binders for human and mouse EGFR by Biacore at 25° C.

| Construct | KD (muEGFR (mouse EGFR), M) |
|---|---|
| 7D12 | 7.2 × 10⁻⁶ |
| 9G8 | 2.9 × 10⁻⁸ |
| 7D12-GS-9G8 | 1.6 × 10⁻⁷ |
| 9G8-GS-7D12 | 5.8 × 10⁻⁸ |

### Example 3: Strategies for Limiting Energy (Hence Affinity) Loss (K′) in Co-Binders

Various linker designs that can minimize linker-related energy loss, i.e. minimizing the K′ factor were explored so that lower-affinity binders can be used for co-binder formation and still achieve maximal affinity gains.

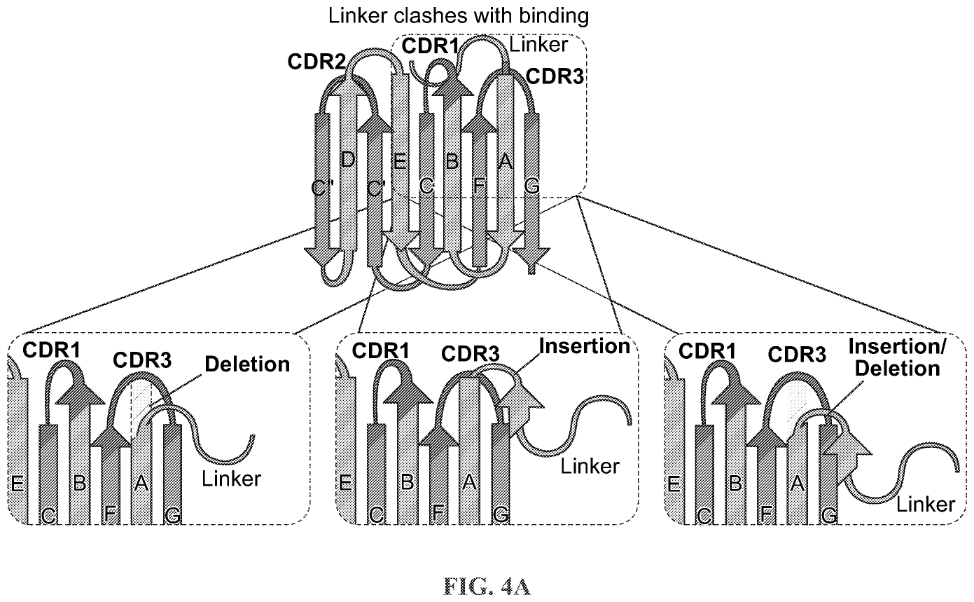

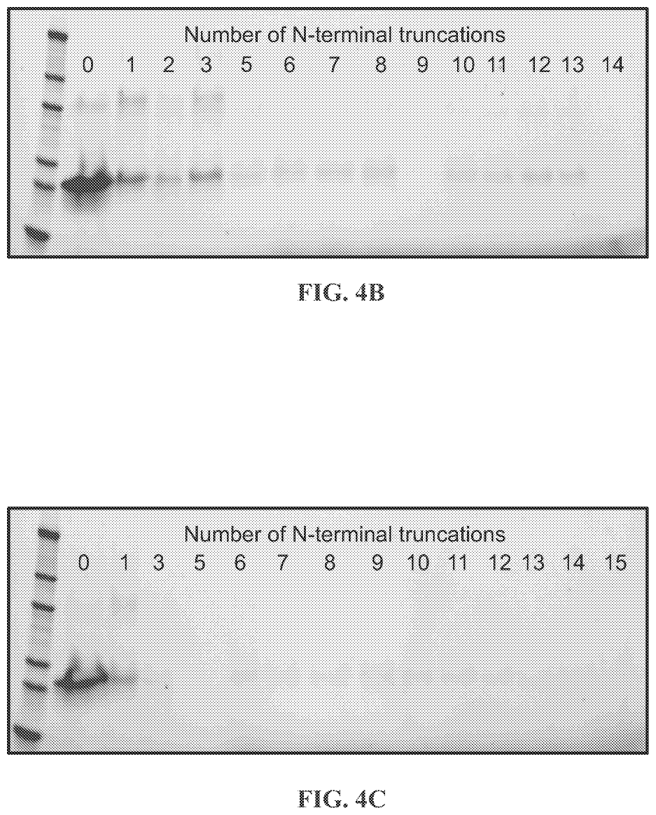

To address the problem of linker proximity interference, three strategies were devised herein for tackling the linker-related energy loss, revolving around a core idea of physically moving the N-terminal linker attachment point away from the antigen binding interface. To achieve this, amino acids can be trimmed from N-terminus of the binder that is to be linked through N-terminus (FIG. 4A); or an amino acids motif can be inserted that would create structural element, such as hairpin, that shifts N-terminus away from the antigen binding interface; or two preceding strategies can be combined, trimming part of the original binder amino acid sequence and inserting an amino acid motif.

A series of HuL6-7D12 and HuL6-9G8 constructs with 1 to 15 residues being truncated at the N-terminus of the second binder was created. Proteins were expressed by yeast secretion strains, purified and then analyzed by SDS-PAGE (FIGS. 4B and 4C). All co-binders containing truncations run at similar mobilities as the non-truncated co-binder, indicating that the produced proteins are intact.

It is important to emphasize that in the above-described strategies, the exact number of amino acids to trim from N-terminus of the binder or the exact amino acid sequence of the inserted motif does not need to be known a priori. Instead, one could create a combinatorial library of solutions, utilizing the described strategies for engineering linker attachment points, from which the most successful binders can be selected using library screening methods such as phage display, yeast display, ribosomal display or comparable. This does not preclude the possibility of creating a universal, engineered linker attachment points common within the same class of antibody scaffolds or across many or all classes of antibody scaffolds.

To further investigate the linker-related affinity loss, we examined the crystal structures of 7D12 and 9G8 in details. We found that both of 7D12 and 9G8 have their N-termini in close vicinity of the EGFR protein surface (FIG. 3E). The Ca atom of the first residue in 9G8 has a distance of 3.8 Å to EGFR, and that of 7D12 has 4.2 Å, similar to the length of one amino acid, leaving no room for free attachment of a linker peptide. It is then expected that addition of a linker to their N-terminus would cause binding interference and affinity loss.

With the understanding that the source of affinity loss is linker proximity to antigen, we devised truncations from the N-terminus of the second binder to move the linker anchor point farther away from the antigen surface.

On top of the truncations at the N-terminus of the second binder, an amino acid motif can be inserted in front of the truncated second binder that would introduce structure flexibility, such as a glycine residue, or create a structural element, such as a hairpin, that shifts N-terminus away from the antigen surface.

To demonstrate these strategies can be used to minimize linker-related affinity loss, we constructed the same HuL6-fusions and co-binders with modified N-termini at the second binder and compared their affinities to the non-modified constructs. For each single binder at the second placement being modified, the first residue was clipped off and connected with a Phe/Thr/Gly motif before being connected to the C-termini of a (GGGS)4 linker. For 7D12, the modified HuL6 fusion, HuL6-GS-FTG-[-1AA]7D12, had a 7-fold increased binding affinity comparing to unmodified HuL6-GS-7D12 (Table 16). For 9G8, the modified HuL6 fusion, HuL6-GS-FTG-[-1AA]9G8, had a 31-fold improvement over HuL6-GS-9G8 (Table 16). In the context of co-binders, N-terminus modified construct 7D12-GS-FTG-[-1AA]9G8 exhibited a 133-fold improved affinity for mouse EGFR, comparing to unmodified reference 7D12-GS-9G8 (Table 16).

TABLE 16
Binding measurements of N-terminus modified
and unmodified HuL6 fusions and co-binders
for human or mouse EGFR by Biacore at 25° C.

| Construct | K_{D} (Human EGFR, M) | Fold Improved |
|---|---|---|
| HuL6-GS-7D12 | 1.3 × 10⁻⁸ | 7 |
| HuL6-GS-FTG-[-1AA]7D12 | 1.8 × 10⁻⁹ | |
| HuL6-GS-9G8 | 1.7 × 10⁻⁸ | 31 |
| HuL6-GS-FTG-[-1AA]9G8 | 5.5 × 10⁻¹⁰ | |
| Construct | K_{D} (Mouse EGFR, | Fold Improved |
| 7D12-GS-9G8 | 1.6 × 10⁻⁷ | 133 |
| 7D12-GS-FTG-[-1AA]9G8 | 1.2 × 10⁻⁹ | |

### Example 4: Linkers Contributing to a Reduction in Energy Loss (K′)

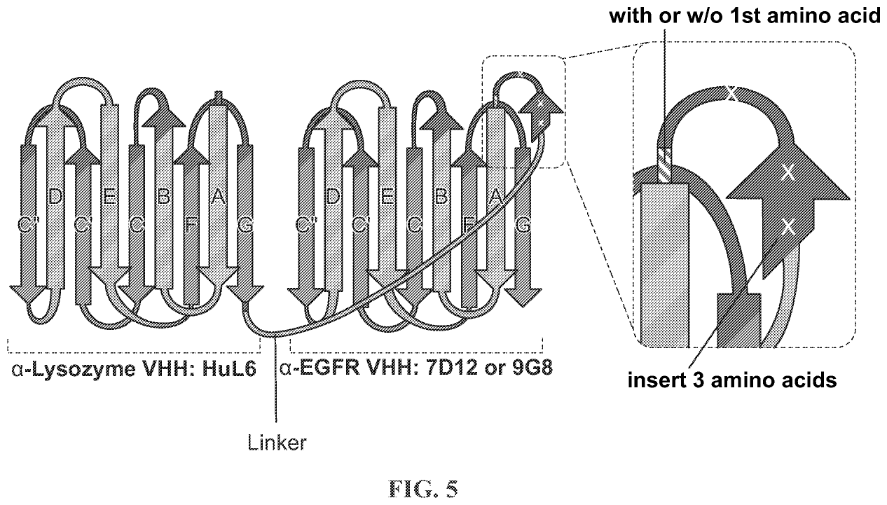

To demonstrate that the energy loss caused by the linker proximity interference and hence K′ can be minimized, four unique co-binder libraries were created, which include the first VHH binder—specifically HuL6 (Dumoulin et al., Protein Sci. 2002 March; 11 (3): 500-15), followed by a linker of four GGGS repeats, followed by three fully randomized amino acids, and ending with the second VHH binder, which was either 9G8 or 7D12, where the first amino acid of the second binder was either unchanged or removed (FIG. 5).

The first and second single binder have intentionally different specificities. HuL6 is specific toward human lysozyme and has no detectable binding to EGFR. In this example the HuL6 serves as a nonfunctional binder to highlight affinity improvement for EGFR binding of 7D12 and 9G8. The four resulting libraries, each with diversity of 8000, underwent a conventional yeast display screening (Chao et. al., Nat Protoc 1 (2): 755-768 (2006)). DNA sequences of the engineered linker were read using Next Generation Sequencing, and translated into amino acid sequences, and unique sequences were counted. Subsequently, sequence enrichment factors were calculated by dividing the count for individual unique sequences in the last round of selection by the count from the previous round. Table 17 lists top 20 most enriched sequences in each library and combinations of libraries.

TABLE 17
Top 20 of triple amino acids motifs from N-terminus of anti-EGFR VHHs (9G8 or 7D12) with or without the VHH's first amino
acid, linked with anti-lysozyme VHH (HuL6) though (GGGS)₄ linker, enriched in a screen for improved binding to EGFR.

| L1: Hul6- | | L2: Hul6- | | L4: Hul6-12GS- | | L5: Hul6-12GS- | | L1&2: Both HuL6- | | L4&5: Both HuL6-12GS- | | L1, L2, L4 | |
|---|---|---|---|---|---|---|---|---|---|---|---|---|---|
| 12GS-XXX-9G8 | | 12GS-XXX-7D12 | | XXX-[-AA]9G8 | | XXX-[-AA]7D12 | | 12GS-7D12 or 9G8 | | [-AA]7D12 or 9G8 | | and L5: All | |
| | Enrich- | | Enrich- | | Enrich- | | Enrich- | | Min | | Min | | Min |
| XXX | ment | XXX | ment | XXX | ment | XXX | ment | XXX | Enrichment | XXX | Enrichment | XXX | Enrich [symbol] |
| HKR | 238.18 | FKR | 223.34 | FRG | 161.22 | GPG | 228.06 | FKR | 171.68 | CCG | 41.26 | YSG | 4.28 |
| FKR | 171.68 | YKR | 220.60 | TYG | 156.37 | FNG | 213.12 | EKR | 68.88 | FWG | 36.92 | WTG | 3.34 |
| MKR | 129.48 | EKR | 136.55 | SYG | 128.18 | FSG | 201.27 | YKR | 62.99 | FLG | 27.72 | FWG | 2.89 |
| CKR | 103.67 | WKR | 61.18 | WRG | 126.53 | FPG | 199.80 | WKR | 61.18 | FTG | 25.32 | YTG | 2.85 |
| QKR | 103.65 | QKR | 46.21 | YYG | 125.09 | FTG | 198.70 | QKR | 46.21 | FQG | 16.37 | YGG | 2.51 |
| VKR | 93.76 | VKR | 45.25 | RYG | 109.15 | YAG | 193.67 | VKR | 45.25 | FVG | 15.54 | FTG | 2.47 |
| RKR | 89.71 | ZLE | 44.24 | TFG | 106.40 | FAG | 190.45 | HKR | 28.24 | FIG | 14.53 | FSG | 2.43 |
| LKR | 84.30 | ZHQ | 43.42 | VYG | 104.35 | WPG | 187.23 | EHY | 13.00 | YGG | 13.51 | YNG | 2.34 |
| KKR | 82.70 | HKR | 28.24 | NZV | 103.42 | HPG | 166.82 | CKR | 10.55 | FMG | 13.05 | HTG | 1.88 |
| WKR | 79.20 | MZL | 22.74 | CCF | 101.18 | CTZ | 164.32 | FRR | 9.59 | YTG | 12.50 | HPG | 1.86 |
| SKR | 78.50 | AMV | 14.41 | FYG | 98.44 | YPG | 151.35 | IKR | 9.50 | FSG | 12.33 | IAG | 1.86 |
| KRG | 70.30 | EHY | 13.00 | HYG | 96.91 | SPG | 147.78 | AKR | 9.50 | YLG | 9.68 | WVG | 1.84 |
| EKR | 68.88 | TYP | 11.83 | QYG | 92.44 | YTG | 140.49 | YRR | 8.89 | YIG | 9.32 | WIG | 1.78 |
| YKR | 62.99 | WAP | 11.55 | KYG | 88.59 | FVG | 136.12 | MKR | 8.62 | ITG | 9.02 | LTG | 1.63 |
| IKR | 62.46 | YMY | 11.29 | IYG | 86.98 | FLG | 135.97 | QNY | 8.27 | VVG | 8.64 | WSG | 1.61 |
| TKR | 62.44 | CKR | 10.55 | CCY | 86.27 | IPG | 127.84 | RKR | 8.18 | FGG | 8.37 | FYG | 1.60 |
| NKR | 60.22 | IYK | 10.34 | FFG | 84.03 | YLG | 124.43 | LKR | 7.89 | YQG | 8.21 | ATG | 1.55 |
| FRR | 58.50 | YTY | 10.08 | QYH | 76.32 | FIG | 123.18 | DKR | 7.77 | IIG | 7.63 | YVG | 1.53 |
| YRR | 57.62 | YYP | 9.85 | GYG | 71.93 | MPG | 120.52 | SKR | 7.16 | YVG | 7.58 | MAG | 1.52 |
| AKR | 54.83 | FRR | 9.59 | MYG | 68.26 | YVG | 120.42 | SGY | 6.62 | YNG | 7.27 | HVG | 1.52 |

[symbol]

indicates data missing or illegible when filed

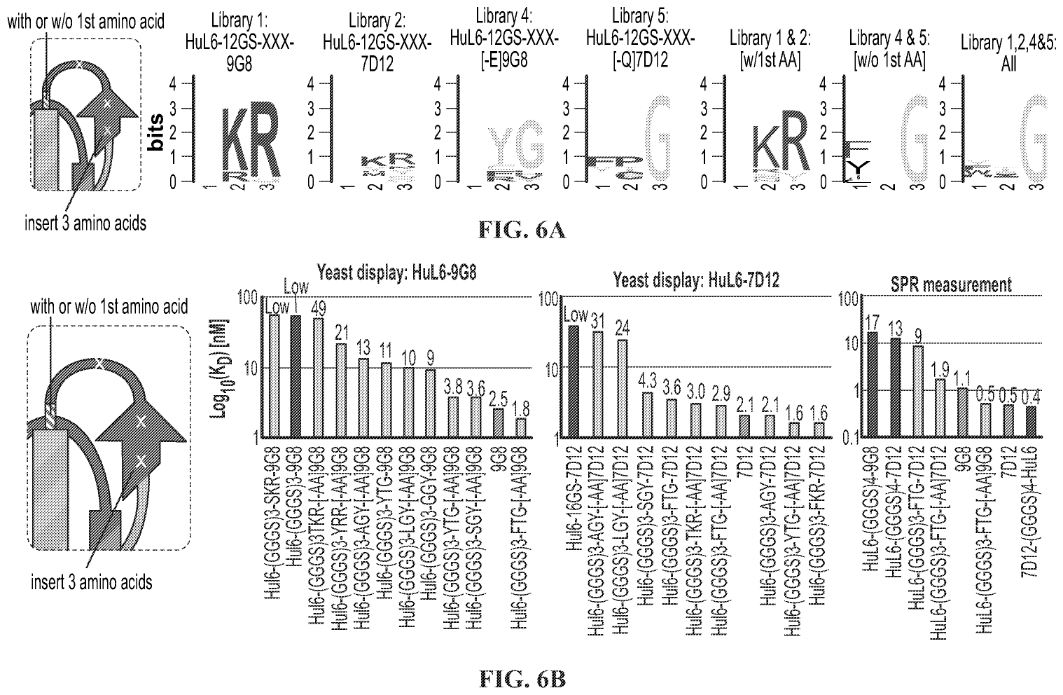

Due to the proximity of the engineered linker sequences to the antigen, it is possible to inadvertently improve the binding not by decreasing the linker-related energy loss, but rather, by increasing binding energy of the individual binders i.e. performing affinity maturation of the binder. To limit that possibility, the sequences that are common among different binders were searched and identified, as it is less likely that the same sequence will convey specificity to different epitopes (Table 17, columns 9-14). In the cases where two or more libraries are compared, enrichment value of the least enriched member was used. Motif analysis of the top 20 binders for each library has uncovered that certain amino acids repeat with frequency higher than would be expected by chance. Furthermore, this is also true when comparing two or more libraries, such as in the case of libraries 1 and 2, 9G8 and 7D12 containing binders with first amino acid preserved (FIG. 6A), libraries 4 and 5, 9G8 and 7D12 containing binders with first amino acid removed (FIG. 6A), as well as in the case of all libraries combined (FIG. 6A). These findings indicate that the selection strategy has identified a finite number of solutions for the linker attachment sites.

### Example 5: Properties of Co-Binders Engineered According to Strategies for Reducing Energy Loss (K′)

To further validate that the identified solutions translate into improved affinities, the dissociation constants of the selected clones were measured using yeast display and SPR (FIG. 6B). These measurements clearly indicate that linking functional binders (9G8 and 7D12) with nonfunctional binder (HuL6) leads to the loss of binding energy (compare red and green bars in FIG. 6B). Furthermore, if the binder is linked through its C terminus, which is facing away from its antigen binding region, it does not lead to the weakening of the K_{D} (compare green and gray bar in FIG. 6B). Finally, a majority of the solutions found through selection leads to improved binding, with the best solutions completely abolishing the observed energy loss attributed to the linker proximity interference (compare yellow to red and green bars in FIG. 6B).

### Example 6: Directed Evolution of Linker Libraries for Co-Binders

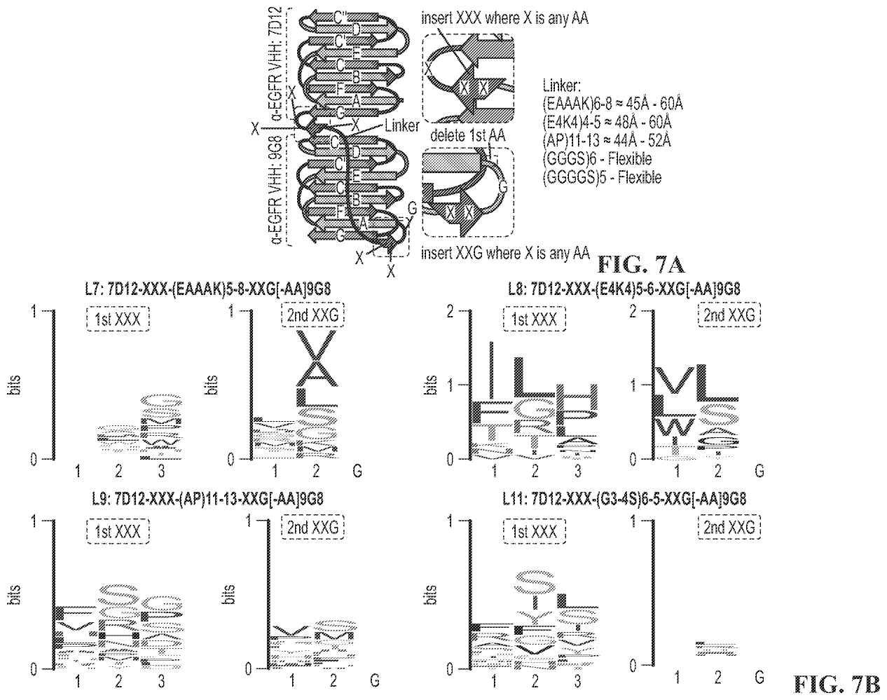

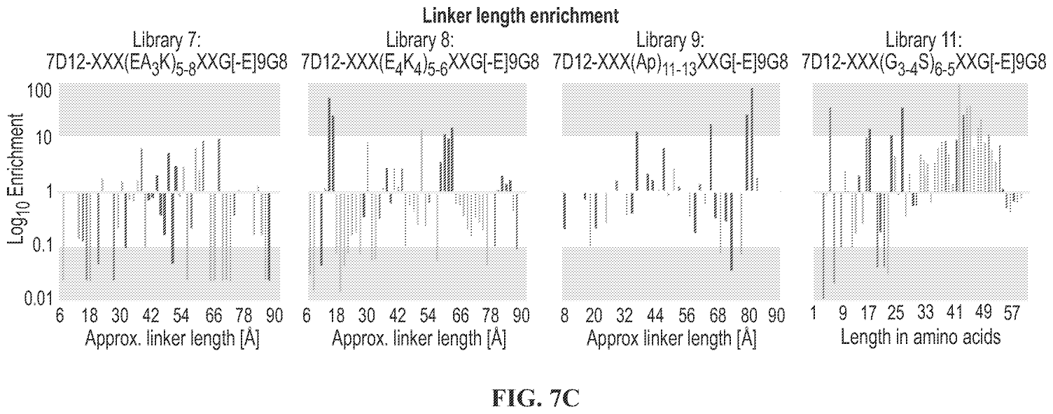

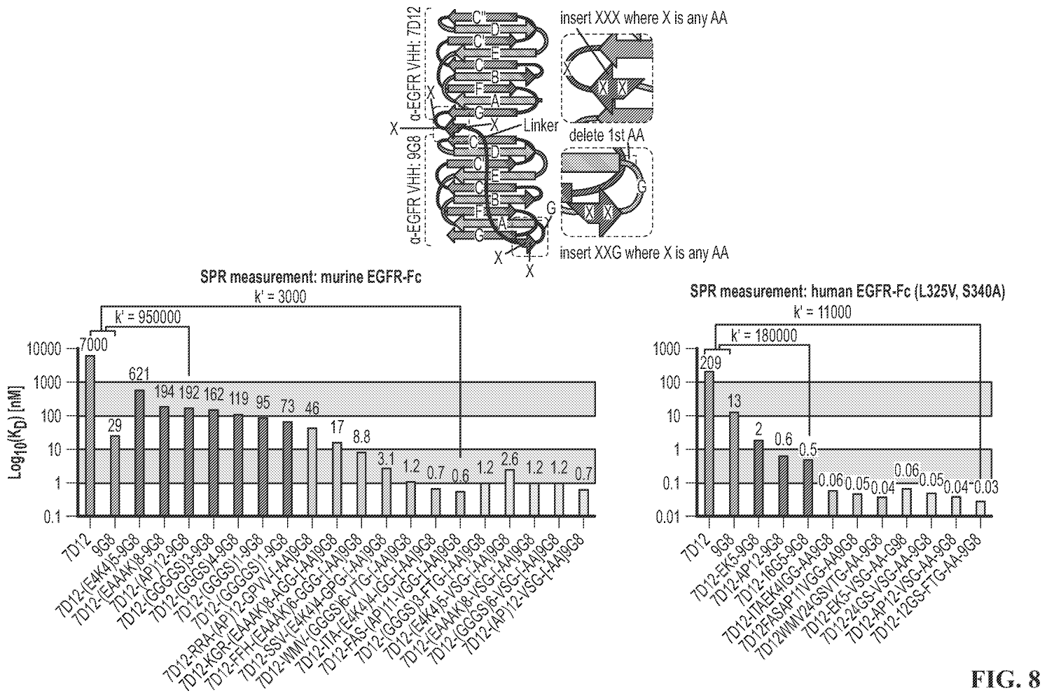

To further demonstrate that engineering of linker attachment sequences can be used to mitigate the energy loss caused by the linker, two anti-EGFR VHH binders (7D12 and 9G8) were linked to create a co-binder. To mitigate the negative linker effects, we have applied the same strategies as described in FIG. 4. Briefly, at the N-terminus of the second binder we removed the first amino acid and inserted XXG randomized sequence, where X stands for any amino acid and G represents glycine. The choice of glycine in that position was dictated by the preference for that amino acid at that position uncovered in FIG. 6A. Moreover, the role of the linker length and rigidity on the affinity of the co-binder was explored. To that end, four different linkers were chosen: two alpha-helix forming motifs EAAAK and E₄K₄ repeats, AP repeat, and G₃₋₄S repeat. As pointed out in FIG. 3B, rigid linkers (i.e. EAAAK, E₄K₄ and AP repeats) need to have right length to not interfere with binding, for that reason we designed different linker lengths spanning 44-60 Å. The distance between 7D12 VHH C-terminus and N-terminus of 9G8 V_{H}H bound to EGFR is approximately 50 Å. A pitch of an α-helix is approximated to be 1.5 Å per amino acid, and 2 Å per amino acid for AP repeat linker. The length of a flexible linker does not appear to influence the gyration radius of the co-binder (Klein et al. Protein Eng Des Sel. 2014 October; 27 (10): 325-30), as such the G3-4S repeat flexible linker was designed to be sufficiently long but limited in length diversity (i.e. 24-25 amino acids). Moreover, another three fully randomized amino acids were inserted at the C-terminus of the first VHH to explore the influence of C-terminal linker attachment on the affinity of the co-binder (FIG. 7A). In total, 4 libraries were created, 6×10⁶-1×10⁷ members each: Library 7—7D12-XXX-(EAAAK)₅₋₈-XXG [-AA]9G8, Library 8—7D12-XXX-(E₄K₄)₅₋₆-XXG [-AA]9G8, Library 9—7D12-XXX-(AP)₁₁₋₁₃-XXG [-AA]9G8, and Library 11—7D12-XXX-(G₃₋₄S)₆₋₅-XXG [-AA]9G8.

The resulting libraries were subjected to conventional yeast display selection (Chao et. al., Nat Protoc 1 (2): 755-768 (2006)). The screen for the strongest binders was performed through 3-4 consecutive rounds of selection with decreasing EGFR antigen concentration in each subsequent round. DNA sequences of engineered linkers were read using Next Generation Sequencing. DNA sequences were trimmed, translated into amino acid sequences, and unique sequences were counted. Sequence enrichment factors were calculated by dividing the count for individual unique sequences in the last round by count from the previous round.

TABLE 18
Top 20 of triple amino acids motifs from N- and C-terminus of anti-EGFR VHH 7D12-9G8 co-binders, linked
by EAAAK, E₄K₄, AP and G₃₋₄S repeats, enriched in a selection for improved binding to EGFR.

| L7: 7D12-XXX-(EAAAK)₅₋₈- | | | | L8: 7D12-XXX-(E₄K₄)₅₋₆- | | | | L9: 7D12-XXX-(AP)₁₁₋₁₃- | | | | L11: 7D12-XXX-(G₃₋₄S)₆₋₅- | | | |
|---|---|---|---|---|---|---|---|---|---|---|---|---|---|---|---|
| XXG[-AA]9G8 | | | | XXG[-AA]9G8 | | | | XXG[-AA]9G8 | | | | XXG[-AA]9G8 | | | |
| Length | 1st | 2nd | Enrich- | Length | 1st | 2nd | Enrich- | Length | 1st | 2nd | Enrich- | Length | 1st | 2nd | Enrich- |
| [Å] | XXX | XXG | ment | [Å] | XXX | XXG | ment | [Å] | XXX | XXG | ment | [AA] | XXX | XXG | ment |
| 60 | IGM | GVG | 32 | 60 | ILH | LLG | 20 | 52 | LFD | PGG | 62 | 24 | PHL | TLG | 140 |
| 60 | FAG | DSG | 25 | 48 | FGP | VSG | 20 | 48 | RSS | SCG | 43 | 24 | FFS | NPG | 121 |
| 60 | GEG | LRG | 24 | 48 | TRL | WLG | 19 | 44 | FHF | VMG | 36 | 24 | RSY | VRG | 110 |
| 60 | SKW | FAG | 21 | 48 | FGP | VSG | 18 | 44 | AHL | GFG | 34 | 24 | FGR | WPG | 108 |
| 60 | ARA | MVG | 20 | 60 | ILH | LLG | 18 | 48 | CSC | KMG | 29 | 24 | FSV | PAG | 105 |
| 60 | MLS | SVG | 19 | 48 | FGP | VSG | 18 | 44 | ISQ | LVG | 27 | 24 | FNI | VSG | 100 |
| 60 | FSR | TKG | 18 | 48 | FGP | VSG | 18 | 52 | VRT | VKG | 24 | 24 | NYS | RGG | 96 |
| 60 | DLG | KMG | 18 | 48 | TRL | WLG | 16 | 44 | VRA | VSG | 23 | 24 | LVK | RAG | 94 |
| 45 | YDG | DGG | 17 | 60 | ILH | LLG | 16 | 44 | FAS | VGG | 22 | 24 | GIT | VEG | 92 |
| 60 | NDS | VSG | 14 | 60 | ILH | LLG | 15 | 52 | VSG | VTG | 22 | 24 | RSV | GTG | 90 |
| 67.5 | TQH | SVG | 14 | 60 | ILH | TAG | 14 | 44 | HSG | FTG | 20 | 24 | RIF | HGG | 88 |
| 60 | SYP | KLG | 12 | 60 | KTI | GIG | 13 | 44 | HGT | GDG | 20 | 24 | TYL | ALG | 88 |
| 60 | VGW | LGG | 12 | 60 | VLY | WEG | 13 | 48 | PRP | NSG | 20 | 24 | SIS | QRG | 86 |
| 52.5 | KSR | VLG | 12 | 48 | IIG | VSG | 13 | 48 | SMP | ALG | 20 | 24 | SVR | SHG | 84 |
| 60 | PGS | LSG | 11 | 60 | NVE | VYG | 13 | 52 | GYG | YSG | 19 | 24 | ESL | LVG | 83 |
| 52.5 | DFP | MAG | 11 | 48 | TRL | WLG | 13 | 44 | PGD | FSG | 18 | 24 | AYE | SQG | 80 |
| 60 | AQL | GAG | 11 | 60 | ILH | LLG | 13 | 48 | FNG | LMG | 18 | 24 | IGI | RRG | 79 |
| 45 | DAI | AVG | 11 | 48 | ITA | IGG | 12 | 44 | FTP | YSG | 17 | 24 | AFL | MZG | 76 |
| 52.5 | IYM | EAG | 11 | 48 | ITA | IGG | 12 | 44 | FNA | HQG | 17 | 10 | MCS | CMG | 76 |
| 60 | LSY | VLG | 10 | 60 | ILH | TAG | 12 | 44 | LGS | MAG | 16 | 24 | RSY | ALG | 74 |

In Table 18, the top 20 most enriched sequences in each library are presented. For all libraries, a significant number of different solutions were discovered, both from perspective of N- and C-terminal attachment sites (FIG. 7B) (N- and C-terminal with regard to the linker), linker lengths (FIG. 7C), and linker compositions (FIG. 8). No obvious enrichment for any particular sequence was observed at the C-terminal attachment sites.

### Example 7: Validation of Affinity and Specificity Improvement from the Engineering of the Linkers and the N-Terminal of the Second Binding Moiety

To validate that observed enrichments translate into improved binding characteristics, a selection of 7D12-9G8 co-binders were expressed and their kinetic characteristics were measured using SPR. Because the affinities of these co-binders for human EGFR had exceeded detection limits of SPR, mouse homologue of EGFR for which the binding strength is significantly reduced was used for the affinity measurement instead (compare FIG. 8 green bars, to FIG. 6B green bars). To establish the baseline, the dissociation constants of several 7D12-9G8 co-binders with different linkers without N- or C-terminal linker attachment engineering (FIG. 8, red bars) were measured. Co-binders lacking N- or C-terminal linker attachment sites did not improve the affinity of the antibody, instead the binding was weakened 2-21 times over the stronger of the two single binders (9G8). This loss is most likely caused by the order of the two single binders. In 7D12-9G8 pair, the stronger binder 9G8 is linked through its N-terminus which increases the probability that a linker will interfere with binding. To determine whether this problem can be resolved by linker attachment site engineering, several of randomly picked solutions from all four libraries were expressed (FIG. 8, orange bars). Majority of engineered solutions lead to improved binding, with the best two 7D12-FAS-(AP)₁₁-VGG-[-AA]9G8 and 7D12-ITA-(E4K4)₄-IGG-[-AA]9G8 achieving 300- to 900-fold improvement over the appropriate co-binders and 40-50 fold improvement over the stronger of the two single binders (9G8). To further test the contributions of N- and C-terminal engineered attachment sites, a single N-terminal sequence recurring among all 4 libraries-VSG [-AA] (Table 18) was selected and tested, and the FTG [-AA] sequence identified in FIG. 6A was also tested (FIG. 8, yellow bars). These N-terminally engineered 7D12-9G8 co-binders (FIG. 8, yellow bars) behave comparable to the best performing, randomly picked, N- and C-terminally engineered co-binders (FIG. 8, orange bars). Furthermore, the binding energy gain among VSG [-AA] co-binders was similar and independent of used linker. Linkers of different types and lengths were observed from the co-binder that showed improved binding affinities (Table 19). To further validate the results, a mutant human EGFR with reduced binding affinity that can be measured more accurately by Biacore was generated through engineering. Again, the same linker designs have achieved similar enhancement of binding affinity as that for mouse EGFR. As another indication of improved linker design, K′ factor can be measured, and they were significantly reduced with the engineered linkers. These findings highlight the importance of engineering the linker attachment sites, especially for the sites proximal to antigen binding pocket, as linker interference is likely the single largest source of the energy loss for linked co-binders. The results demonstrated that the linker sequences and the engineering of the N-terminal sequences of the second binding moiety provided herein for the co-binders, can produce superior improvements in binding energy, greatly reduced K′ factor, and hence produce superior improvements in binding affinity and specificity.

TABLE 19
Linkers used by co-binders that showed
improved binding affinities.

| | Linker |
|---|---|
| | length |
| Linker sequences | (# of aa) |
| APAPAPAPAPAPAPAPAPAPAPAP | 24 |
| EAAAKEAAAKEAAAKEAAAKEAAAKEAAAKEA | 40 |
| AAKEAAAK | |
| EAAAKEAAAKEAAAKEAAAKEAAAKEAAAK | 30 |
| EEEEKKKKEEEEKKKKEEEEKKKKEEEEKKKK | 32 |
| GGGSGGGSGGGSGGGSGGGSGGGS | 24 |
| APAPAPAPAPAPAPAPAPAPAP | 22 |
| GGGSGGGSGGGS | 12 |
| EEEEKKKKEEEEKKKKEEEEKKKKEEEEKKKK | 40 |
| EEEEKKKK | |

### Example 8: Methods of Screening

### Work Flow

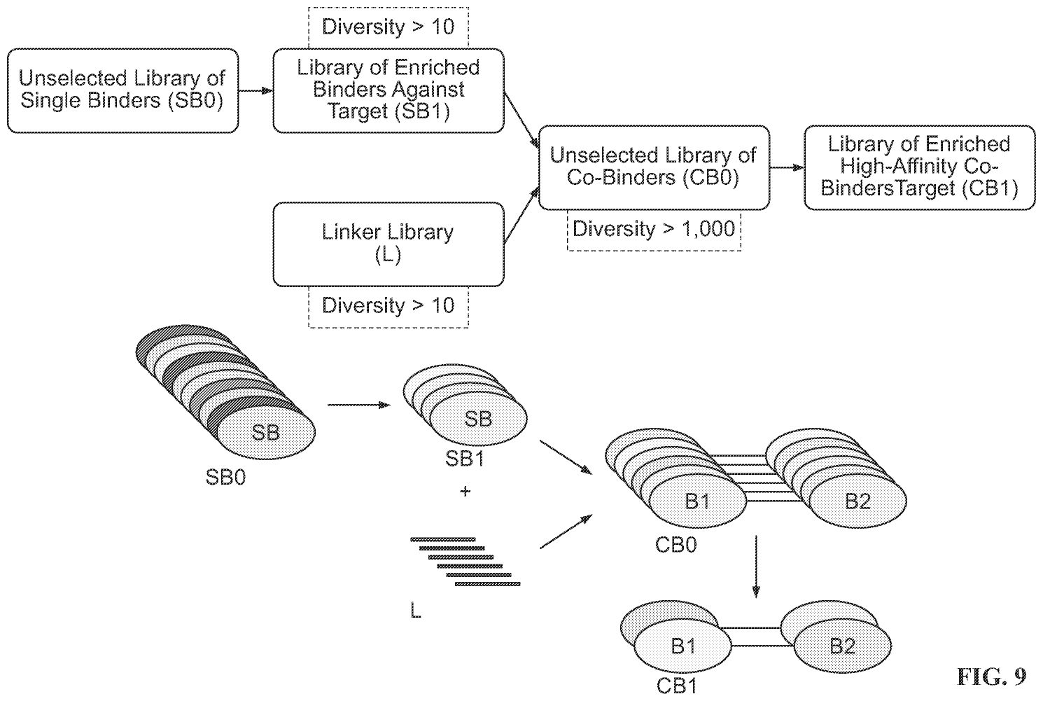

The general workflow is drawn in FIG. 9. Single binder library SB0 is an unselected library comprising single binding moieties. The sequence diversity in SB0 is high, at least in the order of 10⁵, but majority of the members are not expected to have binding towards a target of interest. Therefore, SB0 will first go through the selection for target binding, which results in a binder-enriched single binder library SB1. With a drastically reduced sequence diversity, SB1 is then used for construction of a co-binder library (CB0) by inserting a library of linkers (L) between the randomly paired single binders. CB0 is then further screened to generate the high-affinity co-binder pool CB1. The co-binders in CB1 library can then be sequenced by NGS and top enriched co-binders can be selected for protein production and affinity determination by SPR.

### Libraries of Single Binders and Sorting

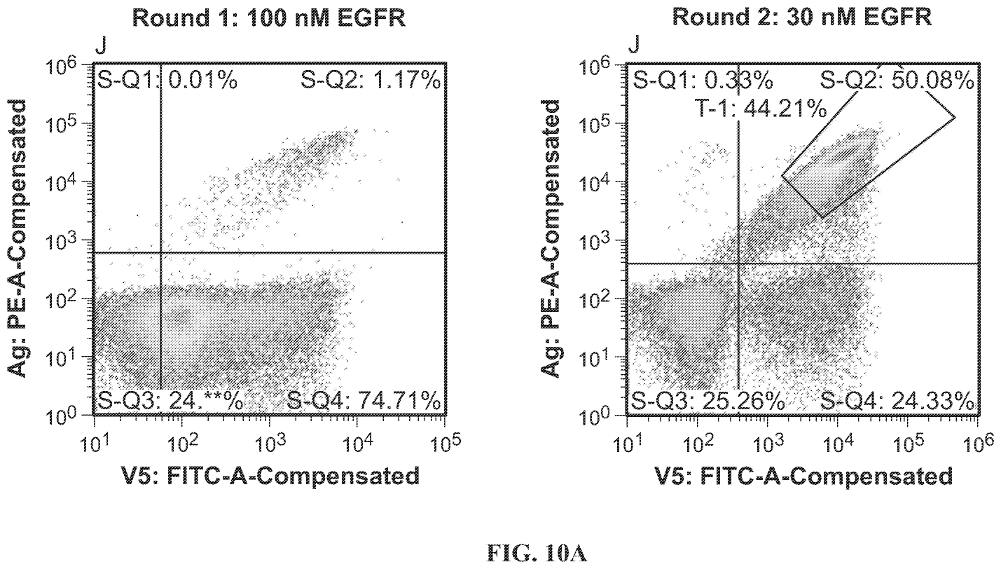

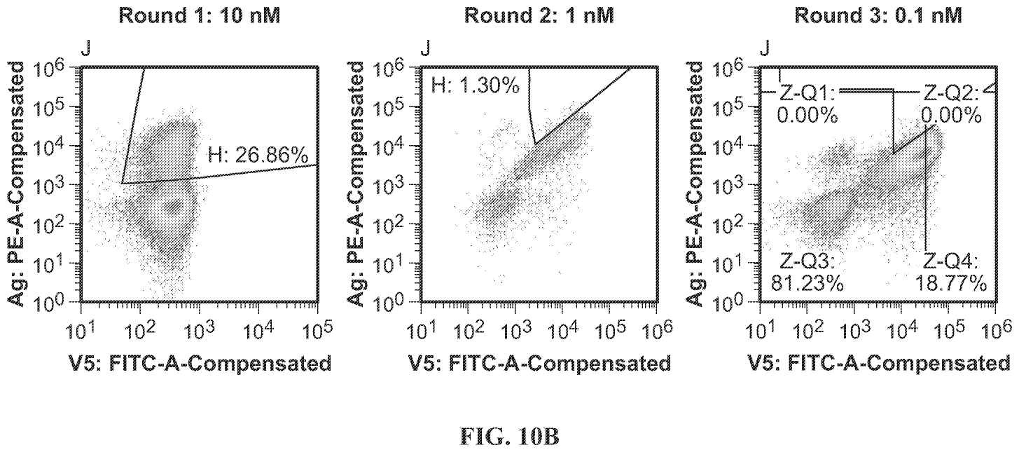

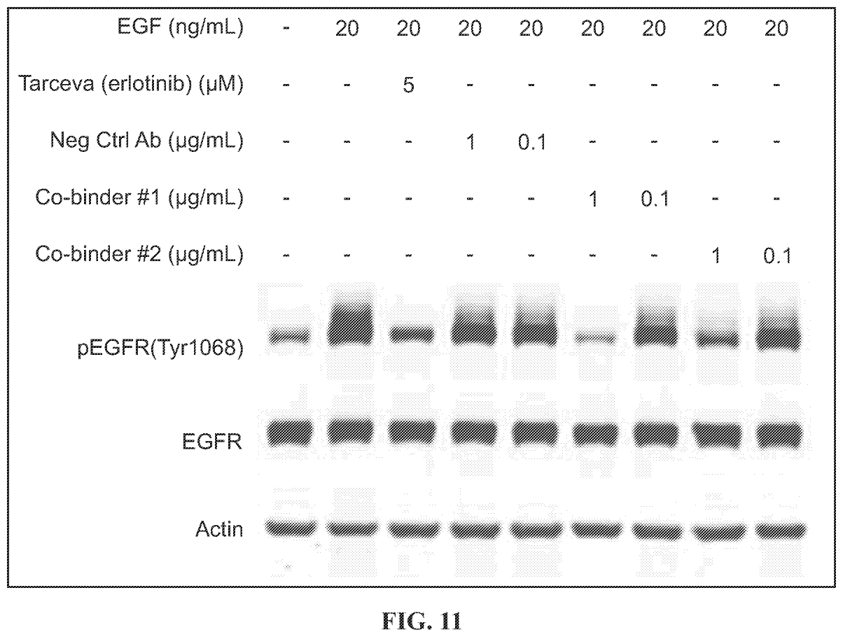

The initial single binder library SB0 can be VHHs or any protein binders that are used with display technologies, including but not limited to well-known antibodies and antibody fragments such as Fab and Fd, single-chain variable fragments (scFv), single-domain antibodies (sdAb)/heavy chain antibodies (HCAb)/V_{H}H, affibodies, affilins, affimers, affitins, alphabodies, anticalins, avimers, DARPins, Forkhead-Associated domains, Fynomers, Kunitz domain peptides, monobodies (Adnectins), minibodies, nanoCLAMPs, any peptides, peptidomimetics, antibody mimetics or other binding scaffolds. Display technologies can also vary among ribosome display, bacteria display, yeast surface display, insect cell surface display, mammalian cell surface display, etc. General methods for constructing various SB0 libraries can be found in publications (Akamatsu et al., J Immunol Methods 327:40-52 (2007); Chao et al., Nat Protoc 1 (2): 755-768 (2006); Daugherty, Cur Opin Struc Biol 17:474-480 (2007); Ernst et al., Nucleic Acids Res 26:1718-1723 (1998); Ho et al., Proc National Acad Sci 103:9637-9642 (2006); Zahnd et al., Nat Methods 4:269: 279 (2007)). As an example, RNA was isolated and VHH genes were extracted from peripheral blood mononuclear cells (PBMC) from naïve llamas or llamas immunized with EGFR following the described protocol (Pardon et al., Nat Protoc 9:674-693 (2014)). The SB0 library was constructed by cloning VHH genes into a yeast surface display vector (Wang et al., Protein Eng Des Sel 18 (7): 337-343 (2005)). Induced SB0 yeast display library was stained with antigens and single binders sorted by fluorescence-activated cell sorting (FACS), for example, as shown in FIG. 10A.

In addition to FACS-based sorting method, other methods can be used to separate binders from non-binders. For example, the target protein can be immobilized to a solid surface, e.g. a plastic surface in an ELISA plate or a magnetic bead surface, either by direct conjugation, hydrophobic adsorption or capture through modifications on the protein, such as biotin, protein- or peptide-tags, etc. Library members that recognize the target protein will remain bound to the solid surface until non-binding members are washed away. Binding members can then be eluted from the solid surface. Separation can also be carried out using magnetic-activated cell sorting (MACS). With these methods, the binders recognizing the target protein can be isolated from other non-binders. The isolated population is enriched with binders and has only a small fraction of the SB0 diversity. It is often necessary to propagate the isolated population to perform more rounds of selection, in order to further enrich the binders and reduce the diversity. Since we intend to discover high-affinity co-binders by leveraging selected single binders, 2-3 rounds of selection may be preferable to produce the SB1 library of reasonable diversity. Table 20 shows 32 top enriched human EGFR binders from an immunized VHH library.

TABLE 20
Frequencies of most abundant CDR3 sequences from
the SB1 VHH yeast surface display library.

| CDR3 sequence | Freq | CDR3 sequence | Freq | CDR3 sequence | Freq |
|---|---|---|---|---|---|
| RNGGGGLFLDY | 25.975297 | RRDGTDY | 0.589631 | KHFSYVEDF | 0.29205 |
| (SEQ ID NO: 164) | | (SEQ ID NO: 175) | | (SEQ ID NO: 186) | |
| VPPLYKSDGVY | 24.622354 | VPPLPR | 0.457434 | VPPFPS | 0.290944 |
| (SEQ ID NO: 165) | | (SEQ ID NO: 176) | | (SEQ ID NO: 187) | |
| DLFGVPTEYPGQEFHP | 12.292094 | LIAGVRY | 0.426459 | RSGGGWSEKLVAYDY | 0.271584 |
| (SEQ ID NO: 166) | | (SEQ ID NO: 177) | | (SEQ ID NO: 188) | |
| QGVIATSLKYLEV | 1.420425 | VMRGVNY | 0.420375 | IFVRGGSYYPADEYDY | 0.259969 |
| (SEQ ID NO: 167) | | (SEQ ID NO: 178) | | (SEQ ID NO: 189) | |
| IFVRGGSFYPADEYDY | 1.393322 | RWGGRLY | 0.413184 | LFHNDY | 0.249459 |
| (SEQ ID NO: 168) | | (SEQ ID NO: 179) | | (SEQ ID NO: 190) | |
| TGVRLNYGSRAEDY | 1.07085 | IFQRGTSYYPADEFDY | 0.409312 | RIGFSSTNNY | 0.235078 |
| (SEQ ID NO: 169) | | (SEQ ID NO: 180) | | (SEQ ID NO: 191) | |
| GYPWDESCDYDN | 0.988987 | SRGPS | 0.384422 | DLFGVPTEYPGQEFHI | 0.223462 |
| (SEQ ID NO: 170) | | (SEQ ID NO: 181) | | (SEQ ID NO: 192) | |
| RNGGGGLLLDY | 0.899381 | MVITTTPEYDY | 0.370594 | VQPLYKSDGVY | 0.217931 |
| (SEQ ID NO: 171) | | (SEQ ID NO: 182) | | (SEQ ID NO: 193) | |
| HHPYETPL | 0.70634 | PPL | 0.338512 | VPPLYKRDGVY | 0.206869 |
| (SEQ ID NO: 172) | | | | (SEQ ID NO: 194) | |
| DSPVRGYVCSDSLSTMDY | 0.651581 | SYSGGYYNFRESQYYS | 0.332428 | DGRGDE | 0.202444 |
| (SEQ ID NO: 173) | | (SEQ ID NO: 184) | | (SEQ ID NO: 195) | |
| PLSRGSMDY | 0.651028 | NLFGVPTEYPGQEFHP | 0.322472 | | |
| (SEQ ID NO: 174) | | (SEQ ID NO: 185) | | | |

### Libraries of Co-Binders

A co-binder library CB0 can be constructed from the single binder library SB1 and the linker library L. The DNA sequences encoding binding members in SB1 are extracted and amplified separately into the first single binders (B1) and the second single binders (B2). B1 connects to the N-terminus of the linker, and B2 connects to the C-terminus. B1 and B2 may share the same pool of binder sequences but differ in the regions that overlap with the yeast surface display vector, which are used for insertion of the co-binder genes.

The linker library of L sequences can be rationally designed to reduce linker-related energy loss and to minimize the K′ factor. In the first example of linker library construction, a part of the design rules learned through the above-described model system study was applied by removing multiple N-terminal residues of the second binders (B2). In addition, rigid and flexible linkers with varying lengths are used in the library L. For flexible linkers, the number of amino acids ranges from 6 to 34 with a combination of GGS, GGGS and GGGGS followed by 2 randomized positions (Table 21). For rigid linkers, the length varies between 12 and 32 (Table 21). Different types of proline rich sequences are used as building blocks, including the XP and XPP motifs. The building blocks are interspersed with two randomized residues linking both binders B1 and binders B2. The randomization is limited to defined amino acid subsets to constrain the sequence diversity of the library CB0.

TABLE 21
Linker sequences used for the construction of library L.

| Rigid Linker Sequence | Length | Diversity |
|---|---|---|
| PXPAPXPAPXP (SEQ ID NO: 196) | 11 | 1728 |
| PXPAPXPAPBPAPXP (SEQ ID NO: 197) | 15 | 3456 |
| PXPAPBPAPUP APBPAPXP (SEQ ID NO: 198) | 19 | 3456 |
| PUPAPAPAPUPAPAPAPUPAPXP (SEQ ID NO: 199) | 23 | 2592 |
| PUPAPAPAPUPAPAPAPUPAPAPAPXP (SEQ ID NO: 200) | 27 | 2592 |
| PJPAPAPAPJPAPAPAPJPAP APAPJPAP XP | 31 | 3072 |
| PJJPTPPPPAPPUPPPPAPPUPXP (SEQ ID NO: 202) | 24 | 6912 |
| PJJPTPPPPAPPUPTPPPPAPPUPTPXP (SEQ ID NO: 203) | 28 | 6912 |
| PUJPTPPPPAPPUPXP (SEQ ID NO: 204) | 16 | 1728 |
| PUJPTPPPPAPPUPTPPPXP (SEQ ID NO: 205) | 20 | 1728 |
| PJJPTPPPPAPPJPTPPPPAPPJPTPPBPXP (SEQ ID NO: 206) | 31 | 6144 |
| PJJPTPPPPAPPJPTPPPPAPPJPTPPPPAPPXP (SEQ ID NO: 207) | 34 | 3072 |
| JOPTPPTPPNPPTPPSPPPZX (SEQ ID NO: 208) | 21 | 1728 |
| JOPTPPTPPNPPSPPPZX (SEQ ID NO: 209) | 18 | 1728 |
| JOPTPPSPPPZX (SEQ ID NO: 210) | 12 | 1728 |
| JOPTPPTPPNPPTZX (SEQ ID NO: 211) | 15 | 1728 |
| JOPTPPTPPNPPTPPSPPTPPZX (SEQ ID NO: 212) | 23 | 1728 |
| JOPTPPTPPNPPTPPSPTPPSPPPZX (SEQ ID NO: 213) | 26 | 1728 |
| JOPTPPTPPNPPTPPSPTPPNPPSPPPZX (SEQ ID NO: 214) | 29 | 1728 |
| JOPTPPTPPNPPTPPSPTPPTPPNPPSPPPZX (SEQ ID NO: 215) | 32 | 1728 |
| GGGSZX (SEQ ID NO: 216) | 6 | 144 |
| GGGGSZX (SEQ ID NO: 217) | 7 | 144 |
| GGSGGSZX (SEQ ID NO: 218) | 8 | 144 |
| GGGSGGSZX (SEQ ID NO: 219) | 9 | 144 |
| GGGSGGGSZX (SEQ ID NO: 220) | 10 | 144 |
| GGSGGSGGSZX (SEQ ID NO: 221) | 11 | 144 |
| GGGGSGGGGSZX (SEQ ID NO: 222) | 12 | 144 |
| GGGSGGGSGGSZX (SEQ ID NO: 223) | 13 | 144 |
| GGGSGGGSGGGSZX (SEQ ID NO: 224) | 14 | 144 |
| GGGGSGGGSGGGSZX (SEQ ID NO: 225) | 15 | 144 |
| GGGGSGGGGSGGGSZX (SEQ ID NO: 226) | 16 | 144 |
| GGGGSGGGGSGGGGSZX (SEQ ID NO: 227) | 17 | 144 |
| GGGSGGGSGGGSGGGSZX (SEQ ID NO: 228) | 18 | 144 |
| GGGGSGGGSGGGSGGGSZX (SEQ ID NO: 229) | 19 | 144 |
| GGSGGSGGSGGSGGSGGSZX (SEQ ID NO: 230) | 20 | 144 |
| GGGGSGGGGSGGGGSGGGSZX (SEQ ID NO: 231) | 21 | 144 |
| GGGSGGGSGGGSGGGSGGGSZX (SEQ ID NO: 232) | 22 | 144 |
| GGGGSGGGGSGGGGSGGGGSZX (SEQ ID NO: 233) | 22 | 144 |
| GGSGGSGGSGGSGGSGGSGGSZX (SEQ ID NO: 234) | 23 | 144 |
| GGGGSGGGGSGGGSGGGSGGGSZX (SEQ ID NO: 235) | 24 | 144 |
| GGGGSGGGGSGGGGSGGGSGGGSZX (SEQ ID NO: 236) | 25 | 144 |
| GGGSGGGSGGGSGGGSGGGSGGGSZX (SEQ ID NO: 237) | 26 | 144 |
| GGGGSGGGGSGGGGSGGGGSGGGGSZX (SEQ ID NO: 238) | 27 | 144 |
| GGGGSGGGGSGGGSGGGSGGGSGGGSZX (SEQ ID NO: 239) | 28 | 144 |
| GGSGGSGGSGGSGGSGGSGGSGGSGGSZX (SEQ ID NO: 240) | 29 | 144 |
| GGGSGGGSGGGSGGGSGGGSGGGSGGGSZX (SEQ ID NO: 241) | 30 | 144 |
| GGGGSGGGSGGGSGGGSGGGSGGGSGGGSZX | 31 | 144 |
| (SEQ ID NO: 242) | | |
| GGGGGGGGSGGGGSGGGGSGGGGSGGGGSZX | 32 | 144 |
| (SEQ ID NO: 243) | | |
| GGGGSGGGGSGGGGSGGGSGGGSGGGSGGGSZX | 33 | 144 |
| (SEQ ID NO: 244) | | |
| GGGSGGGSGGGSGGGSGGGSGGGSGGGSGGGSZX | 34 | 144 |
| (SEQ ID NO: 245) | | |

**Amino acid codes for randomized positions

| | |
|---|---|
| O | A, D, or G |
| J | A, G, S, or T |
| B | A, G, P, or R |
| U | A, G, P, R, S, or T |
| Z | A, D, F, H, I, L, N, P, S, T, V, or Y |
| X | A, D, G, H, I, L, N, P, R, S, T, or V |

The method used for CB0 library enrichment is similar to that used in SB0 library selection, but the stringency of the selections needs to be controlled so that high-affinity co-binders can be differentiated from the weaker ones. Several ways can be adopted to control the selection stringency. First, the concentration of the target protein can be varied as shown in FIG. 10B as an example. The lower the concentration, the higher the stringency. High-affinity binders can retain good target engagement under the high stringency but not the weaker binders. Second, the washing time can be varied. After the library members are incubated with the target protein, unbound library members or proteins need to be washed away. The longer the washing time, the higher the stringency. Generally, weaker binders may dissociate from the target protein during the washing step, but not the high-affinity binders. Third, the time for target protein incubation can also be varied. Strong binders tend to bind targets faster than weaker binders. By limiting the incubation time, high-affinity binders get enriched better than low-affinity ones. After 3-4 rounds of selections with increased stringencies, the resulted library CB1 will be enriched with high-affinity co-binders.

Two methods can be used to determine which co-binders from CB1 will be selected for further analysis. First, clones in CB1 library can be randomly picked and their sequences determined (e.g. Table 22 below). The affinity of the selected co-binders can be analyzed by FACS. In addition, co-binder proteins can be produced, and their binding affinity can be analyzed by SPR (e.g. Table 23 below). Alternatively, NGS analysis can be done for the last two rounds of CB1 selection and sequence enrichment factor can be calculated. The most enriched sequences will be selected for co-binder protein production and SPR analysis to determine their affinity.

### Properties of the Screened Co-Binders

Picked co-binder clones from CB1 library were sequenced by sanger sequencing. Each co-binder sequence contained a single binding domain at the N-terminus and a different binding domain at the C-terminus which are connected by a linker sequence as designed by the linker library shown in the Table 21. No co-binders were found to be formed with two single binding domains that contain the same CDR regions, which indicates that high affinity co-binders are most likely formed by single binders targeting different epitopes.

TABLE 22
Selected sequences of single binder B1 CDR sequences, linker sequences and
single binder B2 CDR sequences of the unique co-binder clones that
were enriched in library CB1.

| Clone | B1 | | | | | | |
|---|---|---|---|---|---|---|---|
| name | CDR1 | B1 CDR2 | B1 CDR3 | Linker | B2 CDR1 | B2 CDR2 | B2 CDR3 |
| 4C11 | AGAFIS | SITNIGDAH | PPL | AGPTPPTPPNPP | RSAWSIINM | RLTPGGSAV | DTPPYGGP |
| | DTVY | YAASAKG | | TAG (SEQ ID | A (SEQ ID | YGEFVKG | DY (SEQ ID |
| | (SEQ ID | (SEQ ID | | NO: 254) | NO: 263) | (SEQ ID | NO: 279) |
| | NO: 246) | NO: 249) | | | | NO: 272) | |
| 3B7 | AGAFIS | SITNIGDAH | PPL | GGPTPPSPPPSP | GIIFSHYTM | LITPGDGTY | KSRSIVNR |
| | DTVY | YAASVKG | | (SEQ ID NO: 255) | G (SEQ ID | YADSVKG | DY (SEQ ID |
| | (SEQ ID | (SEQ ID | | | NO: 264) | (SEQ ID | NO: 280) |
| | NO: 246) | NO: 250) | | | | NO: 273) | |
| 4C12 | AGAFIS | SISNIGDAH | PPL | TPSPAPAPAPGP | GTILRFITM | RITGGGTTL | LAPGRDY |
| | DTVY | YAASVKG | | APAPAPRPAPAP | D (SEQ ID | YANSVKD | (SEQ ID |
| | (SEQ ID | (SEQ ID | | APVP (SEQ ID | NO: 265) | (SEQ ID | NO: 281) |
| | NO: 246) | NO: 251) | | NO: 256) | | NO: 274) | |
| WF4 | AGAFIG | SITNIGDAH | PPL | GPAPAPAPAPRP | RSLSANGV | TITGSGFGH | VPPTPY |
| | DTVY | YAASAKG | | APAPTPGPAPGP | S (SEQ ID | YADSVKG | (SEQ ID |
| | (SEQ ID | (SEQ ID | | (SEQ ID NO: 257) | NO: 266) | (SEQ ID | NO: 282) |
| | NO: 247) | NO: 249) | | | | NO: 275) | |
| 4C7 | AGAFIS | SISNIGDAH | PPL | SPAPAPAPAPRP | GSIFSGNA | LITSDGSTTY | VPPLGR |
| | DTVY | YAASVKG | | APAPAPGPAPGP | MD ((SEQ ID | ADSVKG | (SEQ ID |
| | (SEQ ID | (SEQ ID | | (SEQ ID NO: 258) | NO: 267) | (SEQ ID | NO: 283) |
| | NO: 246) | NO: 251) | | | | NO: 276) | |
| 4B9 | AGAFIS | SITNIGDAH | PPL | APRPAPAPAPGP | ETIFSGNAM | LITSDGSTTY | VPPLPR |
| | DTVY | YAASAKG | | APAPAPPPAPAP | D (SEQ ID | ADSVKG | (SEQ ID |
| | (SEQ ID | (SEQ ID | | APSP (SEQ ID | NO: 268) | (SEQ ID | NO: 284) |
| | NO: 246) | NO: 249) | | NO: 259) | | NO: 276) | |
| 15E2 | GIGFSS | FISSNGRQ | WLVDY | SPSSPTPPPPAPP | AGAFISDTV | SISNIGDAHY | PPL |
| | NTMG | NYADSVK | (SEQ ID | SPTPPPPAPPGPT | Y (SEQ ID | AASVKG | |
| | (SEQ ID | G (SEQ ID | NO: 253) | PDP (SEQ ID | NO: 269) | (SEQ ID | |
| | NO: 248) | NO: 252) | | NO: 260) | | NO: 251) | |
| WF12 | AGAFIS | SISNIGDAH | PPL | GPGPAPAPAPGP | GSIFSTNPIQ | TITGGGGTN | YSYPY |
| | DTVY | YAASVKG | | APAPAPAPAPIP | (SEQ ID | YLDSVKG | (SEQ ID |
| | (SEQ ID | (SEQ ID | | (SEQ ID NO: 261) | NO: 270) | (SEQ ID | NO: 285) |
| | NO: 246) | NO: 251) | | | | NO: 277) | |
| WF17 | AGAFIS | SISNIGDAH | PPL | GDPTPPTPPNPP | GFTFGSLT | SISSGGGTA | AASPANAY |
| | DTVY | YAASVKG | | TPPSPTPPSPPPP | MK (SEQ ID | AYSDSVKG | GIY (SEQ |
| | (SEQ ID | (SEQ ID | | R (SEQ ID | NO: 271) | (SEQ ID | ID NO: 286) |
| | NO: 246) | NO: 251) | | NO: 262) | | NO: 278) | |
| 1E10 | GFTFTD | YFNPNSGY | LSPGGY | GGGGSSGGGSG | RASQGINN | NTNNLQT | LQHNSFPT |
| | YKIH | STYAQKFQ | YVMDA | GGGS (SEQ ID | YLN (SEQ | (SEQ ID | (SEQ ID |
| | (SEQ ID | G (SEQ ID | (SEQ ID | NO: 290) | ID NO: 291) | NO: 292) | NO: 293) |
| | NO: 287) | NO: 288) | NO: 289) | | | | |

The co-binder genes and their component single binders were subcloned into yeast expression vector. Co-binder proteins and component single binder proteins were expressed and purified. The binding affinity of each co-binder and component single binders were measured using surface plasmon resonance method using purified binder proteins (Table 23). The affinities of the picked clones ranged from 0.09 to 12 nM, whereas the affinity of the component single binders ranged weaker and more broadly from 1.2 to 990 nM, which demonstrated that the co-binder screening method provided herein is effective in generating co-binders with synergistic binding. For example, co-binder 3B7 with KD at 1.2 nM is consisted of two single binders with KD in the range of 100 nM, representing 100-fold improvement in affinity. Several co-binders with KD in the range of 100 pM include single binders with KD in the range of 10 nM. Thus, the screening approach provided here can indeed leverage low affinity single binders to create co-binders with affinity and specificity suitable for therapeutic and diagnostic applications. It is also of interest to know that those component single binders are not present in single binder pool with very high frequency. Thus, single binders with the highest affinity is not necessarily the most suitable ones for co-binder generation. This is consistent with the previous observations that linking two high affinity single binders may not necessarily lead to co-binders with greatly improved affinity.

TABLE 23
Affinity characterization of selected co-binders and their component
single binders that were enriched from library CB1.

| Clone name | Co-binder | Single Binder B1 | Single Binder B2 |
|---|---|---|---|
| 4C11 | 3.2 × 10⁻⁰⁹ | 2.1 × 10⁻⁰⁹ | 3.3 × 10⁻⁰⁸ |
| 3B7 | 1.2 × 10⁻⁰⁹ | 1.4 × 10⁻⁰⁷ | 1.3 × 10⁻⁰⁷ |
| 4C12 | 4.2 × 10⁻⁰⁹ | 2.1 × 10⁻⁰⁸ | 3.8 × 10⁻⁰⁸ |
| WF4 | 1.2 × 10⁻⁰⁸ | 1.7 × 10⁻⁰⁷ | 4.6 × 10⁻⁰⁸ |
| 4C7 | 9.0 × 10⁻¹⁰ | 3.8 × 10⁻⁰⁸ | 1.5 × 10⁻⁰⁸ |
| 4B9 | 1.3 × 10⁻¹⁰ | 1.2 × 10⁻⁰⁹ | 3.0 × 10⁻⁰⁷ |
| 15E2 | 3.0 × 10⁻¹⁰ | 7.6 × 10⁻⁰⁸ | 1.5 × 10⁻⁰⁸ |
| WF12 | 2.3 × 10⁻⁰⁹ | 2.0 × 10⁻⁰⁸ | 5.1 × 10⁻⁰⁷ |
| WF17 | 3.3 × 10⁻⁰⁹ | 5.5 × 10⁻⁰⁸ | 5.6 × 10⁻⁰⁸ |
| 1E10 | 9.1 × 10⁻¹¹ | 8.7 × 10⁻⁰⁸ | 9.9 × 10⁻⁰⁷ |

In summary, screening methods capable of robust, scalable and predictable generation of co-binders with synergistic co-binding were provided and validated herein.

Since the screening methods incorporate linker designs that minimize linker-related energy loss, it can leverage low affinity (e.g. 100 nM) single binders targeting expanded repertoire of epitopes in an antigen. Thus, the methods have unexpected and superior success rate for discovering synergistic co-binders than the traditional approach of searching for high affinity single binders first and then linking them together with GS linkers.

### Example 9: Co-Binder Applications

Co-binders with synergistic co-binding possess higher binding affinity and specificity than its component individual binders (e.g. antibody variable domains). As a result, the co-binders can be applied in any applications where single binders can be applied for, including therapeutic, diagnostic and research applications as known by those skilled in the art.

### Conjugated Proteins

Co-binders can be modified by attachment of protein domains, peptidyl tags and extra cysteine residues, or by replacement of amino acids with those artificially synthesized to carry special chemical groups. These modified co-binders are conjugated to chemical molecules such as dyes, enzymes, cytotoxic agents, toxins, radioactive isotopes, nucleic acids, etc.

### Activity Assays

Co-binders can be characterized for their physical or chemical properties and biological functions by various assays known in the art.

### Diagnosis and Imaging

Co-binders can be used to assay protein levels in a biological sample using classical immunohistological methods known to those of skill in the art. One can also study overexpression of one or more antigens of interest by measuring antigen(s) in a biological fluid such as serum, cerebrospinal fluid, urine, saliva or other bodily fluid. In particular, synergistic co-binders can be used as a pair in sandwich immunoassays for detection of disease markers in biological fluids. Labeled synergistic co-binders can also be used for medical imaging such as positron emission tomography (PET) and magnetic resonance imaging (MRI).

### Therapeutic Uses

Co-binders with synergistic co-binding can be used for therapeutic applications. For example, they can be used for the treatment of cancers, allergic or inflammatory diseases, eye diseases, neurodegenerative diseases, etc. Co-binders can be used to enhance the specificity of therapeutic targeting to reduce off-target binding. This is broadly applicable to any treatment and especially useful when using potent therapeutic agents such as chimeric antigen receptor T cells or NK cells and antibody drug conjugates. Co-binder can be further combined with additional functional binders that result in enhanced delivery of therapeutic activity into the brain by crossing the blood brain barrier.

In an exemplary study, two anti-EGFR co-binders showed inhibitory effects on EGF-induced signaling in tumor cells. A431 cells were cultured in serum-free medium for 24 h before being treated with drugs for 1 hr. Cells were then stimulated with EGF for 15 minutes. Stimulated cells were washed afterwards with cold PBS and being lysed, followed by western blot experiments to probe the total and phosphorylated EGFR molecules. An exemplary result is shown in FIG. 11. The result validate that the co-binders against EGFR identified herein can inhibit EGFR signaling.

### Enhancement of Therapeutic Efficacy

A co-binder can be used to enhance the therapeutic efficacy. For example, a single binder may exert weak but active biological functions that does not pass the efficacious or specificity threshold. Combining them with other non-active single binders to form a co-binder can enhance the binding affinity and specificity as well as the biological function so as to pass the efficacious and specificity threshold.

### Elimination of the Synergistic Effect of Co-Binders

The synergistic effect of co-binders is produced through the linkage of two single binders. Such enhanced binding synergy may be eliminated by specific degradation of the linker portion of the co-binder. For example, the linker may contain sequence motifs that are recognized by endopeptidases. Treatment of the co-binder with endopeptidases cleaves the linker peptide and turns the strong co-binder into two weaker single binders. Such treatment may be used in cases where the modulation of the co-binder activity is needed, e.g. chimeric antigen receptor therapies, imaging, etc.

### Example 10: Finding Pairs of Non-Overlapping Epitopes

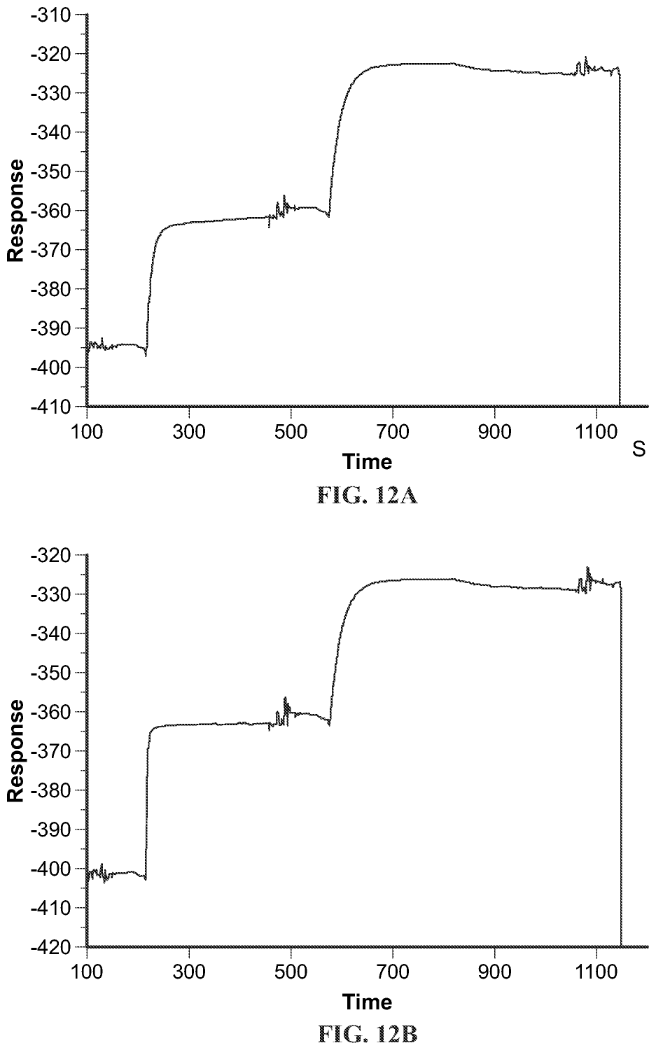

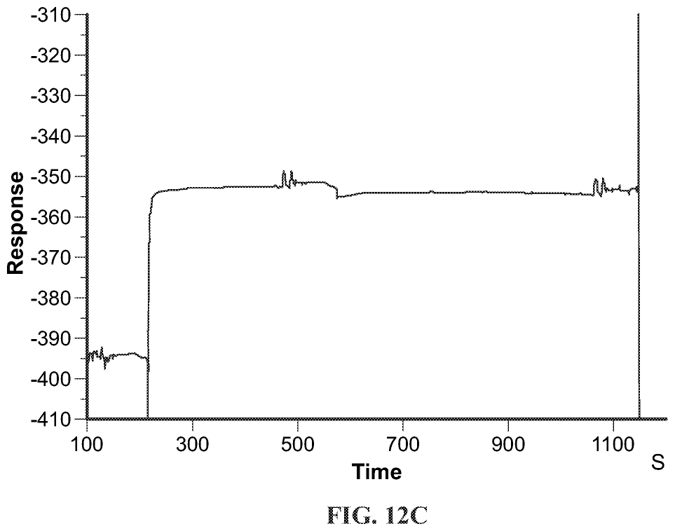

Protein binders (including individual binding moieties and co-binders) can be screened for non-interfering pairs using any conventional method. FIGS. 12A-12C show an example of a screen for non-interfering pairs of protein binders. Briefly, three EGFR binders were subjected to a screen by Surface Plasmon Resonance with their cognate antigen, EGFR, as a ligand. The EGFR binders were flown one after other and the sensogram was observed for changes in signal. FIGS. 12A-12B show two examples of non-interfering EGFR binders. Namely 1E10 form a pair with 15E2 (FIG. 12A), and 7D12-9G8 form a pair with 15E2 (FIG. 12B). However, 7D12-9G8 and 1E10 protein binders do not form a pair as shown by FIG. 12C, because there is no additional binding (no additional response signal) detected when the second EGFR binder was injected.

### Example 11: N-Terminus Proximity is Widely Observed in Antibodies

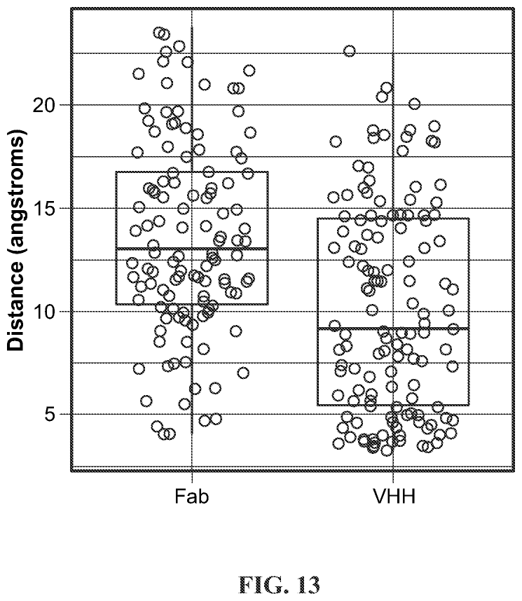

Generally, the paratope of an antigen binding fragment, including VHH and VH or VL of a Fab, is near the N-terminus of such antigen binding fragment. Connecting a linker peptide to the N-terminus of such antigen binding fragment causes energy loss due to the interference of the linker with the binding site of the antibody. To determine the potential of linker interference with the epitope-paratope interaction, we measured the distance of the antibody N-termini to the antigenic surface. 133 unique structures of VHH-antigen complexes, and 60 structures of Fab's with their cognate antigens were picked out of the Protein Data Bank. The distance from the first Ca atom (N-terminus) of VHH or Fab (including VH and VL) to the nearest non-hydrogen atom on the antigen surface were measured and plotted (FIG. 13). Notably, the N-termini of about 25% of the VHHs are less than 5 Å away from the antigen, which are considered as making contacts. Assuming the linker can make a tight β- or γ-turn when being attached to the N-terminus of the 2nd single binder, it will need a minimal radius of 3 Å for making that turn. Therefore, a minimal gap of 8 Å between N-termini and antigen is required to connect the 2nd single binder without experiencing interference from the linker. However, 42% of the VHHs (56 out of 133) are at the risk of clashing due to the N-termini to antigen distance being less than 8 Å. By the same threshold, 9% of the heavy chains and 15% of the light chains in Fab are that close to their antigens. Due to structure conservation, this issue can be generalized to all immunoglobulin (Ig) fold. Ig fold is a common and well conserved scaffold used in antigen receptors, including the canonical antibodies from vertebrates (e.g. IgA, IgD, IgE, IgG, IgM, IgY, IgW etc.), VHH domain of camelids' Heavy-Chain antibodies (HCAbs), V-NAR domain of cartilaginous fishes' Immunoglobulin New Antigen Receptors (IgNARs) as well as the T cell receptors (TCRs).

### Example 12: Generation of Anti-HIV p24 Co-Binders

By using the methods of screening in Example 8, high-affinity anti-HIV p24 co-binders were discovered. Briefly, both naïve and immunized single binder yeast surface display libraries SB0 were constructed and used for selecting p24-binding clones. Based on the selected single-binder library SB1, a co-binder library CB0 was constructed by inserting the same linker library L (Table 21) between B1 and N-terminally truncated B2. To avoid binding interference and thus enhance affinity improvement, zero to three N-terminal residues of B2 were deleted before being connected with the linker sequences. The co-binder library CB0 was subject to multiple rounds of sorting to enrich high-affinity binders. Single yeast clones expressing anti-p24 co-binders were picked and characterized (Table 24).

TABLE 24
Anti-HIV p24 co-binders sequences and their binding
kinetics and affinities measured by Biacore.

| Clone Name | B1 CDR3 | B2 CDR3 | kₒₙ (M⁻¹s⁻¹) | K_{off} (s⁻¹) | K_{D} (M) |
|---|---|---|---|---|---|
| 8A11 | RKDFGDMDY | PTMA | 1.5E6 | 9.5E−6 | 6.4E−12 |
| 8B1 | PTMA | KWQGYREYES | 6.7E5 | 5.7E−5 | 8.5E−11 |
| 8B12 | PTMA | NVGEWRSLESY | 1.2E6 | 1.2E−4 | 1.1E−10 |
| 8D1 | PFAS | RYTSSTYGQL | 9.0E5 | 4.1E−4 | 4.5E−10 |
| 8F2 | RNGGGGLLLDY | PTMA | 3.4E6 | 1.4E−3 | 4.1E−10 |
| 9B5 | PFAS | RGDQ | 1.3E6 | 1.0E−4 | 8.0E−11 |
| 9D6 | KIKLFGSVVTGDGYDY | PTMA | 3.1E6 | 4.0E−5 | 1.3E−11 |
| 9D7 | PFAS | ARTWTEDEY | 1.0E6 | 5.5E−5 | 5.4E−11 |
| 9E7 | RYTSSTYGQL | PTMA | 1.0E6 | 5.5E−5 | 5.4E−11 |
| 9H6 | PTMA | RYTSSTYGQL | 3.6E5 | 2.8E−4 | 7.7E−10 |

### Example 13: Affinity Improvement of Co-Binders Containing N-Terminal Truncations

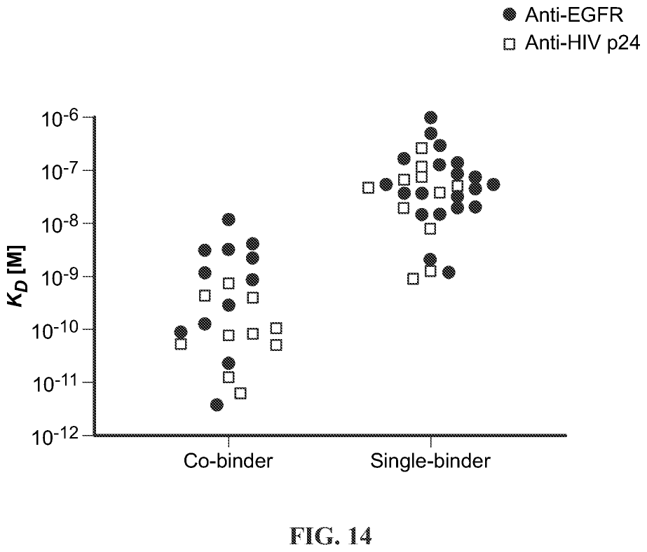

The anti-HIV p24 co-binder genes and their component single binders B1 and B2 were subcloned into yeast expression vector. Co-binder proteins and component single binder proteins were expressed and purified. The binding affinity of each co-binder and component single binders were measured using surface plasmon resonance method using purified binder proteins (Table 25). Overall the co-binders generated with the N-terminal truncation methods showed greatly improved affinities than the single binders (FIG. 14). The median of the combined anti-EGFR and anti-HIV p24 co-binders is 215 pM, which is 200 fold stronger affinity than the median of the combined single binders (48 nM).

TABLE 25
Affinity measurements of selected anti-HIV p24 co-binders
and their component single binders by Biacore.

| Clone Name | Co-binder K_{D} (M) | B1 K_{D} (M) | B2 K_{D} (M) |
|---|---|---|---|
| 8A11 | 6.4E−12 | 3.8E−8 | 9.3E−10 |
| 8B1 | 8.5E−11 | 9.3E−10 | 5.0E−8 |
| 8B12 | 1.1E−10 | 9.3E−10 | 2.0E−8 |
| 8D1 | 4.5E−10 | 1.3E−9 | 8.0E−9 |
| 8F2 | 4.1E−10 | 6.8E−8 | 9.3E−10 |
| 9D7 | 5.4E−11 | 1.3E−9 | 4.8E−8 |
| 9E7 | 5.4E−11 | 8.0E−9 | 9.3E−10 |
| 9H6 | 7.7E−10 | 9.3E−10 | 8.0E−9 |

### Example 14: Additional Examples of High-Affinity Co-Binders Discovered Via the Methods of Screening

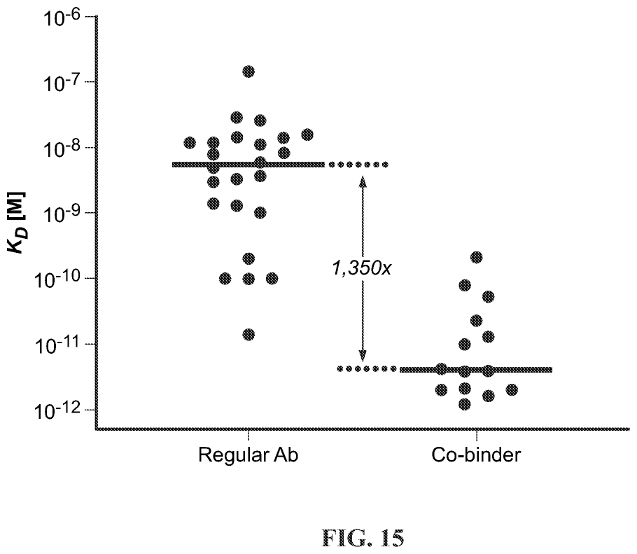

The co-binder library and methods of screening based on N-terminal truncations are universally applicable. By applying the methods, high-affinity co-binders were generated for 14 various targets including EGFR and HIV p24 targets, cancer biomarkers, neural disease biomarkers, cytokines and a couple of therapeutic targets for treating neurodegenerative diseases (FIG. 15). The median affinity of the strongest co-binders for each target is 4 pM measured by Biacore. To note, these measurements were restricted by an off-rate limit of 10⁻⁶ s⁻¹ due to instrument limitation. Therefore, the actual affinity may be higher than what was measured. When compared to the median antibody affinity from the Structural Antibody Database (doi: 10.1093/nar/gkt1043), the co-binders are 1,350 fold stronger than regular, non-engineered antibodies from the database, which demonstrates the effectiveness of high-affinity co-binder generation via the mechanism of N-terminal truncations as well as the associated methods of screening.

1: A co-binder comprising a first binding moiety specifically recognizing a first target site and a second binding moiety specifically recognizing a second target site,
wherein the second binding moiety is a second antibody moiety comprising an antibody variable domain having an N-terminal truncation (“N-terminal truncated antibody variable domain”),
wherein the first binding moiety is connected to the second binding moiety through N-terminus of the N-terminal truncated antibody variable domain optionally via a linker.

2-5: (canceled)

6: The co-binder of claim 1, wherein the first binding moiety is a first antibody binding moiety, and wherein the first antibody moiety is selected from the group consisting of a Fab, an Fv, an scFv, a dsFv, a Fab′, or a (Fab′)2 fragment.

7: The co-binder of claim 1, wherein the first binding moiety is a first antibody binding moiety, and wherein the first antibody moiety is a single domain antibody.

8: The co-binder of claim 1, wherein the second antibody moiety is selected from the group consisting of Fab, an Fv, an scFv, a dsFv, a Fab′, or a (Fab′)2 fragment.

9: The co-binder of claim 8, wherein the N-terminal truncated antibody variable domain is a truncated VH or truncated VL domain.

10: The co-binder of claim 1, wherein the second antibody moiety is a single domain antibody.

11: The co-binder of claim 10, wherein the N-terminal truncated antibody variable domain is a truncated V_{H}H domain.

12: The co-binder of claim 1, wherein the first binding moiety comprises a first V_{H}H domain; wherein the second binding moiety comprises a second V_{H}H domain having an N-terminal truncation (“truncated V_{H}H domain”),
wherein the C-terminus of the first V_{H}H domain is connected to the N-terminus of the second V_{H}H domain via a linker.

13: The co-binder of claim 1, wherein the N-terminal truncation of the N-terminal truncated antibody variable domain is about 1 to about 25 amino acids.

14: (canceled)

15: The co-binder of claim 1, wherein the linker is a peptide linker.

16: The co-binder of claim 15, wherein the C-terminal amino acid of the peptide linker immediately connected to the N-terminal truncated antibody variable domain is R, G, Y, or P.

17: The co-binder of claim 15, wherein the C-terminal three amino acids of the peptide linker immediately connected to the N-terminal truncated antibody variable domain are X₁-X₂-X₃,
wherein X₁ is V, L, W, P, S, G, K, D, F, M, T, N, or R;
X₂ is V, A, L, S, G, R, K, M, C, F, T, P, or E; and
X₃ is G; or
wherein the C-terminal three amino acids of the peptide linker immediately connected to the antibody variable domain of the second binding moiety are X₁-X₂-X₃,
wherein X₁ is any amino acid;
X₂ is K, R, Y, M, G, or N; and
X₃ is R, G, Y, or P.

18: The co-binder of claim 15, wherein the linker comprises (GₓS_{y})ₙ, wherein x is 1 to 5, y is 0 to 5, and n is 1 or more.

19: The co-binder of claim 15, wherein the linker comprises [EAAAK], or [EEEEKKKK]ₙ, wherein n is 1 or more.

20: The co-binder of claim 15, wherein the linker comprises [AP]ₙ, wherein n is 1 or more.

21-22: (canceled)

23: The co-binder of claim 1, wherein the co-binder further comprises a third binding moiety specifically recognizing a third target site.

24-27: (canceled)

28: The co-binder of claim 1, wherein the co-binder is an antibody comprising an Fc region or a chimeric antigen receptor (“CAR”).

29: (canceled)

30: A co-binder comprising a first binding moiety specifically recognizing a first target site and a second binding moiety specifically recognizing a second target site,
wherein the second binding moiety is a second antibody moiety comprising an antibody variable domain;
wherein the first binding moiety is connected to the second binding moiety through N-terminus of the antibody variable domain via a peptide linker;
wherein the C-terminal three amino acids of the peptide linker immediately connected to the antibody variable domain of the second binding moiety are X₁-X₂-X₃,
wherein X₁ is any amino acid;
X₂ is K, R, Y, M, G, or N; and
X₃ is R, G, Y, or P.

31-52: (canceled)

53: A library comprising a plurality of co-binders or a plurality of polynucleotides encoding a plurality of co-binders, each co-binder comprising a first binding moiety specifically recognizing a first target site and a second binding moiety specifically recognizing a second target site, wherein the second binding moiety is a second antibody moiety comprising an antibody variable domain, wherein the first binding moiety is connected to the second binding moiety through N-terminus of the antibody variable domain via a peptide linker, wherein at least two co-binders in the library differ from each other in the peptide linker sequence.

54-57: (canceled)

58: The library of claim 53, wherein the antibody variable domain of the second binding moiety has an N-terminal truncation (“N-terminal truncated antibody variable domain”).

59: A method of screening for a co-binder specifically binding to a second target site at a desired affinity, the method comprising:
(1) contacting the library of claim 53 with a target molecule comprising the second target site to form complexes between the co-binders that specifically bind to the target molecule and the target molecule, and
(2) identifying a co-binder that binds to the second target site with the desired affinity.

60: A method of screening for a co-binder specifically binding to a target molecule at a desired affinity, the method comprising:
(1) contacting the library of claim 53 with the target molecule to form complexes between the co-binders that specifically bind to the target molecule and the target molecule, and
(2) identifying a co-binder that binds to the target molecule with the desired affinity.

61: A method of increasing binding affinity of a control co-binder specifically binding to a target molecule, wherein the control co-binder comprise a first binding moiety specifically recognizing a first target site and a second binding moiety specifically recognizing a second binding target site, wherein the second binding moiety is a second antibody moiety comprising an antibody variable domain, wherein the first binding moiety is connected to the second binding moiety through N-terminus of the antibody variable domain via a linker, wherein the control co-binder comprises a full length antibody variable domain, wherein the binding affinity of the control co-binder to the second target site is lower than that of a second antibody moiety in free state, the method comprising obtaining a co-binder having an N-terminal truncation at the antibody variable domain of the second antibody moiety as compared to the control co-binder.

62: (canceled)

63: The co-binder of claim 53, wherein the second antibody moiety comprising the antibody variable domain has an N-terminal truncation (“N-terminal truncated antibody variable domain”).

64: The co-binder of claim 53, wherein the first binding moiety comprises a first V_{H}H domain, and/or the second binding moiety comprises a second V_{H}H domain.
